(19) United States
(12) Patent Application Publication
(10) Pub. No.: US 2011/0160180 A1
(43) Pub. Date: Jun. 30, 2011

(54) Cannabinoid Receptor Ligands

(75) Inventors: Sara Beha, Montreal (CA); William Brown, Montreal (CA); Shawn Johnstone, Montreal (CA); Ziping Liu, Montreal (CA); Daniel Pagé, Montreal (CA); Miroslaw Tomaszewski, Montreal (CA); Zhong-yong Wei, Sodertalje (SE); Shi Yi Yue, Montreal (CA)

(73) Assignee: ASTRAZENECA AB, Sodertalje (SE)

(21) Appl. No.: 12/673,617

(22) PCT Filed: Aug. 15, 2008
(86) PCT No.: PCT/GB08/50713

§ 371 Date: Mar. 17, 2011

(51) Int. Cl. A61K 31/403; C07D 405/08; C07D 405/14; A61K 31/454; A61P 29/00; A61P 35/00; A61P 25/00; A61P 25/16; A61P 25/28; A61P 25/22; A61P 1/00; A61P 9/00

(52) U.S. Cl. 514/210.18; 548/448; 546/200; 514/411; 514/323

(57) ABSTRACT

Compounds of Formulae (I), or pharmaceutically acceptable salts thereof: wherein R¹, R² and Y are as defined in the specification as well as salts and pharmaceutical compositions including the compounds are prepared. They are useful in therapy, in particular in the management of pain.

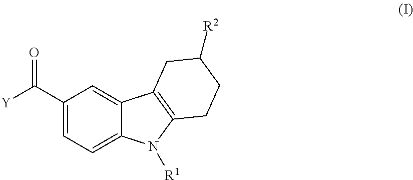

### BACKGROUND OF THE INVENTION

1. Field of the Invention

The invention is related to therapeutic compounds, pharmaceutical compositions containing these compounds, manufacturing processes thereof and uses thereof. Particularly, the present invention is related to compounds that may be effective in treating pain, cancer, multiple sclerosis, Parkinson's disease, Huntington's chorea, Alzheimer's disease, anxiety disorders, gastrointestinal disorders and/or cardiovascular disorders.

2. Discussion of Relevant Technology

Pain management has been studied for many years. It is known that cannabinoid receptor (e.g., CB₁ receptor, CB₂ receptor) ligands including agonists, antagonists and inverse agonists produce relief of pain in a variety of animal models by interacting with CB₁ and/or CB₂ receptors. Generally, CB₁ receptors are located predominately in the central nervous system, whereas CB₂ receptors are located primarily in the periphery and are primarily restricted to the cells and tissues derived from the immune system.

While CB₁ receptor agonists, such as Δ⁹-tetrahydrocannabinol (Δ⁹-THC) and anadamide, are useful in anti-nociception models in animals, they tend to exert undesired CNS side-effects, e.g., psychoactive side effects, the abuse potential, drug dependence and tolerance, etc. These undesired side effects are known to be mediated by the CB₁ receptors located in CNS. There are lines of evidence, however, suggesting that CB₁ agonists acting at peripheral sites or with limited CNS exposure can manage pain in humans or animals with much improved overall in vivo profile.

Therefore, there is a need for new CB₁ receptor ligands such as agonists that may be useful in managing pain or treating other related symptoms or diseases with reduced or minimal undesirable CNS side-effects.

### DESCRIPTION OF THE EMBODIMENTS

The present invention provides CB₁ receptor ligands which may be useful in treating pain and/or other related symptoms or diseases.

The term “Cₘ₋ₙ” or “Cₘ₋ₙ group” refers to any group having m to n carbon atoms.

The term “alkyl” refers to a saturated monovalent straight or branched chain hydrocarbon radical comprising 1 to about 12 carbon atoms. Illustrative examples of alkyls include, but are not limited to, C₁₋₆alkyl groups, such as methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, and hexyl, and longer alkyl groups, such as heptyl, and octyl. An alkyl can be unsubstituted or substituted with one or two suitable substituents.

The term “alkylene” used alone or as suffix or prefix, refers to divalent straight or branched chain hydrocarbon radicals comprising 1 to about 12 carbon atoms, which serves to links two structures together.

The term “alkylidene” used alone or as suffix or prefix, refers to divalent straight or branched chain hydrocarbon radicals comprising 1 to about 12 carbon atoms, which serves to links two structures together and the two radicals are located on the same carbon atom.

The term “alkenyl” refers to a monovalent straight or branched chain hydrocarbon radical having at least one carbon-carbon double bond and comprising at least 2 up to about 12 carbon atoms. The double bond of an alkenyl can be unconjugated or conjugated to another unsaturated group. Suitable alkenyl groups include, but are not limited to C₂₋₆alkenyl groups, such as vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl. An alkenyl can be unsubstituted or substituted with one or two suitable substituents.

The term “cycloalkyl” refers to a saturated monovalent ring-containing hydrocarbon radical comprising at least 3 up to about 12 carbon atoms. Examples of cycloalkyls include, but are not limited to, C₃₋₇cycloalkyl groups, such as cyclopropyl, cyclobutyl, cyclopentyl, cyclohexyl, and cycloheptyl, and saturated cyclic and bicyclic terpenes. A cycloalkyl can be unsubstituted or substituted by one or two suitable substituents. Preferably, the cycloalkyl is a monocyclic ring or bicyclic ring.

The term “cycloalkenyl” refers to a monovalent ring-containing hydrocarbon radical having at least one carbon-carbon double bond and comprising at least 3 up to about 12 carbon atoms.

The term “aryl” refers to a monovalent hydrocarbon radical having one or more polyunsaturated carbon rings having aromatic character, (e.g., 4n+2 delocalized electrons) and comprising 5 up to about 14 carbon atoms.

The term “heterocycle” refers to a ring-containing structure or molecule having one or more multivalent heteroatoms, independently selected from N, O, P and S, as a part of the ring structure and including at least 3 and up to about 20 atoms in the ring(s). Heterocycle may be saturated or unsaturated, containing one or more double bonds, and heterocycle may contain more than one ring. When a heterocycle contains more than one ring, the rings may be fused or unfused. Fused rings generally refer to at least two rings share two atoms there between. Heterocycle may have aromatic character or may not have aromatic character.

The term “heterocyclyl” refers a monovalent radical derived from a heterocycle by removing one hydrogen therefrom.

Heterocyclyl includes, for example, monocyclic heterocyclyls, such as: aziridinyl, oxiranyl, thiiranyl, azetidinyl, oxetanyl, thietanyl, pyrrolidinyl, pyrrolinyl, imidazolidinyl, pyrazolidinyl, pyrazolinyl, dioxolanyl, sulfolanyl, 2,3-dihydrofuranyl, 2,5-dihydrofuranyl, tetrahydropyranyl, thiophanyl, piperidinyl, 1,2,3,6-tetrahydro-pyridinyl, piperazinyl, morpholinyl, thiomorpholinyl, pyranyl, thiopyranyl, 2,3-dihydropyranyl, tetrahydropyranyl, 1,4-dihydropyridinyl, 1,4-dioxanyl, 1,3-dioxanyl, dioxanyl, homopiperidinyl, 2,3,4,7-tetrahydro-1H-azepinyl, homopiperazinyl, 1,3-dioxepanyl, 4,7-dihydro-1,3-dioxepinyl, and hexamethylene oxidyl.

In addition, heterocyclyl includes aromatic heterocyclyls or heteroaryl, for example, pyridinyl, pyrazinyl, pyrimidinyl, pyridazinyl, thienyl, furyl, furazanyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4 oxadiazolyl.

Additionally, heterocyclyl encompasses polycyclic heterocyclyls (including both aromatic or non-aromatic), for example, indolyl, indolinyl, isoindolinyl, quinolinyl, tetrahydroquinolinyl, isoquinolinyl, tetrahydroisoquinolinyl, 1,4-benzodioxanyl, coumarinyl, dihydrocoumarinyl, benzofuranyl, 2,3-dihydrobenzofuranyl, isobenzofuranyl, chromenyl, chromanyl, isochromanyl, xanthenyl, phenoxathiinyl, thianthrenyl, indolizinyl, isoindolyl, indazolyl, purinyl, phthalazinyl, naphthyridinyl, quinoxalinyl, quinazolinyl, cinnolinyl, pteridinyl, phenanthridinyl, perimidinyl, phenanthrolinyl, phenazinyl, phenothiazinyl, phenoxazinyl, 1,2-benzisoxazolyl, benzothiophenyl, benzoxazolyl, benzthiazolyl, benzimidazolyl, benztriazolyl, thioxanthinyl, carbazolyl, carbolinyl, acridinyl, pyrolizidinyl, and quinolizidinyl.

In addition to the polycyclic heterocyclyls described above, heterocyclyl includes polycyclic heterocyclyls wherein the ring fusion between two or more rings includes more than one bond common to both rings and more than two atoms common to both rings. Examples of such bridged heterocycles include quinuclidinyl, diazabicyclo[2.2.1]heptyl; and 7-oxabicyclo[2.2.1]heptyl.

The term “heteroaryl” refers to a heterocyclyl having aromatic character (e.g., 4n+2 delocalized electrons.)

The term “heterocylcoalkyl” refers to a monocyclic or polycyclic ring comprising carbon and hydrogen atoms and at least one heteroatom, preferably, 1 to 3 heteroatoms selected from nitrogen, oxygen, and sulfur, and having no unsaturation. Examples of heterocycloalkyl groups include pyrrolidinyl, pyrrolidino, piperidinyl, piperidino, piperazinyl, piperazino, morpholinyl, morpholino, thiomorpholinyl, thiomorpholino, and pyranyl. A heterocycloalkyl group can be unsubstituted or substituted with one or two suitable substituents. Preferably, the heterocycloalkyl group is a monocyclic or bicyclic ring, more preferably, a monocyclic ring, wherein the ring comprises from 3 to 6 carbon atoms and form 1 to 3 heteroatoms, referred to herein as C₃₋₆heterocycloalkyl.

The term “six-membered” refers to a group having a ring that contains six ring atoms.

The term “five-membered” refers to a group having a ring that contains five ring atoms.

A five-membered ring heteroaryl is a heteroaryl with a ring having five ring atoms wherein 1, 2 or 3 ring atoms are independently selected from N, O and S.

Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl.

A six-membered ring heteroaryl is a heteroaryl with a ring having six ring atoms wherein 1, 2 or 3 ring atoms are independently selected from N, O and S.

Exemplary six-membered ring heteroaryls are pyridyl, pyrazinyl, pyrimidinyl, triazinyl and pyridazinyl.

The term “alkoxy” refers to radicals of the general formula —O—R, wherein R is selected from a hydrocarbon radical. Exemplary alkoxy includes methoxy, ethoxy, propoxy, isopropoxy, butoxy, t-butoxy, isobutoxy, cyclopropylmethoxy, allyloxy, and propargyloxy.

Halogen includes fluorine, chlorine, bromine and iodine.

“RT” or “rt” means room temperature.

In one aspect, an embodiment of the invention provides a compound of formula I, a pharmaceutically acceptable salt thereof, a diastereomer, an enantiomer, or a mixture thereof:

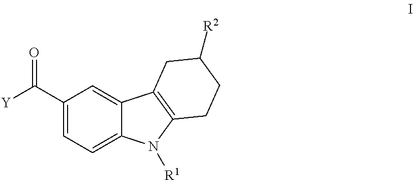

wherein

Y is selected from

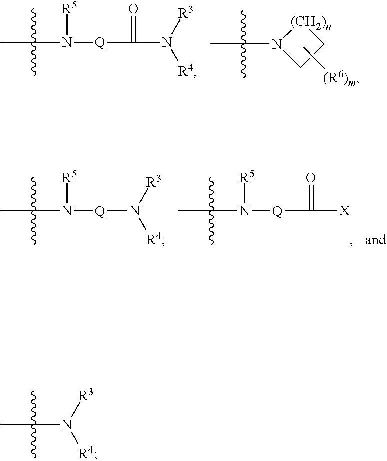

R¹ is selected from —H, C₁₋₆alkyl, C₂₋₆alkenyl, C₃₋₆cycloalkyl, C₃₋₆cycloalkyl-C₁₋₄alkyl, —C(═O)—NR¹⁴R¹⁵, —S(═O)₂—NR¹⁴R¹⁵, S(═O)₂—C₁₋₆alkyl, —S(═O)₂—C₆₋₁₀aryl, —S(═O)₂—C₂₋₅heteroaryl, —C(═O)—C₁₋₆alkyl, C₆₋₁₀aryl-C₁₋₄alkyl and C₂₋₅heteroaryl-C₁₋₄alkyl, wherein said C₁₋₆alkyl, C₂₋₆alkenyl, —S(═O)₂—C₁₋₆alkyl, —S(═O)₂—C₆₋₁₀aryl, —S(═O)₂—C₂₋₅heteroaryl, —C(═)—C₁₋₆alkyl, C₆₋₁₀aryl-C₁₋₄alkyl and C₂₋₅heteroaryl-C₁₋₄alkyl used in defining R¹ are optionally substituted with one or more groups selected from —OR, R, —CO₂H, —CO₂—R, —SO₂—R, halogen, —NO₂, —OH, —NH₂, —NHR, —CN, —C(═O)—NH₂, —C(═O)—NR₂ and —C(═O)—NHR;

R² is selected from C₃₋₆heterocycloalkyl, C₃₋₆heterocycloalkyl-C₁₋₄alkyl, C₃₋₆cycloalkyl, C₃₋₆cycloalkyl-C₁₋₄alkyl, C₁₋₆alkyl, C₂₋₆alkenyl, C₆₋₁₀aryl, C₆₋₁₀aryl-C₁₋₄alkyl, C₂₋₆heteroaryl, C₂₋₆heteroaryl-C₁₋₄alkyl, —C(═O)—C₁₋₆alkyl, —C(═O)—C₃₋₆cycloalkyl and —C(═NH)—C₁₋₆alkyl, wherein said C₃₋₆heterocycloalkyl, C₃₋₆heterocycloalkyl-C₁₋₄alkyl, C₃₋₆cycloalkyl, C₃₋₆cycloalkyl-C₁₋₄alkyl, C₁₋₆alkyl, C₂₋₆alkenyl, C₆₋₁₀aryl, C₆₋₁₀aryl-C₁₋₄alkyl, C₂₋₆heteroaryl, C₂₋₆heteroaryl-C₁₋₄alkyl, —C(═O)—C₁₋₆alkyl, —C(O)—C₃₋₆cycloalkyl and —C(═NH)—C₁₋₆alkyl used in defining R² are optionally substituted with one or more groups selected from —OR, R, NO₂, —CO₂H, —CO₂—R, —SO₂—R, halogen, —OH, —NH₂, —NHR, —CN, —C(═O)—NH₂ and —C(═O)—NHR;

R³ and R⁴ are independently selected from —H, C₃₋₆cycloalkyl, C₃₋₆heterocycloalkyl, C₂₋₅heteroaryl, C₆₋₁₀aryl, C₁₋₆alkyl, C₁₋₆alkoxy, amino, C₁₋₆alkylamino, diC₁₋₆alkylamino, —C(═O)—C₁₋₆alkyl, —C(═O)—O—C₁₋₆alkyl, —C(═O)—C₃₋₆cycloalkyl, —C(═O)—NR¹⁴R¹⁵ and —S(═O)₂—NR¹⁴R¹⁵, wherein said C₃₋₆cycloalkyl, C₃₋₆heterocycloalkyl, C₂₋₅heteroaryl, C₆₋₁₀aryl, C₁₋₆alkyl, C₁₋₆alkoxy, C₁₋₆alkylamino, diC₁₋₆alkylamino, —C(═O)—C₁₋₆alkyl, —C(═O)—O—C₁₋₆alkyl, and —C(═O)—C₃₋₆cycloalkyl used in defining R³ and R⁴ are optionally substituted with one or more groups selected from —OR, R, NO₂, —CO₂H, —CO₂—R, —SO₂—R, halogen, —OH, —NH₂, —NHR, —C(═O)—NH₂, —CN, —C(═O)—NR₂ and —C(O)—NHR;

R⁵ is selected from —H, C₁₋₆alkyl, and C₃₋₆cycloalkyl;

R⁶ is independently selected from —H, —CN, —NO₂, C₁₋₆alkoxy, halogen, C₁₋₆alkyl, —OH, —NH₂, —NHC(═O)R¹² and —C(═O)NR¹²R¹³;

R¹² and R¹³ are independently selected from —H, C₁₋₆alkoxy, C₃₋₆heterocycloalkyl, C₃₋₆cycloalkyl-C₁₋₄alkyl, and C₃₋₆cycloalkyl wherein said C₁₋₆alkyl, C₁₋₆alkoxy, C₃₋₆heterocycloalkyl, C₃₋₆cycloalkyl-C₁₋₄alkyl and C₃₋₆cycloalkyl used in defining R¹² and R¹³ are optionally substituted with one or more halogens or —OH;

R¹⁴ and R¹⁵ are independently selected from —H, C₁₋₆alkyl, C₆₋₁₀aryl, C₆₋₁₀aryl-C₁₋₄alkyl, C₂₋₅heterocyclyl, C₂₋₅heterocyclyl-C₁₋₄alkyl, C₂₋₆alkenyl, C₃₋₆cycloalkyl, and C₃₋₆cycloalkyl-C₁₋₄alkyl, N,N-di(C₁₋₄alkyl)amido-C₁₋₆alkyl, hydroxy-C₁₋₆alkyl and C₁₋₆alkoxy-C₁₋₆alkyl that are optionally substituted with one or groups selected from halogen, —OH, —CN, —NH₂ and methoxy;

Q is independently selected from C₁₋₆alkylene, C₁₋₆alkylidene and

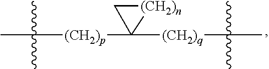

wherein said C₁₋₆alkylene and C₁₋₆alkylidene are optionally substituted with on or more groups selected from —OR, —R, hydroxy-C₁₋₆alkyl, NO₂, —CO₂H, —CO₂—R, —SO₂—R, halogen, —OH, —NH₂, —NHR, —C(═O)—NH₂, —CN, —C(═O)—NR₂ and —C(═O)—NHR;

X is selected from —OH, halogen or —OR;

n is independently selected from 1, 2 and 3;

p, q and m are independently selected from 0, 1, 2 and 3; and

R is independently C₁₋₆alkyl.

In another embodiment, R¹ is selected from C₁₋₆alkyl, C₃₋₆cycloalkyl, C₃₋₆cycloalkyl-C₁₋₄alkyl, and —S(═O)₂—C₁₋₆alkyl;

R² is selected from C₁₋₆alkyl, C₂₋₅heterocycloalkyl and C₃₋₆cycloalkyl wherein said C₁₋₆alkyl, C₂₋₅heterocycloalkyl and C₃₋₆cycloalkyl used in defining R² is optionally substituted with one or more groups selected from —OR, R, NO₂, —CO₂H, —CO₂—R, —SO₂—R, halogen, —OH, —NH₂, —NHR, —CN, —C(═O)—NH₂, and —C(═O)—NHR;

R³ and R⁴ are independently selected from —H, C₃₋₆cycloalkyl, C₃₋₆heterocycloalkyl, C₂₋₅heteroaryl, diC₁₋₆alkylamino, C₁₋₆alkoxy, and C₁₋₆alkyl, wherein said C₃₋₆cycloalkyl, C₃₋₆heterocycloalkyl, C₂₋₅heteroaryl, diC₁₋₆alkylamino, C₁₋₆alkoxy, and C₁₋₆alkyl used in defining R³ is optionally substituted with one or more groups selected from —OR, R, —CO₂H, —CO₂—R, —SO₂—R, halogen, —NO₂, —OH, —NH₂, —NHR, —CN, —C(═O)—NH₂, —C(═O)—NR₂, and —C(═O)—NHR;

R⁵ is selected from —H, C₁₋₆alkyl, and C₃₋₆cycloalkyl;

R⁶ is selected from C₁₋₆alkyl, —OH, —NH₂, —NHC(═O)R¹² and —C(═O)NR¹²R¹³;

R¹² and R¹³ are independently selected from —H, C₁₋₆alkyl, C₁₋₆alkoxy, C₃₋₆heterocycloalkyl, C₃₋₆cycloalkyl-C₁₋₄alkyl, and C₃₋₆cycloalkyl wherein said C₁₋₆alkyl, C₁₋₆alkoxy, C₃₋₆heterocycloalkyl, C₃₋₆cycloalkyl-C₁₋₄alkyl, and C₃₋₆cycloalkyl used in defining R¹² and R¹³ is optionally substituted with one or more haolgens; and

R is independently C₁₋₆alkyl.

In another embodiment, R¹ is selected from methyl, ethyl, 1-propyl, 2-propyl, 1-butyl, 2-butyl, t-butyl, allyl, —S(═O)₂—CH₃, —S(═O)₂—CH₂CH₃, 2-methoxyethyl, tetrahydropyran-4-yl-methyl, 1-propylsulfonyl, 2-propylsulfonyl, cyclopropylsulfonyl, phenyl, phenylsulfonyl, 2-(methoxycarbonyl)-phenylsulfonyl; 2-(hydroxycarbonyl)-phenylsulfonyl, 1-methyl-1H-imidazol-4-yl-sulfonyl, 1H-imidazol-1-yl-sulfonyl, (5-methylisoxazol-4-yl)sulfonyl, morpholin-4-ylcarbonyl, 4-amino-phenyl, —CH₂—C(═O)—N(CH₃)₂, —C(═O)—N(CH₃)₂, —S(═O)₂—N(CH₃)₂, —S(═O)₂—NHCH₂CH₃, —C(═O)—CH₂CH₂CH₃, —CH₂—C(═O)—OCH₃, —CH₂—C(═O)—OCH₂CH₃, —CH₂—CO₂H, benzyl, 4-aminobenzyl, 4-nitrobenzyl, 4-methylsulfonyl-benzyl, 4-methylthio-benzyl, 4-acetylamino-benzyl, 4-methoxy-benzyl, 4-ethoxy-benzyl, 2,6-difluorobenzyl, (6-chloro-1,3-benzodioxol-5-yl)methyl, (5-ethoxycarbonyl)-fur-2-yl-methyl, (2-methyl-1,3-thiazol-4-yl)-methyl, (5-methyl-isoxazol-4-yl)-methyl, pyridin-2-ylmethyl, cyclobutylmethyl, and cyclopropylmethyl.

In a further embodiment, R² is selected from methyl, ethyl, isopropyl, propyl, 2-methyl-propyl, 1-butyl, tert-butyl, 1-pentyl, 1-acetyl-piperidin-4-yl, tetrahydrothien-3-yl, cyclopropylmethyl, cyclobutylmethyl, cyclopentylmethyl, cyclobutyl, cyclopentyl, cyclohexyl, 4-tetrahydro-2H-pyranyl, tetrahydro-thiopyran-4-yl, 2-pyrimidinyl, 1-iminoethyl, 2-pyridinyl, 3,4,5,6-tetrahydropyrdin-2-yl, 3,4-dihydro-2H-pyrrol-5-yl, 2-pyridinyl-methyl, 3-pyridinylmethyl, 4-pyridinylmethyl, 1-methyl-4-piperidinyl, 4-piperidinyl, (6-methyl-pyridin-2-yl)methyl, (2-ethyl-4-methyl-1H-imidazol-5-ylmethyl, tetrahydrofuran-2-yl, tetrahydrofuran-3-yl, tetrahydrofuran-3-ylmethyl, 1-ethyl-1H-pyrazol-4-yl, 1,3-dimethyl-1H-pyrazol-5-yl, (3-methylpyridin-4-ylmethyl, 1,3-oxazol-2-ylmethyl, 1,3-oxazol-5-ylmethyl, 2-(tetrahydro-2H-pyran-4-yl)ethyl, tetrahydro-2H-1-pyran-4-ylmethyl, 2-phenylethyl, 2-methoxybenzyl, 3,3,3-trifluoropropyl, 2,2-difluoroethyl, 2-hydroxycyclopentyl, (1-ethyl-3-methyl-1H-pyrazol-5-yl)methyl, 2,1,3-benzoxadiazol-5-ylmethyl, 3-thienylmethyl, 2-trifluoromethyl-benzyl, 3-methylbutyl, cyclohex-3-en-1-ylmethyl, 2-fluoro-6-methoxybenzyl, 2-phenyl-propyl, 2-ethyl-butyl, cyclobutylcarbonyl, 2,2-difluoropropanoyl, cyclopentylcarbonyl, tetrahydro-2H-pyran-4-ylcarbonyl, cyclopropylcarbonyl, propylcarbonyl, N-ethylaminocarbonyl, N-isopropylaminocarbonyl, cyclopropylsulfonyl, and ethylsulfonyl.

In another embodiment, Y is

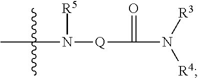

R⁵ is selected from —H, C₁₋₆alkyl, and C₃₋₆cycloalkyl;

R³ and R⁴ are independently selected from —H, C₃₋₆cycloalkyl, C₃₋₆heterocycloalkyl, C₂₋₅heteroaryl, diC₁₋₆alkylamino, C₁₋₆alkoxy, and C₁₋₆alkyl, wherein said C₁₋₆alkyl, C₃₋₆heterocycloalkyl, C₂₋₅heteroaryl, diC₁₋₆alkylamino, C₁₋₆alkoxy, and C₃₋₆cycloalkyl used in defining R³ and R⁴ are optionally substituted with one or more groups selected from —OR, R, NO₂, —CO₂H, —CO₂—R, —SO₂—R, halogen, —OH, —NH₂, —NHR, —C(═O)—NH₂, —CN, and —C(═O)—NHR;

Q is C₁₋₆alkylene or C₁₋₆alkylidene, optionally substituted with one or more —CH₂OH; and

R is C₁₋₆alkyl.

In another embodiment, Y is

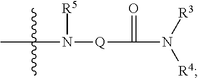

R⁵ is selected from methyl, ethyl, 1-propyl, 2-propyl, 1-butyl, 2-butyl, and t-butyl;

R³ and R⁴ are independently selected from —H, methyl, ethyl, 1-propyl, 2-propyl, 1-butyl, 2-butyl, t-butyl, cyclopropyl, cyclopropylmethyl, cyclobutylmethyl, cyclopentylmethyl, cyclobutyl, cyclopentyl, cyclopropanecarbonitryl, oxetanyl, pyrrolyl, methoxy, dimethylamino, and cyclohexyl, wherein said methyl, ethyl, 1-propyl, 2-propyl, 1-butyl, 2-butyl, t-butyl, cyclopropyl, cyclopropylmethyl, cyclobutylmethyl, cyclopentylmethyl, cyclobutyl, cyclopentyl, cyclopropanecarbonitryl, oxetanyl, pyrrolyl, methoxy, dimethylamino, and cyclohexyl used in defining R³ and R⁴ are optionally substituted with one or more groups selected from —OR, R, NO₂, —CO₂H, —CO₂—R, —SO₂—R, halogen; —OH, —NH₂, —NHR, —C(═O)—NH₂, —CN and —C(═O)—NHR;

Q is C₁₋₆alkylene or C₁₋₆alkylidene, optionally substituted with one or more —CH₂OH; and

R is C₁₋₆alkyl.

In an even further embodiment, Y is

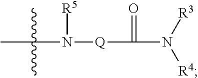

R⁵ is selected from methyl, ethyl, 1-propyl, 2-propyl, 1-butyl, 2-butyl, and t-butyl;

R³ and R⁴ are independently selected from —H, methyl, ethyl, 1-propyl, 2-propyl, 1-butyl, 2-butyl, t-butyl, cyclopropyl, cyclopropylmethyl, cyclobutylmethyl, cyclopentylmethyl, cyclobutyl, cyclopentyl, cyclopropanecarbonitryl, oxetanyl, pyrrolyl, methoxy, dimethylamino, and cyclohexyl wherein said methyl, ethyl, 1-propyl, 2-propyl, 1-butyl, 2-butyl, t-butyl, cyclopropyl, cyclopropylmethyl, cyclobutylmethyl, cyclopentylmethyl, cyclobutyl, cyclopentyl, cyclopropanecarbonitryl, oxetanyl, pyrrolyl, methoxy, dimethylamino, and cyclohexyl used in defining R³ and R⁴ are optionally substituted with one or more groups selected from fluoro, —CN, —OH, and methoxy;

R¹ is selected from methyl, ethyl, —S(═O)₂—CH₃, —S(═O)₂—CH₂CH₃, and 2-propylsulfonyl;

Q is selected from C₁₋₆alkylene, hydroxymethyl-C₁₋₆alkylene, and C₁₋₆alkylidene; and

R² is tetrahydropyranyl.

In another embodiment, Y is

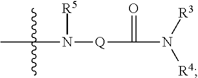

R⁵ is selected from methyl, ethyl, 1-propyl, 2-propyl, 1-butyl, 2-butyl, and t-butyl;

R³ and R⁴ are independently selected from —H, methyl, ethyl, 1-propyl, 2-propyl, 1-butyl, 2-butyl, t-butyl, cyclopropyl, cyclopropylmethyl, cyclobutylmethyl, cyclopentylmethyl, cyclobutyl, cyclopentyl, cyclopropanecarbonitryl and cyclohexyl wherein said methyl, ethyl, 1-propyl, 2-propyl, 1-butyl, 2-butyl, t-butyl, cyclopropyl, cyclopropylmethyl, cyclobutylmethyl, cyclopentylmethyl, cyclobutyl, cyclopentyl, cyclopropanecarbonitryl, and cyclohexyl used in defining R³ and R⁴ are optionally substituted with one or more fluoro;

R¹ is selected from methyl, ethyl, —S(═O)₂—CH₃, —S(═O)₂—CH₂CH₃ and 2-propylsulfonyl;

Q is

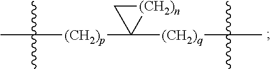

R² is tetrahydropyranyl;

n is selected from 1, 2 and 3; and

p, q are independently selected from 0, 1, 2 and 3.

In a further embodiment, Y is

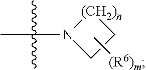

R⁶ is selected from C₁₋₆alkyl, —OH, —NH₂, —NHC(═O)R¹² and —C(═O)NR¹²R¹³ wherein R¹² and R¹³ are independently selected from —H, C₁₋₆alkyl, and C₃₋₆cycloalkyl wherein said C₁₋₆alkyl and C₃₋₆cycloalkyl used in defining R¹² and R¹³ is optionally substituted with one or more halogens; and

n is 1, 2, or 3; and m is 1.

In an even further embodiment, Y is

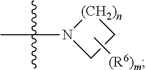

R⁶ is selected from methyl, —OH, —NH₂, —NHC(═O)R¹² and —C(═O)NR¹²R¹³ wherein R¹² and R¹³ are independently selected from —H, C₁₋₆alkyl, and C₃₋₆cycloalkyl wherein said C₁₋₆alkyl and C₃₋₆cycloalkyl used in defining R¹² and R¹³ is optionally substituted with one or more halogens;

R¹ is selected from methyl, ethyl, —S(═O)₂—CH₃, —S(═O)₂—CH₂CH₃, and 2-propylsulfonyl;

R² is selected from C₃₋₆cycloalkyl, tetrahydropyranyl and C₁₋₆alkyl; and

n is 1, 2 or 3; and m is 1.

In another embodiment, Y is

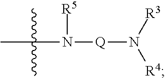

R⁵ is selected from —H, C₁₋₆alkyl, and C₃₋₆cycloalkyl;

R³ and R⁴ are independently selected from —H, C₁₋₆alkyl, —C(═O)—C₁₋₆alkyl, —C(═O)—C₃₋₆cycloalkyl, —C(═O)—NR¹⁴R¹⁵ and —S(═O)—NR¹⁴R¹⁵; wherein said C₁₋₆alkyl, —C(═O)—C₁₋₆alkyl and —C(═O)—C₃₋₆cycloalkyl used in defining R³ and R⁴ is optionally substituted with one or more group selected from —OR, R, —CO₂H, —CO₂—R, —SO₂—R, halogen, —NO₂, —OH, —NH₂, —NHR, —CN, —C(═O)—NH₂, —C(═O)—NR₂ and —C(═O)—NHR;

Q is C₁₋₆alkylene or C₁₋₆alkylidene;

R is C₁₋₆alkyl; and

R¹⁴ and R¹⁵ are independently selected from —H, C₁₋₆alkyl, C₆₋₁₀aryl, C₆₋₁₀aryl-C₁₋₄alkyl, C₃₋₆heterocyclyl, C₃₋₆heterocyclyl-C₁₋₄alkyl, C₂₋₆alkenyl, C₃₋₆cycloalkyl, C₃₋₆cycloalkyl-C₁₋₄alkyl, N,N-di(C₁₋₄alkyl)amido-C₁₋₆alkyl, hydroxy-C₁₋₆alkyl and C₁₋₆alkoxy-C₁₋₆alkyl that are optionally substituted with one or more groups selected from halogen, —OH, —CN, —NH₂ and methoxy.

In another embodiment, Y is

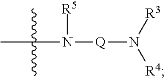

R⁵ is methyl; and

R³ and R⁴ are independently selected from —H, methyl, ethyl, 1-propyl, 2-propyl, 1-butyl, 2-butyl, t-butyl, cyclopropyl, cyclopropylmethyl, cyclobutylmethyl, cyclopentylmethyl, cyclobutyl, cyclopentyl, cyclopropanecarbonitryl, cyclohexyl, —C(═O)cyclopropyl, —CO₂CH₃, and —S(═O)₂NH-cyclopropyl.

In another embodiment, Y is

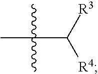

R³ and R⁴ are independently selected from —H, C₁₋₆alkyl, C₁₋₆cycloalkyl, C₃₋₆heterocycloalkyl, wherein said C₁₋₆alkyl, C₁₋₃cycloalkyl, and C₃₋₆heterocycloalkyl are optionally substituted with one or more groups selected from —OR, R, NO₂, —CO₂H, —CO₂—R, —SO₂—R, halogen, —OH, —NH₂, —NHR, —C(═O)—NH₂, —CN, —C(═O)—NR₂ and —C(═O)—NHR; and

R is C₁₋₆alkyl.

In a further embodiment, Y is

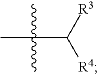

and

R³ and R⁴ are independently selected from —H, methyl, and ethyl wherein said methyl and ethyl are optionally substituted with —OH or halogen.

In another embodiment, R² is tetrahydropyranyl.

In a further embodiment, R² is 4-tetrahydropyranyl.

In another embodiment, a compound of the invention may be selected from:
N-[2-(Cyclopropylamino)-2-oxoethyl]-N,9-dimethyl-3-(tetrahydro-2H-pyran-4-yl)-2,3,4,9-tetrahydro-1H-carbazole-6-carboxamide;
(+)—N-[2-(Cyclopropylamino)-2-oxoethyl]-N,9-dimethyl-3-(tetrahydro-2H-pyran-4-yl)-2,3,4,9-tetrahydro-1H-carbazole-6-carboxamide;
(−)-N-[2-(Cyclopropylamino)-2-oxoethyl]-N,9-dimethyl-3-(tetrahydro-2H-pyran-4-yl)-2,3,4,9-tetrahydro-1H-carbazole-6-carboxamide;
N-Ethyl-N-[2-(ethylamino)-2-oxoethyl]-9-methyl-3-(tetrahydro-2H-pyran-4-yl)-2,3,4,9-tetrahydro-1H-carbazole-6-carboxamide;
N-Ethyl-N-[2-(isopropylamino)-2-oxoethyl]-9-methyl-3-(tetrahydro-2H-pyran-4-yl)-2,3,4,9-tetrahydro-1H-carbazole-6-carboxamide;
N-Cyclopropyl-1-(9-methyl-3-(tetrahydro-2H-pyran-4-yl)-2,3,4,9-tetrahydro-1H-carbazole-6-carbonyl)piperidine-4-carboxamide;
N-Ethyl-N-{2-[(2-fluoroethyl)amino]-2-oxoethyl}-9-methyl-3-(tetrahydro-2H-pyran-4-yl)-2,3,4,9-tetrahydro-1H-carbazole-6-carboxamide;
N-[2-(Cyclopropylamino)-2-oxoethyl]-N-ethyl-9-methyl-3-(tetrahydro-2H-pyran-4-yl)-2,3,4,9-tetrahydro-1H-carbazole-6-carboxamide;
N-Cyclopropyl-2-(1-(9-methyl-3-(tetrahydro-2H-pyran-4-yl)-2,3,4,9-tetrahydro-1H-carbazole-6-carbonyl)azetidin-3-yl)acetamide;
N,9-Dimethyl-N-(2-(methylamino)ethyl)-3-(tetrahydro-2H-pyran-4-yl)-2,3,4,9-tetrahydro-1H-carbazole-6-carboxamide;
N-(2-(3-Cyclopropyl-1-methylureido)ethyl)-N,9-dimethyl-3-(tetrahydro-2H-pyran-4-yl)-2,3,4,9-tetrahydro-1H-carbazole-6-carboxamide;
N-(2-(3-Cyclopropyl-1-methylthioureido)ethyl)-N,9-dimethyl-3-(tetrahydro-2H-pyran-4-yl)-2,3,4,9-tetrahydro-1H-carbazole-6-carboxamide;
N-{2-[(2-Fluoroethyl)amino]-2-oxoethyl}-N,9-dimethyl-3-(tetrahydro-2H-pyran-4-yl)-2,3,4,9-tetrahydro-1H-carbazole-6-carboxamide;
N-Ethyl-9-methyl-N-[2-(methylamino)-2-oxoethyl]-3-(tetrahydro-2H-pyran-4-yl)-2,3,4,9-tetrahydro-1H-carbazole-6-carboxamide;
Methyl, methyl[2-(methyl{[9-methyl-3-(tetrahydro-2H-pyran-4-yl)-2,3,4,9-tetrahydro-1H-carbazol-6-yl]carbonyl}amino)ethyl]carbamate;
N-{2-[(Cyclopropylcarbonyl)(methyl)amino]ethyl}-N,9-dimethyl-3-(tetrahydro-2H-pyran-4-yl)-2,3,4,9-tetrahydro-1H-carbazole-6-carboxamide;
N-Cyclopropyl-1-{[9-methyl-3-(tetrahydro-2H-pyran-4-yl)-2,3,4,9-tetrahydro-1H-carbazol-6-yl]carbonyl}piperidine-3-carboxamide;
N-Cyclopropyl-1-{[9-methyl-3-(tetrahydro-2H-pyran-4-yl)-2,3,4,9-tetrahydro-1H-carbazol-6-yl]carbonyl}azetidine-3-carboxamide;
N-Ethyl-N-{2-[(1-isocyanocyclopropyl)amino]-2-oxoethyl}-9-methyl-3-(tetrahydro-2H-pyran-4-yl)-2,3,4,9-tetrahydro-1H-carbazole-6-carboxamide;
N-Ethyl-N-(2-hydroxyethyl)-9-methyl-3-(tetrahydro-2H-pyran-4-yl)-2,3,4,9-tetrahydro-1H-carbazole-6-carboxamide;
N-(3-(cyclopropylamino)-3-oxopropyl)-N,9-dimethyl-3-(tetrahydro-2H-pyran-4-yl)-2,3,4,9-tetrahydro-1H-carbazole-6-carboxamide;
N-(4-(Cyclopropylamino)-4-oxobutyl)-N-ethyl-9-methyl-3-(tetrahydro-2H-pyran-4-yl)-2,3,4,9-tetrahydro-1H-carbazole-6-carboxamide;
N-(4-(Cyclopropylamino)-4-oxo butyl)-N-methyl-9-methyl-3-(tetrahydro-2H-pyran-4-yl)-2,3,4,9-tetrahydro-1H-carbazole-6-carboxamide;
N-(4-(Methylamino)-4-oxobutyl)-N-methyl-9-methyl-3-(tetrahydro-2H-pyran-4-yl)-2,3,4,9-tetrahydro-1H-carbazole-6-carboxamide;
N-(4-(Ethylamino)-4-oxobutyl)-N-methyl-9-methyl-3-(tetrahydro-2H-pyran-4-yl)-2,3,4,9-tetrahydro-1H-carbazole-6-carboxamide;
N-(4-(2-Fluoroethylamino)-4-oxobutyl)-N-methyl-9-methyl-3-(tetrahydro-2H-pyran-4-yl)-2,3,4,9-tetrahydro-1H-carbazole-6-carboxamide;
3-cyclohexyl-9-methyl-6-[(4-methylpiperidin-1-yl)carbonyl]-2,3,4,9-tetrahydro-1H-carbazole;
3-cyclohexyl-9-ethyl-6-[(4-methylpiperidin-1-yl)carbonyl]-2,3,4,9-tetrahydro-1H-carbazole;
3-cyclohexyl-6-[(4-methylpiperidin-1-yl)carbonyl]-9-(methylsulfonyl)-2,3,4,9-tetrahydro-1H-carbazole;
3-cyclohexyl-9-(ethylsulfonyl)-6-[(4-methylpiperidin-1-yl)carbonyl]-2,3,4,9-tetrahydro-1H-carbazole;
3-cyclohexyl-N-[2-(cyclopropylamino)-2-oxoethyl]-N-methyl-9-(methylsulfonyl)-2,3,4,9-tetrahydro-1H-carbazole-6-carboxamide;
3-cyclohexyl-N-[2-(cyclopropylamino)-2-oxoethyl]-9-(isopropylsulfonyl)-N-methyl-2,3,4,9-tetrahydro-1H-carbazole-6-carboxamide;
N-Ethyl-9-methyl-N-(2-oxo-2-(tetrahydro-2H-pyran-4-ylamino)ethyl)-3-(tetrahydro-2H-pyran-4-yl)-2,3,4,9-tetrahydro-1H-carbazole-6-carboxamide;
N-Ethyl-9-methyl-N-(2-oxo-2-((S)-tetrahydrofuran-3-ylamino)ethyl)-3-(tetrahydro-2H-pyran-4-yl)-2,3,4,9-tetrahydro-1H-carbazole-6-carboxamide;
N-Ethyl-9-methyl-N-(2-oxo-2-((R)-tetrahydrofuran-3-ylamino)ethyl)-3-(tetrahydro-2H-pyran-4-yl)-2,3,4,9-tetrahydro-1H-carbazole-6-carboxamide;
N-Ethyl-9-methyl-N-(2-(oxetan-3-ylamino)-2-oxoethyl)-3-(tetrahydro-2H-pyran-4-yl)-2,3,4,9-tetrahydro-1H-carbazole-6-carboxamide;
N-Ethyl-N-(4-hydroxybutyl)-9-methyl-3-(tetrahydro-2H-pyran-4-yl)-2,3,4,9-tetrahydro-1H-carbazole-6-carboxamide;
N-(2-(Cyanomethylamino)-2-oxoethyl)-N-ethyl-9-methyl-3-(tetrahydro-2H-pyran-4-yl)-2,3,4,9-tetrahydro-1H-carbazole-6-carboxamide;
N-(2-(Cyclopropylamino)-2-oxoethyl)-N-ethyl-3-(tetrahydro-2H-pyran-4-yl)-2,3,4,9-tetrahydro-1H-carbazole-6-carboxamide;
N—((S)-1-(2-Fluoroethylamino)-1-oxopropan-2-yl)-N,9-dimethyl-3-(tetrahydro-2H-pyran-4-yl)-2,3,4,9-tetrahydro-1H-carbazole-6-carboxamide;
N—((S)-1-(Cyclopropylamino)-1-oxopropan-2-yl)-N,9-dimethyl-3-(tetrahydro-2H-pyran-4-yl)-2,3,4,9-tetrahydro-1H-carbazole-6-carboxamide;
N-(4-(Cyclopropylamino)-4-oxobutyl)-9-methyl-3-(tetrahydro-2H-pyran-4-yl)-2,3,4,9-tetrahydro-1H-carbazole-6-carboxamide;
N-(1-(Cyclopropylcarbamoyl)cyclopropyl)-N,9-dimethyl-3-(tetrahydro-2H-pyran-4-yl)-2,3,4,9-tetrahydro-1H-carbazole-6-carboxamide;
N-(2-Fluoroethyl)-9-methyl-3-(tetrahydro-2H-pyran-4-yl)-2,3,4,9-tetrahydro-1H-carbazole-6-carboxamide;
N-Ethyl-N-(4-(2-fluoroethylamino)-4-oxobutyl)-9-methyl-3-(tetrahydro-2H-pyran-4-yl)-2,3,4,9-tetrahydro-1H-carbazole-6-carboxamide;

N—((R)-1-(2-fluoroethylamino)-1-oxopropan-2-yl)-N,9-dimethyl-3-(tetrahydro-2H-pyran-4-yl)-2,3,4,9-tetrahydro-1H-carbazole-6-carboxamide;
N—((R)-1-(ethylamino)-1-oxopropan-2-yl)-N,9-dimethyl-3-(tetrahydro-2H-pyran-4-yl)-2,3,4,9-tetrahydro-1H-carbazole-6-carboxamide;
N-ethyl-N-(2-hydroxypropyl)-9-methyl-3-(tetrahydro-2H-pyran-4-yl)-2,3,4,9-tetrahydro-1H-carbazole-6-carboxamide;
N-(2-(2-Cyanoethylamino)-2-oxoethyl)-N-ethyl-9-(ethylsulfonyl)-3-(tetrahydro-2H-pyran-4-yl)-2,3,4,9-tetrahydro-1H-carbazole-6-carboxamide;
(3S)—N-Cyclopropyl-1-(9-methyl-3-(tetrahydro-2H-pyran-4-yl)-2,3,4,9-tetrahydro-1H-carbazole-6-carbonyl)piperidine-3-carboxamide;
(3S)—N-cyclopropyl-1-(9-methyl-3-(tetrahydro-2H-pyran-4-yl)-2,3,4,9-tetrahydro-1H-carbazole-6-carbonyl)piperidine-3-carboxamide;
N,9-Dimethyl-N-(4-(methylamino)-4-oxobutyl)-3-(tetrahydro-2H-pyran-4-yl)-2,3,4,9-tetrahydro-1H-carbazole-6-carboxamide;
N-Ethyl-N-(2-(2-fluoroethylamino)-2-oxoethyl)-9-methyl-3-(tetrahydro-2H-pyran-4-yl)-2,3,4,9-tetrahydro-1H-carbazole-6-carboxamide;
N-(2-(Cyclopropylamino)-2-oxoethyl)-N-ethyl-9-methyl-3-(tetrahydro-2H-pyran-4-yl)-2,3,4,9-tetrahydro-1H-carbazole-6-carboxamide;
N-(4-(2-Fluoroethylamino)-4-oxobutyl)-N,9-dimethyl-3-(tetrahydro-2H-pyran-4-yl)-2,3,4,9-tetrahydro-1H-carbazole-6-carboxamide;
N-ethyl-N-(2-hydroxyethyl)-9-methyl-3-(tetrahydro-2H-pyran-4-yl)-2,3,4,9-tetrahydro-1H-carbazole-6-carboxamide;
N-Cyclopropyl-1-(9-methyl-3-(tetrahydro-2H-pyran-4-yl)-2,3,4,9-tetrahydro-1H-carbazole-6-carbonyl)pyrrolidine-3-carboxamide;
(3S)—N-Cyclopropyl-1-(9-methyl-3-(tetrahydro-2H-pyran-4-yl)-2,3,4,9-tetrahydro-1H-carbazole-6-carbonyl)pyrrolidine-3-carboxamide;
(3R)—N-Cyclopropyl-1-(9-methyl-3-(tetrahydro-2H-pyran-4-yl)-2,3,4,9-tetrahydro-1H-carbazole-6-carbonyl)pyrrolidine-3-carboxamide;
N-(2-Fluoroethyl)-1-(9-methyl-3-(tetrahydro-2H-pyran-4-yl)-2,3,4,9-tetrahydro-1H-carbazole-6-carbonyl)pyrrolidine-3-carboxamide;
N-Ethyl-1-(9-methyl-3-(tetrahydro-2H-pyran-4-yl)-2,3,4,9-tetrahydro-1H-carbazole-6-carbonyl)pyrrolidine-3-carboxamide;
N-Cyclopropyl-2-((3R)-1-(9-methyl-3-(tetrahydro-2H-pyran-4-yl)-2,3,4,9-tetrahydro-1H-carbazole-6-carbonyl)pyrrolidin-3-yl)acetamide;
N-((3S)-1-(9-Methyl-3-(tetrahydro-2H-pyran-4-yl)-2,3,4,9-tetrahydro-1H-carbazole-6-carbonyl)pyrrolidin-3-yl)cyclopropanecarboxamide;
(3S)—N-(2-Fluoroethyl)-1-(9-methyl-3-(tetrahydro-2H-pyran-4-yl)-2,3,4,9-tetrahydro-1H-carbazole-6-carbonyl)pyrrolidine-3-carboxamide;
(3S)—N-(Cyclopropylmethyl)-1-(9-methyl-3-(tetrahydro-2H-pyran-4-yl)-2,3,4,9-tetrahydro-1H-carbazole-6-carbonyl)pyrrolidine-3-carboxamide;
N-(1-(9-Methyl-3-(tetrahydro-2H-1-pyran-4-yl)-2,3,4,9-tetrahydro-1H-carbazole-6-carbonyl)piperidin-4-yl)cyclopropanecarboxamide;
N-(1-(9-Methyl-3-(tetrahydro-2H-pyran-4-yl)-2,3,4,9-tetrahydro-1H-carbazole-6-carbonyl)piperidin-3-yl)cyclopropanecarboxamide;
(3S)—N-(2,2-Difluoroethyl)-1-(9-methyl-3-(tetrahydro-2H-pyran-4-yl)-2,3,4,9-tetrahydro-1H-carbazole-6-carbonyl)pyrrolidine-3-carboxamide;
(3S)—N-Ethyl-1-(9-methyl-3-(tetrahydro-2H-pyran-4-yl)-2,3,4,9-tetrahydro-1H-carbazole-6-carbonyl)pyrrolidine-3-carboxamide;
N-(1-(9-Methyl-3-(tetrahydro-2H-pyran-4-yl)-2,3,4,9-tetrahydro-1H-carbazole-6-carbonyl)piperidin-3-yl)propionamide;
N-(1-(9-Methyl-3-(tetrahydro-2H-pyran-4-yl)-2,3,4,9-tetrahydro-1H-carbazole-6-carbonyl)piperidin-3-yl)isobutyramide;
2-Cyclopropyl-N-(1-(9-methyl-3-(tetrahydro-2H-pyran-4-yl)-2,3,4,9-tetrahydro-1H-carbazole-6-carbonyl)piperidin-3-yl)acetamide;
N-(4-(2-Hydroxyethylamino)-4-oxobutyl)-N,9-dimethyl-3-(tetrahydro-2H-pyran-4-yl)-2,3,4,9-tetrahydro-1H-carbazole-6-carboxamide;
N-((3S)-1-(9-Methyl-3-(tetrahydro-2H-pyran-4-yl)-2,3,4,9-tetrahydro-1H-carbazole-6-carbonyl)piperidin-3-yl)cyclopropanecarboxamide;
N,9-Dimethyl-N-(4-oxo-4-((S)-tetrahydrofuran-3-ylamino)butyl)-3-(tetrahydro-2H-pyran-4-yl)-2,3,4,9-tetrahydro-1H-carbazole-6-carboxamide;
N,9-Dimethyl-N-(4-(oxetan-3-ylamino)-4-oxobutyl)-3-(tetrahydro-2H-pyran-4-yl)-2,3,4,9-tetrahydro-1H-carbazole-6-carboxamide;
N-(4-(3-Hydroxypropylamino)-4-oxobutyl)-N,9-dimethyl-3-(tetrahydro-2H-pyran-4-yl)-2,3,4,9-tetrahydro-1H-carbazole-6-carboxamide;
N-Ethyl-9-(ethylsulfonyl)-N-(2-(2-hydroxyethylamino)-2-oxoethyl)-3-(tetrahydro-2H-pyran-4-yl)-2,3,4,9-tetrahydro-1H-carbazole-6-carboxamide;
(9-(Ethylsulfonyl)-3-(tetrahydro-2H-pyran-4-yl)-2,3,4,9-tetrahydro-1H-carbazol-6-yl)((R)-3-hydroxypyrrolidin-1-yl)methanone;
(9-(Ethylsulfonyl)-3-(tetrahydro-2H-pyran-4-yl)-2,3,4,9-tetrahydro-1H-carbazol-6-yl)((S)-3-hydroxypyrrolidin-1-yl)methanone;
(9-(Ethylsulfonyl)-3-(tetrahydro-2H-pyran-4-yl)-2,3,4,9-tetrahydro-1H-carbazol-6-yl)((R)-3-hydroxypiperidin-1-yl)methanone;
(9-(Ethylsulfonyl)-3-(tetrahydro-2H-pyran-4-yl)-2,3,4,9-tetrahydro-1H-carbazol-6-yl)(4-hydroxypiperidin-1-yl)methanone;
N6-Ethyl-N6-(2-(ethylamino)-2-oxoethyl)-N9,N9-dimethyl-3-(tetrahydro-2H-pyran-4-yl)-3,4-dihydro-1H-carbazole-6,9(2H)-dicarboxamide;
N-ethyl-N-(2-(ethylamino)-2-oxoethyl)-3-(tetrahydro-2H-pyran-4-yl)-2,3,4,9-tetrahydro-1H-carbazole-6-carboxamide;
Ethyl 2-(6-(ethyl(2-(ethylamino)-2-oxoethyl)carbamoyl)-3-(tetrahydro-2H-pyran-4-yl)-3,4-dihydro-1H-carbazol-9(2H)-yl)acetate;
2-(6-(Ethyl(2-(ethylamino)-2-oxoethyl)carbamoyl)-3-(tetrahydro-2H-pyran-4-yl)-3,4-dihydro-1H-carbazol-9(2H)-yl)acetic acid;
9-(2-(diethylamino)-2-oxoethyl)-N-ethyl-N-(2-(ethylamino)-2-oxoethyl)-3-(tetrahydro-2H-pyran-4-yl)-2,3,4,9-tetrahydro-1H-carbazole-6-carboxamide;
N-Ethyl-N-(2-(ethylamino)-2-oxoethyl)-9-(2-(methylamino)-2-oxoethyl)-3-(tetrahydro-2H-pyran-4-yl)-2,3,4,9-tetrahydro-1H-carbazole-6-carboxamide;
N-Ethyl-N-(2-(ethylamino)-2-oxoethyl)-9-(2-hydroxy-2-methylpropyl)-3-(tetrahydro-2H-pyran-4-yl)-2,3,4,9-tetrahydro-1H-carbazole-6-carboxamide;
N-Ethyl-N-(2-(ethylamino)-2-oxoethyl)-9-(2-hydroxyethyl)-3-(tetrahydro-2H-pyran-4-yl)-2,3,4,9-tetrahydro-1H-carbazole-6-carboxamide;
2-(N-Ethyl-9-(ethylsulfonyl)-3-(tetrahydro-2H-pyran-4-yl)-2,3,4,9-tetrahydro-1H-carbazole-6-carboxamido)acetic acid;
N-Ethyl-9-(ethylsulfonyl)-N-(2-(2-hydroxypropylamino)-2-oxoethyl)-3-(tetrahydro-2H-pyran-4-yl)-2,3,4,9-tetrahydro-1H-carbazole-6-carboxamide;
N-Ethyl-9-(ethylsulfonyl)-N-(2-(2-methoxyethylamino)-2-oxoethyl)-3-(tetrahydro-2H-pyran-4-yl)-2,3,4,9-tetrahydro-1H-carbazole-6-carboxamide;
N-Ethyl-9-(ethylsulfonyl)-N-(2-(oxetan-3-ylamino)-2-oxo ethyl)-3-(tetrahydro-2H-pyran-4-yl)-2,3,4,9-tetrahydro-1H-carbazole-6-carboxamide;
N-[2-(cyclopropylamino)-2-oxoethyl]-9-(cyclopropylmethyl)-N-ethyl-3-(tetrahydro-2H-pyran-4-yl)-2,3,4,9-tetrahydro-1H-carbazole-6-carboxamide;
9-(Cyclopropylmethyl)-N-ethyl-N-(2-(ethylamino)-2-oxoethyl)-3-(tetrahydro-2H-pyran-4-yl)-2,3,4,9-tetrahydro-1H-carbazole-6-carboxamide;
9-Cyclobutyl-N-ethyl-N-(2-(ethylamino)-2-oxoethyl)-3-(tetrahydro-2H-pyran-4-yl)-2,3,4,9-tetrahydro-1H-carbazole-6-carboxamide;
9-Cyclobutyl-N-ethyl-N-(2-(2-fluoroethylamino)-2-oxoethyl)-3-(tetrahydro-2H-pyran-4-yl)-2,3,4,9-tetrahydro-1H-carbazole-6-carboxamide;
9-Cyclobutyl-N-ethyl-N-(2-(isopropylamino)-2-oxoethyl)-3-(tetrahydro-2H-pyran-4-yl)-2,3,4,9-tetrahydro-1H-carbazole-6-carboxamide;
9-Ethyl-N-methyl-N-(4-(methylamino)-4-oxobutyl)-3-(tetrahydro-2H-pyran-4-yl)-2,3,4,9-tetrahydro-1H-carbazole-6-carboxamide;
9-Ethyl-N-(4-(2-fluoroethylamino)-4-oxobutyl)-N-methyl-3-(tetrahydro-2H-pyran-4-yl)-2,3,4,9-tetrahydro-1H-carbazole-6-carboxamide;
N-(4-(2,2-difluoroethylamino)-4-oxobutyl)-9-ethyl-N-methyl-3-(tetrahydro-2H-pyran-4-yl)-2,3,4,9-tetrahydro-1H-carbazole-6-carboxamide;
9-Ethyl-N-methyl-N-(4-oxo-4-(2,2,2-trifluoroethylamino)butyl)-3-(tetrahydro-2H-pyran-4-yl)-2,3,4,9-tetrahydro-1H-carbazole-6-carboxamide;
9-Ethyl-N-(4-(2-hydroxyethylamino)-4-oxobutyl)-N-methyl-3-(tetrahydro-2H-pyran-4-yl)-2,3,4,9-tetrahydro-1H-carbazole-6-carboxamide;
N-Ethyl-N-(2-(ethylamino)-2-oxoethyl)-9-(2-fluoroethyl)-3-(tetrahydro-2H-pyran-4-yl)-2,3,4,9-tetrahydro-1H-carbazole-6-carboxamide;
N-(4-(Ethylamino)-4-oxobutyl)-9-(2-fluoroethyl)-N-methyl-3-(tetrahydro-2H-pyran-4-yl)-2,3,4,9-tetrahydro-1H-carbazole-6-carboxamide;
N-ethyl-9-(ethylsulfonyl)-N-(2-(2-hydroxyethylamino)-2-oxoethyl)-3-(tetrahydro-2H-pyran-4-yl)-2,3,4,9-tetrahydro-1H-carbazole-6-carboxamide;
N-ethyl-9-(ethylsulfonyl)-N-(2-(3-hydroxypropylamino)-2-oxoethyl)-3-(tetrahydro-2H-pyran-4-yl)-2,3,4,9-tetrahydro-1H-carbazole-6-carboxamide;
N-ethyl-9-(ethylsulfonyl)-N-(2-(3-fluoropropylamino)-2-oxoethyl)-3-(tetrahydro-2H-pyran-4-yl)-2,3,4,9-tetrahydro-1H-carbazole-6-carboxamide;
N-ethyl-9-(ethylsulfonyl)-N-(2-(2-fluoroethylamino)-2-oxoethyl)-3-(tetrahydro-2H-pyran-4-yl)-2,3,4,9-tetrahydro-1H-carbazole-6-carboxamide;
2-(N-ethyl-9-(ethylsulfonyl)-3-(tetrahydro-2H-pyran-4-yl)-2,3,4,9-tetrahydro-1H-carbazole-6-carboxamido)acetic acid;
N-(2-(cyclopropylamino)-2-oxoethyl)-9-(ethylsulfonyl)-N-methyl-3-(tetrahydro-2H-pyran-4-yl)-2,3,4,9-tetrahydro-1H-carbazole-6-carboxamide;
(2R)-1-(9-(ethylsulfonyl)-3-(tetrahydro-2H-pyran-4-yl)-2,3,4,9-tetrahydro-1H-carbazole-6-carbonyl)-N-(2-fluoroethyl)pyrrolidine-2-carboxamide;
N-(2-(2,2-difluoroethylamino)-2-oxoethyl)-N-ethyl-9-methyl-3-(tetrahydro-2H-pyran-4-yl)-2,3,4,9-tetrahydro-1H-carbazole-6-carboxamide;
N-ethyl-N-(2-((R)-2-hydroxypropylamino)-2-oxoethyl)-9-methyl-3-(tetrahydro-2H-pyran-4-yl)-2,3,4,9-tetrahydro-1H-carbazole-6-carboxamide;
N-ethyl-N-(2-((S)-2-hydroxypropylamino)-2-oxoethyl)-9-methyl-3-(tetrahydro-2H-pyran-4-yl)-2,3,4,9-tetrahydro-1H-carbazole-6-carboxamide;
N-ethyl-N-(2-(2-methoxyethylamino)-2-oxoethyl)-9-methyl-3-(tetrahydro-2H-pyran-4-yl)-2,3,4,9-tetrahydro-1H-carbazole-6-carboxamide;
N—((R)-1-(cyclopropylamino)-1-oxopropan-2-yl)-N,9-dimethyl-3-(tetrahydro-2H-pyran-4-yl)-2,3,4,9-tetrahydro-1H-carbazole-6-carboxamide;
(R)—N—((S)-1-Hydroxy-5-(oxetan-3-ylamino)-5-oxopentan-2-yl)-N,9-dimethyl-3-(tetrahydro-2H-pyran-4-yl)-2,3,4,9-tetrahydro-1H-carbazole-6-carboxamide;
(R)—N—((S)-5-(2,2-Difluoroethylamino)-1-hydroxy-5-oxopentan-2-yl)-N,9-dimethyl-3-(tetrahydro-2H-pyran-4-yl)-2,3,4,9-tetrahydro-1H-carbazole-6-carboxamide;
(R)—N—((S)-5-(2-Fluoroethylamino)-1-hydroxy-5-oxopentan-2-yl)-N,9-dimethyl-3-(tetrahydro-2H-pyran-4-yl)-2,3,4,9-tetrahydro-1H-carbazole-6-carboxamide;
(R)—N-(4-(Cyanomethylamino)-4-oxobutyl)-N,9-dim ethyl-3-(tetrahydro-2H-pyran-4-yl)-2,3,4,9-tetrahydro-1H-carbazole-6-carboxamide;
(R)—N—((S)-1-Hydroxy-5-(methylamino)-5-oxopentan-2-yl)-N,9-dimethyl-3-(tetrahydro-2H-pyran-4-yl)-2,3,4,9-tetrahydro-1H-carbazole-6-carboxamide;
(R)—N—((S)-1-Hydroxy-5-(isopropylamino)-5-oxopentan-2-yl)-N,9-dimethyl-3-(tetrahydro-2H-pyran-4-yl)-2,3,4,9-tetrahydro-1H-carbazole-6-carboxamide;
(R)—N—((S)-5-(Ethylamino)-1-hydroxy-5-oxopentan-2-yl)-N,9-dimethyl-3-(tetrahydro-2H-pyran-4-yl)-2,3,4,9-tetrahydro-1H-carbazole-6-carboxamide;
(R)—N-(4-(Methoxyamino)-4-oxobutyl)-N,9-dimethyl-3-(tetrahydro-2H-pyran-4-yl)-2,3,4,9-tetrahydro-1H-carbazole-6-carboxamide;
(R)—N-(4-(2,2-Dimethylhydrazinyl)-4-oxobutyl)-N,9-dimethyl-3-(tetrahydro-2H-pyran-4-yl)-2,3,4,9-tetrahydro-1H-carbazole-6-carboxamide;
(R)—N-(4-(2-Methoxyethylamino)-4-oxobutyl)-N,9-dimethyl-3-(tetrahydro-2H-pyran-4-yl)-2,3,4,9-tetrahydro-1H-carbazole-6-carboxamide;
(R)—N-(4-(1H-Pyrrol-1-ylamino)-4-oxobutyl)-N,9-dim ethyl-3-(tetrahydro-2H-pyran-4-yl)-2,3,4,9-tetrahydro-1H-carbazole-6-carboxamide;
(R)—N-Ethyl-N-(4-(2-hydroxyethylamino)-4-oxobutyl)-9-methyl-3-(tetrahydro-2H-pyran-4-yl)-2,3,4,9-tetrahydro-1H-carbazole-6-carboxamide;
(R)—N-(4-(2-Hydroxyethylamino)-4-oxobutyl)-N,9-dimethyl-3-(tetrahydro-2H-pyran-4-yl)-2,3,4,9-tetrahydro-1H-carbazole-6-carboxamide;
   and pharmaceutically acceptable salts thereof.

It will be understood that when compounds of the present invention contain one or more chiral centers, the compounds of the invention may exist in, and be isolated as, enantiomeric or diastereomeric forms, or as a racemic mixture. The present invention includes any possible enantiomers, diastereomers, racemates or mixtures thereof, of a compound of Formula I. The optically active forms of the compound of the invention may be prepared, for example, by chiral chromatographic separation of a racemate, by synthesis from optically active starting materials or by asymmetric synthesis based on the procedures described thereafter.

It will also be appreciated that certain compounds of the present invention may exist as geometrical isomers, for example E and Z isomers of alkenes. The present invention includes any geometrical isomer of a compound of Formula I. It will further be understood that the present invention encompasses tautomers of the compounds of the Formula I.

It will also be understood that certain compounds of the present invention may exist in solvated, for example hydrated, as well as unsolvated forms. It will further be understood that the present invention encompasses all such solvated forms of the compounds of the Formula I.

Within the scope of the invention are also salts of the compounds of the Formula I. Generally, pharmaceutically acceptable salts of compounds of the present invention may be obtained using standard procedures well known in the art, for example by reacting a sufficiently basic compound, for example an alkyl amine with a suitable acid, for example, HCl or acetic acid, to afford a physiologically acceptable anion. It may also be possible to make a corresponding alkali metal (such as sodium, potassium, or lithium) or an alkaline earth metal (such as a calcium) salt by treating a compound of the present invention having a suitably acidic proton, such as a carboxylic acid or a phenol with one equivalent of an alkali metal or alkaline earth metal hydroxide or alkoxide (such as the ethoxide or methoxide), or a suitably basic organic amine (such as choline or meglumine) in an aqueous medium, followed by conventional purification techniques.

In one embodiment, the compound of Formula I above may be converted to a pharmaceutically acceptable salt or solvate thereof, particularly, an acid addition salt such as a hydrochloride, hydrobromide, phosphate, acetate, fumarate, maleate, tartrate, citrate, methanesulphonate or p-toluenesulphonate.

We have now found that the compounds of the invention have activity as pharmaceuticals, in particular as modulators or ligands such as agonists, partial agonists, inverse agonist or antagonists of CB₁ receptors. More particularly, the compounds of the invention exhibit selective activity as agonist of the CB₁ receptors and are useful in therapy, especially for relief of various pain conditions such as chronic pain, neuropathic pain, acute pain, cancer pain, pain caused by rheumatoid arthritis, migraine, visceral pain etc. This list should however not be interpreted as exhaustive. Additionally, compounds of the present invention are useful in other disease states in which dysfunction of CB₁ receptors is present or implicated. Furthermore, the compounds of the invention may be used to treat cancer, multiple sclerosis, Parkinson's disease, Huntington's chorea, Alzheimer's disease, anxiety disorders, obesity, gastrointestinal disorders and cardiovascular disorders. Even furthermore, the compounds of the invention may be useful in enhancing smoking cessation.

Compounds of the invention are useful as immunomodulators, especially for autoimmune diseases, such as arthritis, for skin grafts, organ transplants and similar surgical needs, for collagen diseases, various allergies, for use as anti-tumour agents and anti viral agents.

Compounds of the invention are useful in disease states where degeneration or dysfunction of cannabinoid receptors is present or implicated in that paradigm. This may involve the use of isotopically labelled versions of the compounds of the invention in diagnostic techniques and imaging applications such as positron emission tomography (PET).

Compounds of the invention are useful for the treatment of diarrhoea, depression, anxiety and stress-related disorders such as post-traumatic stress disorders, panic disorder, generalized anxiety disorder, social phobia, and obsessive compulsive disorder, urinary incontinence, premature ejaculation, various mental illnesses, cough, lung oedema, various gastro-intestinal disorders, e.g. constipation, functional gastrointestinal disorders such as Irritable Bowel Syndrome and Functional Dyspepsia, Parkinson's disease and other motor disorders, traumatic brain injury, stroke, cardioprotection following miocardial infarction, obesity, spinal injury and drug addiction, including the treatment of alcohol, nicotine, opioid and other drug abuse and for disorders of the sympathetic nervous system for example hypertension.

Compounds of the invention are useful as an analgesic agent for use during general anaesthesia and monitored anaesthesia care. Combinations of agents with different properties are often used to achieve a balance of effects needed to maintain the anaesthetic state (e.g. amnesia, analgesia, muscle relaxation and sedation). Included in this combination are inhaled anaesthetics, hypnotics, anxiolytics, neuromuscular blockers and opioids.

Also within the scope of the invention is the use of any of the compounds according to the Formula I above, for the manufacture of a medicament for the treatment of any of the conditions discussed above.

A further aspect of the invention is a method for the treatment of a subject suffering from any of the conditions discussed above, whereby an effective amount of a compound according to the Formula I above, is administered to a patient in need of such treatment.

Thus, the invention provides a compound of Formula I or pharmaceutically acceptable salt or solvate thereof, as hereinbefore defined for use in therapy.

In a further aspect, the present invention provides the use of a compound of Formula I or a pharmaceutically acceptable salt or solvate thereof, as hereinbefore defined in the manufacture of a medicament for use in therapy.

In the context of the present specification, the term “therapy” also includes “prophylaxis” unless there are specific indications to the contrary. The term “therapeutic” and “therapeutically” should be contrued accordingly. The term “therapy” within the context of the present invention further encompasses to administer an effective amount of a compound of the present invention, to mitigate either a pre-existing disease state, acute or chronic, or a recurring condition. This definition also encompasses prophylactic therapies for prevention of recurring conditions and continued therapy for chronic disorders.

The compounds of the present invention are useful in therapy, especially for the therapy of various pain conditions including, but not limited to: acute pain, chronic pain, neuropathic pain, back pain, cancer pain, and visceral pain.

In use for therapy in a warm-blooded animal such as a human, the compound of the invention may be administered in the form of a conventional pharmaceutical composition by any route including orally, intramuscularly, subcutaneously, topically, intranasally, intraperitoneally, intrathoracially, intravenously, epidurally, intrathecally, transdermally, intracerebroventricularly and by injection into the joints.

In one embodiment of the invention, the route of administration may be oral, intravenous or intramuscular.

The dosage will depend on the route of administration, the severity of the disease, age and weight of the patient and other factors normally considered by the attending physician, when determining the individual regimen and dosage level at the most appropriate for a particular patient.

For preparing pharmaceutical compositions from the compounds of this invention, inert, pharmaceutically acceptable carriers can be either solid and liquid. Solid form preparations include powders, tablets, dispersible granules, capsules, cachets, and suppositories.

A solid carrier can be one or more substances, which may also act as diluents, flavoring agents, solubilizers, lubricants, suspending agents, binders, or table disintegrating agents; it can also be an encapsulating material.

In powders, the carrier is a finely divided solid, which is in a mixture with the finely divided compound of the invention, or the active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture in then poured into convenient sized moulds and allowed to cool and solidify.

Suitable carriers are magnesium carbonate, magnesium stearate, talc, lactose, sugar, pectin, dextrin, starch, tragacanth, methyl cellulose, sodium carboxymethyl cellulose, a low-melting wax, cocoa butter, and the like.

The term composition is also intended to include the formulation of the active component with encapsulating material as a carrier providing a capsule in which the active component (with or without other carriers) is surrounded by a carrier which is thus in association with it. Similarly, cachets are included.

Tablets, powders, cachets, and capsules can be used as solid dosage forms suitable for oral administration.

Liquid form compositions include solutions, suspensions, and emulsions. For example, sterile water or water propylene glycol solutions of the active compounds may be liquid preparations suitable for parenteral administration. Liquid compositions can also be formulated in solution in aqueous polyethylene glycol solution.

Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art.

Depending on the mode of administration, the pharmaceutical composition will preferably include from 0.05% to 99% w (percent by weight), more preferably from 0.10 to 50% w, of the compound of the invention, all percentages by weight being based on total composition.

A therapeutically effective amount for the practice of the present invention may be determined, by the use of known criteria including the age, weight and response of the individual patient, and interpreted within the context of the disease which is being treated or which is being prevented, by one of ordinary skills in the art.

Within the scope of the invention is the use of any compound of Formula I as defined above for the manufacture of a medicament.

Also within the scope of the invention is the use of any compound of Formula I for the manufacture of a medicament for the therapy of pain.

Additionally provided is the use of any compound according to Formula I for the manufacture of a medicament for the therapy of various pain conditions including, but not limited to: acute pain, chronic pain, neuropathic pain, back pain, cancer pain, and visceral pain.

A further aspect of the invention is a method for therapy of a subject suffering from any of the conditions discussed above, whereby an effective amount of a compound according to the Formula I above, is administered to a patient in need of such therapy.

Additionally, there is provided a pharmaceutical composition comprising a compound of Formula I or a pharmaceutically acceptable salt thereof, in association with a pharmaceutically acceptable carrier.

Particularly, there is provided a pharmaceutical composition comprising a compound of Formula I or a pharmaceutically acceptable salt thereof, in association with a pharmaceutically acceptable carrier for therapy, more particularly for therapy of pain.

Further, there is provided a pharmaceutical composition comprising a compound of Formula I or a pharmaceutically acceptable salt thereof, in association with a pharmaceutically acceptable carrier use in any of the conditions discussed above.

In a further aspect, the present invention provides a method of preparing the compounds of the present invention.

In one embodiment, the invention provides a process for preparing a compound of Formula I comprising: reacting a compound of Formula II with Y—H,

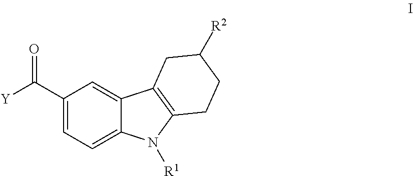

comprising reacting a compound of Formula II with Y—H,

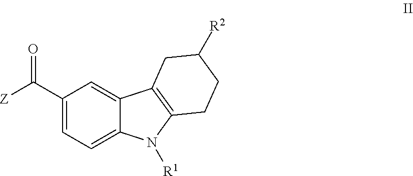

wherein

Y, R¹ and R² are defined above; and

Z is a halogen or —OH.

Optionally, the step of reacting a compound of formula II with a compound of Y—H is carried out in the presence of a coupling reagent, such as HATU, and an amine base, such as DIPEA.

In another embodiment, the invention provides a process for preparing a compound of Formula I

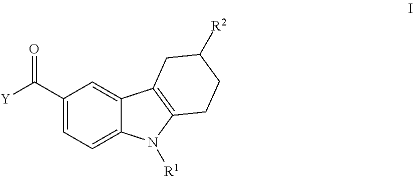

comprising reacting a compound of formula III with R¹—X¹,

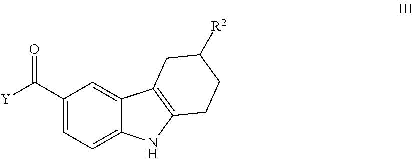

wherein,

X¹ is selected from halogen and OH; and R², R¹ and Y are defined above.

Optionally, the step of reacting a compound of formula III with a compound of R¹—X¹ is carried out in the presence of a base, such as sodium hydride, sodium borohydride, aluminum hydride, sodium aluminum hydride, alkaline metal hydride, alkaline earth metal hydride or equivalence thereof.

### Biological Evaluation

hCB₁ and hCB₂ Receptor Binding

Human CB₁ receptor from Receptor Biology (hCB₁) or human CB₂ receptor from BioSignal (hCB₂) membranes are thawed at 37° C., passed 3 times through a 25-gauge blunt-end needle, diluted in the cannabinoid binding buffer (50 mM Tris, 2.5 mM EDTA, 5 mM MgCl₂, and 0.5 mg/mL BSA fatty acid free, pH 7.4) and aliquots containing the appropriate amount of protein are distributed in 96-well plates. The IC₅₀ of the compounds of the invention at hCB₁ and hCB₂ are evaluated from 10-point dose-response curves done with ³H-CP55,940 at 20000 to 25000 dpm per well (0.17-0.21 nM) in a final volume of 300 μl. The total and non-specific binding are determined in the absence and presence of 0.2 μM of HU210 respectively. The plates are vortexed and incubated for 60 minutes at room temperature, filtered through Unifilters GF/B (presoaked in 0.1% polyethyleneimine) with the Tomtec or Packard harvester using 3 mL of wash buffer (50 mM Tris, 5 mM MgCl₂, 0.5 mg BSA pH 7.0). The filters are dried for 1 hour at 55° C. The radioactivity (cpm) is counted in a TopCount (Packard) after adding 65 μl/well of MS-20 scintillation liquid.

hCB₁ and hCB₂GTPγS Binding

Human CB₁ receptor from Receptor Biology (hCB₁) or human CB₂ receptor membranes (BioSignal) are thawed at 37° C., passed 3 times through a 25-gauge blunt-end needle and diluted in the GTPγS binding buffer (50 mM Hepes, 20 mM NaOH, 100 mM NaCl, 1 mM EDTA, 5 mM MgCl₂, pH 7.4, 0.1% BSA). The EC₅₀ and Eₘₐₓ of the compounds of the invention are evaluated from 10-point dose-response curves done in 300 μl with the appropriate amount of membrane protein and 100000-130000 dpm of GTPg³⁵S per well (0.11-0.14 nM). The basal and maximal stimulated binding is determined in absence and presence of 1 μM (hCB₂) or 10 μM (hCB₁) Win 55,212-2 respectively. The membranes are pre-incubated for 5 minutes with 56.25 μM (hCB2) or 112.5 μM (hCB₁) GDP prior to distribution in plates (15 μM (hCB₂) or 30 μM (hCB₁) GDP final). The plates are vortexed and incubated for 60 minutes at room temperature, filtered on Unifilters GF/B (presoaked in water) with the Tomtec or Packard harvester using 3 ml of wash buffer (50 mM Tris, 5 mM MgCl₂, 50 mM NaCl, pH 7.0). The filters are dried for 1 hour at 55° C. The radioactivity (cpm) is counted in a TopCount (Packard) after adding 65 μl/well of MS-20 scintillation liquid. Antagonist reversal studies are done in the same way except that (a) an agonist dose-response curve is done in the presence of a constant concentration of antagonist, or (b) an antagonist dose-response curve is done in the presence of a constant concentration of agonist.

Based on the above assays, the dissociation constant (Ki) for a particular compound of the invention towards a particular receptor is determined using the following equation:

Ki=IC₅₀/(1+[rad]/Kd),

Wherein

IC₅₀ is the concentration of the compound of the invention at which 50% displacement has been observed;

[rad] is a standard or reference radioactive ligand concentration at that moment; and

Kd is the dissociation constant of the radioactive ligand towards the particular receptor.

Using the above-mentioned assays, the compounds of the invention are found to be active towards human CB₁ receptors.

In addition, certain compounds of the invention are tested using one or more assays shown above and the test results are summarized in Table 1 below.

TABLE 1
Certain Biological Activities of Certain Compounds of the Invention

| | | hCB1 | hCB1 |
|---|---|---|---|
| | hCB1 Ki | EC50 | EMax |
| Compound | (nM) | (nM) | (%) |
| (6-cyclohexyl-9-methyl-5,6,7,8- | 29.5 | 65.1 | 106 |
| tetrahydrocarbazol-3-yl)-(4-methyl- | | | |
| 1-piperidyl)methanone | | | |
| (6-cyclohexyl-9-ethyl-5,6,7,8- | 82.3 | 233.6 | 90 |
| tetrahydrocarbazol-3-yl)-(4-methyl- | | | |
| 1-piperidyl)methanone | | | |
| (6-cyclohexyl-9-methylsulfonyl- | 75.3 | 276.6 | 89 |
| 5,6,7,8-tetrahydrocarbazol-3-yl)-(4- | | | |
| methyl-1-piperidyl)methanone | | | |
| (6-cyclohexyl-9-ethylsulfonyl- | 15.2 | 20 | 107 |
| 5,6,7,8-tetrahydrocarbazol-3-yl)-(4- | | | |
| methyl-1-piperidyl)methanone | | | |
| 6-cyclohexyl-N- | 7.9 | 31.3 | 111 |
| (cyclopropylcarbamoylmethyl)-N- | | | |
| methyl-9-methylsulfonyl-5,6,7,8- | | | |
| tetrahydrocarbazole-3-carboxamide | | | |
| 6-cyclohexyl-N- | 21.2 | 43.9 | 110 |
| (cyclopropylcarbamoylmethyl)-N- | | | |
| methyl-9-propan-2-ylsulfonyl- | | | |
| 5,6,7,8-tetrahydrocarbazole-3- | | | |
| carboxamide | | | |
| N-(cyclopropylcarbamoylmethyl)- | 44.5 | 95.3 | 121 |
| N,9-dimethyl-6-(oxan-4-yl)-5,6,7,8- | | | |
| tetrahydrocarbazole-3-carboxamide | | | |
| N-ethyl-N-(ethylcarbamoylmethyl)- | 17.4 | 33.1 | 121 |
| 9-methyl-6-(oxan-4-yl)-5,6,7,8- | | | |
| tetrahydrocarbazole-3-carboxamide | | | |
| N-ethyl-9-methyl-6-(oxan-4-yl)-N- | 31.3 | 41.7 | 123 |
| (propan-2-ylcarbamoylmethyl)- | | | |
| 5,6,7,8-tetrahydrocarbazole-3- | | | |
| carboxamide | | | |
| N-(cyclopropylcarbamoylmethyl)- | 24.5 | 60.8 | 121 |
| N,9-dimethyl-6-(oxan-4-yl)-5,6,7,8- | | | |
| tetrahydrocarbazole-3-carboxamide | | | |
| N-(cyclopropylcarbamoylmethyl)- | 176.5 | 530.7 | 107 |
| N,9-dimethyl-6-(oxan-4-yl)-5,6,7,8- | | | |
| tetrahydrocarbazole-3-carboxamide | | | |
| N-[3-(cyclopropylcarbamoyl)propyl]- | 7.1 | 16.2 | 113 |
| N,9-dimethyl-6-(oxan-4-yl)-5,6,7,8- | | | |
| tetrahydrocarbazole-3-carboxamide | | | |
| N,9-dimethyl-N-[3- | 53.7 | 78.9 | 121 |
| (methylcarbamoyl)propyl]-6-(oxan- | | | |
| 4-yl)-5,6,7,8-tetrahydrocarbazole-3- | | | |
| carboxamide | | | |
| N-[3-(2- | 28.7 | 49.2 | 112 |
| fluoroethylcarbamoyl)propyl]-N,9- | | | |
| dimethyl-6-(oxan-4-yl)-5,6,7,8- | | | |
| tetrahydrocarbazole-3-carboxamide | | | |
| N-[3-(ethylcarbamoyl)propyl]-N,9- | 36.6 | 63.5 | 111 |
| dimethyl-6-(oxan-4-yl)-5,6,7,8- | | | |
| tetrahydrocarbazole-3-carboxamide | | | |
| N-ethyl-N-(2- | 17.5 | 44.4 | 111 |
| fluoroethylcarbamoylmethyl)-9- | | | |
| methyl-6-(oxan-4-yl)-5,6,7,8- | | | |
| tetrahydrocarbazole-3-carboxamide | | | |
| N-(cyclopropylcarbamoylmethyl)-N- | 7.3 | 6.2 | 106 |
| ethyl-9-methyl-6-(oxan-4-yl)-5,6,7,8- | | | |
| tetrahydrocarbazole-3-carboxamide | | | |
| N-cyclopropyl-1-[9-methyl-6-(oxan- | 117.7 | 214 | 102 |
| 4-yl)5,6,7,8-tetrahydrocarbazole-3- | | | |
| carbonyl]piperidine-4-carboxamide | | | |
| N-cyclopropyl-2-[1-[9-methyl-6- | 2079.1 | | |
| (oxan-4-yl)5,6,7,8- | | | |
| tetrahydrocarbazole-3- | | | |
| carbonyl]azetidin-3-yl]acetamide | | | |
| N-ethyl-N-(2-hydroxyethyl)-9- | 389.6 | | |
| methyl-6-(oxan-4-yl)-5,6,7,8- | | | |
| tetrahydrocarbazole-3-carboxamide | | | |
| N-[2-(cyclopropylcarbamoyl-methyl- | 77.5 | 468.3 | 110 |
| amino)ethyl]-N,9-dimethyl-6-(oxan- | | | |
| 4-yl)-5,6,7,8-tetrahydrocarbazole-3- | | | |
| carboxamide | | | |
| N,9-dimethyl-N-(2- | >8720.0 | | |
| methylaminoethyl)-6-(oxan-4-yl)- | | | |
| 5,6,7,8-tetrahydrocarbazole-3- | | | |
| carboxamide | | | |
| N-[2-(cyclopropylthiocarbamoyl- | 9.9 | 161.4 | 106 |
| methyl-amino)ethyl]-N,9-dimethyl-6- | | | |
| (oxan-4-yl)-5,6,7,8- | | | |
| tetrahydrocarbazole-3-carboxamide | | | |
| N-(2-fluoroethylcarbamoylmethyl)- | 291.2 | 720.7 | 110 |
| N,9-dimethyl-6-(oxan-4-yl)-5,6,7,8- | | | |
| tetrahydrocarbazole-3-carboxamide | | | |
| N-[2-(cyclopropylcarbamoyl)ethyl]- | 655 | | |
| N,9-dimethyl-6-(oxan-4-yl)-5,6,7,8- | | | |
| tetrahydrocarbazole-3-carboxamide | | | |
| N-ethyl-9-methyl-N- | 140.3 | 342.5 | 116 |
| (methylcarbamoylmethyl)-6-(oxan-4- | | | |
| yl)-5,6,7,8-tetrahydrocarbazole-3- | | | |
| carboxamide | | | |
| methyl N-methyl-N-[2-[methyl-[9- | | | |
| methyl-6-(oxan-4-yl)5,6,7,8- | | | |
| tetrahydrocarbazole-3- | | | |
| carbonyl]amino]ethyl]carbamate | | | |
| N-[2-(cyclopropanecarbonyl-methyl- | | | |
| amino)ethyl]-N,9-dimethyl-6-(oxan- | | | |
| 4-yl)-5,6,7,8-tetrahydrocarbazole-3- | | | |
| carboxamide | | | |
| N-[3-(cyclopropylcarbamoyl)propyl]- | 2.8 | 4.9 | 116 |
| N-ethyl-9-methyl-6-(oxan-4-yl)- | | | |
| 5,6,7,8-tetrahydrocarbazole-3- | | | |
| carboxamide | | | |
| N-cyclopropyl-1-[9-methyl-6-(oxan- | 32.4 | 127.5 | 115 |
| 4-yl)5,6,7,8-tetrahydrocarbazole-3- | | | |
| carbonyl]piperidine-3-carboxamide | | | |
| N-cyclopropyl-1-[9-methyl-6-(oxan- | 106.8 | 273.7 | 121 |
| 4-yl)5,6,7,8-tetrahydrocarbazole-3- | | | |
| carbonyl]azetidine-3-carboxamide | | | |
| N-ethyl-N-[3-(2- | 10.1 | 9.2 | 135 |
| fluoroethylcarbamoyl)propyl]-9- | | | |
| methyl-6-(oxan-4-yl)-5,6,7,8- | | | |
| tetrahydrocarbazole-3-carboxamide | | | |
| N-[(1- | >1166.1 | | |
| cyanocyclopropyl)carbamoylmethyl]- | | | |
| N-ethyl-9-methyl-6-(oxan-4-yl)- | | | |
| 5,6,7,8-tetrahydrocarbazole-3- | | | |
| carboxamide | | | |
| N-(2-fluoroethyl)-9-methyl-6-(oxan- | 659.3 | | |
| 4-yl)-5,6,7,8-tetrahydrocarbazole-3- | | | |
| carboxamide | | | |
| (3S)-N-cyclopropyl-1-[9-methyl-6- | 18.9 | 63.5 | 118 |
| (oxan-4-yl)5,6,7,8- | | | |
| tetrahydrocarbazole-3- | | | |
| carbonyl]piperidine-3-carboxamide | | | |
| N-ethyl-N-(2- | 50.3 | 123.8 | 108 |
| fluoroethylcarbamoylmethyl)-9- | | | |
| methyl-6-(oxan-4-yl)-5,6,7,8- | | | |
| tetrahydrocarbazole-3-carboxamide | | | |
| N-(cyclopropylcarbamoylmethyl)-N- | 4.2 | 1.2 | 124 |
| ethyl-9-methyl-6-(oxan-4-yl)-5,6,7,8- | | | |
| tetrahydrocarbazole-3-carboxamide | | | |
| N,9-dimethyl-N-[3- | 576.2 | | |
| (methylcarbamoyl)propyl]-6-(oxan- | | | |
| 4-yl)-5,6,7,8-tetrahydrocarbazole-3- | | | |
| carboxamide | | | |
| N-ethyl-N-(2- | 11.4 | 15.9 | 119 |
| fluoroethylcarbamoylmethyl)-9- | | | |
| methyl-6-(oxan-4-yl)-5,6,7,8- | | | |
| tetrahydrocarbazole-3-carboxamide | | | |
| N-(cyclopropylcarbamoylmethyl)-N- | 17.3 | 40.5 | 118 |
| ethyl-9-methyl-6-(oxan-4-yl)-5,6,7,8- | | | |
| tetrahydrocarbazole-3-carboxamide | | | |
| N,9-dimethyl-N-[3- | 15 | 21 | 102 |
| (methylcarbamoyl)propyl]-6-(oxan- | | | |
| 4-yl)-5,6,7,8-tetrahydrocarbazole-3- | | | |
| carboxamide | | | |
| N-[3-(2- | 182.3 | 376.6 | 121 |
| fluoroethylcarbamoyl)propyl]-N,9- | | | |
| dimethyl-6-(oxan-4-yl)-5,6,7,8- | | | |
| tetrahydrocarbazole-3-carboxamide | | | |
| N-[3-(2- | 9.5 | 16 | 131 |
| fluoroethylcarbamoyl)propyl]-N,9- | | | |
| dimethyl-6-(oxan-4-yl)-5,6,7,8- | | | |
| tetrahydrocarbazole-3-carboxamide | | | |
| N-[3-(cyclopropylcarbamoyl)propyl]- | 1444.9 | | |
| 9-methyl-6-(oxan-4-yl)-5,6,7,8- | | | |
| tetrahydrocarbazole-3-carboxamide | | | |
| N-[1- | 371.3 | | |
| (cyclopropylcarbamoyl)cyclopropyl]- | | | |
| N,9-dimethyl-6-(oxan-4-yl)-5,6,7,8- | | | |
| tetrahydrocarbazole-3-carboxamide | | | |
| N-ethyl-N-(2-hydroxyethyl)-9- | 976.9 | | |
| methyl-6-(oxan-4-yl)-5,6,7,8- | | | |
| tetrahydrocarbazole-3-carboxamide | | | |
| N-ethyl-N-(2-hydroxyethyl)-9- | 395.7 | | |
| methyl-6-(oxan-4-yl)-5,6,7,8- | | | |
| tetrahydrocarbazole-3-carboxamide | | | |
| N-cyclopropyl-1-[9-methyl-6-(oxan- | 26 | 18.5 | 121 |
| 4-yl)5,6,7,8-tetrahydrocarbazole-3- | | | |
| carbonyl]pyrrolidine-3-carboxamide | | | |
| (3S)-N-cyclopropyl-1-[9-methyl-6- | 59 | 353.1 | 99 |
| (oxan-4-yl)5,6,7,8- | | | |
| tetrahydrocarbazole-3- | | | |
| carbonyl]piperidine-3-carboxamide | | | |
| (3S)-N-cyclopropyl-1-[9-methyl-6- | 21.3 | 56.5 | 117 |
| (oxan-4-yl)5,6,7,8- | | | |
| tetrahydrocarbazole-3- | | | |
| carbonyl]piperidine-3-carboxamide | | | |
| N-ethyl-9-ethylsulfonyl-N-(2- | 48.6 | 76.4 | 111 |
| hydroxyethylcarbamoylmethyl)-6- | | | |
| (oxan-4-yl)-5,6,7,8- | | | |
| tetrahydrocarbazole-3-carboxamide | | | |
| N-[(1S)-1- | >3529.0 | | |
| (cyclopropylcarbamoyl)ethyl]-N,9- | | | |
| dimethyl-6-(oxan-4-yl)-5,6,7,8- | | | |
| tetrahydrocarbazole-3-carboxamide | | | |
| N-ethyl-9-ethylsulfonyl-N-(3- | 45.8 | 53.3 | 126 |
| hydroxypropylcarbamoylmethyl)-6- | | | |
| (oxan-4-yl)-5,6,7,8- | | | |
| tetrahydrocarbazole-3-carboxamide | | | |
| N-ethyl-9-ethylsulfonyl-N-(3- | 24 | 33 | 120 |
| fluoropropylcarbamoylmethyl)-6- | | | |
| (oxan-4-yl)-5,6,7,8- | | | |
| tetrahydrocarbazole-3-carboxamide | | | |
| N-(cyanomethylcarbamoylmethyl)- | 332.8 | | |
| N-ethyl-9-methyl-6-(oxan-4-yl)- | | | |
| 5,6,7,8-tetrahydrocarbazole-3- | | | |
| carboxamide | | | |
| N-[(1S)-1-(2- | >1403.9 | | |
| fluoroethylcarbamoyl)ethyl]-N,9- | | | |
| dimethyl-6-(oxan-4-yl)-5,6,7,8- | | | |
| tetrahydrocarbazole-3-carboxamide | | | |
| N-(cyclopropylcarbamoylmethyl)-9- | 18.1 | 18.8 | 116 |
| ethylsulfonyl-N-methyl-6-(oxan-4- | | | |
| yl)-5,6,7,8-tetrahydrocarbazole-3- | | | |
| carboxamide | | | |
| (3S)-N-cyclopropyl-1-[9-methyl-6- | 10.3 | 13.1 | 134 |
| (oxan-4-yl)5,6,7,8- | | | |
| tetrahydrocarbazole-3- | | | |
| carbonyl]pyrrolidine-3-carboxamide | | | |
| (3S)-N-cyclopropyl-1-[9-methyl-6- | 77.1 | 159.7 | 127 |
| (oxan-4-yl)5,6,7,8- | | | |
| tetrahydrocarbazole-3- | | | |
| carbonyl]pyrrolidine-3-carboxamide | | | |
| (3R)-N-cyclopropyl-1-[9-methyl-6- | >1403.9 | | |
| (oxan-4-yl)5,6,7,8- | | | |
| tetrahydrocarbazole-3- | | | |
| carbonyl]pyrrolidine-3-carboxamide | | | |
| (3R)-N-cyclopropyl-1-[9-methyl-6- | 1919.6 | | |
| (oxan-4-yl)5,6,7,8- | | | |
| tetrahydrocarbazole-3- | | | |
| carbonyl]pyrrolidine-3-carboxamide | | | |
| N-(2-fluoroethyl)-1-[9-methyl-6- | 125.7 | 262.5 | 121 |
| (oxan-4-yl)5,6,7,8- | | | |
| tetrahydrocarbazole-3- | | | |
| carbonyl]pyrrolidine-3-carboxamide | | | |
| N-[(1R)-1- | 19.6 | 29.6 | 120 |
| (cyclopropylcarbamoyl)ethyl]-N,9- | | | |
| dimethyl-6-(oxan-4-yl)-5,6,7,8- | | | |
| tetrahydrocarbazole-3-carboxamide | | | |
| N-ethyl-1-[9-methyl-6-(oxan-4- | 163.3 | 324.4 | 119 |
| yl)5,6,7,8-tetrahydrocarbazole-3- | | | |
| carbonyl]pyrrolidine-3-carboxamide | | | |
| 9-(cyclopropylmethyl)-N-ethyl-N- | 65.2 | 157.5 | 90 |
| (ethylcarbamoylmethyl)-6-(oxan-4- | | | |
| yl)-5,6,7,8-tetrahydrocarbazole-3- | | | |
| carboxamide | | | |
| N-(cyclopropylcarbamoylmethyl)-9- | 21.2 | 34.2 | 91 |
| (cyclopropylmethyl)-N-ethyl-6- | | | |
| (oxan-4-yl)-5,6,7,8- | | | |
| tetrahydrocarbazole-3-carboxamide | | | |
| [9-ethylsulfonyl-6-(oxan-4-yl)- | | >3157.5 | >89 |
| 5,6,7,8-tetrahydrocarbazol-3-yl]- | | | |
| [(3R)-3-hydroxypyrrolidin-1- | | | |
| yl]methanone | | | |
| [9-ethylsulfonyl-6-(oxan-4-yl)- | | 4557.5 | 105 |
| 5,6,7,8-tetrahydrocarbazol-3-yl]- | | | |
| [(3S)-3-hydroxypyrrolidin-1- | | | |
| yl]methanone | | | |
| N-ethyl-9-ethylsulfonyl-N-(2- | 12.9 | 12.9 | 107 |
| fluoroethylcarbamoylmethyl)-6- | | | |
| (oxan-4-yl)-5,6,7,8- | | | |
| tetrahydrocarbazole-3-carboxamide | | | |
| N-cyclopropyl-2-[(3R)-1-[9-methyl- | 35.5 | 69.2 | 115 |
| 6-(oxan-4-yl)5,6,7,8- | | | |
| tetrahydrocarbazole-3- | | | |
| carbonyl]pyrrolidin-3-yl]acetamide | | | |
| [9-ethylsulfonyl-6-(oxan-4-yl)- | | 1760.8 | 104 |
| 5,6,7,8-tetrahydrocarbazol-3-yl]- | | | |
| [(3R)-3-hydroxy-1- | | | |
| piperidyl]methanone | | | |
| [9-ethylsulfonyl-6-(oxan-4-yl)- | 155.2 | 351.7 | 107 |
| 5,6,7,8-tetrahydrocarbazol-3-yl]-(4- | | | |
| hydroxy-1-piperidyl)methanone | | | |
| N-[(3S)-1-[9-methyl-6-(oxan-4- | | 1154 | 116 |
| yl)5,6,7,8-tetrahydrocarbazole-3- | | | |
| carbonyl]pyrrolidin-3- | | | |
| yl]cyclopropanecarboxamide | | | |
| N-[(1R)-1-(2- | 157.2 | 352.5 | 107 |
| fluoroethylcarbamoyl)ethyl]-N,9- | | | |
| dimethyl-6-(oxan-4-yl)-5,6,7,8- | | | |
| tetrahydrocarbazole-3-carboxamide | | | |
| (3S)-N-(2-fluoroethyl)-1-[9-methyl- | 30.5 | 91 | 119 |
| 6-(oxan-4-yl)5,6,7,8- | | | |
| tetrahydrocarbazole-3- | | | |
| carbonyl]pyrrolidine-3-carboxamide | | | |
| (3S)-N-(2-fluoroethyl)-1-[9-methyl- | | 1826.1 | 113 |
| 6-(oxan-4-yl)5,6,7,8- | | | |
| tetrahydrocarbazole-3- | | | |
| carbonyl]pyrrolidine-3-carboxamide | | | |
| 9-cyclobutyl-N-ethyl-N- | 113.5 | 233.3 | 82 |
| (ethylcarbamoylmethyl)-6-(oxan-4- | | | |
| yl)-5,6,7,8-tetrahydrocarbazole-3- | | | |
| carboxamide | | | |
| 9-cyclobutyl-N-ethyl-N-(2- | 130.7 | 279.1 | 80 |
| fluoroethylcarbamoylmethyl)-6- | | | |
| (oxan-4-yl)-5,6,7,8- | | | |
| tetrahydrocarbazole-3-carboxamide | | | |
| 9-cyclobutyl-N-ethyl-6-(oxan-4-yl)- | 151.6 | 292.9 | 70 |
| N-(propan-2-ylcarbamoylmethyl)- | | | |
| 5,6,7,8-tetrahydrocarbazole-3- | | | |
| carboxamide | | | |
| N-[(1R)-1-(ethylcarbamoyl)ethyl]- | 77.1 | 237.1 | 112 |
| N,9-dimethyl-6-(oxan-4-yl)-5,6,7,8- | | | |
| tetrahydrocarbazole-3-carboxamide | | | |
| N-ethyl-N-(4-hydroxybutyl)-9- | 72.3 | 274.7 | 122 |
| methyl-6-(oxan-4-yl)-5,6,7,8- | | | |
| tetrahydrocarbazole-3-carboxamide | | | |
| (2R)-1-[9-ethylsulfonyl-6-(oxan-4- | | 1704 | >107 |
| yl)5,6,7,8-tetrahydrocarbazole-3- | | | |
| carbonyl]-N-(2- | | | |
| fluoroethyl)pyrrolidine-2- | | | |
| carboxamide | | | |
| (3S)-N-(cyclopropylmethyl)-1-[9- | 108.8 | 250.6 | 117 |
| methyl-6-(oxan-4-yl)5,6,7,8- | | | |
| tetrahydrocarbazole-3- | | | |
| carbonyl]pyrrolidine-3-carboxamide | | | |
| N-ethyl-9-ethylsulfonyl-N-(2- | 27 | 66.9 | 115 |
| hydroxyethylcarbamoylmethyl)-6- | | | |
| (oxan-4-yl)-5,6,7,8- | | | |
| tetrahydrocarbazole-3-carboxamide | | | |
| N-ethyl-9-ethylsulfonyl-6-(oxan-4- | 25.1 | 51.4 | 112 |
| yl)-N-(2,2,2- | | | |
| trifluoroethylcarbamoylmethyl)- | | | |
| 5,6,7,8-tetrahydrocarbazole-3- | | | |
| carboxamide | | | |
| N-ethyl-9-ethylsulfonyl-N-(2- | 173.5 | 429.7 | 111 |
| hydroxyethylcarbamoylmethyl)-6- | | | |
| (oxan-4-yl)-5,6,7,8- | | | |
| tetrahydrocarbazole-3-carboxamide | | | |
| N-(2,2- | 17.1 | 39.9 | 116 |
| difluoroethylcarbamoylmethyl)-N- | | | |
| ethyl-9-methyl-6-(oxan-4-yl)-5,6,7,8- | | | |
| tetrahydrocarbazole-3-carboxamide | | | |
| 9-ethyl-N-methyl-N-[3- | 59.2 | 146.2 | 121 |
| (methylcarbamoyl)propyl]-6-(oxan- | | | |
| 4-yl)-5,6,7,8-tetrahydrocarbazole-3- | | | |
| carboxamide | | | |
| 9-ethyl-N-[3-(2- | 25.8 | 60.3 | 119 |
| fluoroethylcarbamoyl)propyl]-N- | | | |
| methyl-6-(oxan-4-yl)-5,6,7,8- | | | |
| tetrahydrocarbazole-3-carboxamide | | | |
| N-[3-(2,2- | 45.2 | 98.3 | 114 |
| difluoroethylcarbamoyl)propyl]-9- | | | |
| ethyl-N-methyl-6-(oxan-4-yl)- | | | |
| 5,6,7,8-tetrahydrocarbazole-3- | | | |
| carboxamide | | | |
| 9-ethyl-N-methyl-6-(oxan-4-yl)-N- | 110.4 | 228.3 | 98 |
| [3-(2,2,2- | | | |
| trifluoroethylcarbamoyl)propyl]- | | | |
| 5,6,7,8-tetrahydrocarbazole-3- | | | |
| carboxamide | | | |
| 9-ethyl-N-[3-(2- | 236 | 597.4 | 119 |
| hydroxyethylcarbamoyl)propyl]-N- | | | |
| methyl-6-(oxan-4-yl)-5,6,7,8- | | | |
| tetrahydrocarbazole-3-carboxamide | | | |
| N-ethyl-N-(ethylcarbamoylmethyl)- | >3478.3 | >30000.0 | >42 |
| N′,N′-dimethyl-6-(oxan-4-yl)-5,6,7,8- | | | |
| tetrahydrocarbazole-3,9- | | | |
| dicarboxamide | | | |
| N-ethyl-9-methyl-6-(oxan-4-yl)-N- | 63 | 127.8 | 117 |
| (oxetan-3-ylcarbamoylmethyl)- | | | |
| 5,6,7,8-tetrahydrocarbazole-3- | | | |
| carboxamide | | | |
| N-[1-[9-methyl-6-(oxan-4-yl)5,6,7,8- | >3478.3 | >30000.0 | >30 |
| tetrahydrocarbazole-3-carbonyl]-4- | | | |
| piperidyl]cyclopropanecarboxamide | | | |
| N-ethyl-N-(ethylcarbamoylmethyl)- | 201.9 | 437.9 | 109 |
| 9-(2-fluoroethyl)-6-(oxan-4-yl)- | | | |
| 5,6,7,8-tetrahydrocarbazole-3- | | | |
| carboxamide | | | |
| N-[1-[9-methyl-6-(oxan-4-yl)5,6,7,8- | 26.7 | 40.6 | 115 |
| tetrahydrocarbazole-3-carbonyl]-3- | | | |
| piperidyl]cyclopropanecarboxamide | | | |
| N-ethyl-N-(ethylcarbamoylmethyl)- | | >30000.0 | >9 |
| 9-(methylcarbamoylmethyl)-6-(oxan- | | | |
| 4-yl)-5,6,7,8-tetrahydrocarbazole-3- | | | |
| carboxamide | | | |
| N-ethyl-N-(2- | 219.3 | 456.8 | 113 |
| methoxyethylcarbamoylmethyl)-9- | | | |
| methyl-6-(oxan-4-yl)-5,6,7,8- | | | |
| tetrahydrocarbazole-3-carboxamide | | | |
| 9-(diethylcarbamoylmethyl)-N-ethyl- | | >10000.0 | >87 |
| N-(ethylcarbamoylmethyl)-6-(oxan- | | | |
| 4-yl)-5,6,7,8-tetrahydrocarbazole-3- | | | |
| carboxamide | | | |
| N-[3-(ethylcarbamoyl)propyl]-9-(2- | 237.1 | 371.9 | 109 |
| fluoroethyl)-N-methyl-6-(oxan-4-yl)- | | | |
| 5,6,7,8-tetrahydrocarbazole-3- | | | |
| carboxamide | | | |
| N-ethyl-N-[[(2R)-2- | 182.4 | 318.4 | 124 |
| hydroxypropyl]carbamoylmethyl]-9- | | | |
| methyl-6-(oxan-4-yl)-5,6,7,8- | | | |
| tetrahydrocarbazole-3-carboxamide | | | |
| (3S)-N-(2,2-difluoroethyl)-1-[9- | 49.5 | 82.6 | 125 |
| methyl-6-(oxan-4-yl)5,6,7,8- | | | |
| tetrahydrocarbazole-3- | | | |
| carbonyl]pyrrolidine-3-carboxamide | | | |
| N-ethyl-9-methyl-6-(oxan-4-yl)-N- | 217.8 | 524.5 | 127 |
| [[(3R)-oxolan-3- | | | |
| yl]carbamoylmethyl]-5,6,7,8- | | | |
| tetrahydrocarbazole-3-carboxamide | | | |
| N-ethyl-9-methyl-6-(oxan-4-yl)-N- | 58.1 | 100 | 122 |
| [[(3S)-oxolan-3- | | | |
| yl]carbamoylmethyl]-5,6,7,8- | | | |
| tetrahydrocarbazole-3-carboxamide | | | |
| (3S)-N-ethyl-1-[9-methyl-6-(oxan-4- | 69.9 | 102.6 | 122 |
| yl)5,6,7,8-tetrahydrocarbazole-3- | | | |
| carbonyl]pyrrolidine-3-carboxamide | | | |
| N-ethyl-N-(ethylcarbamoylmethyl)- | | >30000.0 | >34 |
| 9-(2-hydroxy-2-methyl-propyl)-6- | | | |
| (oxan-4-yl)-5,6,7,8- | | | |
| tetrahydrocarbazole-3-carboxamide | | | |
| N-ethyl-N-(ethylcarbamoylmethyl)- | | >3333.3 | >83 |
| 9-(2-hydroxyethyl)-6-(oxan-4-yl)- | | | |
| 5,6,7,8-tetrahydrocarbazole-3- | | | |
| carboxamide | | | |
| N-ethyl-9-methyl-6-(oxan-4-yl)-N- | | >1111.1 | >88 |
| (oxan-4-ylcarbamoylmethyl)-5,6,7,8- | | | |
| tetrahydrocarbazole-3-carboxamide | | | |
| N-(2-cyanoethylcarbamoylmethyl)- | 115.4 | 119.9 | 115 |
| N-ethyl-9-ethylsulfonyl-6-(oxan-4- | | | |
| yl)-5,6,7,8-tetrahydrocarbazole-3- | | | |
| carboxamide | | | |
| N-[1-[9-methyl-6-(oxan-4-yl)5,6,7,8- | 63.2 | 263.4 | 122 |
| tetrahydrocarbazole-3-carbonyl]-3- | | | |
| piperidyl]propanamide | | | |
| 2-methyl-N-[1-[9-methyl-6-(oxan-4- | 157.1 | 400.8 | 118 |
| yl)5,6,7,8-tetrahydrocarbazole-3- | | | |
| carbonyl]-3-piperidyl]propanamide | | | |
| N-ethyl-N-[[(2S)-2- | | | |
| hydroxypropyl]carbamoylmethyl]-9- | | | |
| methyl-6-(oxan-4-yl)-5,6,7,8- | | | |
| tetrahydrocarbazole-3-carboxamide | | | |
| N-[3-(2- | 90.9 | 155.1 | 122 |
| hydroxyethylcarbamoyl)propyl]-N,9- | | | |
| dimethyl-6-(oxan-4-yl)-5,6,7,8- | | | |
| tetrahydrocarbazole-3-carboxamide | | | |
| N-[(3S)-1-[9-methyl-6-(oxan-4- | 43.4 | 87.2 | 119 |
| yl)5,6,7,8-tetrahydrocarbazole-3- | | | |
| carbonyl]-3- | | | |
| piperidyl]cyclopropanecarboxamide | | | |
| N,9-dimethyl-6-(oxan-4-yl)-N-[3- | | | |
| (oxetan-3-ylcarbamoyl)propyl]- | | | |
| 5,6,7,8-tetrahydrocarbazole-3- | | | |
| carboxamide | | | |
| N,9-dimethyl-6-(oxan-4-yl)-N-[3- | | | |
| [[(3S)-oxolan-3- | | | |
| yl]carbamoyl]propyl]-5,6,7,8- | | | |
| tetrahydrocarbazole-3-carboxamide | | | |
| N-[3-(3- | | | |
| hydroxypropylcarbamoyl)propyl]- | | | |
| N,9-dimethyl-6-(oxan-4-yl)-5,6,7,8- | | | |
| tetrahydrocarbazole-3-carboxamide | | | |
| 2-cyclopropyl-N-[1-[9-methyl-6- | | | |
| (oxan-4-yl)5,6,7,8- | | | |
| tetrahydrocarbazole-3-carbonyl]-3- | | | |
| piperidyl]acetamide | | | |
| N-ethyl-9-ethylsulfonyl-6-(oxan-4- | | | |
| yl)-N-(oxetan-3-ylcarbamoylmethyl)- | | | |
| 5,6,7,8-tetrahydrocarbazole-3- | | | |
| carboxamide | | | |
| N-ethyl-9-ethylsulfonyl-N-(2- | | | |
| methoxyethylcarbamoylmethyl)-6- | | | |
| (oxan-4-yl)-5,6,7,8- | | | |
| tetrahydrocarbazole-3-carboxamide | | | |
| N-ethyl-9-ethylsulfonyl-N-(2- | | | |
| hydroxypropylcarbamoylmethyl)-6- | | | |
| (oxan-4-yl)-5,6,7,8- | | | |
| tetrahydrocarbazole-3-carboxamide | | | |
| N-ethyl-N-(2-hydroxypropyl)-9- | | | |
| methyl-6-(oxan-4-yl)-5,6,7,8- | | | |
| tetrahydrocarbazole-3-carboxamide | | | |
| (R)-N-((S)-1-Hydroxy-5-(oxetan-3- | | 201 | 119 |
| ylamino)-5-oxopentan-2-yl)-N,9- | | | |
| dimethyl-3-(tetrahydro-2H-pyran-4- | | | |
| yl)-2,3,4,9-tetrahydro-1H-carbazole- | | | |
| 6-carboxamide | | | |
| (R)-N-((S)-5-(2,2- | | 107 | 120 |
| Difluoroethylamino)-1-hydroxy-5- | | | |
| oxopentan-2-yl)-N,9-dimethyl-3- | | | |
| (tetrahydro-2H-pyran-4-yl)-2,3,4,9- | | | |
| tetrahydro-1H-carbazole-6- | | | |
| carboxamide | | | |
| (R)-N-((S)-5-(2-Fluoroethylamino)- | 26 | 61 | 118 |
| 1-hydroxy-5-oxopentan-2-yl)-N,9- | | | |
| dimethyl-3-(tetrahydro-2H-pyran-4- | | | |
| yl)-2,3,4,9-tetrahydro-1H-carbazole- | | | |
| 6-carboxamide | | | |
| (R)-N-(4-(Cyanomethylamino)-4- | | 71 | 110 |
| oxobutyl)-N,9-dimethyl-3- | | | |
| (tetrahydro-2H-pyran-4-yl)-2,3,4,9- | | | |
| tetrahydro-1H-carbazole-6- | | | |
| carboxamide | | | |
| (R)-N-((S)-1-Hydroxy-5- | | 101 | 124 |
| (methylamino)-5-oxopentan-2-yl)- | | | |
| N,9-dimethyl-3-(tetrahydro-2H- | | | |
| pyran-4-yl)-2,3,4,9-tetrahydro-1H- | | | |
| carbazole-6-carboxamide | | | |
| (R)-N-((S)-1-Hydroxy-5- | | 133 | 129 |
| (isopropylamino)-5-oxopentan-2-yl)- | | | |
| N,9-dimethyl-3-(tetrahydro-2H- | | | |
| pyran-4-yl)-2,3,4,9-tetrahydro-1H- | | | |
| carbazole-6-carboxamide | | | |
| (R)-N-((S)-5-(Ethylamino)-1- | 36 | 79 | 126 |
| hydroxy-5-oxopentan-2-yl)-N,9- | | | |
| dimethyl-3-(tetrahydro-2H-pyran-4- | | | |
| yl)-2,3,4,9-tetrahydro-1H-carbazole- | | | |
| 6-carboxamide | | | |
| (R)-N-(4-(Methoxyamino)-4- | | 291 | 104 |
| oxobutyl)-N,9-dimethyl-3- | | | |
| (tetrahydro-2H-pyran-4-yl)-2,3,4,9- | | | |
| tetrahydro-1H-carbazole-6- | | | |
| carboxamide | | | |
| (R)-N-(4-(2,2-Dimethylhydrazinyl)- | | 917 | 96 |
| 4-oxobutyl)-N,9-dimethyl-3- | | | |
| (tetrahydro-2H-pyran-4-yl)-2,3,4,9- | | | |
| tetrahydro-1H-carbazole-6- | | | |
| carboxamide | | | |
| (R)-N-(4-(2-Methoxyethylamino)-4- | | 1210 | 93 |
| oxobutyl)-N,9-dimethyl-3- | | | |
| (tetrahydro-2H-pyran-4-yl)-2,3,4,9- | | | |
| tetrahydro-1H-carbazole-6- | | | |
| carboxamide | | | |
| (R)-N-(4-(1H-Pyrrol-1-ylamino)-4- | | 1050 | 89 |
| oxobutyl)-N,9-dimethyl-3- | | | |
| (tetrahydro-2H-pyran-4-yl)-2,3,4,9- | | | |
| tetrahydro-1H-carbazole-6- | | | |
| carboxamide | | | |
| (R)-N-Ethyl-N-(4-(2- | | 31 | 113 |
| hydroxyethylamino)-4-oxobutyl)-9- | | | |
| methyl-3-(tetrahydro-2H-pyran-4-yl)- | | | |
| 2,3,4,9-tetrahydro-1H-carbazole-6- | | | |
| carboxamide | | | |
| (R)-N-(4-(2-Hydroxyethylamino)-4- | 69 | 173 | 120 |
| oxobutyl)-N,9-dimethyl-3- | | | |
| (tetrahydro-2H-pyran-4-yl)-2,3,4,9- | | | |
| tetrahydro-1H-carbazole-6- | | | |
| carboxamide | | | |

### EXAMPLES

### Example 1

### N-[2-(Cyclopropylamino)-2-oxoethyl]-N,9-dimethyl-3-(tetrahydro-2H-pyran-4-yl)-2,3,4,9-tetrahydro-1H-carbazole-6-carboxamide

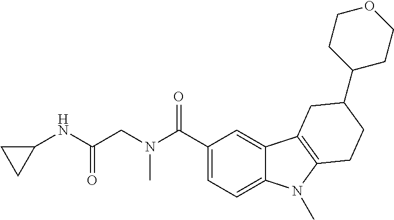

### Step A. N-[2-(Cyclopropylamino)-2-oxoethyl]-N,9-dimethyl-3-(tetrahydro-2H-pyran-4-yl)-2,3,4,9-tetrahydro-1H-carbazole-6-carboxamide

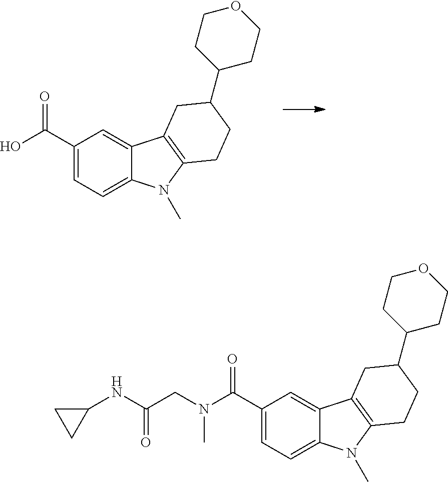

HATU (145 mg, 0.38 mmol) and N¹-cyclopropyl-N²-methylglycinamide (50 mg, 0.38 mmol) were added to a solution of 9-methyl-3-(tetrahydro-2H-pyran-4-yl)-2,3,4,9-tetrahydro-1H-carbazole-6-carboxylic acid (100 mg, 0.32 mmol) and DIPEA (89 μL, 0.48 mmol) in DMF (15 mL). The reaction mixture was stirred for 2 hrs. and the solvent was concentrated. The product was purified by preparative reverse-phase HPLC using an acetonitrile gradient 10 to 80% in water to provide the TFA salt of the title compound as white solid (47 mg, 27%). ¹H NMR (400 MHz, CDCl₃) δ 0.48-0.65 (m, 2H), 0.80 (q, J=6.64 Hz, 2H), 1.34-1.67 (m, 5H), 1.75 (t, J=10.35 Hz, 2H), 2.07-2.25 (m, 1H), 2.30-2.53 (m, 1H), 2.58-2.97 (m, 4H), 3.14 (s, 3H), 3.42 (t, J=11.72 Hz, 2H), 3.64 (s, 3H), 3.93-4.24 (m, 4H), 7.16-7.35 (m, 2H), 7.62 (s, 1H); MS (ESI) (M+H)⁺424.2.

### Step B. 4-[4-(Benzyloxy)phenyl]tetrahydro-2H-1-pyran-4-ol

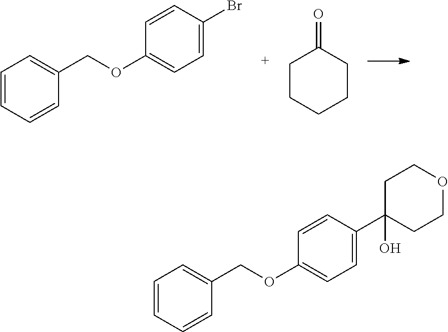

A solution of 1-(benzyloxy)-4-bromobenzene (12.6 g, 48.1 mmol) in THF (50 mL) was slowly added to a mixture of magnesium (1.06 g, 43.7 mmol) and THF (10 mL) at room temperature. Iodine (50 mg, 0.2 mmol) was added. The reaction mixture was heated to 70° C. until the magnesium disappearance (4 hrs.). The reaction mixture was cooled to ambient temperature and tetrahydro-4H-pyran-4-one (4.38 g, 43.7 mmol) was slowly added. The solution was stirred overnight and quenched with cold 0.5M HCl (200 mL). The product was extracted with EtOAc and purified by normal-phase MPLC using EtOAc 30 to 90% gradient in hexane to provide the title compound as white solid (6.87 g, 50%). ¹H NMR (400 MHz, CDCl₃) δ 1.64-1.75 (m, 2H), 2.07-2.21 (m, 2H), 3.81-3.98 (m, 4H), 5.03-5.10 (m, 2H), 6.94-7.01 (m, 3H), 7.29-7.47 (m, 6H).

### Step C. 4-[4-(Benzyloxy)phenyl]-3,6-dihydro-2H-pyran

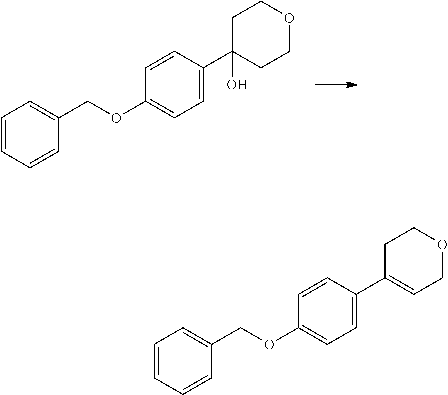

A solution of 4-[4-(benzyloxy)phenyl]tetrahydro-2H-pyran-4-ol (6.80 g, 23.9 mmol) in toluene (100 mL) under molecular sieves (5 g) was heated to reflux (123° C.) for 18 hrs. The solvent was concentrated and the product was purified by normal-phase MPLC using EtOAc 30 to 90% gradient in hexane to provide the title compound as white solid (4.55 g, 71%). ¹H NMR (400 CDCl₃) δ 2.44-2.54 (m, 2H), 3.92 (t, J=5.47 Hz, 2H), 4.31 (q, J=2.99 Hz, 2H), 5.07 (s, 2H), 5.99-6.06 (m, 1H), 6.90-6.99 (m, 3H), 7.29-7.36 (m, 3H), 7.36-7.46 (m, 3H).

### Step D. 4-(Tetrahydro-2H-pyran-4-yl)phenol

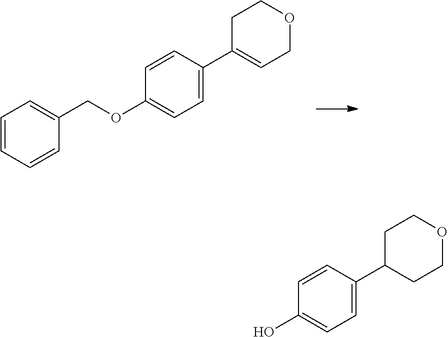

A solution of 4-[4-(benzyloxy)phenyl]-3,6-dihydro-2H-pyran (4.50 g, 16.8 mmol) in EtOH (150 mL) was treated with Pd/C 10% in a Parr hydrogenation apparatus under a 50 PSI hydrogen atmosphere for 3 days. The mixture was filtered on a celite pad and the solvent was concentrated to provide the title compound as grey solid (2.72 g, 90%). ¹H NMR (400 MHz, CDCl₃) δ 1.61-1.87 (m, 4H), 2.59-2.78 (m, 1H), 3.54 (td, J=11.13, 3.52 Hz, 2H), 4.00-4.16 (m, 2H), 6.79 (d, J=8.59 Hz, 2H), 7.09 (d, J=8.59 Hz, 2H).

### Step E. 4-(Tetrahydro-2H-pyran-4-yl)cyclohexanol

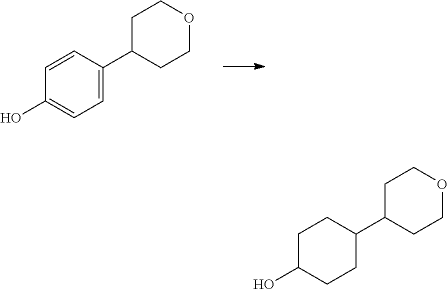

A mixture of 4-(tetrahydro-2H-pyran-4-yl)phenol (2.60 g, 14.5 mmol), Pd/C 10% (0.77 g, 0.72 mmol), sodium formate (14.8 g, 0.21 mol) and water (50 mL) was heated to 105° C. for 18 hrs. The reaction mixture was filtered and the filtrate was extracted with ethyl acetate. The residue was thoroughly washed with ethyl acetate. The ethyl acetate extracts were combined and concentrated to provide the title compound as white solid (2.47 g, 92%). ¹H NMR (400 MHz, CDCl₃) δ 0.88-1.13 (m, 2H), 1.13-1.44 (m, 5H), 1.44-1.67 (m, 3H), 1.66-1.87 (m, 2H), 1.91-2.11 (m, 2H), 3.23-3.45 (m, 2H), 3.47-3.61 (m, 1H), 3.87-4.08 (m, 2H).

### Step F. 4-(Tetrahydro-2H-pyran-4-yl)cyclohexanone

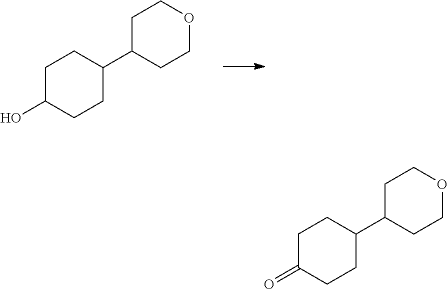

Concentrated H₂SO₄ (3 mL) was slowly added to a solution of CrO₃ (3.0 g, 30 mmol) in water (9 mL) at 0° C. The resulting solution was added drop wise to a solution of 4-(tetrahydro-2H-pyran-4-yl)cyclohexanol (2.41 g, 13.0 mmol) in acetone (70 mL) at 0° C. The reaction mixture was stirred for 1 hr. at 0° C. and ethyl ether (300 mL) was added. The solution was washed with brine, water and dried over anhydrous MgSO₄. The solvent was concentrated to provide the title compound as white solid (2.02 g, 85%). ¹H NMR (400 MHz, CDCl₃) δ 1.31-1.51 (m, 5H), 1.51-1.59 (m, 1H), 1.59-1.70 (m, J=9.96, 9.96 Hz, 2H), 2.00-2.18 (m, 2H), 2.23-2.49 (m, 4H), 3.38 (t, J=11.33 Hz, 2H), 3.93-4.07 (m, 2H).

### Step G. 3-(Tetrahydro-2H-pyran-4-yl)-2,3,4,9-tetrahydro-1H-carbazole-6-carboxylic acid

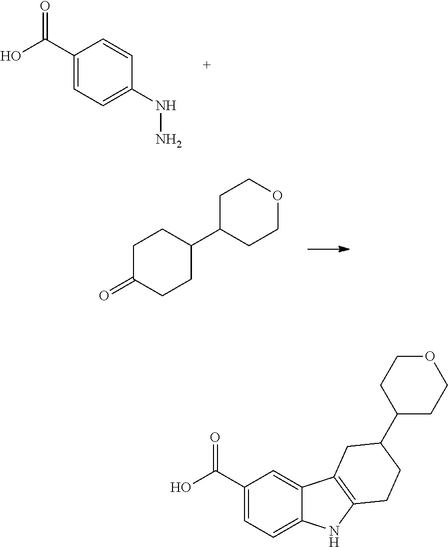

A mixture of 4-hydrazinobenzoic acid (0.83 g, 5.48 mmol) and 4-(tetrahydro-2H-pyran-4-yl)cyclohexanone (1.00 g, 5.48 mmol) in dioxane (35 mL) and concentrated HCl (8 mL) was heated to 100° C. for 3 hrs. The reaction mixture was concentrated to dryness and recovered in NaOH 2M (50 mL). The solution was cooled in an ice bath and slowly acidified to pH 4 using concentrated HCl. The precipitate was collected and dried to provide the title compound as brown solid (1.21 g, 74%). MS (ESI) (M+H)⁺ 300.1.

### Step H. Methyl 9-methyl-3-(tetrahydro-2H-pyran-4-yl)-2,3,4,9-tetrahydro-1H-carbazole-6-carboxylate

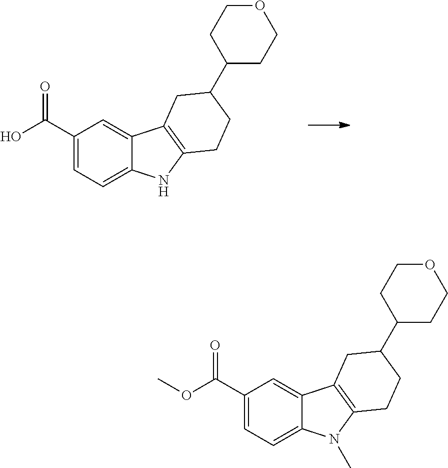

Sodium hydride (1.5 g, 36.7 mmol) was added to a solution of 3-(tetrahydro-2H-pyran-4-yl)-2,3,4,9-tetrahydro-1H-carbazole-6-carboxylic acid (1.10 g, 3.67 mmol) in DMF (150 mL) under nitrogen atmosphere. The reaction mixture was stirred for 1.5 hr. and methyl iodide (2.4 mL, 36.7 mmol) was added. The mixture was stirred for 1.5 hr., cooled to 0° C. and quenched with NH₄Cl saturated solution. The mixture was concentrated to dryness and recovered in water. The product was extracted with EtOAc and purified by normal-phase MPLC using EtOAc 20 to 70% gradient in heptane to provide the title compound as white solid (0.60 g, 50%). ¹H NMR (400 MHz, CDCl₃) δ 1.38-1.68 (m, 5H), 1.76 (d, J=11.72 Hz, 2H), 2.07-2.22 (m, 1H), 2.43 (dd, J=15.23, 8.59 Hz, 1H), 2.61-2.98 (m, 3H), 3.42 (t, J=11.72 Hz, 2H), 3.64 (s, 3H), 3.93 (s, 3H), 4.04 (dd, J=11.13, 3.71 Hz, 2H), 7.24 (d, J=8.59 Hz, 1H), 7.86 (dd, J=8.59, 1.56 Hz, 1H), 8.23 (d, J=1.17 Hz, 1H); MS (ESI) (M+H)⁺ 328.1.

### Step I. 9-Methyl-3-(tetrahydro-2H-pyran-4-yl)-2,3,4,9-tetrahydro-1H-carbazole-6-carboxylic acid

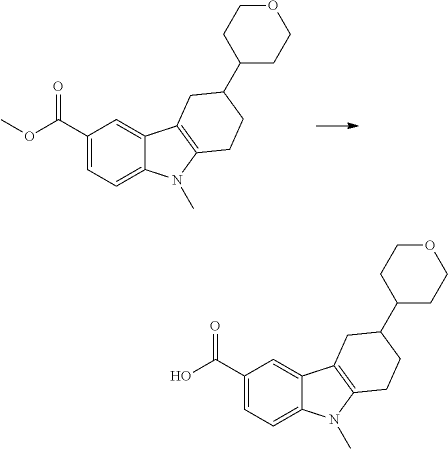

A mixture of methyl 9-methyl-3-(tetrahydro-2H-pyran-4-yl)-2,3,4,9-tetrahydro-1H-carbazole-6-carboxylate (0.57 g, 1.74 mmol), MeOH (30 mL) and NaOH 2M (40 mL) was heated to 85° C. for 4 hrs. The solution was concentrated to 40 mL, cooled to 0° C. and acidified to pH 4 using concentrated HCl. The precipitate was collected and dried to provide the title compounds as white solid (0.48 g, 88%). ¹H NMR (400 MHz, CD₃SOCD₃) δ 1.20-1.40 (m, 2H), 1.41-1.61 (m, 3H), 1.63-1.78 (m, 2H), 1.98-2.15 (m, 1H), 2.34 (dd, J=14.84, 7.81 Hz, 1H), 2.55-2.73 (m, 1H), 2.73-2.90 (m, 2H), 3.19-3.36 (m, 2H), 3.62 (s, 3H), 3.80-3.96 (m, 2H), 7.39 (d, J=8.59 Hz, 1H), 7.67 (dd, J=8.59, 1.56 Hz, 1H), 8.04 (d, J=1.56 Hz, 1H); MS (ESI) (M+H)⁺ 314.1.

### Example 2 & 3

### (R)—N-[2-(Cyclopropylamino)-2-oxoethyl]-N,9-dimethyl-3-(tetrahydro-2H-pyran-4-yl)-2,3,4,9-tetrahydro-1H-carbazole-6-carboxamide and (S)—N-[2-(Cyclopropylamino)-2-oxoethyl]-N,9-dimethyl-3-(tetrahydro-2H-pyran-4-yl)-2,3,4,9-tetrahydro-1H-carbazole-6-carboxamide

N-(2-(Cyclopropylamino)-2-oxoethyl)-N,9-dimethyl-3-(tetrahydro-2H-pyran-4-yl)-2,3,4,9-tetrahydro-1H-carbazole-6-carboxamide (490 mg, 1.16 mmol) was separated by preparative chiral HPLC using a Gilson system equipped with a Chiracel AD column, 5 cm ID×50 cm L, 20u, 35% EtOH/hexanes with 0.1% diethylamine v/v; 100 mL/min, 60 min run, at rt in two runs (245 mg loadings).

(R)—N-[2-(Cyclopropylamino)-2-oxoethyl]-N,9-dimethyl-3-(tetrahydro-2H-pyran-4-yl)-2,3,4,9-tetrahydro-1H-carbazole-6-carboxamide (isomer 1, 206 mg):

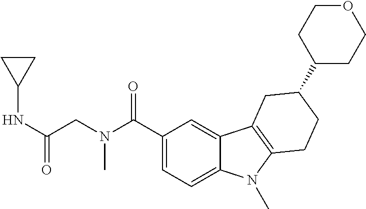

¹H NMR (400 MHz, CHLOROFORM-D) δ 0.53-0.58 (m, 2H), 0.77-0.84 (m, 2H), 1.44-1.50 (m, 2H), 1.52-1.58 (m, 2H), 1.58-1.63 (m, 2H), 1.70-1.80 (m, 2H), 2.12-2.19 (m, 1H), 2.36-2.46 (m, 1H), 2.65-2.72 (m, 1H), 2.72-2.78 (m, 1H), 2.79-2.91 (m, 2H), 3.15 (s, 3H), 3.38-3.46 (m, 2H), 3.64 (s, 3H), 4.05 (dd, J=11.33, 3.12 Hz, 2H), 4.09 (s, 1H), 6.96 (s, 1H), 7.25 (s, 2H), 7.62 (s, 1H); Rf=4.11; MS (ESI) (M+H)=424.2; [α]_{D}=+53.6° (1.007, CDCl₃); Anal. Calc. For C₂₅H₃₃N₃O₃+0.5 EtOH C, 69.93 H, 8.12 N, 9.41 found: C, 69.46 H, 8.09 N, 9.85.

(S)—N-[2-(Cyclopropylamino)-2-oxoethyl]-N,9-dimethyl-3-(tetrahydro-2H-pyran-4-yl)-2,3,4,9-tetrahydro-1H-carbazole-6-carboxamide (isomer 2, 220 mg):

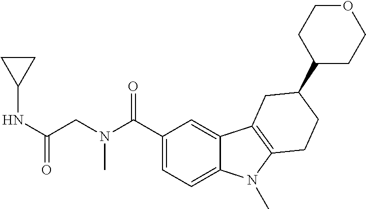

¹H NMR (400 MHz, CHLOROFORM-D) δ 0.53-0.58 (m, 2H), 0.77-0.84 (m, 2H), 1.44-1.50 (m, 2H), 1.52-1.58 (m, 2H), 1.58-1.63 (m, 2H), 1.70-1.80 (m, 2H), 2.12-2.19 (m, 1H), 2.36-2.46 (m, 1H), 2.65-2.72 (m, 1H), 2.72-2.78 (m, 1H), 2.79-2.91 (m, 2H), 3.15 (s, 3H), 3.38-3.46 (m, 2H), 3.64 (s, 3H), 4.05 (dd, J=11.33, 3.12 Hz, 2H), 4.09 (s, 1H), 6.96 (s, 1H), 7.25 (s, 2H), 7.62 (s, 1H); Rf=5.54; MS (ESI) (M+H)=424.2; [α]_{D}=−51.2° (0.997, CDCl₃); Anal. Calc. For C₂₅H₃₃N₃O₃+0.7 EtOH C, 69.57 H, 8.23 N, 9.22. found: C, 68.85 H, 7.47 N, 9.52.

Chiral analytical HPLC: ChiraPak AD column, 30% EtOH/hexanes, 1 mL/min, 30 min run, 25° C.

### Example 4

### N-Ethyl-N-[2-(ethylamino)-2-oxoethyl]-9-methyl-3-(tetrahydro-2H-pyran-4-yl)-2,3,4,9-tetrahydro-1H-carbazole-6-carboxamide

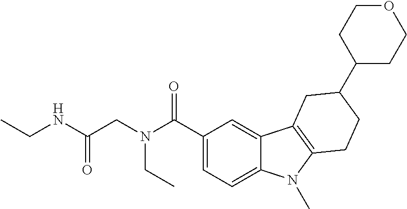

### Step A. N-ethyl-N-[2-(ethylamino)-2-oxoethyl]-9-methyl-3-(tetrahydro-2H-pyran-4-yl)-2,3,4,9-tetrahydro-1H-carbazole-6-carboxamide

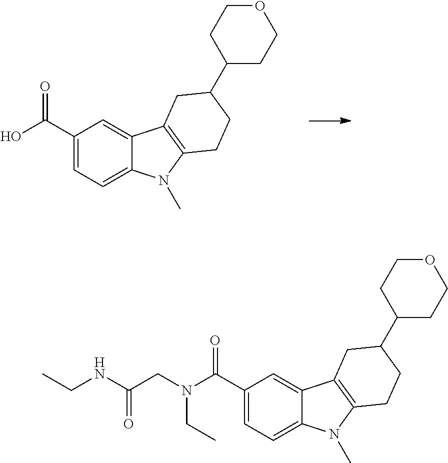

Following the procedure of example 1 step A and using N¹,N²-diethylglycinamide (50 mg, 0.38 mmol) provided the title compound as white solid (83 mg, 48%). 1H NMR NMR (400 MHz, CDCl₃) δ 1.19 (q, J=7.29 Hz, 6H), 1.38-1.67 (m, 5H), 1.69-1.82 (m, 2H), 2.09-2.21 (m, 1H), 2.33-2.48 (m, J=14.26, 7.62 Hz, 1H), 2.62-2.77 (m, 1H), 2.77-2.92 (m, 2H), 3.27-3.38 (m, 2H), 3.38-3.47 (m, 2H), 3.51 (q, J=6.12 Hz, 2H), 3.64 (s, 3H), 4.05 (dd, J=11.33, 3.12 Hz, 2H), 4.09-4.19 (m, 2H), 7.17-7.31 (m, 2H), 7.58 (s, 1H); MS (ESI) (M+H)⁺ 426.2.

### Example 5

### N-Ethyl-N-[2-(isopropylamino)-2-oxoethyl]-9-methyl-3-(tetrahydro-2H-pyran-4-yl)-2,3,4,9-tetrahydro-1H-carbazole-6-carboxamide

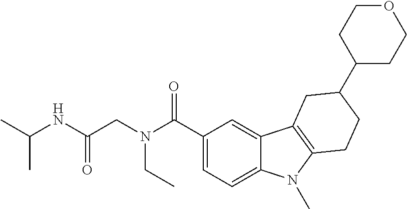

### Step A. N-Ethyl-N-[2-(isopropylamino)-2-oxoethyl]-9-methyl-3-(tetrahydro-2H-pyran-4-yl)-2,3,4,9-tetrahydro-1H-carbazole-6-carboxamide

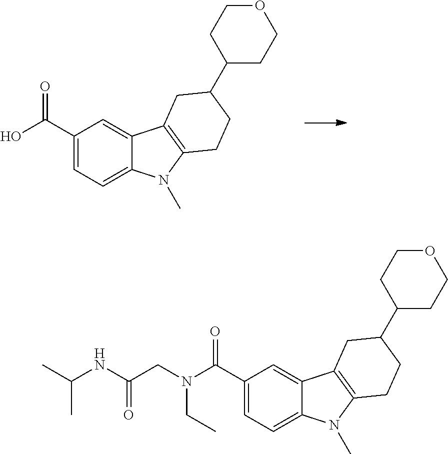

Following the procedure of example 1 step A and using N²-ethyl-N¹-isopropylglycinamide (50 mg, 0.38 mmol) provided the title compound as white solid (75 mg, 42%). ¹H NMR (400 MHz, CDCl₃) δ 1.09-1.27 (m, 8H), 1.38-1.67 (m, 5H), 1.74 (t, J=11.33 Hz, 3H), 2.08-2.21 (m, 1H), 2.41 (dd, J=15.23, 8.59 Hz, 1H), 2.61-2.75 (m, 1H), 2.77-2.90 (m, 2H), 3.35-3.47 (m, 2H), 3.51 (q, J=7.03 Hz, 2H), 3.64 (s, 3 H), 3.95-4.18 (m, 5H), 7.18-7.25 (m, 2H), 7.56 (s, 1H); MS (ESI) (M+H)⁺ 440.2.

### Example 6

### N-Cyclopropyl-1-(9-methyl-3-(tetrahydro-2H-pyran-4-yl)-2,3,4,9-tetrahydro-1H-carbazole-6-carbonyl)piperidine-4-carboxamide

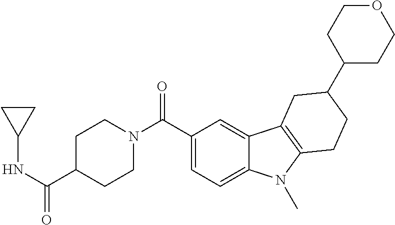

### Step A: N-Cyclopropyl-1-(9-methyl-3-(tetrahydro-2H-pyran-4-yl)-2,3,4,9-tetrahydro-1H-carbazole-6-carbonyl)piperidine-4-carboxamide

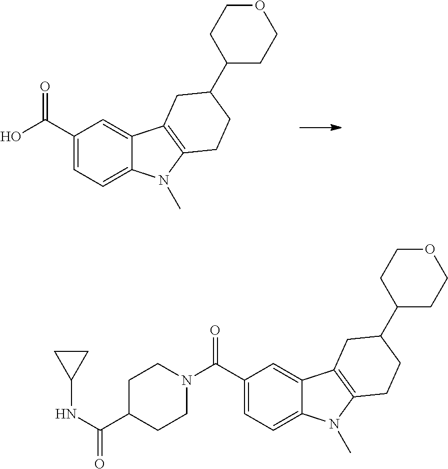

9-Methyl-3-(tetrahydro-2H-pyran-4-yl)-2,3,4,9-tetrahydro-1H-carbazole-6-carboxylic acid (150 mg, 0.48 mmol), HATU (218 mg, 0.57 mmol) and N-cyclopropylpiperidine-4-carboxamide, HCl (147 mg, 0.72 mmol) were stirred in DMF (10 mL) containing N,N-diisopropylethylamine (0.250 mL, 1.44 mmol) at 23° C. for 1 h. The solvent was evaporated and the residue was dissolved in EtOAc. The organic phase was washed with saturated aqueous sodium bicarbonate, brine and dried over anhydrous sodium sulfate. The product was purified by reversed-phase HPLC using a 40-60% CH₃CN/H₂O gradient. The fractions were evaporated, dissolved in EtOAc and washed with saturated aqueous sodium bicarbonate solution, brine and evaporated (130 mg, 56%). ¹H NMR (400 MHz, CHLOROFORM-D) δ 0.45-0.51 (m, 2H), 0.76-0.82 (m, 2H), 1.42-1.51 (m, 2H), 1.52-1.60 (m, 3H), 1.69-1.84 (m, 6H), 2.11-2.19 (m, 1H), 2.23-2.33 (m, 1H), 2.36-2.45 (m, 1H), 2.65-2.76 (m, 2H), 2.78-2.86 (m, 2H), 2.87-2.97 (m, 2 H), 3.42 (t, J=11.72 Hz, 3H), 3.63 (s, 3H), 4.05 (dd, J=11.13, 3.71 Hz, 2H), 5.65 (s, 1 H), 7.18-7.24 (m, 2H), 7.55 (s, 1H); MS (ESI) (M+H)⁺=464.2.

### Step B: N-Cyclopropylpiperidine-4-carboxamide hydrochloride

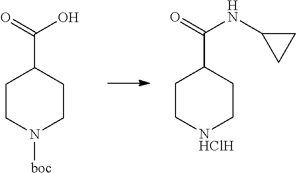

Boc-Isonipecotic acid (500 mg, 2.18 mmol), cyclopropylamine (0.180 mL, 2.61 mmol) and HATU (995 mg, 2.61 mmol) were stirred in 10 mL of DMF containing DIPEA (0.570 mL, 3.27 mmol) at 23° C. for 1 h. The solvent was evaporated. The residue was dissolved in EtOAc and washed with 5% KHSO₄, saturated aqueous NaHCO₃, brine and dried over anhydrous Na₂SO₄. The solvent was evaporated. The residue was dissolved in 20 mL of 1M HCl/AcOH and stirred at rt for 3 h. The solvent was evaporated. The product was precipitated in ether, filtered and dried (450 mg, 99%). ¹H NMR (400 MHz, METHANOL-D4) δ 0.41-0.49 (m, 2H), 0.67-0.75 (m, 2H), 1.79-1.89 (m, 2H), 1.86-1.94 (m, 2H), 2.37-2.48 (m, 1H), 2.59-2.67 (m, 1H), 2.92-3.04 (m, 2H), 3.35-3.45 (m, 2H).

### Example 7

### N-Ethyl-N-{2-[(2-fluoroethyl)amino]-2-oxoethyl}-9-methyl-3-(tetrahydro-2H-pyran-4-yl)-2,3,4,9-tetrahydro-1H-carbazole-6-carboxamide

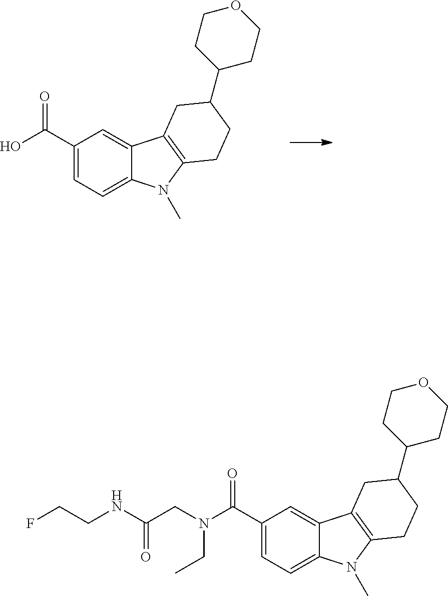

9-Methyl-3-(tetrahydro-2H-pyran-4-yl)-2,3,4,9-tetrahydro-1H-carbazole-6-carboxylic acid (150 mg, 0.48 mmol), HATU (236 mg, 0.62 mmol) and N²-ethyl-N¹-(2-fluoroethyl)glycinamide HCl (177 mg, 0.96 mmol) were stirred in DMF (10 mL) containing N,N-diisopropylethylamine (0.210 mL, 1.20 mmol) at 23° C. for 1 h. The solvent was evaporated and the residue was dissolved in EtOAc. The organic phase was washed with saturated aqueous sodium bicarbonate, brine and dried over anhydrous sodium sulfate. The product was purified by silica gel flash chromatography using EtOAc as eluent (105 mg, 50%). ¹H NMR (400 MHz, CHLOROFORM-D) δ 1.20 (t, J=6.84 Hz, 3H), 1.43-1.51 (m, 2H), 1.51-1.59 (m, 2H), 1.75 (t, J=9.57 Hz, 2H), 2.12-2.18 (m, 1H), 2.21-2.30 (m, 1H), 2.37-2.45 (m, 1H), 2.65-2.76 (m, 2H), 2.79-2.90 (m, 2H), 3.42 (t, J=11.13 Hz, 2H), 3.51 (q, J=5.86 Hz, 2H), 3.59 (q, J=5.21 Hz, 1H), 3.63-3.69 (m, 4H), 4.04 (d, J=10.55 Hz, 2H), 4.16 (s, 2H), 4.46 (t, J=4.88 Hz, 1H), 4.58 (t, J=4.88 Hz, 1H), 7.22-7.26 (m, 2H,) 7.59 (s, 1H); MS (ESI) (M+H)⁺=444.2.

### Example 8

N-[2-(Cyclopropylamino)-2-oxoethyl]-N-ethyl-9-methyl-3-(tetrahydro-2H-pyran-4-yl)-2,3,4,9-tetrahydro-1H-carbazole-6-carboxamide

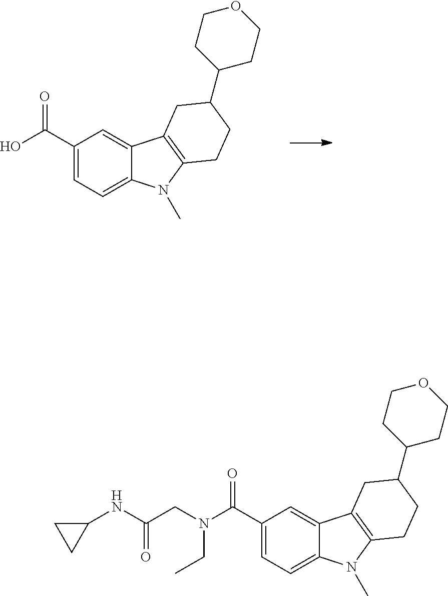

9-Methyl-3-(tetrahydro-2H-pyran-4-yl)-2,3,4,9-tetrahydro-1H-carbazole-6-carboxylic acid (150 mg, 0.48 mmol), HATU (236 mg, 0.62 mmol) and N¹-cyclopropyl-N²-ethylglycinamide HCl (171 mg, 0.96 mmol) were stirred in DMF (10 mL) containing N,N-diisopropylethylamine (0.210 mL, 1.20 mmol) at 23° C. for 1 h. The solvent was evaporated and the residue was dissolved in EtOAc. The organic phase was washed with saturated aqueous sodium bicarbonate, brine and dried over anhydrous sodium sulfate. The product was purified by silica gel flash chromatography using EtOAc as eluent (109 mg, 52%). ¹H NMR (400 MHz, CHLOROFORM-D) δ 0.52-0.58 (m, 2H), 0.77-0.84 (m, 2H), 1.19 (t, J=7.03 Hz, 3H), 1.43-1.51 (m, 2H), 1.51-1.59 (m, 3H), 1.70-1.79 (m, 2H), 2.12-2.19 (m, 1H), 2.21-2.29 (m, 1H), 2.37-2.46 (m, 1H), 2.67-2.78 (m, 2H), 2.79-2.89 (m, 2H), 3.38-3.46 (m, 2H), 3.49 (q, J=7.03 Hz, 2H), 3.64 (s, 3H), 4.05 (dd, J=11.72, 3.12 Hz, 2H,) 4.09 (s, 2H), 7.23-7.28 (m, 2H), 7.56 (s, 1H); MS (ESI) (M+H)=483.3.

### Example 9

### N-Cyclopropyl-2-(1-(9-methyl-3-(tetrahydro-2H-pyran-4-yl)-2,3,4,9-tetrahydro-1H-carbazole-6-carbonyl)azetidin-3-yl)acetamide

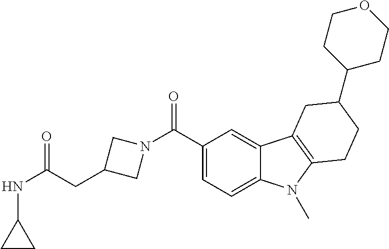

### Step A: N-Cyclopropyl-2-(1-(9-methyl-3-(tetrahydro-2H-pyran-4-yl)-2,3,4,9-tetrahydro-1H-carbazole-6-carbonyl)azetidin-3-yl)acetamide

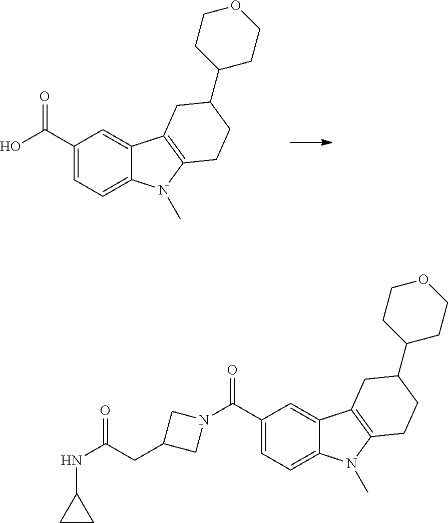

9-Methyl-3-(tetrahydro-2H-pyran-4-yl)-2,3,4,9-tetrahydro-1H-carbazole-6-carboxylic acid (125 mg, 0.40 mmol), HATU (182 mg, 0.48 mmol) and 2-(azetidin-3-yl)-N-cyclopropylacetamide HCl (91 mg, 0.48 mmol) were stirred in DMF (5 mL) containing N,N-diisopropylethylamine (0.174 mL, 1.00 mmol) at 23° C. for 1 h. The solvent was evaporated. The residue was dissolved in EtOAc and washed with saturated aqueous NaHCO₃, brine and dried over anhydrous Na₂SO₄. The product was purified by reversed-phase HPLC using 30-50% CH₃CN/H₂O. The fractions were evaporated and extracted with CH₂Cl₂ (3×). The organic phase was washed with brine and dried over anhydrous Na₂SO₄ (35 mg, 20%). ¹H NMR (400 MHz, CHLOROFORM-D) δ 0.44-0.52 (m, 2H), 0.71-0.78 (m, 2H), 1.42-1.50 (m, 2H), 1.51-1.55 (m, 1H), 1.56-1.61 (m, 2H), 1.71-1.78 (m, 2H), 2.11-2.18 (m, 1H), 2.35-2.44 (m, 1H), 2.48 (d, J=7.81 Hz, 2H), 2.62-2.73 (m, 2H), 2.78 (s, 1H), 2.81-2.90 (m, 2H), 3.00-3.10 (m, 1H), 3.42 (t, J=11.72 Hz, 2H), 3.62 (s, 3H), 3.85 (s, 1H), 4.04 (dd, J=10.94, 2.73 Hz, 2H), 4.30-4.38 (m, 1H), 4.53-4.60 (m, 1H), 6.02 (s, 1H), 7.20 (d, J=8.59 Hz, 1H), 7.45 (d, J=8.59 Hz, 1H), 7.79 (s, 1H); MS (EST) (M+H)⁺=450.2.

### Step B: 2-Azetidin-3-yl-N-cyclopropylacetamide hydrochloride

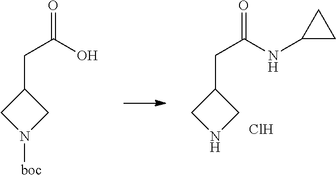

2-(1-(tert-Butoxycarbonyl)azetidin-3-yl)acetic acid (125 mg, 0.58 mmol), cyclopropylamine (0.060 mL, 0.87 mmol) and HATU (265 mg, 0.70 mmol) were stirred in DMF (5 mL) containing DIPEA (0.203 mL, 1.16 mmol) at 23° C. for 1 h. The solvent was evaporated. The residue was dissolved in EtOAc and washed with 5% aqueous KHSO₄, saturated aqueous sodium bicarbonate, brine and dried over anhydrous sodium sulfate. The solvent was evaporated. The product was then stirred in 1M HCl/AcOH (5 mL) at 23° C. for 2 h. The solvent was evaporated. The product was crashed in ether and the ether layer was decanted. The final product was dried under vacuum (100 mg, 90%). ¹H NMR (400 MHz, METHANOL-D4) δ 0.40-0.52 (m, 2H), 0.62-0.74 (m, 2H), 1.35 (dd, J=6.64, 4.30 Hz, 1H), 2.51 (d, J=7.42 Hz, 2H), 3.14-3.23 (m, 1H), 3.81-3.96 (m, 2H), 4.10 (t, J=9.96 Hz, 2H).

### Example 10

### N,9-Dimethyl-N-(2-(methylamino)ethyl)-3-(tetrahydro-2H-pyran-4-yl)-2,3,4,9-tetrahydro-1H-carbazole-6-carboxamide

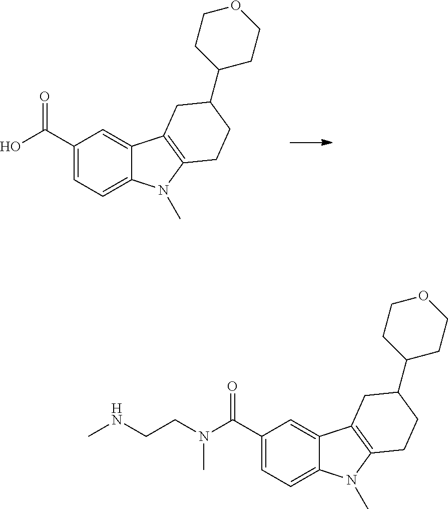

HATU (789 mg, 2.07 mmol) was added to a stirring DMF (25 mL) solution of 9-methyl-3-(tetrahydro-2H-pyran-4-yl)-2,3,4,9-tetrahydro-1H-carbazole-6-carboxylic acid (500 mg, 1.60 mmol), N,N′-dimethylethylenediamine (0.849 mL, 7.98 mmol) and N,N-diisopropylethylamine (0.417 mL, 2.39 mmol) and was stirred at 23° C. for 1 h. The solvent was evaporated. The residue was dissolved in EtOAc and washed with saturated aqueous NaHCO₃, brine and dried over anhydrous Na₂SO₄. The product was purified by reversed-phase HPLC using 30-50% CH₃CN/H₂O and lyophilized (320 mg, 40%). ¹H NMR (400 MHz, METHANOL-D4) δ 1.38-1.48 (m, 2H), 1.53-1.61 (m, 3H), 1.77 (t, J=14.26 Hz, 2H), 2.13-2.20 (m, 1H), 2.35-2.43 (m, 1H), 2.64-2.73 (m, 1H), 2.75 (s, 3H), 2.81-2.85 (m, 1H), 2.85-2.90 (m, 1H), 3.11 (s, 3H), 3.29-3.33 (m, 2H), 3.37-3.47 (m, 2H), 3.63 (s, 3H), 3.82 (t, J=5.66 Hz, 2H), 3.95-3.97 (m, 1H), 3.98-4.00 (m, 1H), 7.22-7.27 (m, 1H), 7.32-7.36 (m, 1H), 7.61 (s, 1H); MS (ESI) (M+H)⁺=384.2.

### Example 11

### N-(2-(3-Cyclopropyl-1-methylureido)ethyl)-N,9-dimethyl-3-(tetrahydro-2H-pyran-4-yl)-2,3,4,9-tetrahydro-1H-carbazole-6-carboxamide

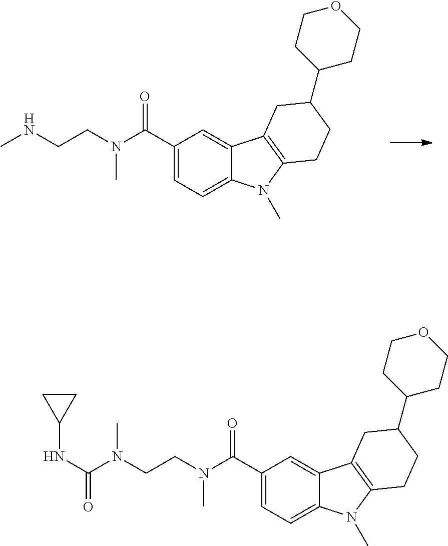

To a solution of triphosgene (59.6 mg, 0.20 mmol) in dichloromethane (2 mL) at 0° C. under N₂ was added a solution of cyclopropylamine (0.042 mL, 0.60 mmol) and N,N-diisopropylethylamine (0.175 mL, 1.00 mmol) in dichloromethane (5 mL) dropwise. The solution was stirred at 0° C. for 15 min. A solution of N,9-dimethyl-N-(2-(methylamino)ethyl)-3-(tetrahydro-2H-pyran-4-yl)-2,3,4,9-tetrahydro-1H-carbazole-6-carboxamide trifluoroacetic acid salt (100 mg, 0.20 mmol) in dichloromethane (5 mL) was added dropwise. After stirring at rt for 30 min, the reaction mixture was washed with saturated aqueous NaHCO₃, brine and dried over anhydrous Na₂SO₄. The product was purified by reversed-phase HPLC using 30-50% CH₃CN/H₂O and lyophilized (59 mg, 62%). ¹H NMR (400 MHz, METHANOL-D4) S 0.26-0.48 (m, 2H), 0.58 (d, 2H), 1.37-1.49 (m, 2H), 1.52-1.61 (m, 3H), 1.77 (t, J=11.52 Hz, 2H), 2.12-2.20 (m, 1H), 2.34-2.42 (m, 1H), 2.47-2.57 (m, 1H), 2.63-2.75 (m, 1H), 2.83 (s, 1H), 2.88 (m, 3H), 3.05-3.12 (m, 3H), 3.35 (s, 1H), 3.38-3.46 (m, 2H), 3.57-3.61 (m, 2H), 3.62 (s, 3H), 3.66-3.72 (m, 1H), 3.97 (dd, J=10.94, 2.73 Hz, 2H), 7.10-7.16 (m, 1H), 7.30 (d, J=7.81 Hz, 1H), 7.44-7.48 (m, 1H); MS (ESI) (M+H)⁺=467.3.

### Example 12

### N-(2-(3-Cyclopropyl-1-methylthioureido)ethyl)-N,9-dimethyl-3-(tetrahydro-2H-pyran-4-yl)-2,3,4,9-tetrahydro-1H-carbazole-6-carboxamide

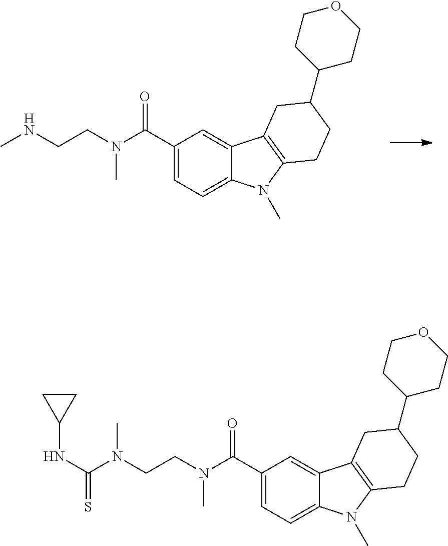

N,9-Dimethyl-N-(2-(methylamino)ethyl)-3-(tetrahydro-2H-pyran-4-yl)-2,3,4,9-tetrahydro-1H-carbazole-6-carboxamide trifluoroacetic acid salt (100 mg, 0.20 mmol) was dissolved in dichloromethane (10 mL) containing N,N-diisopropylethylamine (0.053 mL, 0.30 mmol) at 0° C. Cyclopropyl isothiocyanate (0.024 mL, 0.26 mmol) was added dropwise and the solution was stirred at rt for 1 h. The solution was washed with saturated aqueous NaHCO₃, brine and dried over anhydrous Na₂SO₄. The product was purified by reversed-phase HPLC using 30-50% CH₃CN/H₂O and lyophilized (80 mg, 82%). ¹H NMR (400 MHz, METHANOL-D4) (rotomers) δ 0.42 (s, 0.6H), 0.57 (s, 1.4H), 0.63 (s, 0.6H), 0.71 (s, 1.4H), 1.38-1.48 (m, 2H), 1.56 (s, 3H), 1.71-1.82 (m, 2H), 2.16 (s, 1H), 2.39 (s, 1H), 2.67 (s, 2H), 2.85 (d, J=16.41 Hz, 2H), 2.96 (s, 0.6H), 3.07-3.18 (m, 5.4H), 3.41 (t, J=10.55 Hz, 2H), 3.61 (s, 3H), 3.69 (s, 0.6H), 3.75 (s, 1.4H), 3.97 (d, J=10.94 Hz, 2.7H), 4.17 (s, 1.3H), 7.09 (s, 0.4H), 7.19 (s, 0.6H), 7.29 (d, J=6.64 Hz, 1H), 7.45 (s, 0.4H), 7.57 (s, 0.6H); MS (ESI) (M+H)⁺)=483.3.

### Example 13

### N-{2-[(2-Fluoroethyl)amino]-2-oxoethyl}-N,9-dimethyl-3-(tetrahydro-2H-pyran-4-yl)-2,3,4,9-tetrahydro-1H-carbazole-6-carboxamide

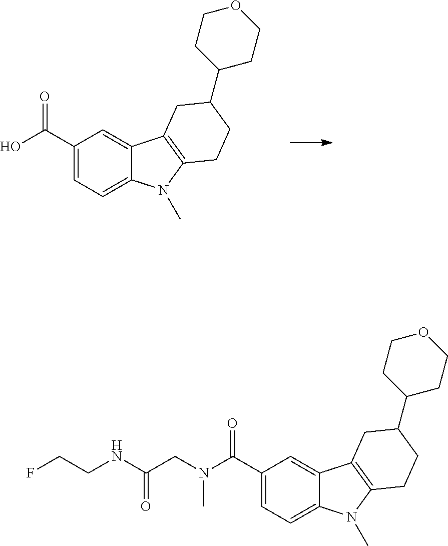

9-Methyl-3-(tetrahydro-2H-pyran-4-yl)-2,3,4,9-tetrahydro-1H-carbazole-6-carboxylic acid (150 mg, 0.48 mmol), HATU (218 mg, 0.57 mmol) and 2-(2-fluoroethylamino)-N-methyl-2-oxoethanaminium chloride (98 mg, 0.57 mmol) were stirred in DMF (10 mL) containing N,N-diisopropylethylamine (0.208 mL, 1.20 mmol) at 23° C. for 1 h. The solvent was evaporated. The residue was dissolved in EtOAc and washed with saturated aqueous NaHCO₃, brine and dried over anhydrous Na₂SO₄. The product was purified by reversed-phase HPLC using 40-60% CH₃CN/H₂O and lyophilized (150 mg, 73%). ¹H NMR (400 MHz, METHANORL-D4) δ 1.34-1.47 (m, 2H), 1.54 (s, 3H), 1.75 (t, J=13.28 Hz, 2H), 2.14 (s, 1H), 2.36 (s, 1H), 2.63-2.72 (m, 1H), 2.77-2.86 (m, 2H), 3.09 (s, 3H), 3.36-3.45 (m, 2H), 3.48 (s, 1H), 3.54 (s, 1H), 3.61 (s, 3H), 3.97 (dd, J=11.33, 3.91 Hz, 2H), 4.02 (s, 1H), 4.20 (s, 1H), 4.39 (s, 1H), 4.51 (s, 1H), 7.20 (s, 1 H), 7.30 (d, J=8.20 Hz, 1H), 7.54 (d, J=30.08 Hz, 1H); MS (ESI) (M+H)⁺=430.2.

### Example 14

### N-Ethyl-9-methyl-N-[2-(methylamino)-2-oxoethyl]-3-(tetrahydro-2H-pyran-4-yl)-2,3,4,9-tetrahydro-1H-carbazole-6-carboxamide

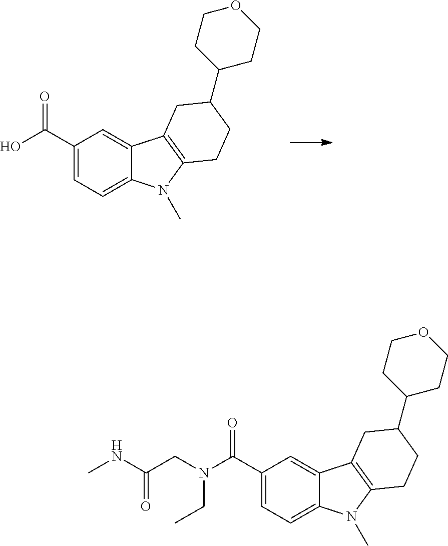

9-Methyl-3-(tetrahydro-2H-pyran-4-yl)-2,3,4,9-tetrahydro-1H-carbazole-6-carboxylic acid (150 mg, 0.48 mmol), N-ethyl-2-(methylamino)-2-oxoethanaminium chloride (88 mg, 0.57 mmol) and O-(7-azabenzotriazol-1-yl)-N,N,N′,N′-tetramethyluronium hexafluorophosphate (218 mg, 0.57 mmol) were stirred in DMF (10 mL) containing N,N-diisopropylethylamine (0.208 mL, 1.20 mmol) at 23° C. for 1 h. The solvent was evaporated. The residue was dissolved in EtOAc and washed with saturated aqueous NaHCO₃, brine and dried over anhydrous Na₂SO₄. The product was purified by reversed-phase HPLC using 40-60% CH₃CN/H₂O and lyophilized (141 mg, 72%). NMR (400 MHz, METHANOL-D4) δ 1.15 (s, 3H), 1.35-1.46 (m, 2H), 1.50-1.59 (m, 3H), 1.75 (t, J=13.67 Hz, 2H), 2.14 (d, J=4.69 Hz, 1H), 2.31-2.41 (m, 1H), 2.62-2.70 (m, 1H), 2.75 (s, 3H), 2.77-2.88 (m, 2H), 3.36-3.45 (m, 2H), 3.45-3.57 (m, 2 H), 3.61 (s, 3H), 3.97 (dd, J=10.94, 3.52 Hz, 2H), 4.10 (s, 2H), 7.19 (s, 1H), 7.30 (d, J=7.81 Hz, 1H), 7.54 (s, 1H); MS (ESI) (M+H)⁺=412.3.

### Example 15

### Methyl, methyl[2-(methyl{[9-methyl-3-(tetrahydro-2H-pyran-4-yl)-2,3,4,9-tetrahydro-1H-carbazol-6-yl]carbonyl}amino) ethyl]carbamate

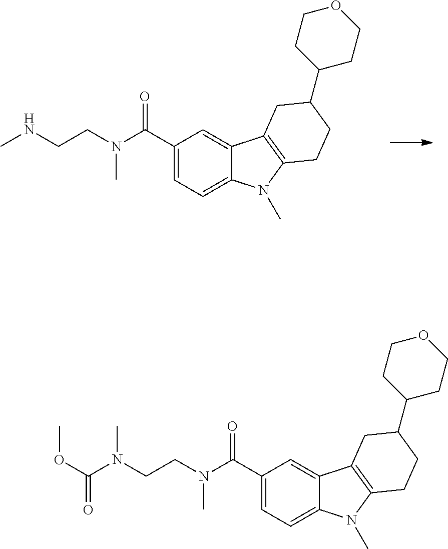

N,9-Dimethyl-N-(2-(methylamino)ethyl)-3-(tetrahydro-2H-pyran-4-yl)-2,3,4,9-tetrahydro-1H-carbazole-6-carboxamide, trifluoroacetic acid salt (65 mg, 0.13 mmol) was dissolved in DCM (5 mL) containing N,N-diisopropylethylamine (0.057 mL, 0.33 mmol). Methyl chloroformate (0.012 mL, 0.16 mmol) was added dropwise and the solution was stirred at 23° C. for 1 h. The solution was washed with saturated aqueous NaHCO₃, brine and dried over anhydrous Na₂SO₄. The product was purified by reversed-phase HPLC using 40-60% CH₃CN/H₂O and lyophilized (50 mg, 87%). ¹H NMR (400 MHz, METHANOL-D4) δ 1.36-1.49 (m, 2H), 1.53-1.62 (m, 3H), 1.77 (t, J=12.50 Hz, 2H), 2.13-2.21 (m, 1H), 2.34-2.43 (m, 1H), 2.49 (s, 1H), 2.62-2.74 (m, 1H), 2.80-2.90 (m, 2H), 2.95-3.02 (m, 2H), 3.03-3.13 (m, 3H), 3.33 (s, 2H), 3.37-3.46 (m, 2H), 3.50-3.61 (m, 2H), 3.62 (s, 3H), 3.66-3.78 (m, 3H), 3.98 (dd, J=10.94, 3.52 Hz, 2H), 7.10 (s, 1H), 7.31 (dd, J=8.40, 2.15 Hz, 1H), 7.38-7.46 (m, 1H);

### Example 16

### N-{2-[(Cyclopropylcarbonyl)(methyl)amino]ethyl}-N,9-dimethyl-3-(tetrahydro-2H-pyran-4-yl)-2,3,4,9-tetrahydro-1H-carbazole-6-carboxamide

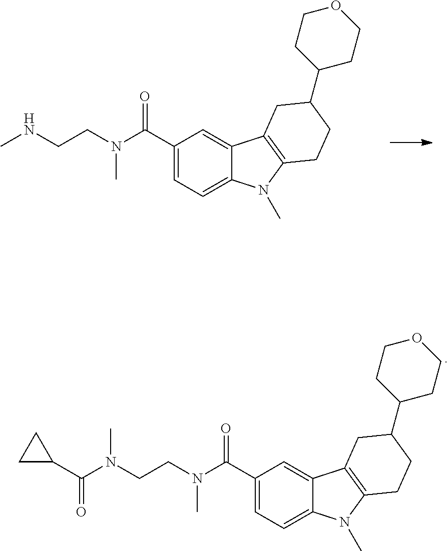

N,9-Dimethyl-N-(2-(methylamino)ethyl)-3-(tetrahydro-2H-pyran-4-yl)-2,3,4,9-tetrahydro-1H-carbazole-6-carboxamide trifluoroacetic acid salt (100 mg, 0.20 mmol) was dissolved in DCM (10 mL) containing N,N-diisopropylethylamine (0.088 mL, 0.50 mmol). Cyclopropanecarbonyl chloride (0.022 mL, 0.24 mmol) was added dropwise and the solution was stirred at 23° C. for 1 h. The solution was washed with saturated aqueous NaHCO₃, brine and dried over anhydrous Na₂SO₄. The product was purified by reversed-phase HPLC using 30-50% CH₃CN/H₂O and lyophilized (90 mg, 99%). ¹H NMR (400 MHz, METHANOL-D4) (rotomers) δ 0.13 (s, 0.25H), 0.48 (s, 0.25H), 0.77 (s, 1.75H), 0.89 (s, 1.75 H,) 1.38-1.49 (m, 2H), 1.52-1.61 (m, 3H), 1.78 (t, J=11.91 Hz, 2H), 1.89 (s, 0.25H), 2.09 (s, 0.25H), 2.13-2.21 (m, 1H), 2.32-2.44 (m, 1H), 2.62-2.75 (m, 2H), 2.80-2.89 (m, 2H), 3.04 (s, 2H), 3.07-3.17 (m, 2H), 3.37-3.47 (m, 2.25H), 3.58 (s, 0.5H), 3.63 (s, 3.5H), 3.72-3.81 (m, 2H), 3.85-3.91 (m, 0.25H), 3.98 (dd, J=11.13, 3.71 Hz, 2H), 7.06-7.17 (m, 1H), 7.26-7.36 (m, 1H), 7.38-7.52 (m, 1H); MS (ESI) (M+H)⁺=452.2.

### Example 17

### N-Cyclopropyl-1-{[9-methyl-3-(tetrahydro-2H-pyran-4-yl)-2,3,4,9-tetrahydro-1H-carbazol-6-yl]carbonyl}piperidine-3-carboxamide

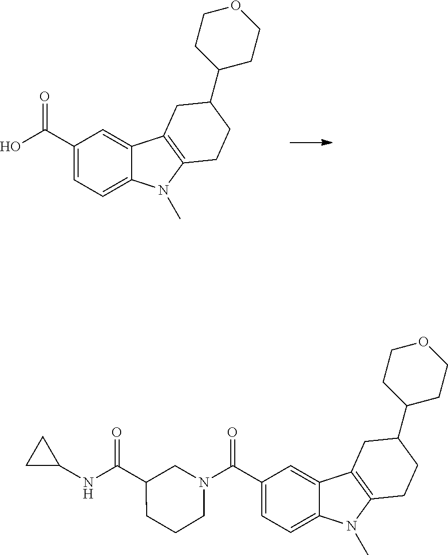

9-Methyl-3-(tetrahydro-2H-pyran-4-yl)-2,3,4,9-tetrahydro-1H-carbazole-6-carboxylic acid (100 mg, 0.32 mmol), HATU (146 mg, 0.38 mmol) and 3-(cyclopropylcarbamoyl)piperidinium 2,2,2-trifluoroacetate (108 mg, 0.38 mmol) were stirred in DMF (10 mL) containing N,N-diisopropylethylamine (0.139 mL, 0.80 mmol) at 23° C. for 1 h. The solvent was evaporated. The residue was dissolved in EtOAc and washed with aqueous saturated NaHCO₃, brine and dried over anhydrous Na₂SO₄. The product was purified by reversed-phase HPLC using 40-60% CH₃CN/H₂O and lyophilized (45 mg, 30%). ¹H NMR (400 MHz, METHANOL-D4) δ 0.45 (s, 2H), 0.68 (s, 2H), 1.36-1.50 (m, 3H), 1.51-1.62 (m, 4H), 1.77 (t, J=12.50 Hz, 3H), 1.88-1.96 (m, 1H), 2.11-2.22 (m, 1H), 2.32-2.44 (m, 2H), 2.63-2.74 (m, 2H), 2.78-2.90 (m, 2H), 3.12 (s, 2H), 3.37-3.50 (m, 2H), 3.63 (s, 3H), 3.80 (s, 1H), 3.98 (dd, J=11.33, 3.12 Hz, 2H), 4.46 (s, 1H), 7.12 (dd, J=8.20, 1.56 Hz, 1H), 7.31 (d, J=8.20 Hz, 1H), 7.46 (d, J=0.78 Hz, 1H). MS (ESI) (M+H)⁺=464.2.

### Example 18

### Step A: N-Cyclopropyl-1-{[9-methyl-3-(tetrahydro-2H-pyran-4-yl)-2,3,4,9-tetrahydro-1H-carbazol-6-yl]carbonyl}azetidine-3-carboxamide

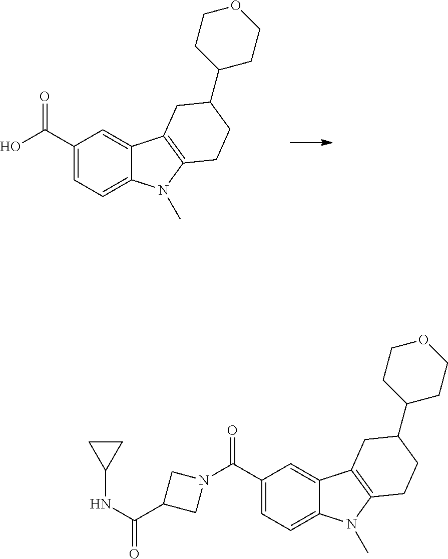

9-Methyl-3-(tetrahydro-2H-pyran-4-yl)-2,3,4,9-tetrahydro-1H-carbazole-6-carboxylic acid (125 mg, 0.40 mmol), HATU (182 mg, 0.48 mmol) and 3-(cyclopropylcarbamoyl)azetidinium chloride (85 mg, 0.48 mmol) were stirred in DMF (5 mL) containing N,N-diisopropylethylamine (0.174 mL, 1.00 mmol) at 23° C. for 1 h. The solvent was evaporated. The residue was dissolved in EtOAc and washed with saturated aqueous NaHCO₃, brine and dried over anhydrous Na₂SO₄. The product was purified by reversed-phase HPLC using 30-50% CH₃CN/H₂O and lyophilized (55 mg, 32%). ¹H NMR (400 MHz, DMSO-D6) δ 0.34-0.41 (m, 2H), 0.57-0.63 (m, 2H), 1.27-1.38 (m, 2H), 1.46-1.58 (m, 3H), 1.71 (d, J=13.28 Hz, 2H), 2.04-2.13 (m, 1H), 2.33 (m, 1H), 2.59-2.70 (m, 2H), 2.76-2.88 (m, 2H), 3.25-3.35 (m, 3H), 3.61 (s, 3H), 3.89 (dd, J=10.94, 3.12 Hz, 2H), 4.01 (s, 1H), 4.09 (s, 1H), 4.29 (s, 1H), 4.40 (s, 1H), 7.36 (d, J=1.17 Hz, 1H), 7.36 (s, 1H), 7.67 (s, 1H), 8.07 (d, J=3.91 Hz, 1H). MS (ESI) (M+H)⁺=436.2.

### Step B: 3-[(Cyclopropylamino)carbonyl]azetidinium chloride

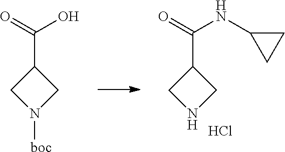

1-Boc-azetidine-3-carboxylic acid (125 mg, 0.62 mmol), O-(7-azabenzotriazol-1-yl)-N,N,N′,N′-tetramethyluronium hexafluorophosphate (283 mg, 0.75 mmol) and cyclopropylamine (0.052 mL, 0.75 mmol) were stirred in DMF (8 mL) containing N,N-diisopropylethylamine (0.162 mL, 0.93 mmol) at 23° C. for 1 h. The solvent was evaporated. The residue was dissolved in EtOAc and washed with saturated aqueous NaHCO₃, 5% KHSO₄, brine and dried over anhydrous Na₂SO₄. The solvent was evaporated. The residue was dissolved in 1M Hydrogen chloride (3.11 mL, 3.11 mmol) in AcOH and stirred at 23° C. for 2 h. The solvent was evaporated. The residue was rinsed twice with ether and dried under vacuum (109 mg, 99%). ¹H NMR (400 MHz, METHANOL-D4) δ 0.43-0.51 (m, 2H), 0.67-0.78 (m, 2H), 2.64-2.72 (m, 1H), 3.47-3.59 (m, 1H), 4.15 (d, J=7.81 Hz, 4H).

### Example 19

### Step A: N-Ethyl-N-{2-[(1-isocyanocyclopropyl)amino]-2-oxoethyl}-9-methyl-3-(tetrahydro-2H-pyran-4-yl)-2,3,4,9-tetrahydro-1H-carbazole-6-carboxamide

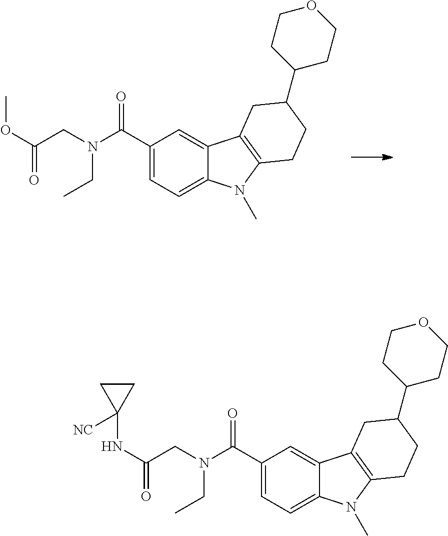

Methyl 2-(N-ethyl-9-methyl-3-(tetrahydro-2H-pyran-4-yl)-2,3,4,9-tetrahydro-1H-carbazole-6-carboxamido)acetate (130 mg, 0.32 mmol) was stirred in dioxane (5 mL) containing lithium hydroxide (0.630 mL, 0.63 mmol) (1M/water) at 50° C. for 2 h. The solvent was evaporated. The residue was dissolved in DMF (5.00 mL) containing N,N-diisopropylethylamine (0.137 mL, 0.79 mmol) and 1-amino-1-cyclopropancarbonitirle HCl (44.8 mg, 0.38 mmol) along with HATU (144 mg, 0.38 mmol) were added. The solution was stirred at rt for 2 h. The solvent was evaporated. The residue was dissolved in EtOAc and washed with aqueous saturated NaHCO₃ solution, brine and dried over anhydrous Na₂SO₄. The product was purified by reversed-phase HPLC using 50-70% CH₃CN/H₂O and lyophilized (80 mg, 55%). ¹H NMR (400 MHz, METHANOL-D4) δ 1.07-1.30 (m, 5H), 1.39-1.51 (m, 5H), 1.56 (s, 3H), 1.77 (t, J=11.52 Hz, 2H), 2.15 (m, 1H), 2.38 (m, 1H), 2.62-2.74 (m, 1H), 2.78-2.90 (m, 2H), 3.36-3.46 (m, 3H), 3.49-3.59 (m, 1H), 3.62 (s, 2H), 3.97 (dd, J=10.94, 3.52 Hz, 3H), 4.08 (s, 1H), 7.17 (s, 1H), 7.31 (d, J=8.20 Hz, 1H), 7.43-7.58 (m, 1H); MS (ESI) (M+H)⁺=463.3.

### Step B: N-Ethyl-2-methoxy-2-oxoethanaminium chloride

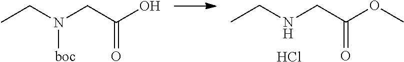

2-(tert-Butoxycarbonyl(ethyl)amino)acetic acid (500 mg, 2.46 mmol) was dissolved in MeOH (10 mL) at 0° C. (Trimethylsilyl)diazomethane (3.69 mL, 7.38 mmol) was added dropwise to the stirring solution until a light yellow color persisted. The solution was then stirred at rt for 20 min. The solvent was evaporated. The residue was dissolved in EtOAc and washed with aqueous saturated NaHCO₃, brine and dried over anhydrous Na₂SO₄. The solvent was evaporated. The product was then stirred in 1M HCl/AcOH (12.30 mL, 12.30 mmol) at rt for 2 h. The solvent was evaporated. The residue was washed with ether and the ether was decanted. The product was dried under vacuum (307 mg, 81%). ¹H NMR (400 MHz, METHANOL-D4) δ 1.30 (t, J=7.23 Hz, 3H), 3.10 (q, J=7.16 Hz, 2H), 3.82 (s, 3H), 3.96 (s, 2H).

### Step C: Methyl N-ethyl-N-{[9-methyl-3-(tetrahydro-2H-pyran-4-yl)-2,3,4,9-tetrahydro-1H-carbazol-6-yl]carbonyl}glycinate

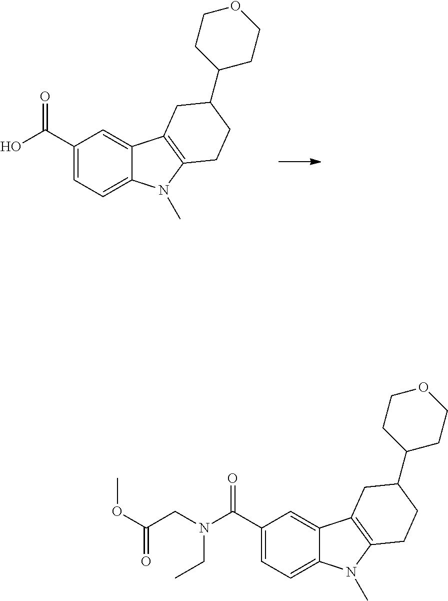

9-Methyl-3-(tetrahydro-2H-pyran-4-yl)-2,3,4,9-tetrahydro-1H-carbazole-6-carboxylic acid (100 mg, 0.32 mmol), HATU (146 mg, 0.38 mmol) and N-ethyl-2-methoxy-2-oxoethanaminium chloride (58.8 mg, 0.38 mmol) were stirred in DMF (8 mL) containing N,N-diisopropylethylamine (0.139 mL, 0.80 mmol) at 23° C. for 1 h. The solvent was evaporated. The residue was dissolved in EtOAc and washed with aqueous saturated NaHCO₃ solution, brine and dried over anhydrous Na₂SO₄. The product was purified by flash chromatography using a gradient form 50% EtOAc/heptane to 100% EtOAc (130 mg, 99%). ¹H NMR (400 MHz, METHANOL-D4) δ 1.10-1.17 (m, 2H), 1.20 (d, J=5.47 Hz, 2H), 1.38-1.49 (m, 1H), 1.53-1.62 (m, 3H), 1.77 (t, J=13.67 Hz, 2H), 2.14-2.20 (m, 1H), 2.34-2.44 (m, 1H), 2.64-2.75 (m, 1H), 2.79-2.91 (m, 2H), 3.37-3.47 (m, 3H), 3.58 (s, 1H), 3.63 (s, 3H), 3.66-3.71 (m, 1H), 3.76 (s, 2H), 3.98 (dd, J=11.33, 3.52 Hz, 2H), 4.22 (s, 2H), 7.16 (d, J=8.98 Hz, 1H), 7.33 (d, J=8.20 Hz, 1H), 7.49 (s, 1H); MS (ESI) (M+H)⁺=413.28.

### Example 20

### N-Ethyl-N-(2-hydroxyethyl)-9-methyl-3-(tetrahydro-2H-pyran-4-yl)-2,3,4,9-tetrahydro-1H-carbazole-6-carboxamide

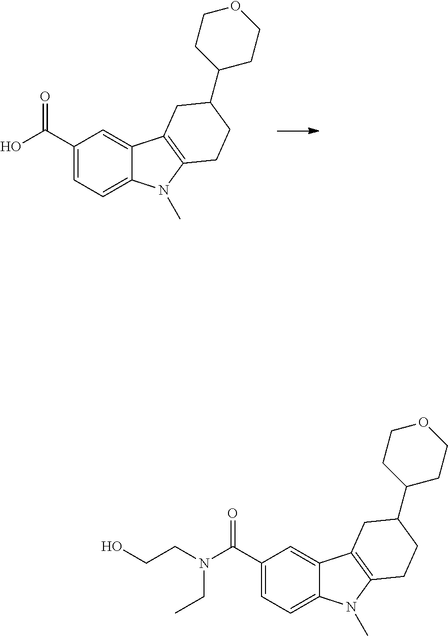

To a solution of 2-(ethylamino)ethanol (133 mg, 1.49 mmol), 9-methyl-3-(tetrahydro-2H-pyran-4-yl)-2,3,4,9-tetrahydro-1H-carbazole-6-carboxylic acid (156 mg, 0.50 mmol) and DIPEA (0.5 mL) in DMF (5 mL) was added HATU (228 mg, 0.6 mmol) at 0 C. The reaction mixture was stirred for 2 hrs. at r.t and the solvent was concentrated. The product was purified by preparative reverse-phase HPLC using an acetonitrile gradient 20 to 50% in water to provide the title compound as white solid (128 mg, 67%). ¹H NMR (400 MHz, METHANOL-D4) δ 1.11 (m, 2H), 1.25 (m, 1H), 1.35-1.46 (m, 2H), 1.49-1.59 (m, 3H), 1.74 (m, 2H), 2.09-2.18 (m, 1H), 2.31-2.40 (m, 1H), 2.65 (m, 1H), 2.77-2.86 (m, 2H), 3.36-3.47 (m, 4H), 3.53-3.65 (m, 6H), 3.72-3.84 (m, 1H), 3.96 (m, 2H), 7.12 (d, J=8.20 Hz, 1H), 7.30 (d, J=8.20 Hz, 1H), 7.46 (s, 1H); MS (ESI) (M+H)⁺ 385.25.

### Example 21

### N-(3-(cyclopropylamino)-3-oxopropyl)-N,9-dimethyl-3-(tetrahydro-2H-pyran-4-yl)-2,3,4,9-tetrahydro-1H-carbazole-6-carboxamide

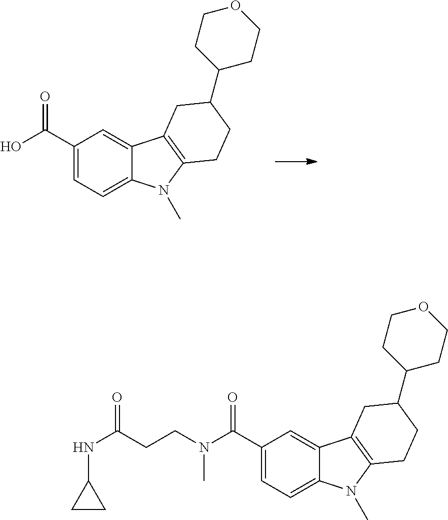

To a solution of 3-(cyclopropylamino)-N-methyl-3-oxopropan-1-aminium chloride (178 mg, 1.00 mmol), 9-methyl-3-(tetrahydro-2H-pyran-4-yl)-2,3,4,9-tetrahydro-1H-carbazole-6-carboxylic acid (156 mg, 0.50 mmol) and DIPEA (0.5 mL) in DMF (5 mL) was added HATU (228 mg, 0.6 mmol) at 0 C. The reaction mixture was stirred for 2 hrs. at r.t and the solvent was concentrated. The product was purified by preparative reverse-phase HPLC using an acetonitrile gradient 20 to 50% in water to provide the title compound as white solid (8 mg, 3.4%). ¹H NMR (400 MHz, METHANOL-D4) δ 0.47 (m, 2H), 0.68 (m, 2H), 1.43 (m, 2H), 1.52-1.61 (m, 4H), 1.77 (m, 2H), 2.12-2.19 (m, 1H), 2.40 (m, 1H), 2.50 (m, 1H), 2.68 (m, 1H), 2.82-2.89 (m, 2H), 3.03 (s, 3H), 3.37-3.46 (m, 3H), 3.62 (s, 3H), 3.75 (m, 2H), 3.98 (m, 2H), 7.12 (dd, J=8.0, 1.6 Hz, 1H), 7.31 (d, J=8.0 Hz, 1H), 7.45 (s, 1H); MS (ESI) (M+H)⁺ 483.3.

### Example 22

### N-(4-(Cyclopropylamino)-4-oxobutyl)-N-ethyl-9-methyl-3-(tetrahydro-2H-pyran-4-yl)-2,3,4,9-tetrahydro-1H-carbazole-6-carboxamide

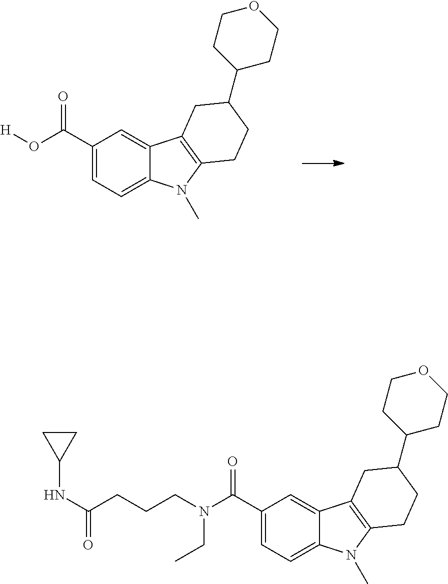

To a solution of 4-(cyclopropylamino)-N-ethyl-4-oxobutan-1-aminium chloride (206 mg, 995.56 μmol), 9-methyl-3-(tetrahydro-2H-pyran-4-yl)-2,3,4,9-tetrahydro-1H-carbazole-6-carboxylic acid (156 mg, 497.78 μmol) and DIPEA (0.5 mL) in DMF (5 mL) was added HATU (228 mg, 0.6 mmol) at 0 C. The reaction mixture was stirred for 2 hrs. at r.t and the solvent was concentrated. The product was purified by preparative reverse-phase HPLC using an acetonitrile gradient 20 to 50% in water to provide the title compound as white solid (118 mg, 49%). ¹H NMR (400 MHz, METHANOL-D4) δ 0.2-0.75 (m, 4H), 1.11 (m, 2H),1.25 (m, 1H), 1.46 (m, 2H), 1.57 (m, 3H), 1.77 (m, 3H), 1.92 (m, 2H), 2.09-2.28 (m, 2H), 2.31-2.42 (m, 1H), 2.65 (m, 1H), 2.77-2.86 (m, 2H), 3.28-3.60 (m, 7H), 3.62 (s, 3H), 3.98 (dd, J=11.3, 3.5 Hz, 2H), 7.08 (d, J=8.0 Hz, 1H), 7.31 (d, J=8.0 Hz, 1H), 7.41 (s, 1H); MS (ESI) (M+H)⁺ 466.2.

### Example 23

### N-(4-(Cyclopropylamino)-4-oxobutyl)-N-methyl-9-methyl-3-(tetrahydro-2H-pyran-4-yl)-2,3,4,9-tetrahydro-1H-carbazole-6-carboxamide

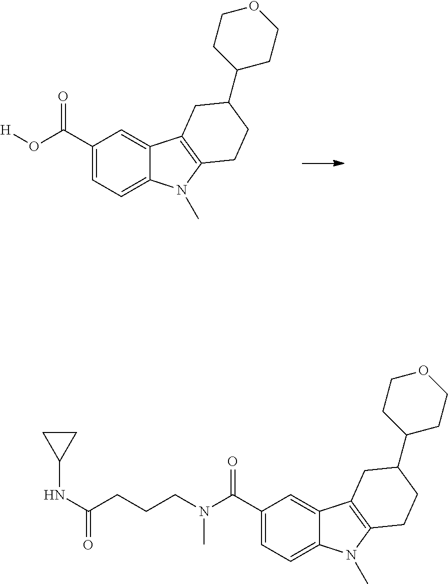

To a solution of 4-(cyclopropylamino)-N-methyl-4-oxobutan-1-aminium chloride (134 mg, 0.7 mmol), 9-methyl-3-(tetrahydro-2H-pyran-4-yl)-2,3,4,9-tetrahydro-1H-carbazole-6-carboxylic acid (156 mg, 0.5 mmol) and DIPEA (0.5 mL) in DMF (5 mL) was added HATU (228 mg, 0.6 mmol) at 0 C. The reaction mixture was stirred for 2 hrs. at r.t and the solvent was concentrated. The product was purified by preparative reverse-phase HPLC using an acetonitrile gradient 20 to 50% in water to provide the title compound as white solid (69 mg, 31%). ¹H NMR (400 MHz, METHANOL-D4) δ 0.2-0.70 (m, 4H), 1.43 (m, 2H), 1.56 (m, 3H), 1.76 (m, 3H), 1.93 (m, 2H), 2.09-2.28 (m, 2H), 2.31-2.42 (m, 1H), 2.65 (m, 1H), 2.77-2.86 (m, 2H), 3.03 (m, 3H), 3.28-3.60 (m, 5H), 3.62 (s, 3H), 3.97 (dd, J=11.3, 3.5 Hz, 2H), 7.12 (s 1H), 7.30 (d, J=8.0 Hz, 1H), 7.46 (s, 1H); MS (ESI) (M+H)⁺ 452.2913.

### Example 24

### N-(4-(Methylamino)-4-oxobutyl)-N-methyl-9-methyl-3-(tetrahydro-2H-pyran-4-yl)-2,3,4,9-tetrahydro-1H-carbazole-6-carboxamide

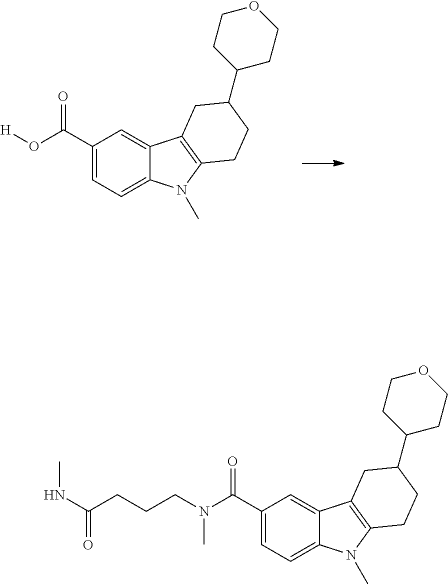

To a solution of 4-(methylamino)-N-methyl-4-oxobutan-1-aminium chloride (176 mg, 1.0 mmol), 9-methyl-3-(tetrahydro-2H-pyran-4-yl)-2,3,4,9-tetrahydro-1H-carbazole-6-carboxylic acid (156 mg, 0.5 mmol) and DIPEA (0.5 mL) in DMF (5 mL) was added HATU (228 mg, 0.6 mmol) at 0 C. The reaction mixture was stirred for 2 hrs. at r.t and the solvent was concentrated. The product was purified by preparative reverse-phase HPLC using an acetonitrile gradient 20 to 50% in water to provide the title compound as white solid (104 mg, 49%). ¹H NMR (400 MHz, METHANOL-D4) δ 1.43 (m, 2H), 1.56 (m, 3H), 1.76 (m, 2H), 1.93 (m, 3H), 2.158 (m, 1H), 2.26 (m, 1H), 2.38 (m, 1H), 2.52 (m, 1H), 2.68 (m, 3H), 2.77-2.86 (m, 2H), 3.03 (m, 3H), 3.40 (m, 3H), 3.56 (m, 1H), 3.62 (s, 3H), 3.97 (dd, J=11.3, 3.5 Hz, 2H), 7.12 (s 1H), 7.30 (d, J=8.0 Hz, 1H), 7.46 (s, 1H); MS (ESI) (M+H)⁺ 426.2757.

### Example 25

### N-(4-(Ethylamino)-4-oxobutyl)-N-methyl-9-methyl-3-(tetrahydro-2H-pyran-4-yl)-2,3,4,9-tetrahydro-1H-carbazole-6-carboxamide

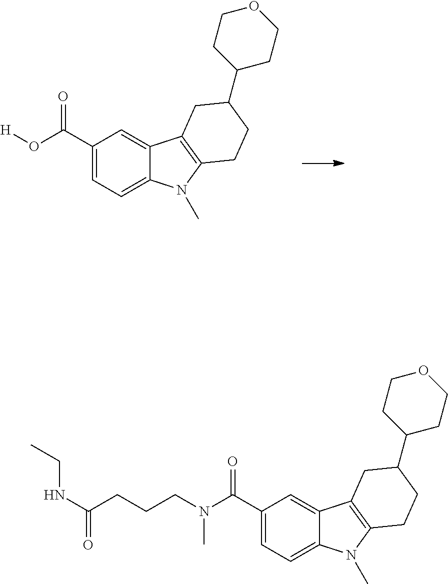

To a solution of 4-(ethylamino)-N-methyl-4-oxobutan-1-aminium chloride (185 mg, 1.5 mmol), 9-methyl-3-(tetrahydro-2H-pyran-4-yl)-2,3,4,9-tetrahydro-1H-carbazole-6-carboxylic acid (156 mg, 0.5 mmol) and DIPEA (0.5 mL) in DMF (5 mL) was added HATU (228 mg, 0.6 mmol) at 0 C. The reaction mixture was stirred for 2 hrs. at r.t and the solvent was concentrated. The product was purified by preparative reverse-phase HPLC using an acetonitrile gradient 20 to 50% in water to provide the title compound as white solid (143 mg, 65%). ¹H NMR (400 MHz, METHANOL-D4) δ 0.8-1.12 (m, 3H), 1.43 (m, 2H), 1.52 (m, 3H), 1.76 (m, 2H), 1.93 (m, 3H), 2.13 (m, 1H), 2.25 (m, 1H), 2.34 (m, 1H), 2.65 (m, 1H), 2.77-2.86 (m, 2H), 2.9-3.25 (m, 5H), 3.3-3.60 (m, 4H), 3.60 (s, 3H), 3.97 (m, 2H), 7.12 (s 1H), 7.29 (d, J=8.0 Hz, 1H), 7.46 (s, 1H); MS (ESI) (M+H)⁺ 440.2913.

### Example 26

### N-(4-(2-Fluoroethylamino)-4-oxobutyl)-N-methyl-9-methyl-3-(tetrahydro-2H-pyran-4-yl)-2,3,4,9-tetrahydro-1H-carbazole-6-carboxamide

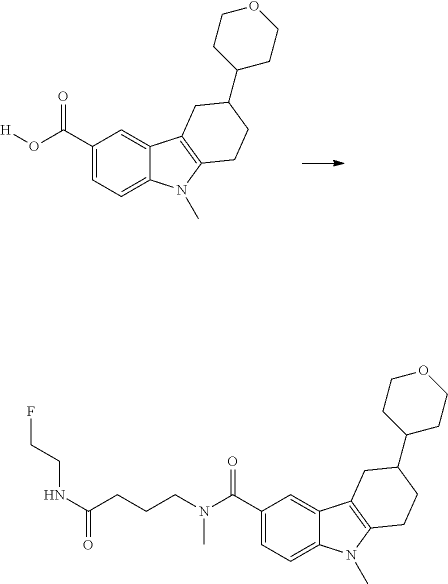

To a solution of N-methyl-4-(2-fluoroethylamino)-4-oxobutan-1-aminium chloride (320 mg, 1.5 mmol), 9-methyl-3-(tetrahydro-2H-pyran-4-yl)-2,3,4,9-tetrahydro-1H-carbazole-6-carboxylic acid (156 mg, 0.5 mmol) and DIPEA (0.5 mL) in DMF (5 mL) was added HATU (228 mg, 0.6 mmol) at 0 C. The reaction mixture was stirred for 2 hrs. at r.t and the solvent was concentrated. The product was purified by preparative reverse-phase HPLC using an acetonitrile gradient 20 to 50% in water to provide the title compound as white solid (89 mg, 39%). ¹H NMR (400 MHz, METHANOL-D4) δ 1.43 (m, 2H), 1.56 (m, 3H), 1.76 (m, 2H), 1.93 (m, 3H), 2.13 (m, 1H), 2.33 (m, 2H), 2.65 (m, 1H), 2.83 (m, 2H), 3.03 (m, 3H), 3.15-3.60 (m, 4H), 3.62 (s, 3H), 3.97 (dd, J=11.3, 3.5 Hz, 2H), 4.1-4.5 (m, 2H), 7.12 (s 1H), 7.30 (d, J=8.0 Hz, 1H), 7.46 (s, 1H); MS (ESI) (M+H)⁺ 458.2819.

### Example 27

### 3-cyclohexyl-9-methyl-6-[(4-methylpiperidin-1-yl)carbonyl]-2,3,4,9-tetrahydro-1H-carbazole

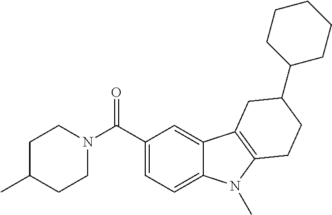

### Step A 3-cyclohexyl-9-methyl-6-[(4-methylpiperidin-1-yl)carbonyl]-2,3,4,9-tetrahydro-1H-carbazole

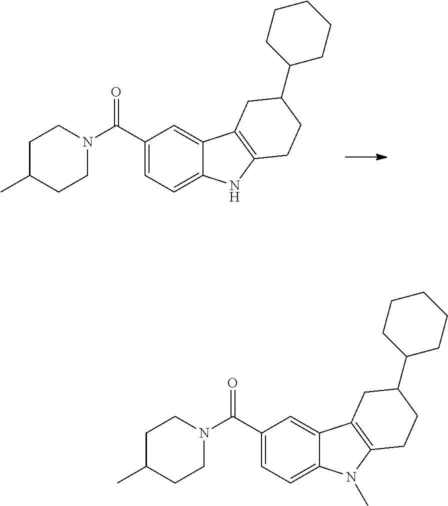

Sodium hydride (60 mg, 1.5 mmol) was added to a solution of 3-cyclohexyl-6-[(4-methylpiperidin-1-yl)carbonyl]-2,3,4,9-tetrahydro-1H-carbazole (120 mg, 0.32 mmol) (see following steps B, and C for preparation) in THF (10 mL). Stirring for 1 h at room temperature, methyl iodide (135 mg, 0.95 mmol) was added. The reaction mixture was stirred overnight at room temperature, quenched with NaHCO₃ (5 mL), diluted with EtOAc (100 mL), washed with water (10 mL) and NaCl (10 mL), and then dried over Na₂SO₄. The crude product was purified by MPLC on silica gel using Hex/EtOAc (1:1) to give 117 mg (94%) of a white solid as the title compound. ¹H NMR (400 MHz, CHLOROFORM-D) δ 0.98 (d, J=6.25 Hz, 3H), 1.03-1.43 (m, 10H), 1.50-1.73 (m, 5 H), 1.75-1.90 (m, 5H), 2.03-2.17 (m, 1H), 2.34-2.48 (m, 1H), 2.60-2.73 (m, 1H), 2.75-3.01 (m, 4H), 3.63 (s, 3H), 7.19-7.23 (m, 2H), 7.56 (s, 1H); MS (APPI) (M+H)⁺=393.2; Anal. Calcd for C₂₆H₃₆N₂O₃+0.25 MeOH: C, 78.71; H, 9.31; N, 6.99. Found: C, 78.73; H, 9.30; N, 7.02.

### Step B: 3-cyclohexyl-2,3,4,9-tetrahydro-1H-carbazole-6-carboxylic acid

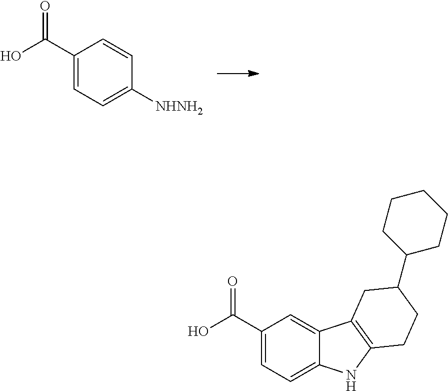

4-Hydrazinobenzoic acid (0.76 g, 5.0 mmol) and 4-cyclohexyl cyclohexanone (0.99 g, 5.5 mmol) in dioxane (15 ml) and concentrated hydrochloric acid (1.5 ml) were heated overnight at reflux. Upon evaporation, the residue was dissolved in EtOAc (200 mL), washed with water (2×20 mL), NaCl (2×20 mL) and dried over Na₂SO₄. After concentration, 1.67 g of a brown solid was obtained, which was used directly for next step without further purification. MS (APPI) (M+H)⁺=298.23.

### Step C: 3-cyclohexyl-6-[(4-methylpiperidin-1-yl)carbonyl]-2,3,4,9-tetrahydro-1H-carbazole

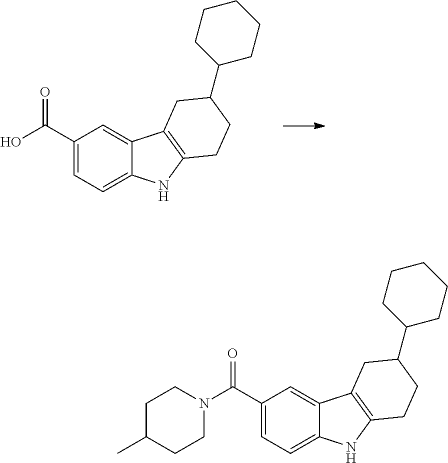

DIPEA (0.65 g, 0.89 mL, 5.0 mmol) was added to a solution of 3-cyclohexyl-2,3,4,9-tetrahydro-1H-carbazole-6-carboxylic acid (0.83 g, 2.5 mmol) and 4-methylpiperidine (0.50 g, 0.60 mL, 5.0 mmol) in DMF (15 mL). Stirring for 20 min, HATU (1.43 g, 3.75 mmol) was added at 0° C. The mixture was stirred overnight at room temperature, quenched with water (100 mL) and extracted with EtOAc (3×50 mL). The combined organic phase was washed with water (2×50 mL), NaCl (2×50 mL) and dried over Na₂SO₄. The crude product was purified by MPLC on silica gel using Hex/EtOAc (1:1) to give 0.54 g (57%) of a light yellow solid as the title compound. ¹H NMR (400 MHz, CHLOROFORM-D) δ 0.98 (d, J=6.25 Hz, 3H), 1.05-1.43 (m, 11H), 1.58-1.74 (m, 5 H), 1.79 (d, J=13.67 Hz, 5H), 1.99-2.14 (m, 1H), 2.40 (m, 1H), 2.67-3.09 (m, 4H), 7.13-7.19 (m, 1H), 7.24 (s, 1H), 7.54 (s, 1H), 7.80 (s, 1H).

### Example 28

### 3-cyclohexyl-9-ethyl-6-[(4-methylpiperidin-1-yl)carbonyl]-2,3,4,9-tetrahydro-1H-carbazole

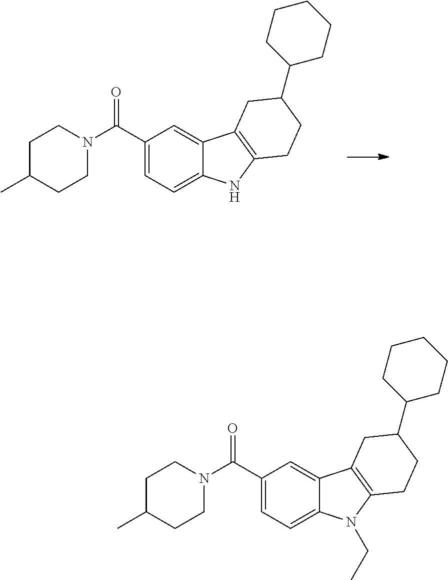

Following the procedure for Step A in Example 27, using 3-cyclohexyl-6-[(4-methylpiperidin-1-yl)carbonyl]-2,3,4,9-tetrahydro-1H-carbazole (120 mg, 0.32 mmol), sodium hydride (60 mg, 1.5 mmol) and ethyl iodide (149 mg, 0.95 mmol) in THF (10 mL). The crude product was purified by MPLC on silica gel using Hex/EtOAc (1:1) to give 104 mg (81%) of a white solid as the title compound. ¹H NMR (400 MHz, CHLOROFORM-D) δ 0.98 (d, J=6.25 Hz, 3H), 1.04-1.29 (m, 10H), 1.32 (t, J=7.23 Hz, 3H), 1.59-1.73 (m, 5H), 1.74-1.89 (m, 5H), 2.03-2.15 (m, 1H), 2.42 (dd, J=13.87, 8.40 Hz, 1H), 2.60-2.75 (m, 1H), 2.75-3.00 (m, 4H), 4.02-4.14 (m, 2H), 7.16-7.25 (m, 2H), 7.55 (s, 1H); MS (APPI) (M+H)⁺=407.3

### Example 29

### 3-cyclohexyl-6-[(4-methylpiperidin-1-yl)carbonyl]-9-(methylsulfonyl)-2,3,4,9-tetrahydro-1H-carbazole

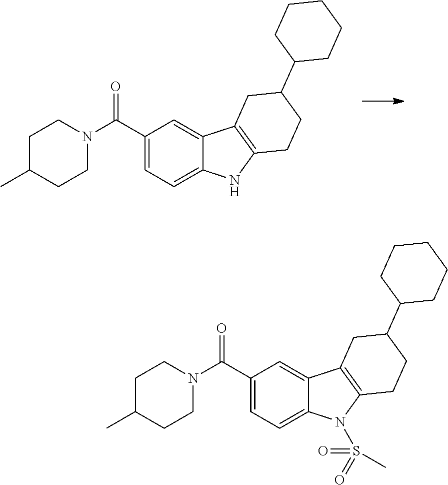

Following the procedure for Step A in Example 27, using 3-cyclohexyl-6-[(4-methylpiperidin-1-yl)carbonyl]-2,3,4,9-tetrahydro-1H-carbazole (120 mg, 0.32 mmol), sodium hydride (60 mg, 1.5 mmol) and methanesulfonyl chloride (73 mg, 0.64 mmol) in THF (10 mL). The crude product was purified by MPLC on silica gel using CH₂Cl₂/EtOAc (5:1) to give 67 mg (46%) of a white solid as the title compound. ¹H NMR (400 MHz, CHLOROFORM-D) δ 0.99 (d, J=6.64 Hz, 3H), 1.02-1.39 (m, 10H), 1.47-1.74 (m, 5H), 1.74-1.89 (m, 5H), 2.03-2.15 (m, 1H), 2.28-2.40 (m, 1H), 2.68-2.88 (m, 3H), 2.98 (s, 3H), 3.08-3.18 (m, 1H), 3.62-3.88 (m, 0.5H), 4.59-4.83 (m, 0.5H), 7.29 (dd, J=8.59, 1.56 Hz, 1H), 7.50 (s, 1H), 7.97 (d, J=8.59 Hz, 1H); MS (APPI) (M+H)⁺=457.3; Anal. Calcd for C₂₆H₃₆N₂O₃S+0.2H₂O: C, 67.85; H, 7.97; N, 6.09. Found: C, 67.85; H, 7.90; N, 6.14.

### Example 30

### 3-cyclohexyl-9-(ethylsulfonyl)-6-[(4-methylpiperidin-1-yl)carbonyl]-2,3,4,9-tetrahydro-1H-carbazole

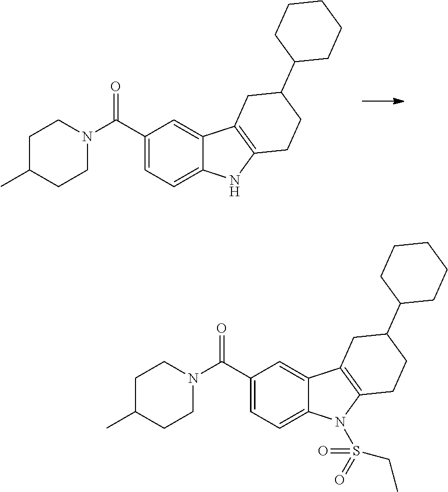

Following the procedure for Step A in Example 27, using 3-cyclohexyl-6-[(4-methylpiperidin-1-yl)carbonyl]-2,3,4,9-tetrahydro-1H-carbazole (120 mg, 0.32 mmol), sodium hydride (60 mg, 1.5 mmol) and ethanesulfonyl chloride (82 mg, 0.64 mmol) in THF (10 mL). The crude product was purified by MPLC on silica gel using CH₂Cl₂/EtOAc (10:1) to give 80 mg (53%) of a white solid as the title compound. ¹H NMR (400 MHz, CHLOROFORM-D) δ 0.99 (d, J=6.25 Hz, 3H), 1.02-1.15 (m, 3H), 1.19 (t, J=7.42 Hz, 3H), 1.22-1.37 (m, 5H), 1.46-1.73 (m, 5H), 1.74-1.92 (m, 5H), 2.03-2.16 (m, 1H), 2.27-2.42 (m, 1H), 2.66-3.07 (m, 4H), 3.09-3.17 (m, 1H), 3.21 (q, J=7.42 Hz, 2H), 3.66-3.92 (m, 1H), 4.59-4.85 (m, 1H), 7.24-7.31 (m, 1H), 7.49 (d, J=0.78 Hz, 1H), 7.95 (d, J=8.59 Hz, 1H); MS (APPI) (M+H)⁺=471.3; Anal. Calcd for C₂₇H₃₈N₂O₃S: C, 68.90; H, 8.14; N, 5.95. Found: C, 68.73; H, 7.80; N, 6.30.

### Example 31

### 3-cyclohexyl-N-[2-(cyclopropylamino)-2-oxoethyl]-N-methyl-9-(methylsulfonyl)-2,3,4,9-tetrahydro-1H-carbazole-6-carboxamide

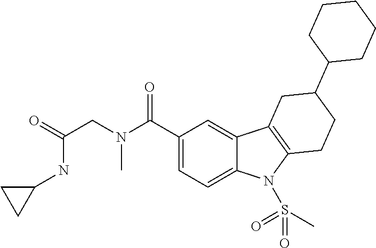

### Step A: 3-cyclohexyl-N-[2-(cyclopropylamino)-2-oxoethyl]-N-methyl-9-(methylsulfonyl)-2,3,4,9-tetrahydro-1H-carbazole-6-carboxamide

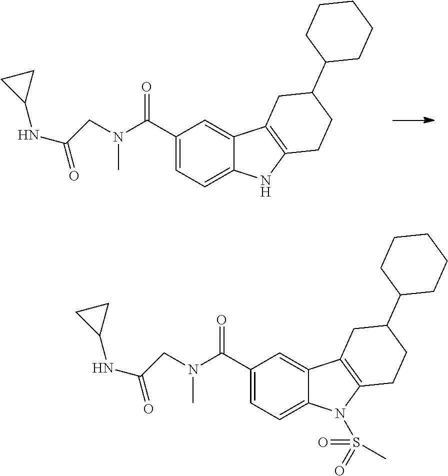

Following the procedure for Step A in Example 27, using 3-cyclohexyl-N-[2-(cyclopropylamino)-2-oxoethyl]-N-methyl-2,3,4,9-tetrahydro-1H-carbazole-6-carboxamide (121 mg, 0.30 mmol) (see following step B for preparation), sodium hydride (119 mg, 3.0 mmol) and methanesulfonyl chloride (340 mg, 3.0 mmol) in DMF (6 mL). The crude product was purified by MPLC on silica gel using EtOAc and then reverse-phase HPLC using high pH column 50-70% MeCN/H₂O to give 50 mg (34%) of a white solid as the title compound. ¹H NMR (400 MHz, METHANOL-D4) δ 0.36-0.44 (m, 1H), 0.48-0.58 (m, 1H), 0.70 (dd, J=10.55, 6.25 Hz, 2H), 1.02-1.39 (m, 6H), 1.44-1.72 (m, 3H), 1.74-1.92 (m, 4H), 2.03-2.19 (m, 1H), 2.26-2.45 (m, 1H), 2.58-2.88 (m, 2H), 3.02-3.18 (m, 7H), 3.89 (s, 1.5H), 4.13 (s, 1.5H), 7.30 (d, J=8.59 Hz, 0.5H), 7.37 (d, J=8.59 Hz, 0.5H), 7.50 (s, 0.5H), 7.61 (s, 0.5H), 7.95 (d, J=8.59 Hz, 0.5H), 7.98 (d, J=8.98 Hz, 0.5H); MS (APPI) (M+H)⁺=486.2.

### Step B: 3-cyclohexyl-N-[2-(cyclopropylamino)-2-oxoethyl]-N-methyl-2,3,4,9-tetrahydro-1H-carbazole-6-carboxamide

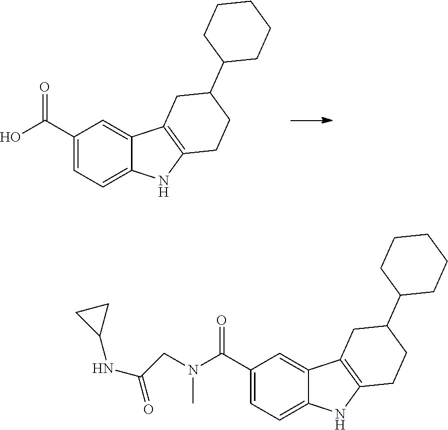

Following the procedure for Step C in Example 27, using 3-Cyclohexyl-2,3,4,9-tetrahydro-1H-carbazole-6-carboxylic acid (0.42 g, 1.25 mmol), N¹-cyclopropyl-N²-methylglycinamide (0.24 g, 1.88 mmol), DIPEA (0.33 g, 0.44 mL, 2.5 mmol) and HATU (0.72 g, 1.89 mmol) in DMF (10 mL). The crude product was purified by MPLC on silica gel using EtOAc to give 0.34 g (67%) of colorless syrup as the title compound. 111 NMR (400 MHz, CHLOROFORM-D) δ 0.51-0.60 (m, 2H), 0.76-0.87 (m, 2H), 1.01-1.44 (m, 6H), 1.54-1.74 (m, 2H), 1.74-1.90 (m, 4H), 1.99-2.13 (m, 2H), 2.40 (m, 1H), 2.70-2.79 (m, 4H), 3.15 (s, 3H), 4.03-4.16 (m, 2H), 7.17-7.25 (m, 1H), 7.28 (s, 1 H), 7.60 (s, 1H), 7.89 (s, 1H), 8.02 (s, 1H); MS (APPI) (M+H)⁺=408.29.

### Example 32

### 3-cyclohexyl-N-[2-(cyclopropylamino)-2-oxoethyl]-9-(isopropylsulfonyl)-N-methyl-2,3,4,9-tetrahydro-1H-carbazole-6-carboxamide

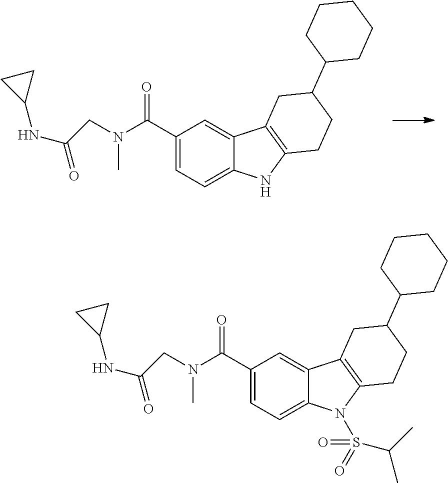

Following the procedure for Step A in Example 27, using 3-cyclohexyl-N-[2-(cyclopropylamino)-2-oxoethyl]-N-methyl-2,3,4,9-tetrahydro-1H-carbazole-6-carboxamide (121 mg, 0.30 mmol), sodium hydride (119 mg, 3.0 mmol) and isopropylsulfonyl chloride (423 mg, 3.0 mmol) in DMF (6 mL). The crude product was purified by MPLC on silica gel using EtOAc and then reverse-phase HPLC using high pH column 50-70% MeCN/H₂O to give 53 mg (35%) of a white solid as the title compound. ¹H NMR (400 MHz, CHLOROFORM-D) δ 0.49-0.61 (m, 2H), 0.74-0.90 (m, 2H), 0.99-1.38 (m, 11H), 1.44-1.63 (m, 4H), 1.64-1.90 (m, 3H), 2.03-2.15 (m, 1H), 2.25-2.45 (m, 1H), 2.62-2.94 (m, 3H), 3.05-3.23 (m, 4H), 3.35-3.58 (m, 1H), 3.94-4.21 (m, 2H), 6.62-6.79 (m, 1H), 7.32 (d, J=9.37 Hz, 1H), 7.57 (s, 1H), 7.97 (d, J=7.81 Hz, 1H); MS (APPI) (M+H)⁺=514.2.

### Example 33

### N-Ethyl-9-methyl-N-(2-oxo-2-(tetrahydro-2H-pyran-4-ylamino)ethyl)-3-(tetrahydro-2H-pyran-4-yl)-2,3,4,9-tetrahydro-1H-carbazole-6-carboxamide

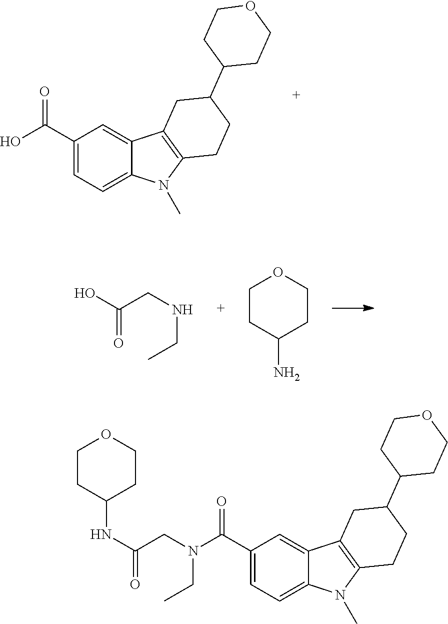

To a solution of 9-methyl-3-(tetrahydro-2H-pyran-4-yl)-2,3,4,9-tetrahydro-1H-carbazole-6-carboxylic acid (63 mg, 0.20 mmol) and DIPEA (1.0 mL) in DMF (2 mL) was added HATU (114 mg, 0.3 mmol) at 0° C. After 1 hr at r.t, 2-(ethylamino)acetic acid (22.80 mg, 0.22 mmol) was added. The reaction mixture was stirred for 2 hr at r.t, and followed by addition of tetrahydro-2H-pyran-4-amine, HCl (55.3 mg, 0.40 mmol) and HATU (114 mg, 0.3 mmol). The reaction mixture was stirred for additional 2 hr at r.t and the solvent was concentrated. The product was purified by preparative reverse-phase HPLC (high pH) using an acetonitrile gradient 20 to 40% in water N-ethyl-9-methyl-N-(2-oxo-2-(tetrahydro-2H-pyran-4-ylamino)ethyl)-3-(tetrahydro-2H-pyran-4-yl)-2,3,4,9-tetrahydro-1H-carbazole-6-carboxamide (8.0 mg, 8.3%). ¹H NMR (400 MHz, METHANOL-D4) δ ppm 1.19 (m, 3H), 1.38-1.48 (m, 3H), 1.56 (m, 4H), 1.76 (m, 4H), 2.11-2.20 (m, 1 H), 2.37 (m, 1H), 2.67 (m, 1H), 2.76-2.88 (m, 2H), 3.37-3.49 (m, 6H), 3.62 (s, 3H), 3.89 (m, 3H), 3.97 (m, 3H), 4.09 (m, 1H), 7.18 (s, 1H), 7.30 (d, J=8.20 Hz, 1H), 7.50 (s, 1H). MS (ESI) (M+H)⁺ 482.2.

### Example 34

### N-Ethyl-9-methyl-N-(2-oxo-2((S)-tetrahydrofuran-3-ylamino)ethyl)-3-(tetrahydro-2H-pyran-4-yl)-2,3,4,9-tetrahydro-1H-carbazole-6-carboxamide

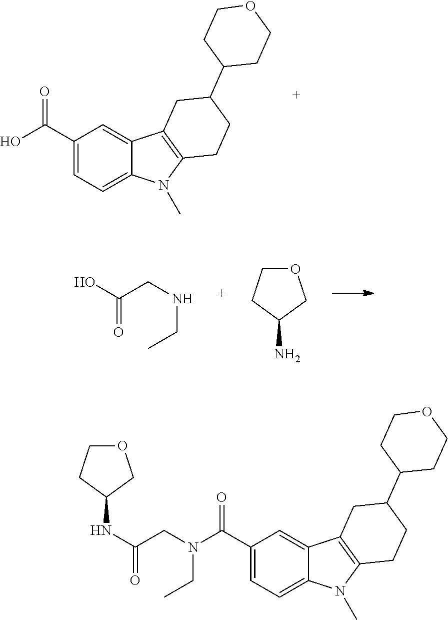

To a solution of 9-methyl-3-(tetrahydro-2H-pyran-4-yl)-2,3,4,9-tetrahydro-1H-carbazole-6-carboxylic acid (63 mg, 0.20 mmol) and DIPEA (1.0 mL) in DMF (2 mL) was added HATU (114 mg, 0.3 mmol) at 0° C. After 1 hr at r.t, 2-(ethylamino)acetic acid (22.80 mg, 0.22 mmol) was added. The reaction mixture was stirred for 2 hr at r.t, and followed by addition of (S)-tetrahydrofuran-3-amine (35.0 mg, 0.40 mmol) and HATU (114 mg, 0.3 mmol). The reaction mixture was stirred for additional 2 hr at r.t and the solvent was concentrated. The product was purified by preparative reverse-phase HPLC (high pH) using an acetonitrile gradient 20 to 40% in water N-ethyl-9-methyl-N-(2-oxo-2((S)-tetrahydro furan-3-ylamino)ethyl)-3-(tetrahydro-2H-pyran-4-yl)-2,3,4,9-tetrahydro-1H-carbazole-6-carboxamide (8.0 mg, 8.5%). ¹H NMR (400 MHz, METHANOL-D4) δ ppm 1.14 (m, 3H), 1.36-1.48 (m, 2H), 1.56 (m, 4H), 1.76 (m, 3H), 1.84 (m, 1H), 2.13 (m, 2H), 2.37 (m, 1H), 2.67 (m, 1H), 2.76-2.88 (m, 2H), 3.37-3.46 (m, 3H), 3.52 (m, 2H), 3.61 (s, 3H), 3.79 (m, 2H), 3.97 (dd, J=10.94, 3.52 Hz, 2H), 4.11 (m, 1H), 4.38 (m, 1H), 7.17 (s, 1H), 7.30 (d, J=7.81 Hz, 1H), 7.51 (s, 1H). MS (ESI) (M+H)⁺ 468.2.

### Example 35

### N-Ethyl-9-methyl-N-(2-oxo-2-((R)-tetrahydrofuran-3-ylamino)ethyl)-3-(tetrahydro-2H-pyran-4-yl)-2,3,4,9-tetrahydro-1H-carbazole-6-carboxamide

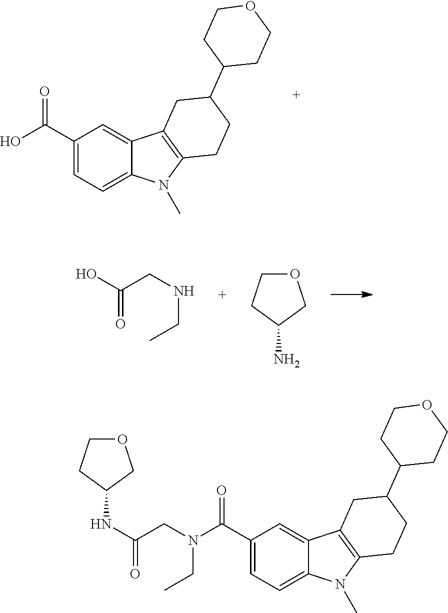

To a 9-methyl-3-(tetrahydro-2H-pyran-4-yl)-2,3,4,9-tetrahydro-1H-carbazole-6-carboxylic acid (63 mg, 0.20 mmol) and DIPEA (1.0 mL) in DMF (2 mL) was added HATU (114 mg, 0.3 mmol) at 0° C. After 1 hr at r.t, 2-(ethylamino)acetic acid (22.80 mg, 0.22 mmol) was added. The reaction mixture was stirred for 2 hr at r.t, and followed by addition of (R)-tetrahydrofuran-3-amine (35.0 mg, 0.40 mmol) and HATU (114 mg, 0.3 mmol). The reaction mixture was stirred for additional 2 hr at r.t and the solvent was concentrated. The product was purified by preparative reverse-phase HPLC (high pH) using an acetonitrile gradient 20 to 40% in water N-ethyl-9-methyl-N-(2-oxo-2((R)-tetrahydrofuran-3-ylamino)ethyl)-3-(tetrahydro-2H-pyran-4-yl)-2,3,4,9-tetrahydro-1H-carbazole-6-carboxamide (9.0 mg, 9.6%). 1H NMR (400 MHz, METHANOL-D4) δ ppm 1.14 (m, 3H), 1.36-1.48 (m, 2H), 1.56 (m, 4H), 1.76 (m, 3H), 1.84 (m, 1H), 2.13 (m, 2H), 2.37 (m, 1H), 2.67 (m, 1H), 2.76-2.88 (m, 2H), 3.37-3.46 (m, 3H), 3.52 (m, 2H), 3.61 (s, 3H), 3.79 (m, 2H), 3.97 (dd, J=10.94, 3.52 Hz, 2H), 4.11 (m, 1H), 4.38 (m, 1H), 7.17 (s, 1H), 7.30 (d, J=7.81 Hz, 1H), 7.51 (s, 1H). MS (ESI) (M+H)⁺ 468.2. HRMS calcd for (C₂₅H₃₆N₂O₃+H)⁺ , 468.2857. found, 468.2856.

### Example 36

### N-Ethyl-9-methyl-N-(2-(oxetan-3-ylamino)-2-oxoethyl)-3-(tetrahydro-2H-pyran-4-yl)-2,3,4,9-tetrahydro-1H-carbazole-6-carboxamide

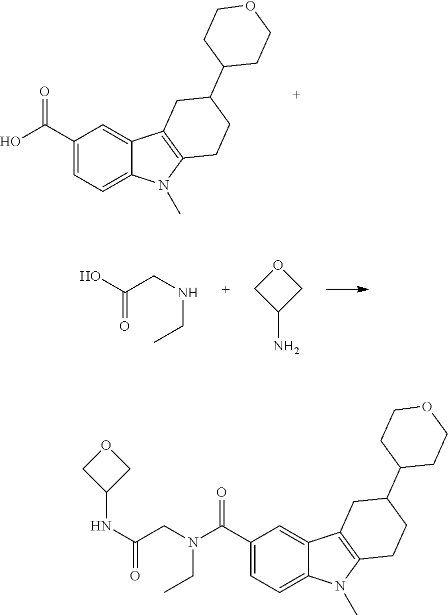

To a solution of 9-methyl-3-(tetrahydro-2H-pyran-4-yl)-2,3,4,9-tetrahydro-1H-carbazole-6-carboxylic acid (63.0 mg, 0.20 mmol) and DIPEA (1.0 mL) in DMF (2 mL) was added HATU (114 mg, 0.3 mmol) at 0° C. After 1 hr at r.t, 2-(ethylamino)acetic acid (22.80 mg, 0.22 mmol) was added. The reaction mixture was stirred for 2 hr at r.t, and followed by addition of oxetan-3-amine, HCl (44.0 mg, 0.40 mmol) and HATU (114 mg, 0.3 mmol). The reaction mixture was stirred for additional 2 hr at r.t and the solvent was concentrated. The product was purified by preparative reverse-phase HPLC using an acetonitrile gradient 30 to 50% in water and purified by high pH HPLC (20-40) again to provide the title compound N-ethyl-9-methyl-N-(2-(oxetan-3-ylamino)-2-oxoethyl)-3-(tetrahydro-2H-pyran-4-yl)-2,3,4,9-tetrahydro-1H-carbazole-6-carboxamide (34.0 mg, 37.3%). ¹H NMR (400 MHz, METHANOL-D4) δ ppm 1.10-1.22 (m, 3H), 1.34-1.45 (m, 2H), 1.47-1.57 (m, 3H), 1.73 (t, J=12.11 Hz, 2H), 2.14 (m, 1H), 2.35 (m, 1H), 2.67 (m, 1H), 2.79 (m, 2H), 3.32-3.43 (m, 3H), 3.43-3.60 (m, 2H), 3.59 (s, 3H), 3.96 (dd, J=11.33, 3.12 Hz, 2H), 3.90-4.20 (m, 2H), 4.35-4.65 (m, 2H), 4.78-5.0 (m, 2H), 7.16 (m, 1H), 7.30 (d, J=8.0 Hz, 1H), 7.52 (m, 1H); MS (ESI) (M+H)⁺ 454.2

### Example 37

### N-Ethyl-N-(4-hydroxybutyl)-9-methyl-3-(tetrahydro-2H-pyran-4-yl)-2,3,4,9-tetrahydro-1H-carbazole-6-carboxamide

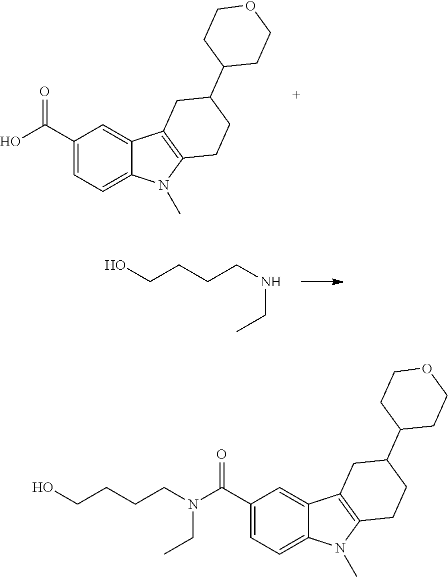

To a solution of 4-(ethylamino)butan-1-ol (47.1 mg, 0.40 mmol), 9-methyl-3-(tetrahydro-2H-pyran-4-yl)-2,3,4,9-tetrahydro-1H-carbazole-6-carboxylic acid (63.0 mg, 0.20 mmol) and DIPEA (0.3 mL) in DMF (2 mL) was added HATU (114 mg, 0.3 mmol) at 0° C. The reaction mixture was stirred for 2 hr at r.t and the solvent was concentrated. The product was purified by preparative reverse-phase HPLC using an acetonitrile gradient 30 to 50% in water to provide the title compound N-ethyl-N-(4-hydroxybutyl)-9-methyl-3-(tetrahydro-2H-pyran-4-yl)-2,3,4,9-tetrahydro-1H-carbazole-6-carboxamide (52.0 mg, 62.7%) as a white solid. 1H NMR (400 MHz, METHANOL-D4) δ ppm 1.12 (m, 2H), 1.26 (m, 2H), 1.34-1.45 (m, 2H), 1.54 (m, 3H), 1.68 (m, 2H), 1.74 (m, 3H), 2.14 (m, 1H), 2.37 (m, 1H), 2.67 (m, 1H), 2.82 (m, 2H), 3.34-3.6 (m, 8H), 3.61 (s, 3H), 3.98 (d, J=8.20 Hz, 2H), 7.11 (d, J=8.0 Hz, 1H), 7.31 (d, J=8.0 Hz, 1H), 7.43 (s, 1H); FIRMS calcd for (C₂₅H₃₅N₂O₃+H)⁺, 413.2799. found, 413.2791.

### Example 38

### N-(2-(Cyanomethylamino)-2-oxoethyl)-N-ethyl-9-methyl-3-(tetrahydro-2H-pyran-4-yl)-2,3,4,9-tetrahydro-1H-carbazole-6-carboxamide

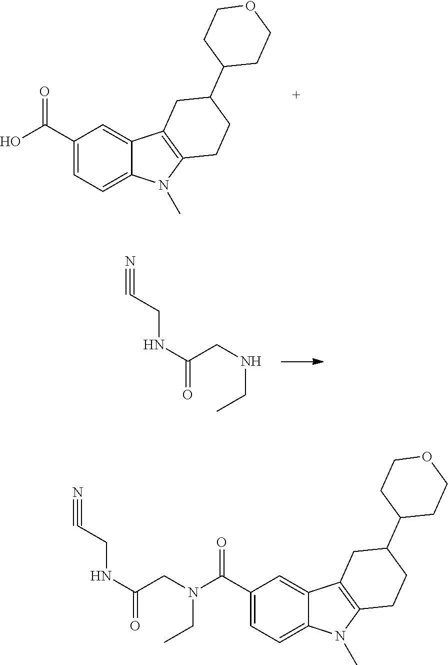

To a solution of N-(cyanomethyl)-2-(ethylamino)acetamide, trifluoroacetic acid (153 mg, 0.60 mmol), 9-methyl-3-(tetrahydro-2H-pyran-4-yl)-2,3,4,9-tetrahydro-1H-carbazole-6-carboxylic acid (94 mg, 0.30 mmol) and DIPEA (0.5 mL) in DMF (3 mL) was added HATU (228 mg, 0.6 mmol) at 0° C. The reaction mixture was stirred for 2 hr at r.t and the solvent was concentrated. The product was purified by preparative reverse-phase HPLC using an acetonitrile gradient 30, to 50% in water to provide N-(2-(cyanomethylamino)-2-oxoethyl)-N-ethyl-9-methyl-3-(tetrahydro-2H-pyran-4-yl)-2,3,4,9-tetrahydro-1H-carbazole-6-carboxamide (14.0 mg, 10.7%) as a white solid: ¹H NMR (400 MHz, METHANOL-D4) δ ppm 1.16 (m, 3H), 1.40 (m, 2H), 1.54 (m, 3H), 1.75 (t, J=12.11 Hz, 2H), 2.14 (m, 1H), 2.37 (m, 1H), 2.67 (m, 1H), 2.80 (m, 2H), 3.32-3.60 (m, 4H), 3.61 (s, 3H), 3.96 (d, J=8.59 Hz, 2H), 4.17 (m, 4H), 7.18 (s, 1H), 7.30 (d, J=7.81 Hz, 1H), 7.54 (s, 1H); MS (ESI) (M+H)⁺ 437.3;

### Example 39

N-(2-(Cyclopropylamino)-2-oxoethyl)-N-ethyl-3-(tetrahydro-2H-pyran-4-yl)-2,3,4,9-tetrahydro-1H-carbazole-6-carboxamide

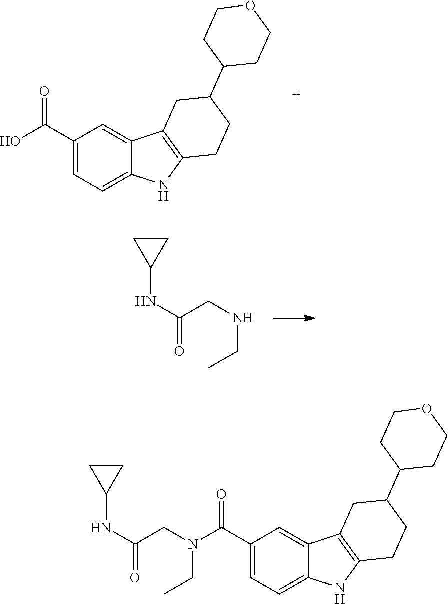

To a solution of 2-(cyclopropylamino)-N-ethyl-2-oxoethanaminium, Chloride (107 mg, 0.60 mmol), 3-(tetrahydro-2H-pyran-4-yl)-2,3,4,9-tetrahydro-1H-carbazole-6-carboxylic acid (90 mg, 0.30 mmol) and DIPEA (0.5 mL) in DMF (3 mL) was added HATU (190 mg, 0.5 mmol) at 0° C. The reaction mixture was stirred for 2 hr at r.t and the solvent was concentrated. The product was purified by preparative reverse-phase HPLC using an acetonitrile gradient 30 to 50% in water to provide the title compound N-(2-(cyclopropylamino)-2-oxoethyl)-N-ethyl-3-(tetrahydro-2H-pyran-4-yl)-2,3,4,9-tetrahydro-1H-carbazole-6-carboxamide (26.0 mg, 20.42%) as white solid. ¹H NMR (400 MHz, METHANOL-D4) δ ppm 0.4-0.6 (m, 2H), 0.70 (m, 2H), 1.14 (m, 3H), 1.38-1.50 (m, 2H), 1.50-1.61 (m, 3H), 1.76 (m, 2H), 2.09 (m, 1H), 2.36 (m, 1H), 2.65 (m, 1H), 2.71-2.83 (m, 3H), 3.37-3.48 (m, 4H), 3.90-4.12 (m, 4H), 7.10 (s, 1H), 7.25 (d, J=8.0 Hz, 1H), 7.48 (s, 1H); HRMS calcd for (C₂₅H₃₃N₃O₃+H)⁺ , 424.2595. found, 424.2594.

### Example 40

### N—((S)-1-(2-Fluoroethylamino)-1-oxopropan-2-yl)-N,9-dimethyl-3-(tetrahydro-2H-pyran-4-yl)-2,3,4,9-tetrahydro-1H-carbazole-6-carboxamide

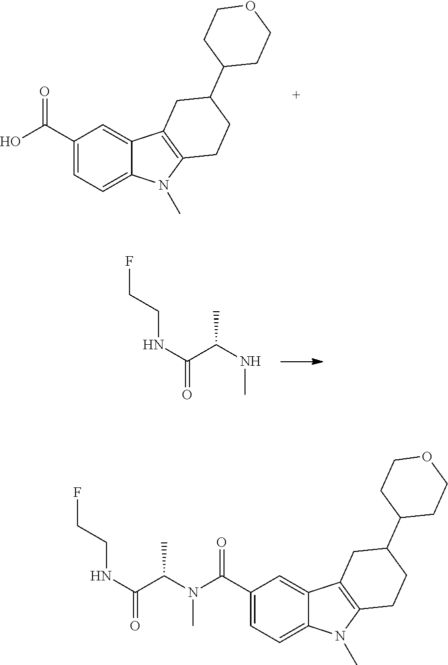

To a solution of (S)-1-(2-fluoroethylamino)-N-methyl-1-oxopropan-2-aminium, chloride (111 mg, 0.60 mmol), 9-methyl-3-(tetrahydro-2H-pyran-4-yl)-2,3,4,9-tetrahydro-1H-carbazole-6-carboxylic acid (94 mg, 0.30 mmol) and DIPEA (0.5 mL) in DMF (3 mL) was added HATU (228 mg, 0.6 mmol) at 0° C. The reaction mixture was stirred for 2 hrs. at r.t and the solvent was concentrated. The product was purified by preparative reverse-phase HPLC using an acetonitrile gradient 30 to 50% in water to provide the title compound N—((S)-1-(2-fluoroethylamino)-1-oxopropan-2-yl)-N,9-dimethyl-3-(tetrahydro-2H-pyran-4-yl)-2,3,4,9-tetrahydro-1H-carbazole-6-carboxamide (18.0 mg, 13.5%) as white solid. ¹H NMR (400 MHz, METHANOL-D4) δ ppm 1.42 (m, 5H), 1.49-1.59 (m, 3H), 1.75 (m, 2H), 2.14 (m, 1H), 2.37 (m, 1H), 2.66 (m, 1H), 2.77-2.87 (m, 2H), 3.00 (s, 3H), 3.35-3.45 (m, 2H), 3.47 (t, J=4.88 Hz, 1H), 3.54 (t, J=4.88 Hz, 1H), 3.61 (s, 3H), 3.96 (dd, J=11.13, 3.71 Hz, 2H), 4.40 (t, J=5.08 Hz, 1H), 4.52 (t, J=5.08 Hz, 1H), 4.8 (m, 1H), 7.18 (d, J=8.0 Hz, 1H), 7.31 (d, J=8.0 Hz, 1H), 7.52 (s, 1H); MS (ESI) (M+H)⁺ 444.2; HRMS calcd for (C₂₅H₃₄FN₃O₃+H)⁺ , 444.2657. found, 444.2662.

### Example 41

### N—((S)-1-(Cyclopropylamino)-1-oxopropan-2-yl)-N,9-dimethyl-3-(tetrahydro-2H-pyran-4-yl)-2,3,4,9-tetrahydro-1H-carbazole-6-carboxamide

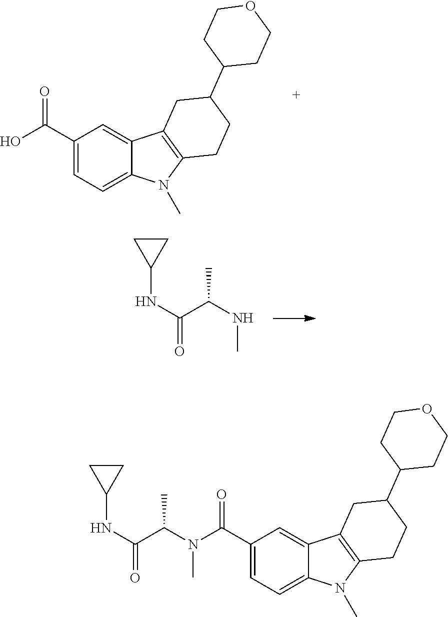

To a solution of ((S)-1-(cyclopropylamino)-N-methyl-1-oxopropan-2-aminium, Chloride (107 mg, 0.60 mmol), 9-methyl-3-(tetrahydro-2H-pyran-4-yl)-2,3,4,9-tetrahydro-1H-carbazole-6-carboxylic acid (94 mg, 0.30 mmol) and DIPEA (0.5 mL) in DMF (3 mL) was added HATU (228 mg, 0.6 mmol) at 0° C. The reaction mixture was stirred for 2 hr at r.t and the solvent was concentrated. The product was purified by preparative reverse-phase HPLC using an acetonitrile gradient 30 to 50% in water to provide the title compound N—((S)-1-(cyclopropylamino)-1-oxopropan-2-yl)-N,9-dimethyl-3-(tetrahydro-2H-pyran-4-yl)-2,3,4,9-tetrahydro-1H-carbazole-6-carboxamide (75 mg, 57%) as white solid. ¹H NMR (400 MHz, METHANOL-D4) δ ppm 0.51 (m, 2H), 0.68-0.75 (m, 2 H), 1.41 (m, 5H), 1.54 (m, 3H), 1.75 (m, 2H), 2.14 (m, 1H), 2.37 (m, 1H), 2.62-2.71 (m, 2H), 2.77-2.86 (m, 2H), 3.01 (s, 3H), 3.36-3.44 (m, 2H), 3.61 (s, 3H), 3.97 (dd, J=11.13, 3.32 Hz, 2H), 4.56 (m, 1H), 7.16 (s, 1H), 7.30 (d, J=8.59 Hz, 1H), 7.49 (d, J=1.95 Hz, 1H); MS (ESI) (M+H)⁺ 438.3. HRMS calcd for (C₂₆H₃₅N₃O₃+H)⁺ , 438.2751. found, 438.2746.

### Example 42

### N-(4-(Cyclopropylamino)-4-oxobutyl)-9-methyl-3-(tetrahydro-2H-pyran-4-yl)-2,3,4,9-tetrahydro-1H-carbazole-6-carboxamide

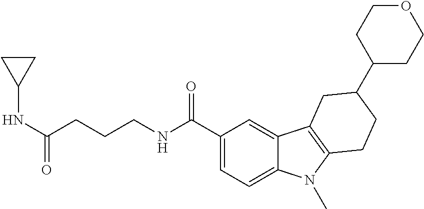

### Step A: N-(4-(Cyclopropylamino)-4-oxobutyl)-9-methyl-3-(tetrahydro-2H-pyran-4-yl)-2,3,4,9-tetrahydro-1H-carbazole-6-carboxamide

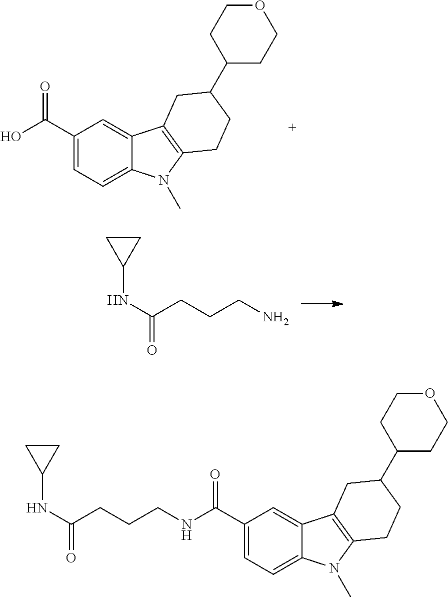

To a solution of 4-(cyclopropylamino)-4-oxobutan-1-aminium chloride (179 mg, 1.00 mmol), 9-methyl-3-(tetrahydro-2H-pyran-4-yl)-2,3,4,9-tetrahydro-1H-carbazole-6-carboxylic acid (157 mg, 0.50 mmol) and DIPEA (0.5 mL) in DMF (5 mL) was added HATU (228 mg, 0.6 mmol) at 0° C. The reaction mixture was stirred for 2 hr at r.t and the solvent was concentrated. The residue was dissolved in EtOAc, and was washed with NH₄OH (2 N), brine and dried over anhydrous sodium sulfate. The product was purified by preparative reverse-phase HPLC using an acetonitrile gradient 30 to 50% in water to provide the title compound as white solid (68 mg, 31%). ¹H NMR (400 MHz, METHANOL-D4) δ ppm 0.45 (m, 2H), 0.66 (m, 2H), 1.37-1.49 (m, 2H), 1.51-1.61 (m, 3H), 1.77 (m, 2H), 1.85-1.92 (m, 2H), 2.13-2.24 (m, 3H), 2.42 (m, 1H), 2.70 (m, 1H), 2.80-2.89 (m, 2H), 3.35-3.50 (m, 5H), 3.62 (s, 3H), 3.98 (dd, J=11.3, 3.5 Hz, 2H), 7.29 (d, J=8.6 Hz, 1H), 7.58 (d, J=8.6 Hz, 1H), 7.93 (s, 1H); MS (ESI) (M+H)⁺438.3.

### Step B: 4-(cyclopropylamino)-4-oxobutan-1-aminium chloride

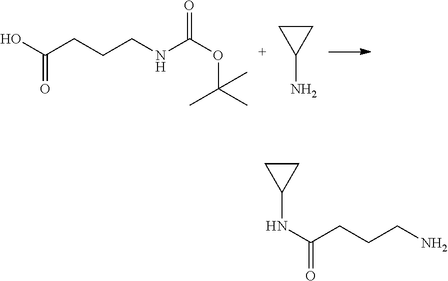

To a solution of cyclopropylamine (1.14 g, 20 mmol), 4-(tert-butoxycarbonylamino)butanoic acid (2.030 g, 9.99 mmol), and DIPEA (3 mL) in DMF (30 mL) was added HATU (4.56 g, 12 mmol) at 0° C. The reaction mixture was stirred for 2 hr at r.t and the solvent was concentrated. The residue was dissolved in EtOAc, and was washed with NH₄OH (2 N), brine and dried over anhydrous sodium sulfate. Removal of solvents provided a residue that was dissolved in 50 mL of 1M HCl/AcOH and stirred at rt for 3 h. The solvent was evaporated to give the desired product, which was used directly in the next step.

### Example 43

### N-(1-(Cyclopropylcarbamoyl)cyclopropyl)-N,9-dimethyl-3-(tetrahydro-2H-pyran-4-yl)-2,3,4,9-tetrahydro-1H-carbazole-6-carboxamide

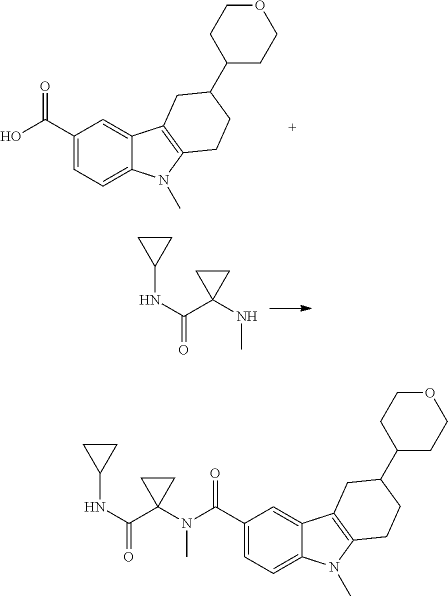

To a solution of 1-(cyclopropylcarbamoyl)-N-methylcyclopropanaminium chloride (191 mg, 1.00 mmol), 9-methyl-3-(tetrahydro-2H-pyran-4-yl)-2,3,4,9-tetrahydro-1H-carbazole-6-carboxylic acid (157 mg, 0.50 mmol) and DIPEA (0.5 mL) in DMF (5 mL) was added HATU (228 mg, 0.6 mmol) at 0° C. The reaction mixture was stirred for 2 hr at r.t and the solvent was concentrated. The product was purified by preparative reverse-phase HPLC using an acetonitrile gradient 30 to 50% in water to provide the title compound as white solid (7 mg, 3%). ¹H NMR (400 MHz, METHANOL-D4) δ ppm 0.71 (m, 2H), 0.78 (m, 2H), 1.13 (m, 1H), 1.32-1.36 (m, 2H), 1.38-1.49 (m, 4H), 1.57 (m, 3H), 1.73-1.82 (m, 2H), 2.17 (m, 1H), 2.37 (m, 1H), 2.60-2.75 (m, 2H), 2.80-2.89 (m, 1H), 3.38-3.46 (m, 2H), 3.62 (s, 3H), 3.98 (dd, J=11.3, 3.7 Hz, 2H), 7.21 (d, J=8.6 Hz, 1H), 7.28 (d, J=8.6 Hz, 1H), 7.53 (s, 1H); MS (ESI) (M+H)⁺ 450.2.

### Example 44

### N-(2-Fluoroethyl)-9-methyl-3-(tetrahydro-2H-pyran-4-yl)-2,3,4,9-tetrahydro-1H-carbazole-6-carboxamide

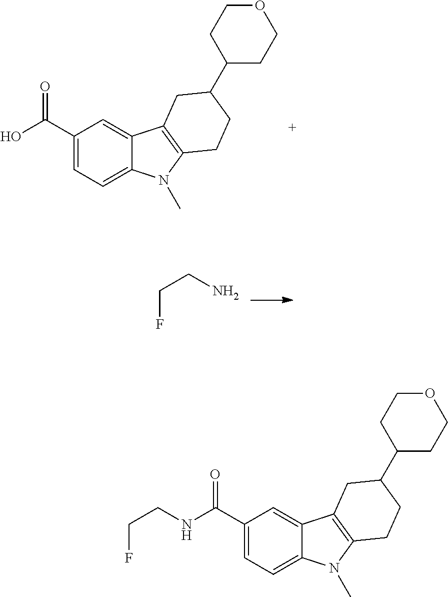

To a solution of 2-fluoroethanaminium chloride (199 mg, 2.00 mmol), 9-methyl-3-(tetrahydro-2H-pyran-4-yl)-2,3,4,9-tetrahydro-1H-carbazole-6-carboxylic acid (313 mg, 1.00 mmol) and DIPEA (0.8 mL) in DMF (8 mL) was added HATU (456 mg, 1.2 mmol) at 0° C. The reaction mixture was stirred for 2 hr at r.t and the solvent was concentrated. The product was purified by preparative reverse-phase HPLC using an acetonitrile gradient 20 to 50% in water to provide the title compound as white solid (268 mg, 75%). ¹H NMR (400 MHz, METHANOL-D4) δ ppm 1.34-1.45 (m, 2H), 1.46-1.56 (m, 3H), 1.73 (m, 2H), 2.08-2.16 (m, 1H), 2.31-2.39 (m, 1H), 2.64 (m, 1H), 2.79 (m, 2H), 3.35-3.43 (m, 2H), 3.59 (s, 3H), 3.63 (t, J=5.27 Hz, 1H), 3.69 (t, J=5.27 Hz, 1H), 3.96 (dd, J=10.94, 3.91 Hz, 2H), 4.48 (t, J=5.08 Hz, 1H), 4.60 (t, J=5.27 Hz, 1H), 7.27 (d, J=8.59 Hz, 1H), 7.60 (dd, J=8.59, 1.95 Hz, 1H), 7.95 (d, J=1.56 Hz, 1H); MS (ESI) (M+H)⁺ 359.2135.

### Example 45

### N-Ethyl-N-(4-(2-fluoroethylamino)-4-oxobutyl)-9-methyl-3-(tetrahydro-2H-pyran-4-yl)-2,3,4,9-tetrahydro-1H-carbazole-6-carboxamide

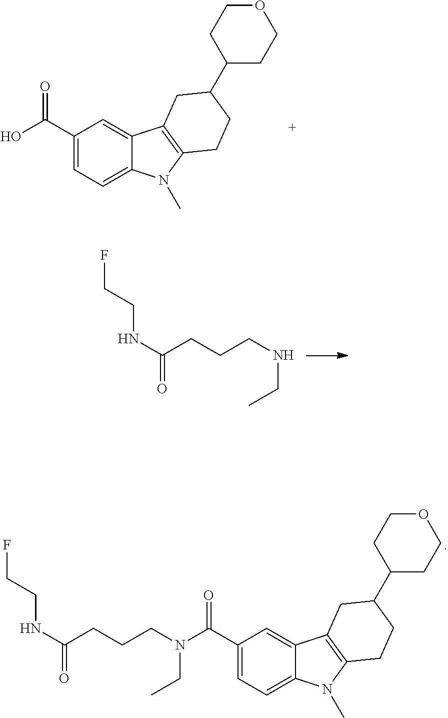

To a solution of N-ethyl-4-(2-fluoroethylamino)-4-oxobutan-1-aminium chloride (213 mg, 1.00 mmol), 9-methyl-3-(tetrahydro-2H-pyran-4-yl)-2,3,4,9-tetrahydro-1H-carbazole-6-carboxylic acid (157 mg, 0.50 mmol) and DIPEA (0.5 mL) in DMF (5 mL) was added HATU (228 mg, 0.6 mmol) at 0° C. The reaction mixture was stirred for 2 hr at r.t and the solvent was concentrated. The product was purified by preparative reverse-phase HPLC using an acetonitrile gradient 20 to 50% in water after three times to provide the title compound N-ethyl-N-(4-(2-fluoroethylamino)-4-oxobutyl)-9-methyl-3-(tetrahydro-2H-pyran-4-yl)-2,3,4,9-tetrahydro-1H-carbazole-6-carboxamide (22.86%) as white solid (54 mg). ¹H NMR (400 MHz, METHANOL-D4) δ ppm 1.05-1.31 (m, 3 H), 1.35-1.46 (m, 2H), 1.48-1.58 (m, 3H), 1.74 (t, J=12.50 Hz, 2H), 1.80-2.08 (m, 3 H), 2.14 (m, 1H), 2.20-2.40 (m, 2H), 2.60-2.71 (m, 1H), 2.77-2.86 (m, 2H), 3.15-3.60 (m, 8H) 3.50 (s, 2H), 3.60 (s, 3H), 3.96 (dd, J=10.94, 3.52 Hz, 2H), 4.10-4.50 (m, 2H), 7.08 (d, J=8.20 Hz, 1H), 7.30 (d, J=8.20 Hz, 1H), 7.41 (s, 1H); MS (ESI) (M+H)⁺ 472.2.

### Example 46

### N—((R)-1-(2-fluoroethylamino)-1-oxopropan-2-yl)-N,9-dimethyl-3-(tetrahydro-2H-pyran-4-yl)-2,3,4,9-tetrahydro-1H-carbazole-6-carboxamide

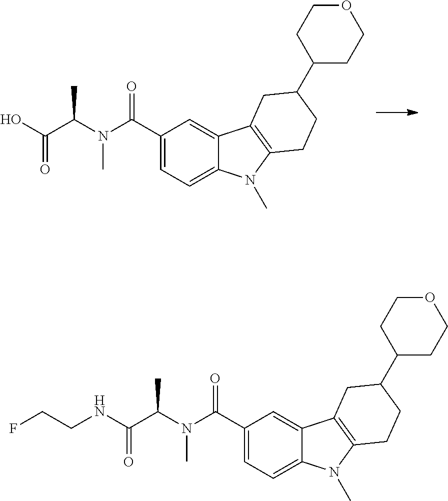

HATU (55.8 mg, 0.15 mmol) and 2-Fluoroethylamine hydrochloride (11.24 mg, 0.11 mmol) were added slowly at 0° C. to a solution of (2R)-2-(N,9-dimethyl-3-(tetrahydro-2H-pyran-4-yl)-2,3,4,9-tetrahydro-1H-carbazole-6-carboxamido)propanoic acid (45 mg, 0.11 mmol) and N,N-diisopropylethylamine (0.059 mL, 0.34 mmol) in DMF (0.896 mL). Reaction mixture was stirred at room temperature for 4 hours. The solvent was then removed in vacuo to provide the crude compound as yellow oil. The N—((R)-1-(2-fluoroethylamino)-1-oxopropan-2-yl)-N,9-dimethyl-3-(tetrahydro-2H-pyran-4-yl)-2,3,4,9-tetrahydro-1H-carbazole-6-carboxamide (66.2%) was purified by Prep-LCMS reverse-phase using a low pH 40-60% ACN/water system. ¹H NMR (400 MHz, CD₃OD) δ ppm 1.36-1.50 (m, 7H), 1.50-1.64 (m, 5H), 1.77 (t, J=13.28 Hz, 2H), 2.10-2.23 (m, 1H), 2.29-2.47 (m, 1H), 2.59-2.75 (m, 1H), 2.79-2.91 (m, 2H), 3.02 (s, 3H), 3.37-3.47 (m, 2H), 3.49 (t, J=4.88 Hz, 1H), 3.56 (t, J=4.88 Hz, 1H), 3.63 (s, 3H), 3.98 (dd, J=11.33, 3.91 Hz, 2H), 4.42 (t, J=4.88 Hz, 1H), 4.54 (t, J=5.08 Hz, 1H), 7.11-7.25 (m, 1H); [M+H]+ calc.=444.2657, [M+H]+ obs.=444.2670.

### Example 47

### N4R)-1-(ethylamino)-1-oxopropan-2-yl)-N,9-dimethyl-3-(tetrahydro-2H-pyran-4-yl)-2,3,4,9-tetrahydro-1H-carbazole-6-carboxamide

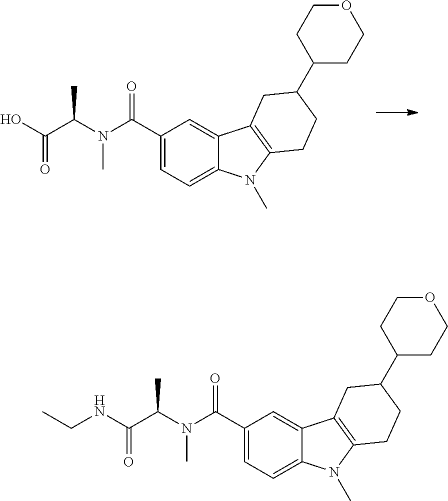

HATU (71.8 mg, 0.19 mmol) and ethylamine hydrochloride (15.39 mg, 0.19 mmol) were added slowly at 0° C. to a solution of (2R)-2-(N,9-dimethyl-3-(tetrahydro-2H-pyran-4-yl)-2,3,4,9-tetrahydro-1H-carbazole-6-carboxamido)propanoic acid (37.6 mg, 0.09 mmol) and N,N-diisopropylethylamine (0.049 mL, 0.28 mmol) in DMF (0.749 mL). Reaction mixture was stirred at room temperature for an O/N. The solvent was then removed in vacuo to provide the crude compound as yellow oil. The N—((R)-1-(ethylamino)-1-oxopropan-2-yl)-N,9-dimethyl-3-(tetrahydro-2H-pyran-4-yl)-2,3,4,9-tetrahydro-1H-carbazole-6-carboxamide (71.5%) was purified by Prep-HPLC reverse-phase using a low pH 60-80% ACN/water system. ¹H NMR (400 MHz, CD₃OD-D4) δ ppm 1.14 (t, J=7.03 Hz, 3H), 1.36-1.51 (m, 6H), 1.51-1.66 (m, 4H), 1.71-1.86 (m, 2H), 2.13-2.23 (m, 1H), 2.32-2.47 (m, 1H), 2.63-2.78 (m, 1H), 2.80-2.92 (m, 2H), 3.02 (s, 3H), 3.24 (m, 2H), 3.38-3.51 (m, 2H), 3.64 (s, 3H), 3.99 (dd, J=11.33, 2.34 Hz, 2H), 7.14-7.26 (m, 1H), 7.30-7.38 (m, 1H), 7.47-7.58 (m, 1H); [M+H]+ calc.=426.2751, [M+H]+ obs.=426.2749.

### Example 48

### N-ethyl-N-(2-hydroxypropyl)-9-methyl-3-(tetrahydro-2H-pyran-4-yl)-2,3,4,9-tetrahydro-1H-carbazole-6-carboxamide

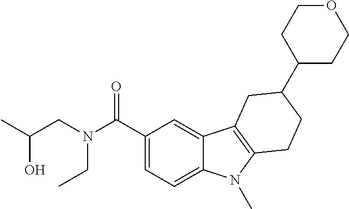

### Step A. N-ethyl-N-(2-hydroxypropyl)-9-methyl-3-(tetrahydro-2H-pyran-4-yl)-2,3,4,9-tetrahydro-M-carbazole-6-carboxamide

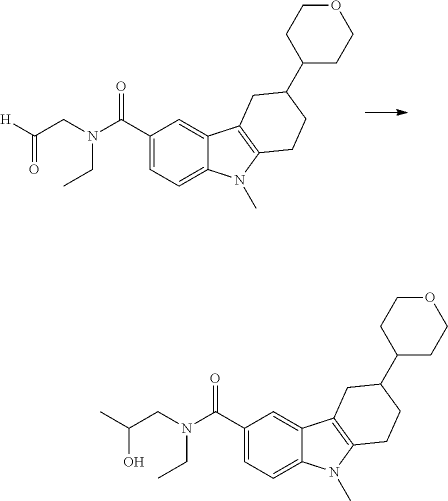

A solution of N-ethyl-9-methyl-N-(2-oxoethyl)-3-(tetrahydro-2H-pyran-4-yl)-2,3,4,9-tetrahydro-1H-carbazole-6-carboxamide (103.7 mg, 0.27 mmol) in TI-IF (25 mL) was cooled to −78° C. Methylmagnesium chloride (0.136 mL, 0.41 mmol) 3M in THF was added slowly and the reaction mixture was stirred for 2 h; then the mixture was allowed to reach room temperature slowly. After 14 h the reaction mixture was filtered, and the solvent was evaporated in vacuo. The N-ethyl-N-(2-hydroxypropyl)-9-methyl-3-(tetrahydro-2H-pyran-4-yl)-2,3,4,9-tetrahydro-1H-carbazole-6-carboxamide (21.4 mg, 16%) was purified by Prep-HPLC reverse-phase using a low pH 40-60% ACN/water system. ¹H NMR (400 MHz, CD₃OD-D4) δ ppm 0.88-1.00 (m, 1H), 1.02-1.16 (m, 2H), 1.18-1.31 (m, 3H), 1.35-1.50 (m, 3H), 1.51-1.63 (m, 3H), 1.78 (t, J=13.67 Hz, 2 H), 2.11-2.24 (m, 1H), 2.33-2.46 (m, 1H), 2.62-2.77 (m, 1H), 2.78-2.91 (m, 2H), 3.32-3.55 (m, 6H), 3.59-3.68 (m, 3H), 3.99 (dd, J=11.13, 3.71 Hz, 2H), 4.08-4.26 (m, 1H), 7.08-7.22 (m, 1H), 7.29-7.38 (m, 1H), 7.42-7.53 (m, 1H); MS (ESI) (M+H)+ 399.4.

### Step B. N-ethyl-N-(2-hydroxyethyl)-9-methyl-3-(tetrahydro-2H-pyran-4-yl)-2,3,4,9-tetrahydro-1H-carbazole-6-carboxamide

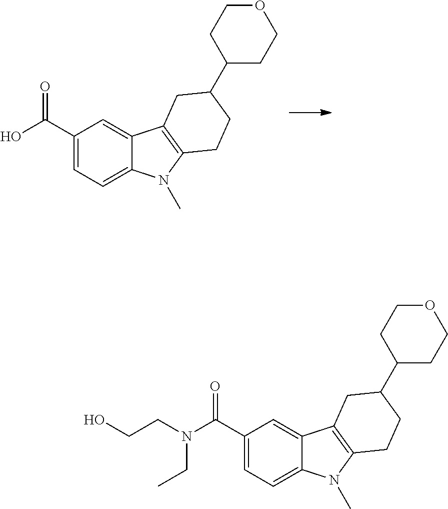

HATU (485 mg, 1.28 mmol) and 2-(Ethylamino)ethanol (0.124 mL, 1.28 mmol) were added slowly at 0° C. to a solution of 9-methyl-3-(tetrahydro-2H-pyran-4-yl)-2,3,4,9-tetrahydro-1H-carbazole-6-carboxylic acid (200 mg, 0.64 mmol) and N,N-diisopropylethylamine (0.334 mL, 1.92 mmol) in DMF (6.877 mL). Reaction mixture was stirred at room temperature for 2 h. The solvent was then removed in vacuo to provide the crude compound as yellow oil. The N-ethyl-N-(2-hydroxyethyl)-9-methyl-3-(tetrahydro-2H-pyran-4-yl)-2,3,4,9-tetrahydro-1H-carbazole-6-carboxamide (179 mg, 56.2%) was purified by Prep-HPLC reverse-phase using a low pH 50-70% ACN/water system. ¹H NMR (400 MHz, CD₃OD-D4) δ ppm 1.04-1.32 (m, 3H), 1.30-1.47 (m, 2H), 1.46-1.59 (m, 3H), 1.74 (t, J=13.67 Hz, 2H), 2.07-2.17 (m, 1H), 2.35 (dd, J=15.23, 7.81 Hz, 1H), 2.59-2.71 (m, 1H), 2.76-2.86 (m, 2H), 3.35-3.45 (m, 3H), 3.45-3.57 (m, 3H), 3.60 (s, 3H), 3.61-3.71 (m, 2H), 3.75-3.88 (m, 1H), 3.97 (dd, J=11.13, 4.10 Hz, 2H), 7.11-7.18 (m, 1H), 7.26-7.33 (m, 1H), 7.46-7.50 (m, 1H); MS (ESI) (M+H+ 385.4.

### Step C. N-ethyl-N-(2-hydroxyethyl)-9-methyl-3-(tetrahydro-2H-pyran-4-yl)-2,3,4,9-tetrahydro-1H-carbazole-6-carboxamide

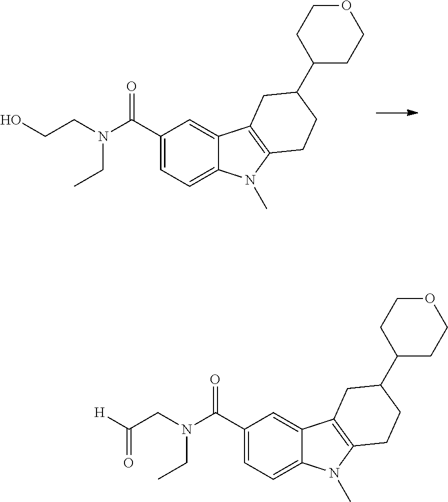

Stirred 500.0 mg of 4 Å molecular sieves in dry DCM (2.048 mL), N-ethyl-N-(2-hydroxyethyl)-9-methyl-3-(tetrahydro-2H-pyran-4-yl)-2,3,4,9-tetrahydro-1H-carbazole-6-carboxamide (100.0 mg, 0.26 mmol) and 4-Methylmorpholine-4-oxide (76 mg, 0.65 mmol) (20.0 mL) for 30 minutes. Tetrapropylammonium perruthenate (4.57 mg, 0.01 mmol) was then added. After 4 h of stirring at room temperature, the reaction mixture was filtered and the solvent evaporated in vacuo. The N-ethyl-9-methyl-N-(2-oxoethyl)-3-(tetrahydro-2H-pyran-4-yl)-2,3,4,9-tetrahydro-1H-carbazole-6-carboxamide (56.3 mg, 56.6%) was purified by flash column with a DCM/MeOH to provide the title compound as oil. MS (ESI) (M+H)+ 383.3.

### Example 49

### N-(2-(2-Cyanoethylamino)-2-oxoethyl)-N-ethyl-9-(ethylsulfonyl)-3-(tetrahydro-2H-pyran-4-yl)-2,3,4,9-tetrahydro-1H-carbazole-6-carboxamide

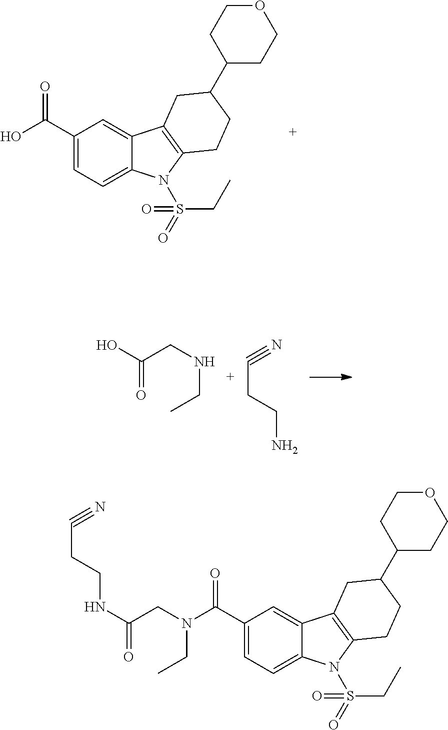

To a solution of 9-(ethylsulfonyl)-3-(tetrahydro-2H-pyran-4-yl)-2,3,4,9-tetrahydro-1H-carbazole-6-carboxylic acid (117 mg, 0.30 mmol) and DIPEA (1.0 mL) in DMF (2 mL) was added HATU (125 mg, 0.33 mmol) at 0° C. After 1 hr at r.t., 2-(ethylamino)acetic acid (33.9 mg, 0.33 mmol) was added. The reaction mixture was stirred for 2 hr at r.t, and followed by addition of 3-aminopropanenitrile (41.9 mg, 0.60 mmol) and HATU (125 mg, 0.33 mmol). The reaction mixture was stirred for additional 2 hr at r.t and the solvent was concentrated. The product was purified by preparative reverse-phase HPLC (high pH) using an acetonitrile gradient 20 to 40% in water to provide N-(2-(2-cyanoethylamino)-2-oxoethyl)-N-ethyl-9-(ethylsulfonyl)-3-(tetrahydro-2H-pyran-4-yl)-2,3,4,9-tetrahydro-1H-carbazole-6-carboxamide (66.0 mg, 41.8%). ¹H NMR (400 MHz, METHANOL-D4) δ ppm 1.12 (m, 5H), 1.23 (m, 1H), 1.41 (m, 2H), 1.56 (m, 3H), 1.71-1.81 (m, 2H), 2.13 (m, 1H), 2.35 (m, 1H), 2.60-2.71 (m, 2H), 2.83 (m, 2H), 3.14 (m, 1H), 3.26-3.36 (m, 3H), 3.42 (m, 4H), 3.62 (m, 1H), 3.98 (m, 3H), 4.17 (m, 1H), 7.73 (m, 1H), 7.63 (m, 0.5H), 7.97 (m, 1H), 8.43 (m, 1H). HRMS calcd for (C₂₇H₃₆N₄O₅S+H)⁺ , 529.24792. found, 529.24811.

### Example 50

### Step A: (3S)—N-Cyclopropyl-1-(9-methyl-3-(tetrahydro-2H-pyran-4-yl)-2,3,4,9-tetrahydro-1H-carbazole-6-carbonyl)piperidine-3-carboxamide

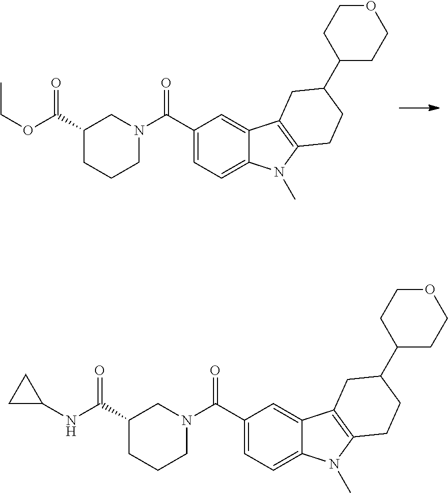

(3S)-Ethyl 1-(9-methyl-3-(tetrahydro-2H-pyran-4-yl)-2,3,4,9-tetrahydro-1H-carbazole-6-carbonyl)piperidine-3-carboxylate (140 mg, 0.31 mmol) was dissolved in dioxane (8 mL) containing lithium hydroxide (0.619 mL, 0.62 mmol) (1M) and was stirred at 23° C. for 3 h. The solvent was evaporated. The residue was dissolved in DMF (8.00 mL) containing N,N-diisopropylethylamine (0.135 mL, 0.77 mmol). HATU (141 mg, 0.37 mmol) and cyclopropylamine (0.026 mL, 0.37 mmol) were added and the solution was stirred at 23° C. for 1 h. The solvent was evaporated. The residue was dissolved in EtOAc and washed with aqueous saturated NaHCO₃, brine and dried over anhydrous Na₂SO₄. The product was purified by reversed-phase HPLC using 50-70% B and lyophilized. Yield: 85 mg (59%) (Purification: Gilson system equipped with Luna C-18 column, 250×50 mm, 15 u Mobile phase: A: H₂O with 0.05% TFA v/v; B: CH₃CN; 55 mL/min, 30 min run, rt): ¹H NMR (400 MHz, METHANOL-D4) δ 0.45 (s, 2H), 0.67 (s, 2H), 1.34-1.49 (m, 3H), 1.50-1.62 (m, 4H), 1.76 (m, 4H), 1.87-1.95 (m, 1H), 2.11-2.21 (m, 1 H), 2.29-2.44 (m, 2H), 2.60-2.72 (m, 1H), 2.76-2.91 (m, 2H), 3.12 (s, 1H), 3.35-3.47 (m, 2H), 3.62 (s, 3H), 3.79 (s, 1H), 3.97 (dd, J=10.94, 3.52 Hz, 2H), 4.45 (s, 1H), 7.12 (dd, J=8.40, 1.76 Hz, 1H), 7.31 (d, J=8.59 Hz, 1H), 7.46 (d, J=1.17 Hz, 1H); MS (ESI) (M+H)⁺=464.2.

### Step B: (3S)-Ethyl 1-(9-methyl-3-(tetrahydro-2H-pyran-4-yl)-2,3,4,9-tetrahydro-1H-carbazole-6-carbonyl)piperidine-3-carboxylate

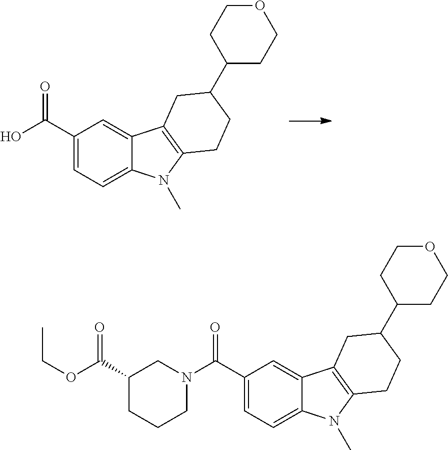

9-Methyl-3-(tetrahydro-2H-pyran-4-yl)-2,3,4,9-tetrahydro-1H-carbazole-6-carboxylic acid (100 mg, 0.32 mmol), HATU (146 mg, 0.38 mmol) and (S)-(+)-nipecotic acid ethyl ester (0.059 mL, 0.38 mmol) were stirred in DMF (5 mL) containing N,N-diisopropylethylamine (0.083 mL, 0.48 mmol) at rt for 1 h. The solvent was evaporated. The residue was dissolved in EtOAc and washed with aqueous saturated NaHCO₃, brine and dried over anhydrous Na₂SO₄. The product was purified by flash using 20-80% EtOAc/heptane gradient. Yield: 140 mg (97%); ¹H NMR (400 MHz, DMSO-D6) δ 1.05-1.15 (m, 2H), 1.21-1.36 (m, 3H), 1.40-1.53 (m, 4H), 1.56-1.75 (m, 4H), 1.90-1.98 (m, 1H), 2.01-2.09 (m, 1H), 2.24-2.34 (m, 1H), 2.48-2.57 (m, 1H), 2.58-2.67 (m, 1H), 2.69-2.85 (m, 2H), 3.01 (t, J=10.74 Hz, 1H), 3.12 (s, 1H), 3.22-3.31 (m, 2H), 3.35 (s, 3H), 3.58 (s, 3H), 3.86 (dd, J=10.94, 3.52 Hz, 2H), 3.96-4.05 (m, 1H), 7.05 (d, J=8.59 Hz, 1H), 7.34 (d, J=8.20 Hz, 1H), 7.39 (d, J=1.17 Hz, 1H); MS (ESI) (M+H)⁺=453.30.

### Example 51

### (3S)—N-cyclopropyl-1-(9-methyl-3-(tetrahydro-2H-pyran-4-yl)-2,3,4,9-tetrahydro-1H-carbazole-6-carbonyl)piperidine-3-carboxamide (Isomers 1 and 2)

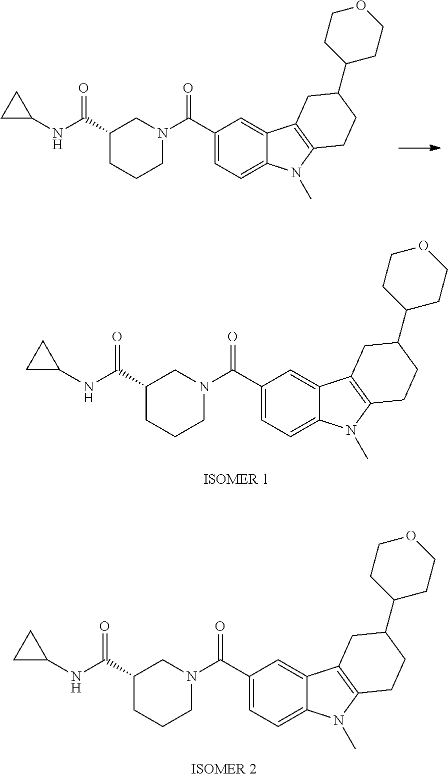

(3S)—N-Cyclopropyl-1-(9-methyl-3-(tetrahydro-2H-pyran-4-yl)-2,3,4,9-tetrahydro-1H-carbazole-6-carbonyl)piperidine-3-carboxamide (80 mg, 0.17 mmol) was separated by chiral HPLC. Chiral Purification: Gilson system equipped with a Chiracel AD column, 5 cm ID×50 cm L, 20 u using 20% iso-propanol/hexanes with 0.1% diethylamine v/v; 100 mL/min, 60 min run, rt. Chiral analytical HPLC: ChiraPak AD column, 20% iso-propanol/hexanes, 1 mL/min, 30 min run, 25° C.

Yield: Isomer 1: 35 mg (44%)
Isomer 2: 40 mg (50%)

Isomer 1: ¹H NMR (400 MHz, METHANOL-D4) δ 0.45 (s, 2H), 0.68 (s, 2H), 1.35-1.49 (m, 3H), 1.50-1.64 (m, 4H), 1.76 (t, J=12.89 Hz, 4H), 1.85-1.97 (m, 1H), 2.10-2.22 (m, 1H), 2.30-2.44 (m, 2H), 2.57-2.74 (m, 1H), 2.83 (t, J=14.45 Hz, 2H), 3.06-3.20 (m, 1H), 3.35-3.49 (m, 2H), 3.62 (s, 3H), 3.81 (s, 1H), 3.98 (dd, J=11.13, 3.71 Hz, 2H), 4.46 (s, 1H), 7.12 (dd, J=8.59, 1.56 Hz, 1H), 7.31 (d, J=7.81 Hz, 1H), 7.46 (d, J=1.17 Hz, 1H); Chiral HPLC k′=4.88; MS (ESI) (M+H)⁺=464.2; accurate mass: (M+H)=464.290.

Isomer 2: ¹H NMR (400 MHz, METHANOL-D4) δ 0.44 (s, 2H), 0.67 (s, 2H), 1.35-1.49 (m, 3H), 1.50-1.65 (m, 4H), 1.76 (t, J=13.48 Hz, 4H), 1.86-1.96 (m, 1H), 2.11-2.20 (m, 1H), 2.30-2.44 (m, 2H), 2.61-2.74 (m, 1H), 2.77-2.90 (m, 2H), 3.13 (s, 1H), 3.36-3.49 (m, 2H), 3.62 (s, 3H), 3.82 (s, 1H), 3.97 (dd, J=11.13, 3.71 Hz, 2H), 4.45 (s, 1H), 7.12 (dd, J=8.59, 1.56 Hz, 1H), 7.31 (d, J=8.20 Hz, 1H), 7.45 (d, J=1.17 Hz, 1H); Chiral HPLC k′=6.34; MS (ESI) (M+H)⁺=464.2; accurate mass: (M+H)=464.292.

### Example 52

### (R)—N,9-dimethyl-N-(4-(methylamino)-4-oxobutyl)-3-(tetrahydro-2H-pyran-4-yl)-2,3,4,9-tetrahydro-1H-carbazole-6-carboxamide and (S)—N,9-dimethyl-N-(4-(methylamino)-4-oxobutyl)-3-(tetrahydro-2H-pyran-4-yl)-2,3,4,9-tetrahydro-1H-carbazole-6-carboxamide

Chiral separation of N,9-dimethyl-N-(4-(methylamino)-4-oxobutyl)-3-(tetrahydro-2H-pyran-4-yl)-2,3,4,9-tetrahydro-1H-carbazole-6-carboxamide (80 mg, 0.19 mmol) was done as the following: Gilson system equipped with a Chiracel AD column, 5 cm ID×50 cm L, 20 u using 45% EtOH/hexanes with 0.1% diethylamine v/v; 100 mL/min, 60 min run, rt. Chiral analytical HPLC: ChiraPak AD column, 40% EtOH/hexanes, 1 mL/min, 30 min run, 25° C.

(R)—N,9-dimethyl-N-(4-(methylamino)-4-oxobutyl)-3-(tetrahydro-2H-pyran-4-yl)-2,3,4,9-tetrahydro-1H-carbazole-6-carboxamide (isomer 1, 30 mg, 37.5%):

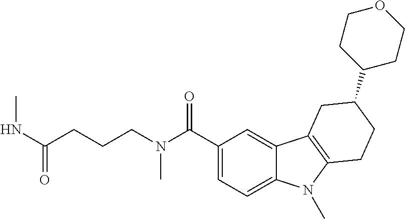

¹H NMR (400 MHz, METHANOL-D4) δ 1.37-1.50 (m, 2H), 1.52-1.61 (m, 3H), 1.77 (t, J=12.70 Hz, 2H), 1.86 (s, 1H), 1.96 (s, 2H), 2.13-2.20 (m, 1H), 2.26 (s, 1H), 2.34-2.44 (m, 1H), 2.49 (s, 1H), 2.64-2.73 (m, 2H), 2.79-2.90 (m, 2H), 3.04 (s, 3H), 3.36-3.47 (m, 3H), 3.55 (s, 1H), 3.63 (s, 3H), 3.98 (dd, J=10.94, 3.52 Hz, 2H), 7.13 (s, 1H), 7.31 (d, J=8.20 Hz, 1H), 7.46 (s, 1H); MS (ESI) (M+H)⁺=426.2; Chiral HPLC k′=3.20.

### Recrystallization:

(R)—N,9-dimethyl-N-(4-(methylamino)-4-oxobutyl)-3-(tetrahydro-2H-pyran-4-yl)-2,3,4,9-tetrahydro-1H-carbazole-6-carboxamide (0.98 g, 2.30 mmol) was dissolved in MeCN (1 mL) at room temperature. After stirring for 5 min, white solids were formed and collected. The white solids were recrystallized from MeCN solution (5 mL) to provide long rod crystals (827 mg, 84%). m.p. 134-136° C.; [α]_{D}=+55.1° (1.00, CDCl₃); HRMS m/z calcd for C₂₆H₃₆N₃O₃ [M+H]+ 426.2751. found 426.2749.

X-Ray study of the crystal is carried under the following condition and using the following parameters:

| Empirical formula | C25H35N3O3 | |
|---|---|---|
| Formula weight | 425.56 | |
| Temperature | 200 K | |
| Wavelength | 1.54178 Å | |
| Crystal system | Monoclinic | |
| Space group | P2 [symbol] | |
| Unit cell dimensions | a = 9.4497(3) | Å α = 90° |
| | b = 8.8381(3) | Å β = 100.982(1)° |
| | c = 13.8567(5) | Å γ = 90° |

| | |
|---|---|
| Volume | 1136.08(7)Å³ |
| Z | 2 |
| Density (calculated) | 1.244 g/cm³ |
| Absorption coefficient | 0.652 mm⁻¹ |
| F(000) | 460 |
| Crystal size | 0.25 × 0.14 × 0.13 mm |
| Theta range for data | 3.25 to 72.35° |
| collection | |
| Index ranges | −11 ≦ h ≦ 11, −9 ≦ k ≦ 10, −16 ≦ l ≦ 17 |
| Reflections collected | 14414 |
| Independent reflections | 3991 [Rᵢₙₜ = 0.037] |
| Absorption correction | Semi-empirical from equivalents |
| Max. and min. | 0.9187 and 0.8085 |
| transmission | |
| Refinement method | Full-matrix least-squares on F² |
| Data/restraints/ | 3991/1/284 |
| parameters | |
| Goodness-of-fit on F² | 1.136 |
| Final R indices | R₁ = 0.0439, wR₂ = 0.1002 |
| [I > 2sigma(I)] | |
| R indices (all data) | R₁ = 0.0441, wR₂ = 0.1004 |
| Absolute structure | −0.09(17) |
| parameter | |
| Extinction coefficient | 0.129(4) |
| Largest diff. peak and hole | 0.240 and −0.448 e/Å³ |

[symbol]

indicates data missing or illegible when filed

The X-ray results for the crystal are summarized in Tables 2 and 3:

TABLE 2
Atomic coordinates (×10⁴) and equivalent isotropic
displacement parameters (Å² × 10³) for C25H35N3O3.
U_{eq} is defined as one third of the trace of the orthogonalized
Uij tensor.

| | x | y | z | U_{eq} |
|---|---|---|---|---|
| N(1) | 5408(1) | 5382(2) | 2045(1) | 30(1) |
| C(2) | 6032(2) | 6811(2) | 2097(1) | 29(1) |
| C(3) | 5891(2) | 7893(2) | 1256(1) | 35(1) |
| C(4) | 6221(2) | 9498(2) | 1665(1) | 35(1) |
| C(5) | 7637(2) | 9538(2) | 2433(1) | 30(1) |
| C(6) | 7437(2) | 8608(2) | 3341(1) | 32(1) |
| C(7) | 6682(1) | 7134(2) | 3045(1) | 29(1) |
| C(8) | 6441(1) | 5845(2) | 3629(1) | 27(1) |
| C(9) | 6811(2) | 5486(2) | 4633(1) | 29(1) |
| C(10) | 6382(2) | 4113(2) | 4967(1) | 30(1) |
| C(11) | 5540(2) | 3089(2) | 4312(1) | 32(1) |
| C(12) | 5141(2) | 3418(2) | 3315(1) | 31(1) |
| C(13) | 5627(1) | 4781(2) | 2983(1) | 28(1) |
| C(14) | 4670(2) | 4633(2) | 1160(1) | 41(1) |
| C(15) | 8173(2) | 11159(2) | 2706(1) | 30(1) |
| C(16) | 8243(2) | 12146(2) | 1801(1) | 40(1) |
| C(17) | 8906(2) | 13684(2) | 2080(1) | 44(1) |
| O(18) | 10309(1) | 13576(2) | 2669(1) | 50(1) |
| C(19) | 10233(3) | 12797(2) | 3558(2) | 58(1) |
| C(20) | 9670(2) | 11184(2) | 3369(1) | 43(1) |
| C(21) | 6745(2) | 3796(2) | 6056(1) | 30(1) |
| O(21) | 6502(1) | 4751(2) | 6652(1) | 42(1) |
| N(22) | 7329(1) | 2429(2) | 6350(1) | 33(1) |
| C(22) | 7884(3) | 1344(2) | 5722(1) | 53(1) |
| C(23) | 7475(2) | 1990(2) | 7382(1) | 34(1) |
| C(24) | 8939(1) | 2336(2) | 8017(1) | 30(1) |
| C(25) | 8962(2) | 1819(2) | 9075(1) | 33(1) |
| C(26) | 10378(1) | 2154(2) | 9765(1) | 28(1) |
| O(26) | 10956(1) | 3418(1) | 9794(1) | 35(1) |
| N(27) | 10946(1) | 1006(2) | 10342(1) | 32(1) |
| C(27) | 12236(2) | 1218(2) | 11098(1) | 44(1) |

TABLE 3
Hydrogen coordinates (×10⁴) and isotropic displacement
parameters (Å² × 10³) for C25H35N3O3.

| | x | y | z | U_{eq} |
|---|---|---|---|---|
| H(3A) | 4901 | 7852 | 862 | 42 |
| H(3B) | 6575 | 7616 | 825 | 42 |
| H(4A) | 5417 | 9852 | 1972 | 42 |
| H(4B) | 6301 | 10195 | 1118 | 42 |
| H(5) | 8393 | 9021 | 2137 | 35 |
| H(6A) | 8392 | 8401 | 3756 | 38 |
| H(6B) | 6867 | 9203 | 3736 | 38 |
| H(9) | 7352 | 6181 | 5081 | 35 |
| H(11) | 5242 | 2161 | 4557 | 38 |
| H(12) | 4560 | 2741 | 2876 | 38 |
| H(14A) | 3628 | 4780 | 1092 | 61 |
| H(14B) | 4889 | 3548 | 1203 | 61 |
| H(14C) | 4993 | 5064 | 587 | 61 |
| H(15) | 7480 | 11643 | 3074 | 36 |
| H(16A) | 8820 | 11616 | 1378 | 48 |
| H(16B) | 7256 | 12284 | 1415 | 48 |
| H(17A) | 8961 | 14256 | 1474 | 52 |
| H(17B) | 8275 | 14256 | 2444 | 52 |
| H(19A) | 9590 | 13360 | 3918 | 70 |
| H(19B) | 11205 | 12766 | 3978 | 70 |
| H(20A) | 9619 | 10693 | 4003 | 51 |
| H(20B) | 10350 | 10597 | 3053 | 51 |
| H(22A) | 7213 | 492 | 5578 | 80 |
| H(22B) | 8827 | 973 | 6059 | 80 |
| H(22C) | 7986 | 1839 | 5106 | 80 |
| H(23A) | 7292 | 890 | 7413 | 40 |
| H(23B) | 6723 | 2519 | 7663 | 40 |
| H(24A) | 9130 | 3436 | 8006 | 36 |
| H(24B) | 9704 | 1805 | 7750 | 36 |
| H(25A) | 8172 | 2330 | 9324 | 39 |
| H(25B) | 8779 | 717 | 9076 | 39 |
| H(27) | 10535 | 110 | 10264 | 39 |
| H(27A) | 13046 | 1505 | 10788 | 66 |
| H(27B) | 12465 | 272 | 11463 | 66 |
| H(27C) | 12062 | 2019 | 11550 | 66 |

The molecular structure is shown in the following diagram.

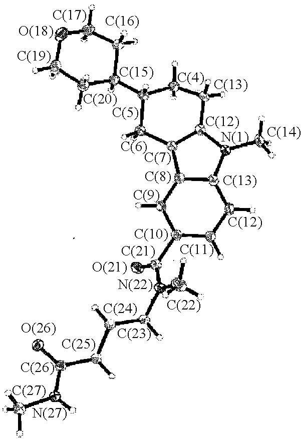

(S)—N,9-dimethyl-N-(4-(methylamino)-4-oxobutyl)-3-(tetrahydro-2H-pyran-4-yl)-2,3,4,9-tetrahydro-1H-carbazole-6-carboxamide (isomer 2, 30 mg, 37.5%):

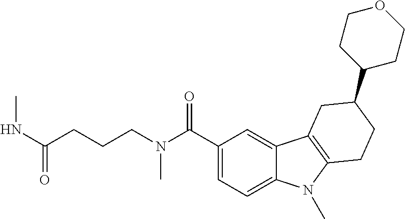

¹H NMR (400 MHz, METHANOL-D4) δ 1.38-1.50 (m, 2H), 1.53-1.60 (m, 3H), 1.78 (t, J=12.30 Hz, 2H), 1.85 (s, 1H), 1.96 (s, 2H), 2.13-2.20 (m, 1H), 2.26 (s, 1H), 2.39 (dd, J=16.21, 6.84 Hz, 1H), 2.49 (s, 1H), 2.65-2.74 (m, 2H), 2.80-2.89 (m, 2H), 3.04 (s, 3H), 3.37-3.47 (m, 3H), 3.55 (s, 1H), 3.63 (s, 3H), 3.98 (dd, J=11.33, 3.91 Hz, 2 H), 7.12 (s, 1H) 7.31 (d, J=8.59 Hz, 1H), 7.46 (s, 1H); MS (ESI) (M+H)⁺=426.2; Chiral HPLC k′=4.79.

### Example 53

### (R)—N-Ethyl-N-(2-(2-fluoroethylamino)-2-oxoethyl)-9-methyl-3-(tetrahydro-2H-pyran-4-yl)-2,3,4,9-tetrahydro-1H-carbazole-6-carboxamide and (S)—N-Ethyl-N-(2-(2-fluoroethylamino)-2-oxoethyl)-9-methyl-3-(tetrahydro-2H-pyran-4-yl)-2,3,4,9-tetrahydro-1H-carbazole-6-carboxamide.

Chiral separation of N-ethyl-N-(2-(2-fluoroethylamino)-2-oxoethyl)-9-methyl-3-(tetrahydro-2H-pyran-4-yl)-2,3,4,9-tetrahydro-1H-carbazole-6-carboxamide (75 mg, 0.17 mmol) was done as the following: Gilson system equipped with a Chiracel AD column, 5 cm ID×50 cm L, 20 u using 45% EtOH/hexanes with 0.1% diethylamine v/v; 100 mL/min, 60 min run, rt. Chiral analytical HPLC: ChiraPak AD column, 40% EtOH/hexanes, 1 mL/min, 30 min run, 25° C.

(R)—N-Ethyl-N-(2-(2-fluoroethylamino)-2-oxoethyl)-9-methyl-3-(tetrahydro-2H-pyran-4-yl)-2,3,4,9-tetrahydro-1H-carbazole-6-carboxamide (isomer 1, 25 mg, 33%):

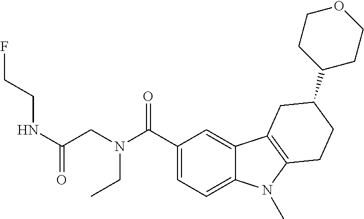

¹H NMR (400 MHz, METHANOL-D4) δ 1.10-1.26 (m, 3H), 1.38-1.48 (m, 2H), 1.53-1.62 (m, 3H), 1.77 (t, J=13.28 Hz, 2H), 2.12-2.20 (m, 1H), 2.38 (s, 1H), 2.64-2.74 (m, 1H), 2.83 (t, J=14.84 Hz, 2H), 3.38-3.44 (m, 3H), 3.47 (s, 1H), 3.53 (s, 2H), 3.63 (s, 3H), 3.98 (dd, J=11.13, 3.32 Hz, 2H), 4.03 (s, 1H), 4.15 (s, 1H), 4.39 (s, 1H), 4.51 (s, 1H), 7.18 (d, J=7.42 Hz, 1H), 7.31 (d, J=8.20 Hz, 1H), 7.52 (s, 1H); MS (ESI) (M+H)⁺=444.2; Chiral HPLC k′=4.42.

(S)—N-Ethyl-N-(2-(2-fluoroethylamino)-2-oxoethyl)-9-methyl-3-(tetrahydro-2H-pyran-4-yl)-2,3,4,9-tetrahydro-1H-carbazole-6-carboxamide (isomer 2, 25 mg, 33%):

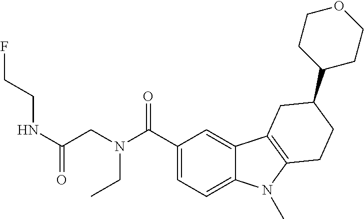

¹H NMR (400 MHz, METHANOL-D4) δ 1.12-1.25 (m, 3H), 1.37-1.49 (m, 2H), 1.52-1.61 (m, 3H), 1.77 (t, J=13.28 Hz, 2H), 2.13-2.20 (m, 1H), 2.36 (dd, J=10.35, 4.49 Hz, 1H), 2.63-2.73 (m, 1H), 2.77-2.89 (m, 2H), 3.36-3.46 (m, 3H), 3.47 (s, 1H), 3.53 (s, 2H), 3.62 (s, 3H), 3.98 (dd, J=11.33, 3.52 Hz, 2H), 4.04 (s, 1H), 4.15 (s, 1H), 4.39 (s, 1H), 4.51 (s, 1H), 7.18 (d, J=6.64 Hz, 1H), 7.31 (d, J=8.20 Hz, 1H), 7.53 (s, 1 H); MS (ESI) (M+H)⁺=444.2; Chiral HPLC k′=5.83

### Example 54

### (R)—N-(2-(Cyclopropylamino)-2-oxoethyl)-N-ethyl-9-methyl-3-(tetrahydro-2H-pyran-4-yl)-2,3,4,9-tetrahydro-1H-carbazole-6-carboxamide and (S)—N-(2-(Cyclopropylamino)-2-oxoethyl)-N-ethyl-9-methyl-3-(tetrahydro-2H-pyran-4-yl)-2,3,4,9-tetrahydro-1H-carbazole-6-carboxamide

Chiral separation of N-(2-(cyclopropylamino)-2-oxoethyl)-N-ethyl-9-methyl-3-(tetrahydro-2H-pyran-4-yl)-2,3,4,9-tetrahydro-1H-carbazole-6-carboxamide (85 mg, 0.19 mmol) was done as the following: Gilson system equipped with a Chiracel AD column, 5 cm ID×50 cm L, 20 u using 30% EtOH/hexanes with 0.1% diethylamine v/v; 100 mL/min, 60 min run, rt. Chiral analytical HPLC: ChiraPak AD column, 25% EtOH/hexanes, 1 mL/min, 30 min run, 25° C.

(R)—N-(2-(Cyclopropylamino)-2-oxoethyl)-N-ethyl-9-methyl-3-(tetrahydro-2H-pyran-4-yl)-2,3,4,9-tetrahydro-1H-carbazole-6-carboxamide (isomer 1, 25 mg, 29%):

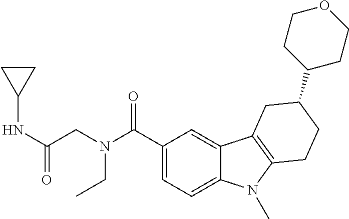

¹H NMR (400 MHz, METHANOL-D4) δ 0.39-0.55 (m, 2H), 0.70 (d, J=5.47 Hz, 2H), 1.11-1.22 (m, 3H), 1.36-1.49 (m, 2H), 1.56 (s, 3H), 1.77 (t, J=12.50 Hz, 2H), 2.13-2.20 (m, 1H), 2.37 (dd, J=15.23, 5.08 Hz, 1H), 2.62-2.73 (m, 2H), 2.77-2.89 (m, 2 H), 3.36-3.46 (m, 3H), 3.53 (s, 1H), 3.62 (s, 3H), 3.93 (s, 1H), 3.98 (dd, J=11.13, 3.32 Hz, 2H), 4.06 (s, 1H), 7.17 (s, 1H), 7.30 (d, J=8.20 Hz, 1H), 7.51 (s, 1H); MS (ESI) (M+H)⁺=438.3; Chiral HPLC k′=3.86.

(S)—N-(2-(Cyclopropylamino)-2-oxoethyl)-N-ethyl-9-methyl-3-(tetrahydro-2H-pyran-4-yl)-2,3,4,9-tetrahydro-1H-carbazole-6-carboxamide (isomer 2, 26 mg, 31%):

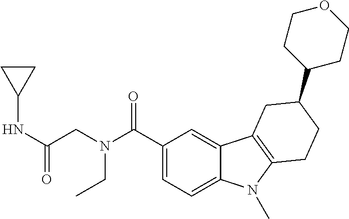

¹H NMR (400 MHz, METHANOL-D4) δ 0.37-0.56 (m, 2H), 0.70 (d, J=5.86 Hz, 2H), 1.09-1.24 (m, 3H), 1.37-1.49 (m, 2H), 1.56 (s, 3H), 1.77 (t, J=12.50 Hz, 2H), 2.12-2.22 (m, 1H), 2.32-2.43 (m, 1H), 2.61-2.73 (m, 2H), 2.77-2.90 (m, 2H), 3.36-3.46 (m, 3H), 3.53 (s, 1H), 3.62 (s, 3H), 3.93 (s, 1H), 3.98 (dd, J=11.13, 3.32 Hz, 2H), 4.07 (s, 1H), 7.18 (s, 1H), 7.30 (d, J=7.42 Hz, 1H), 7.51 (s, 1H); MS (ESI) (M+H)⁺=438.3; Chrial HPLC k′=4.81.

### Example 55

### (R)—N-(4-(2-Fluoroethylamino)-4-oxobutyl)-N,9-dimethyl-3-(tetrahydro-2H-pyran-4-yl)-2,3,4,9-tetrahydro-1H-carbazole-6-carboxamide and (S)—N-(4-(2-Fluoroethylamino)-4-oxobutyl)-N,9-dimethyl-3-(tetrahydro-2H-pyran-4-yl)-2,3,4,9-tetrahydro-1H-carbazole-6-carboxamide.

Chiral separation of N-(4-(2-fluoroethylamino)-4-oxobutyl)-N,9-dimethyl-3-(tetrahydro-2H-pyran-4-yl)-2,3,4,9-tetrahydro-1H-carbazole-6-carboxamide (55 mg, 0.12 mmol) was done as the following: Gilson system equipped with a Chiracel AD column, 5 cm ID×50 cm L, 20 u using 20% iPrOH/hexanes with 0.1% diethylamine v/v; 100 mL/min, 60 min run, rt. Chiral analytical HPLC: ChiraPak AD column, 20% iPrOH/hexanes, 1 mL/min, 30 min run, 25° C.

(R)—N-(4-(2-Fluoroethylamino)-4-oxobutyl)-N,9-dimethyl-3-(tetrahydro-2H-pyran-4-yl)-2,3,4,9-tetrahydro-1H-carbazole-6-carboxamide (isomer 1, 27 mg, 49%):

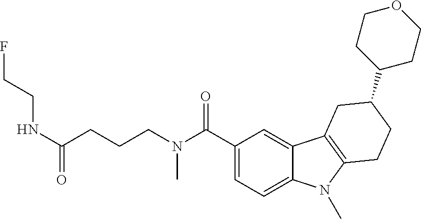

¹H NMR (400 MHz, METHANOL-D4) δ 1.38-1.49 (m, 2H), 1.52-1.62 (m, 3H), 1.77 (t, J=12.50 Hz, 2H), 1.87 (s, 1H), 1.99 (s, 2H), 2.12-2.21 (m, 1H), 2.31 (s, 1H), 2.34-2.44 (m, 1H), 2.63-2.75 (m, 1H), 2.79-2.91 (m, 2H), 3.04 (s, 3H), 3.15-3.25 (m, 1H), 3.35-3.47 (m, 3H), 3.50 (s, 1H), 3.57 (s, 1H), 3.63 (s, 3H), 3.98 (dd, J=11.13, 3.71 Hz, 2H), 4.17 (s, 0.5H), 4.29 (s, 0.5H), 4.37 (s, 0.5H), 4.49 (s, 0.5H), 7.12 (s, 1H), 7.31 (d, J=8.59 Hz, 1H), 7.46 (s, 1 H); MS (ESI) (M+H)⁺=458.3; Chiral HPLC k′=7.76.

(S)—N-(4-(2-Fluoroethylamino)-4-oxobutyl)-N,9-dimethyl-3-(tetrahydro-2H-pyran-4-yl)-2,3,4,9-tetrahydro-1H-carbazole-6-carboxamide (isomer 2, 24 mg, 43%):

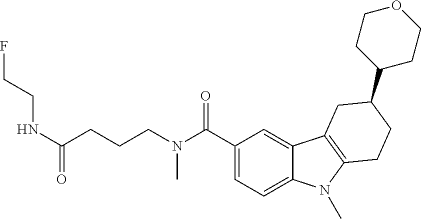

¹H NMR (400 MHz, METHANOL-D4) δ 1.37-1.50 (m, 2H), 1.51-1.61 (m, 3H), 1.77 (t, J=12.11 Hz, 2H), 1.87 (s, 1H), 1.98 (s, 2H), 2.12-2.20 (m, 1H), 2.31 (s, 1H), 2.38 (dd, J=15.62, 7.03 Hz, 1H), 2.63-2.75 (m, 1H), 2.84 (dd, J=12.11, 2.73 Hz, 2H), 3.04 (s, 3H), 3.21 (s, 1H), 3.36-3.47 (m, 3H), 3.50 (s, 1H), 3.57 (s, 1H), 3.63 (s, 3H), 3.98 (dd, J=11.33, 3.91 Hz, 2H), 4.18 (s, 0.5H), 4.29 (s, 0.5H), 4.37 (s, 0.5H), 4.49 (s, 0.5H), 7.12 (s, 1H), 7.31 (d, J=8.20 Hz, 1H), 7.46 (s, 1H); MS (ESI) (M+H)⁺=458.3; Chiral HPLC k′=9.46.

### Example 56

### (R)—N-ethyl-N-(2-hydroxyethyl)-9-methyl-3-(tetrahydro-2H-pyran-4-yl)-2,3,4,9-tetrahydro-1H-carbazole-6-carboxamide and (S)—N-ethyl-N-(2-hydroxyethyl)-9-methyl-3-(tetrahydro-2H-pyran-4-yl)-2,3,4,9-tetrahydro-1H-carbazole-6-carboxamide.

Chiral separation of N-ethyl-N-(2-hydroxyethyl)-9-methyl-3-(tetrahydro-2H-pyran-4-yl)-2,3,4,9-tetrahydro-1H-carbazole-6-carboxamide (90 mg, 0.23 mmol) was done as the following: Gilson system equipped with a Chiracel AD column, 5 cm ID×50 cm L, 20 u using 15% 1:1 MeOH:iPrOH/hexanes with 0.1% diethylamine v/v; 100 mL/min, 60 min run, rt. Chiral analytical HPLC: ChiraPak AD column, 15% MeOH:iPrOH/hexanes, 1 mL/min, 30 min run, 25° C. Products needed to be repurified by reversed-phase HPLC as their NMR showed the presence of impurities. Reversed-phase purification: Gilson system equipped with Luna C-18 column, 250×21.2 mm, 15 u. Mobile phase: 30-50% B; A: H₂O with 0.05% TFA v/v; B: CH₃CN; 30 mL/min, 25 min run, rt.

(R)—N-ethyl-N-(2-hydroxyethyl)-9-methyl-3-(tetrahydro-2H-pyran-4-yl)-2,3,4,9-tetrahydro-1H-carbazole-6-carboxamide (isomer 1, 25 mg, 28%):

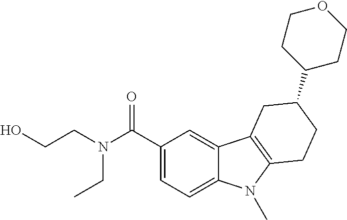

¹H NMR (400 MHz, METHANOL-D4) δ 1.11 (s, 2 H), 1.23 (s, 1H), 1.37-1.49 (m, 2H), 1.51-1.62 (m, 3H), 1.77 (t, J=13.28 Hz, 2H), 2.13-2.20 (m, 1H), 2.32-2.43 (m, 1H), 2.62-2.74 (m, 1H), 2.78-2.89 (m, 2H), 3.36-3.50 (m, 4H), 3.54-3.66 (m, 6H), 3.80 (s, 1H), 3.98 (dd, J=11.13, 4.10 Hz, 2H), 7.12 (dd, J=8.20, 1.56 Hz, 1H), 7.31 (d, J=8.20 Hz, 1H), 7.46 (d, J=0.78 Hz, 1H); chiral HPLC k′=5.68; MS (ESI) (M+H)⁺=385.2; accurate mass: (M+H)=385.248.

(S)—N-ethyl-N-(2-hydroxyethyl)-9-methyl-3-(tetrahydro-2H-pyran-4-yl)-2,3,4,9-tetrahydro-1H-carbazole-6-carboxamide (isomer 2, 25 mg, 28%):

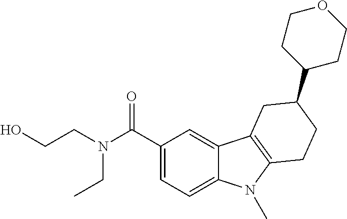

¹H NMR (400 MHz, METHANOL-D4) δ 1.11 (s, 2H), 1.19-1.28 (m, 1H), 1.36-1.48 (m, 2H), 1.51-1.60 (m, 3H), 1.76 (t, J=13.28 Hz, 2H), 2.11-2.20 (m, 1H), 2.37 (dd, J=15.62, 7.03 Hz, 1H), 2.61-2.73 (m, 1H), 2.78-2.89 (m, 2H), 3.35-3.51 (m, 4H), 3.62 (s, 6H), 3.80 (s, 1H), 3.97 (dd, J=11.52, 3.71 Hz, 2H), 7.12 (dd, J=8.20, 1.56 Hz, 1H), 7.31 (d, J=8.20 Hz, 1H), 7.46 (d, J=0.78 Hz, 1H); chiral HPLC k′=6.93; MS (ESI) (M+H)⁺=385.2; accurate mass: (M+H)=385.248.

### Example 57

### N-Cyclopropyl-1-(9-methyl-3-(tetrahydro-2H-pyran-4-yl)-2,3,4,9-tetrahydro-1H-carbazole-6-carbonyl)pyrrolidine-3-carboxamide

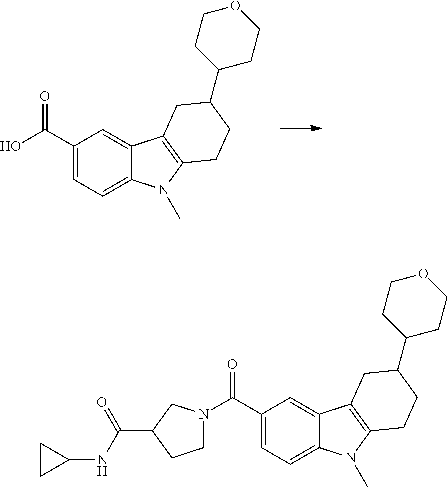

9-Methyl-3-(tetrahydro-2H-pyran-4-yl)-2,3,4,9-tetrahydro-1H-carbazole-6-carboxylic acid (100 mg, 0.32 mmol), HATU (146 mg, 0.38 mmol) and 3-(cyclopropylcarbamoyl)pyrrolidinium chloride (73.0 mg, 0.38 mmol) were stirred in DMF (10 mL) containing N,N-diisopropylethylaminc (0.139 mL, 0.80 mmol) at 23° C. for 1 h. The solvent was evaporated. The residue was dissolved in EtOAc and washed with aqueous saturated NaHCO₃ solution, brine and dried over anhydrous Na₂SO₄. The product was purified by reversed-phase HPLC using 30-50% and lyophilized. Purification: Gilson system equipped with Luna C-18 column, 250×21.2 mm, 15 u. Mobile phase: A: H₂O with 0.05% TFA v/v; B: CH₃CN; 30 mL/min, 25 min run, rt. Yield: 40 mg (28%). ¹H NMR (400 MHz, METHANOL-D4) δ 0.04 (m, 2H), 0.29 (m, 2 H), 0.96-1.09 (m, 2H), 1.10-1.20 (m, 3H), 1.36 (t, J=12.70 Hz, 2H), 1.61-1.70 (m, 1 H), 1.72-1.80 (m, 2H), 1.92-2.03 (m, 1H), 2.19-2.33 (m, 2H), 2.39-2.50 (m, 2.5H), 2.56-2.65 (m, 0.5H), 2.96-3.07 (m, 2H), 3.16-3.24 (m, 4H), 3.25-3.41 (m, 3 H), 3.57 (dd, J=11.13, 3.71 Hz, 2H), 6.83-6.88 (m, 1H), 6.89-6.93 (m, 1H), 7.20 (d, J=0.78 Hz, 1H); MS (ESI) (M+H)⁺=450.2; accurate mass: (M+H)=450.275.

### Example 58

### Step A: (3S)—N-Cyclopropyl-1-(9-methyl-3-(tetrahydro-2H-pyran-4-yl)-2,3,4,9-tetrahydro-1H-carbazole-6-carbonyl)pyrrolidine-3-carboxamide (Isomers 1 and 2)

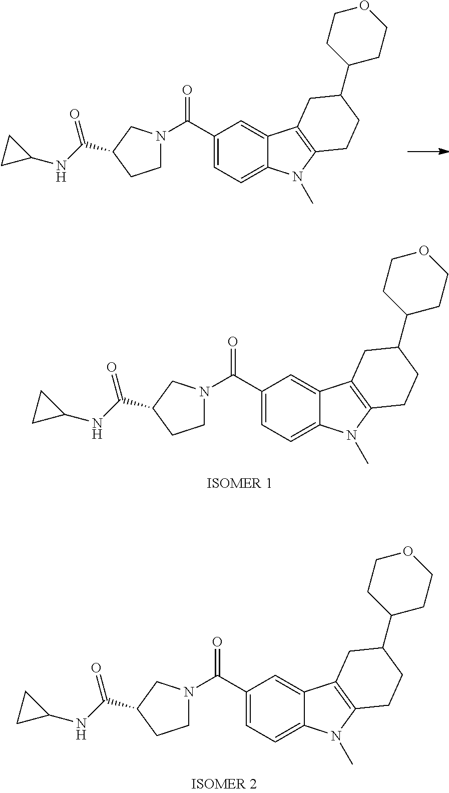

Chiral separation of (3S)—N-cyclopropyl-1-(9-methyl-3-(tetrahydro-2H-pyran-4-yl)-2,3,4,9-tetrahydro-1H-carbazole-6-carbonyl)pyrrolidine-3-carboxamide (100 mg, 0.22 mmol). Chiral Purification: Gilson system equipped with a Chiracel AD column, 5 cm ID×50 cm L, 20 u using 35% EtOH/hexanes with 0.1% diethylamine v/v; 100 mL/min, 60 min run, rt. Chiral analytical HPLC: ChiraPak AD column, 40% EtOH/hexanes, 1 mL/min, 30 min run, 25° C.

Yield: Isomer 1: 42 mg (42%)
Isomer 2: 42 mg (42%)

Isomer 1: ¹H NMR (400 MHz, METHANOL-D4) δ 0.40 (s, 1H), 0.48 (s, 1H), 0.69 (dd, J=23.44, 6.25 Hz, 2H), 1.37-1.50 (m, 2H), 1.52-1.63 (m, 3H), 1.77 (t, J=12.70 Hz, 2 H), 2.01-2.10 (m, 1H), 2.12-2.21 (m, 2H), 2.33-2.43 (m, 1H), 2.55-2.63 (m, 0.5H), 2.63-2.74 (m, 1H), 2.79-2.89 (m, 3H), 2.96-3.06 (m, 0.5H), 3.37-3.47 (m, 2 H), 3.54-3.66 (m, 4H), 3.67-3.82 (m, 3H), 3.98 (dd, J=11.13, 3.71 Hz, 2H), 7.24-7.28 (m, 1H), 7.28-7.33 (m, 1H), 7.60 (s, 1H); chiral HPLC k′=2.33; MS (ESI) (M+H)⁺=450.2; accurate mass: (M+H)=450.275.

Isomer 2: ¹H NMR (400 MHz, METHANOL-D4) δ 0.40 (s, 1H), 0.48 (s, 1H), 0.68 (dd, J=23.63, 6.84 Hz, 2H), 1.37-1.49 (m, 2H), 1.52-1.62 (m, 3H), 1.77 (t, J=12.50 Hz, 2 H), 2.01-2.09 (m, 1H), 2.12-2.21 (m, 2H), 2.39 (dd, J=15.62, 7.03 Hz, 1H), 2.55-2.63 (m, 0.5H), 2.64-2.74 (m, 1H), 2.80-2.90 (m, 3H), 2.97-3.06 (m, 0.5H), 3.37-3.46 (m, 2H), 3.55-3.65 (m, 4H), 3.67-3.80 (m, 3H), 3.98 (dd, J=11.33, 3.52 Hz, 2 H), 7.23-7.28 (m, 1H), 7.29-7.33 (m, 1H), 7.60 (d, J=1.17 Hz, 1H); chiral HPLC k′=3.60; MS (ESI) (M+H)⁺=450.2; accurate mass: (M+H)=450.275.

### Step B: (S)—N-Cyclopropylpyrrolidine-3-carboxamide hydrochloride

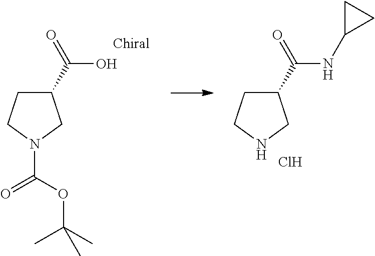

(S)-1-N-Boc-beta-proline (500 mg, 2.32 mmol), cyclopropylamine (0.193 mL, 2.79 mmol) and O-(7-azabenzotriazol-1-yl)-N,N,N′,N′-tetramethyluronium hexafluorophosphate (1060 mg, 2.79 mmol) were stirred in DMF (10 mL) containing N,N-diisopropylethylamine (0.607 mL, 3.48 mmol) at 23° C. for 1 h. The solvent was evaporated. The residue was dissolved in EtOAc and washed with 5% aqueous KHSO₄, aqueous saturated NaHCO₃, brine and dried over anhydrous Na₂SO₄. The solvent was evaporated. The residue was stirred in hydrogen chloride (1.16E+04 μL, 11.60 mmol) (1M in AcOH) at 23° C. for 2-3 h. The solvent was evaporated. The residue was washed with ether and the ether layer was decanted. The product was dried under vacuum overnight. The product was used directly for the next step. MS (ESI) (M+H)⁺=255.21 (Boc product). Yield: 450 mg (100%)

### Step C: (3S)—N-Cyclopropyl-1-(9-methyl-3-(tetrahydro-2H-pyran-4-yl)-2,3,4,9-tetrahydro-1H-carbazole-6-carbonyl)pyrrolidine-3-carboxamide

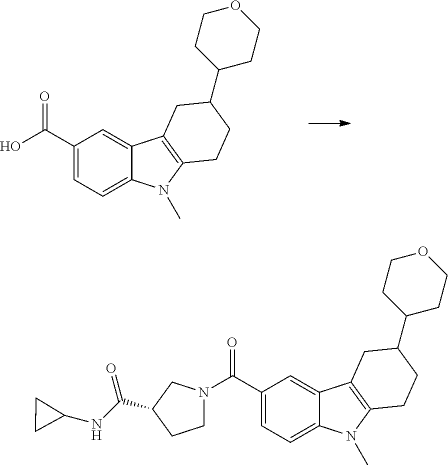

9-Methyl-3-(tetrahydro-2H-pyran-4-yl)-2,3,4,9-tetrahydro-1H-carbazole-6-carboxylic acid (125 mg, 0.40 mmol), (S)—N-cyclopropylpyrrolidine-3-carboxamide hydrochloride (91 mg, 0.48 mmol) and HATU (182 mg, 0.48 mmol) were stirred in DMF (10 mL) containing N,N-diisopropylethylamine (0.174 mL, 1.00 mmol) at 23° C. for 1 h. LC/MS showed presence of 1:1 product with HATU intermediate. Another 1.2 eq of (S)—N-cyclopropylpyrrolidine-3-carboxamide hydrochloride was added along with 1.5 eq of DIPEA and solution was stirred at rt for another 2 h. The solvent was evaporated. The residue was dissolved in EtOAc and washed with aqueous saturated NaHCO₃, brine and dried over anhydrous Na₂SO₄. The product was purified by reversed-phase HPLC using 30-50% B and lyophilized. Purification: Gilson system equipped with Luna C-18 column, 250×21.2 mm, 15 u. Mobile phase: A: H₂O with 0.05% TFA v/v; B: CH₃CN; 30 mL/min, 25 min run, rt. Yield: 110 mg (61%); ¹H NMR (400 MHz, METHANOL-D4) δ 0.40 (s, 1H), 0.48 (s, 1H), 0.68 (dd, J=24.22, 6.64 Hz, 2H), 1.36-1.48 (m, 2H), 1.55 (s, 3H), 1.76 (t, J=12.50 Hz, 2H), 2.00-2.07 (m, 1H), 2.12-2.19 (m, 2H), 2.33-2.42 (m, 1H), 2.59 (s, 0.5H), 2.63-2.74 (m, 1H), 2.77-2.89 (m, 2H), 2.96-3.04 (m, 0.5H), 3.36-3.46 (m, 2H), 3.55-3.66 (m, 5H), 3.67-3.82 (m, 3H), 3.97 (dd, J=11.33, 2.73 Hz, 2H), 7.24-7.34 (m, 2H), 7.60 (s, 1H); MS (ESI) (M+H)⁺=450.45.

### Example 59

### Step A: (3R)—N-Cyclopropyl-1-(9-methyl-3-(tetrahydro-2H-pyran-4-yl)-2,3,4,9-tetrahydro-1H-carbazole-6-carbonyl)pyrrolidine-3-carboxamide (Isomers 1 and 2)

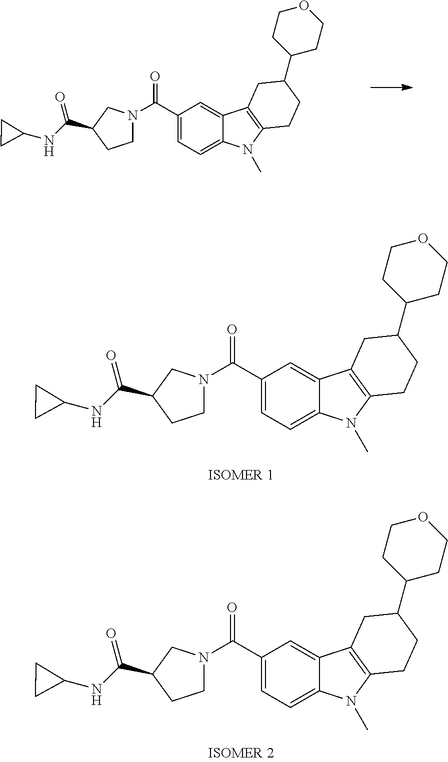

Chiral separation of (3R)—N-cyclopropyl-1-(9-methyl-3-(tetrahydro-2H-pyran-4-yl)-2,3,4,9-tetrahydro-1H-carbazole-6-carbonyl)pyrrolidine-3-carboxamide (90 mg, 0.20 mmol). Chiral Purification: Gilson system equipped with a Chiracel AD column, 5 cm ID×50 cm L, 20 u using 35% EtOH/hexanes with 0.1% diethylamine v/v; 100 mL/min, 60 min run, rt. Chiral analytical HPLC: ChiraPak AD column, 40% EtOH/hexanes, 1 mL/min, 30 min run, 25° C.

Yield: Isomer 1: 35 mg (39%)
Isomer 2: 35 mg (39%)

Isomer 1: ¹H NMR (400 MHz, METHANOL-D4) δ 0.40 (s, 1H), 0.48 (s, 1H), 0.68 (dd, J=23.44, 7.03 Hz, 2H), 1.36-1.48 (m, 2H), 1.51-1.61 (m, 3H), 1.76 (t, J=12.70 Hz, 2 H), 2.01-2.07 (m, 1H), 2.12-2.21 (m, 2H), 2.32-2.42 (m, 1H), 2.55-2.62 (m, 0.5H), 2.63-2.72 (m, 1H), 2.79-2.91 (m, 3H), 2.97-3.05 (m, 0.5H), 3.36-3.46 (m, 2 H), 3.55-3.65 (m, 4H), 3.66-3.79 (m, 3H), 3.97 (dd, J=11.13, 3.71 Hz, 2H), 7.24-7.28 (m, 1H), 7.28-7.33 (m, 1H), 7.60 (s, 1H); chiral HPLC k′=4.34; MS (ESI) (M+H)⁺=450.2; accurate mass: (M+H)=450.275.

Isomer 2: ¹H NMR (400 MHz, METHANOL-D4) δ 0.40 (s, 1H), 0.48 (s, 1H), 0.69 (dd, J=23.63, 6.45 Hz, 2H), 1.36-1.49 (m, 2H), 1.52-1.61 (m, 3H), 1.77 (t, J=12.50 Hz, 2 H), 2.01-2.09 (m, 1H), 2.12-2.21 (m, 2H), 2.34-2.44 (m, 1H), 2.59 (m, 0.5H), 2.63-2.74 (m, 1H), 2.80-2.91 (m, 3H), 2.96-3.05 (m, 0.5H), 3.36-3.47 (m, 2H), 3.56-3.66 (m, 4H), 3.68-3.82 (m, 3H), 3.98 (dd, J=11.13, 3.71 Hz, 2H), 7.24-7.28 (m, 1H), 7.28-7.33 (m, 1H), 7.60 (d, J=1.17 Hz, 1H); chiral HPLC k′=5.95; MS (ESI) (M+H)⁺=450.2; accurate mass: (M+H)=450.275.

### Step B: (R)—N-Cyclopropylpyrrolidine-3-carboxamide hydrochloride

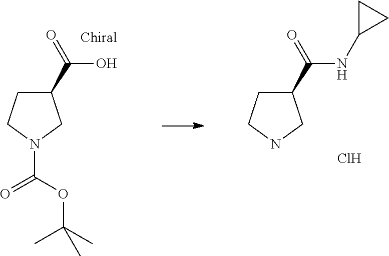

(R)-1-N-Boc-beta-proline (500 mg, 2.32 mmol), cyclopropylamine (0.193 mL, 2.79 mmol) and HATU (1060 mg, 2.79 mmol) were stirred in DMF (10 mL) containing N,N-diisopropylethylamine (0.607 mL, 3.48 mmol) at 23° C. for 1 h. The solvent was evaporated. The residue was dissolved in EtOAc and washed with 5% aqueous KHSO₄, aqueous saturated NaHCO₃, brine and dried over anhydrous Na₂SO₄. The solvent was evaporated. The residue was stirred in hydrogen chloride (1.16E+04 μL, 11.60 mmol) (1M in AcOH) at 23° C. for 2-3 h. The solvent was evaporated. The residue was washed with ether and the ether layer was decanted. The product was dried under vacuum overnight and used directly for the next step. MS (ESI) (M+H)⁺=255.21 (Boc product). Yield: 460 mg (104%)

### Step C: (3R)—N-Cyclopropyl-1-(9-methyl-3-(tetrahydro-2H-pyran-4-yl)-2,3,4,9-tetrahydro-1H-carbazole-6-carbonyl)pyrrolidine-3-carboxamide

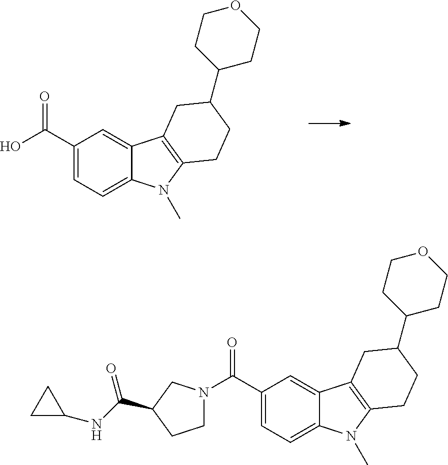

9-Methyl-3-(tetrahydro-2H-pyran-4-yl)-2,3,4,9-tetrahydro-1H-carbazole-6-carboxylic acid (125 mg, 0.40 mmol), (R)—N-cyclopropylpyrrolidine-3-carboxamide hydrochloride (91 mg, 0.48 mmol) and HATU (182 mg, 0.48 mmol) were stirred in DMF (10 mL) containing N,N-diisopropylethylamine (0.174 mL, 1.00 mmol) at 23° C. for 1 h. LC/MS showed presence of 1:1 product with HATU intermediate. Another 1.2 eq of (R)—N-cyclopropylpyrrolidine-3-carboxamide hydrochloride was added along with 1.5 eq of DIPEA and solution was stirred at rt for another 2 h. The solvent was evaporated. The residue was dissolved in EtOAc and washed with aqueous saturated NaHCO₃, brine and dried over anhydrous Na₂SO₄. The product was purified by reversed-phase HPLC using 30-50% B and lyophilized. Purification: Gilson system equipped with Luna C-18 column, 250×21.2 mm, 15 u. Mobile phase: A: H₂O with 0.05% TFA v/v; B: CH₃CN; 30 mL/min, 25 min run, rt. Yield: 90 mg (50%); ¹H NMR (400 MHz, METHANOL-D4) δ 0.40 (s, 1H), 0.48 (s, 1H), 0.69 (dd, J=24.02, 6.84 Hz, 2H), 1.36-1.49 (m, 2H), 1.52-1.61 (m, 3H), 1.77 (t, J=12.70 Hz, 2H), 2.01-2.10 (m, 1H), 2.12-2.20 (m, 2H), 2.33-2.43 (m, 1H), 2.56-2.62 (m, 0.5H), 2.63-2.73 (m, 2H), 2.80-2.89 (m, 2H), 2.98-3.05 (m, 0.5H), 3.36-3.46 (m, 2H), 3.58-3.64 (m, 4H), 3.66-3.82 (m, 3H), 3.98 (dd, J=11.13, 3.32 Hz, 2H), 7.24-7.28 (m, 1H), 7.29-7.34 (m, 1H), 7.60 (s, 1H); MS (ESI) (M+H)⁺=450.50.

### Example 60

### Step A: N-(2-Fluoroethyl)-1-(9-methyl-3-(tetrahydro-2H-pyran-4-yl)-2,3,4,9-tetrahydro-1H-carbazole-6-carbonyl)pyrrolidine-3-carboxamide

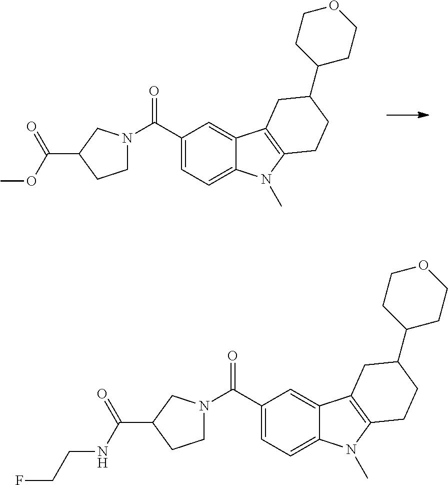

Methyl 1-(9-methyl-3-(tetrahydro-2H-pyran-4-yl)-2,3,4,9-tetrahydro-1H-carbazole-6-carbonyl)pyrrolidine-3-carboxylate (100 mg, 0.24 mmol) was stirred in dioxane (5 mL) containing lithium hydroxide (0.471 mL, 0.47 mmol) (1M) at 23° C. for 1 h. The solvent was evaporated. The residue was dissolved in DMF (5.00 mL) containing N,N-diisopropylethylamine (0.103 mL, 0.59 mmol) and 2-fluoroethylamine hydrochloride (28.1 mg, 0.28 mmol) along with O-(7-azabenzotriazol-1-yl)-N,N,N′,N′-tetramethyluronium hexafluorophosphate (107 mg, 0.28 mmol) were added. The solution was stirred at 23° C. for 1 h. The solution was concentrated. The residue was dissolved in EtOAc and washed with saturated aqueous NaHCO₃, brine and dried over anhydrous Na₂SO₄. The product was purified by reversed-phase HPLC using 30-50% B and lyophilized. Purification: Gilson system equipped with Luna C-18 column, 250×21.2 mm, 15 u. Mobile phase: A: H₂O with 0.05% TFA v/v; B: CH₃CN; 30 mL/min, 25 min run, rt. Yield: 63 mg (59%); ¹H NMR (400 MHz, METHANOL-D4) δ 1.35-1.45 (m, 2 H), 1.54 (s, 3H), 1.74 (t, J=12.50 Hz, 2H), 2.00-2.09 (m, 1H), 2.10-2.22 (m, 2H), 2.30-2.41 (m, 1H), 2.60-2.71 (m, 1H), 2.77-2.87 (m, 2H), 2.92-3.02 (m, 0.5H), 3.06-3.15 (m, 0.5H), 3.35-3.47 (m, 3H), 3.52 (s, 1H), 3.60 (s, 4H), 3.67-3.85 (m, 3H), 3.96 (d, J=8.20 Hz, 2H), 4.35 (m, 1H), 4.42-4.54 (m, 1H), 7.23-7.33 (m, 2H), 7.60 (s, 1H); MS (ESI) (M+H)⁺=456.2; accurate mass: (M+H)=456.265.

### Step B: Methyl 1-(9-methyl-3-(tetrahydro-2H-pyran-4-yl)-2,3,4,9-tetrahydro-1H-carbazole-6-carbonyl)pyrrolidine-3-carboxylate

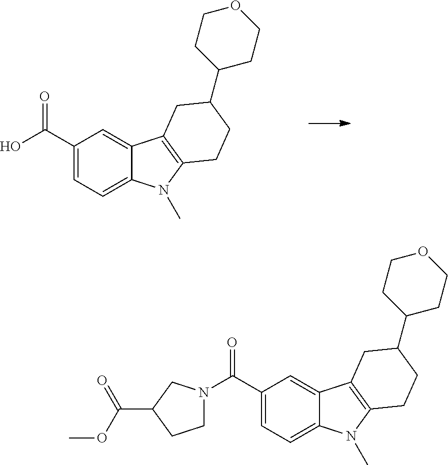

9-Methyl-3-(tetrahydro-2H-pyran-4-yl)-2,3,4,9-tetrahydro-1H-carbazole-6-carboxylic acid (100 mg, 0.32 mmol), methyl 3-pyrrolidinecarboxylate (49.5 mg, 0.38 mmol) and HATU (146 mg, 0.38 mmol) were stirred in DMF (5 mL) containing N,N-diisopropylethylamine (0.083 mL, 0.48 mmol) at 23° C. for 2 h. The solvent was evaporated. The residue was dissolved in EtOAc and washed with saturated aqueous NaHCO₃, brine and dried over anhydrous Na₂SO₄. The product was purified by flash using EtOAc as eluent. Yield: 110 mg (81%); ¹H NMR (400 MHz, METHANOL-D4) δ 1.36-1.48 (m, 2 ED, 1.51-1.61 (m, 3H), 1.76 (t, J=12.50 Hz, 2H), 2.07-2.18 (m, 2H), 2.19-2.29 (m, 1H), 2.32-2.43 (m, 1H), 2.61-2.73 (m, 1H), 2.79-2.88 (m, 2H), 3.07-3.17 (m, 0.5H), 3.20-3.28 (m, 0.5H), 3.36-3.46 (m, 2H), 3.62 (s, 3H), 3.63-3.74 (m, 4H), 3.73-3.84 (m, 2H), 3.97 (dd, J=10.94, 3.52 Hz, 2H), 7.23-7.28 (m, 1H), 7.28-7.33 (m, 1H), 7.59 (s, 1H); MS (ESI) (M+H)⁺=425.43.

### Example 61

### N-Ethyl-1-(9-methyl-3-(tetrahydro-2H-pyran-4-yl)-2,3,4,9-tetrahydro-1H-carbazole-6-carbonyl)pyrrolidine-3-carboxamide

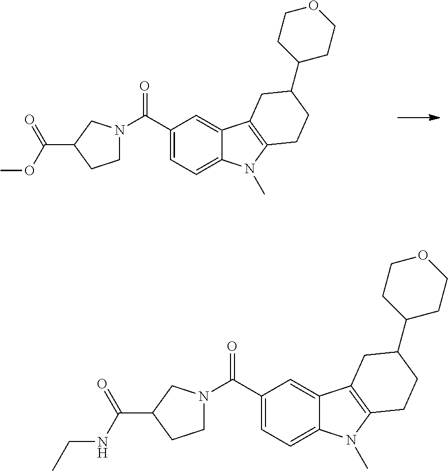

Methyl 1-(9-methyl-3-(tetrahydro-2H-pyran-4-yl)-2,3,4,9-tetrahydro-1H-carbazole-6-carbonyl)pyrrolidine-3-carboxylate (85 mg, 0.20 mmol) was stirred in dioxane (5 mL) containing lithium hydroxide (0.400 mL, 0.40 mmol) (1M) at 23° C. for 1 h. The solvent was evaporated. The residue was dissolved in DMF (5 mL) containing N,N-diisopropylethylamine (0.087 mL, 0.50 mmol) and ethylamine (0.120 mL, 0.24 mmol) (2M/THF) along with HATU (114 mg, 0.30 mmol) were added. The solution was stirred at 23° C. for 2 h. The solvent was evaporated. The residue was dissolved in EtOAc and washed with aqueous saturated NaHCO₃, brine and dried over anhydrous Na₂SO₄. The product was purified by reversed-phase HPLC using 30-50% B and lyophilized. Purification: Gilson system equipped with Luna C-18 column, 250×21.2 mm, 15 u. Mobile phase: A: H₂O with 0.05% TFA v/v; B: CH₃CN; 30 mL/min, 25 min run, rt. Yield: 50 mg (57%); ¹H NMR (400 MHz, DMSO-D6) δ 0.90-1.07 (m, 2H), 1.25-1.38 (m, 2H), 1.42-1.56 (m, 3H), 1.70 (t, J=11.72 Hz, 2H,) 1.92-2.07 (m, 3H), 2.26-2.38 (m, 1H), 2.58-2.70 (m, 1H), 2.74-2.88 (m, 3H), 3.01 (d, 2H), 3.29 (t, J=11.72 Hz, 2 H), 3.45-3.58 (m, 5H), 3.61 (s, 3H), 3.89 (d, J=7.81 Hz, 2H), 7.23 (d, J=8.20 Hz, 1H), 7.35 (d, J=8.20 Hz, 1H), 7.56 (s, 1H), 7.84-8.05 (m, 1H); MS (ESI) (M+H)⁺=438.3; accurate mass: (M+H)=438.274.

### Example 62

### Step A: N-Cyclopropyl-2-((3R)-1-(9-methyl-3-(tetrahydro-2H-pyran-4-yl)-2,3,4,9-tetrahydro-1H-carbazole-6-carbonyl)pyrrolidin-3-yl)acetamide

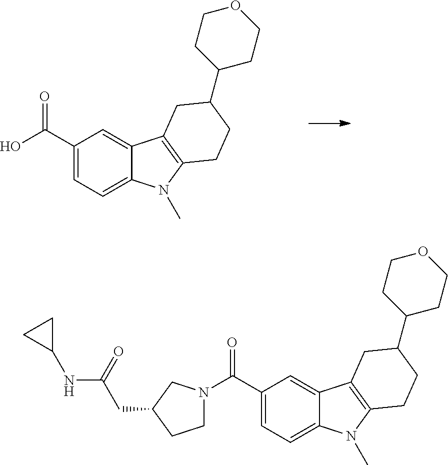

9-Methyl-3-(tetrahydro-2H-pyran-4-yl)-2,3,4,9-tetrahydro-1H-carbazole-6-carboxylic acid (100 mg, 0.32 mmol), (R)—N-cyclopropyl-2-(pyrrolidin-3-yl)acetamide hydrochloride (65.3 mg, 0.32 mmol) and O-(7-azabenzotriazol-1-yl)-N,N,N′,N′-tetramethyluronium hexafluorophosphate (182 mg, 0.48 mmol) were stirred in DMF (5 mL) containing N,N-diisopropylethylamine (0.167 mL, 0.96 mmol) at 23° C. for 2 h. The solvent was evaporated. The residue was dissolved in EtOAc and washed with aqueous saturated NaHCO₃, brine and dried over anhydrous Na₂SO₄. The product was purified by reversed-phase HPLC using 30-50% B and lyophilized. Purification: Gilson system equipped with Luna C-18 column, 250×21.2 mm, 15 u. Mobile phase: A: H₂O with 0.05% TFA v/v; B: CH₃CN; 30 mL/min, 25 min run, rt. Yield: 50 mg (34%); ¹H NMR (400 MHz, METHANOL-D4) δ 0.27-0.34 (m, 0.5H), 0.36-0.43 (m, 0.5H), 0.46-0.52 (m, 1H), 0.59-0.66 (m, 1H), 0.69-0.75 (m, 1H), 1.37-1.49 (m, 2H), 1.51-1.61 (m, 3.5H), 1.63-1.71 (m, 0.5H), 1.76 (t, J=12.89 Hz, 2H), 1.98-2.08 (m, 0.5H), 2.11-2.18 (m, 2H), 2.20-2.26 (m, 0.5H), 2.27-2.32 (m, 0.5H), 2.34-2.43 (m, 1H), 2.47-2.57 (m, 1H), 2.62-2.73 (m, 2H), 2.80-2.88 (m, 2H), 3.21-3.29 (m, 1H), 3.37-3.47 (m, 2H), 3.55-3.61 (m, 1H), 3.63 (s, 3H), 3.65-3.73 (m, 1H), 3.75-3.83 (m, 0.5H), 3.99 (dd, J=11.13, 3.71 Hz, 2H), 7.24-7.29 (m, 1H), 7.30-7.33 (m, 1H), 7.61 (d, J=5.08 Hz, 1H); MS (ESI) (M+H)⁺=464.2; Accurate mass: (M+H)=464.290.

### Step B: (R)—N-Cyclopropyl-2-(pyrrolidin-3-yl)acetamide hydrochloride

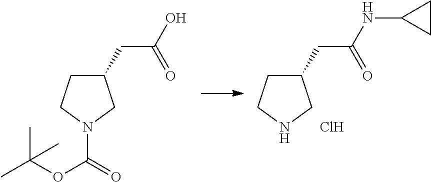

(R)-2-(1-(tert-Butoxycarbonyl)pyrrolidin-3-yl)acetic acid (500 mg, 2.18 mmol), HATU (995 mg, 2.62 mmol) and cyclopropylamine (0.181 mL, 2.62 mmol) were stirred in DMF (10 mL) at 23° C. for 1 h. The solvent was evaporated. The residue was dissolved in EtOAc and washed with 5% aqueous KHSO₄, saturated aqueous NaHCO₃, brine and dried over anhydrous Na₂SO₄. The solvent was evaporated. The product was then dissolved in Hydrogen chloride (10.90 mL, 10.90 mmol) (1M/AcOH) and stirred at 23° C. for 3-4 h. The solvent was evaporated and the product was dried under vacuum overnight. The product was used directly for the next step. Yield: 400 mg (90%).

### Example 63

### Step A: N-((3S)-1-(9-Methyl-3-(tetrahydro-2H-pyran-4-yl)-2,3,4,9-tetrahydro-1H-carbazole-6-carbonyl)pyrrolidin-3-yl)cyclopropanecarboxamide

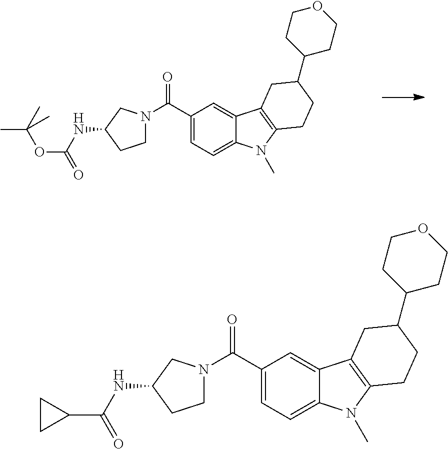

tert-Butyl (3S)-1-(9-methyl-3-(tetrahydro-2H-pyran-4-yl)-2,3,4,9-tetrahydro-1H-carbazole-6-carbonyl)pyrrolidin-3-ylcarbamate (150 mg, 0.31 mmol) was stirred in hydrogen chloride (6.23 mL, 6.23 mmol) (1M in AcOH) at 23° C. for 2-3 h. The solvent was evaporated. The product was rinsed twice with ether and the ether layer was decanted. The product was dried under vacuum. The residue was dissolved in CH₂Cl₂ (5 mL) containing triethylamine (0.217 mL, 1.56 mmol) and cyclopropanecarbonyl chloride (0.034 mL, 0.37 mmol) was added. The solution was stirred at 23° C. for 1 h. The solution was washed with saturated aqueous NaHCO₃, brine and dried over anhydrous Na₂SO₄. The product was purified by reversed-phase HPLC using 30-50% B and lyophilized. Purification: Gilson system equipped with Luna C-18 column, 250×21.2 mm, 15 u. Mobile phase: A: H₂O with 0.05% TFA v/v; B: CH₃CN; 30 mL/min, 25 min run, rt. Yield: 80 mg (57%); ¹H NMR (400 MHz, METHANOL-D4) δ 0.65-0.82 (m, 3 H), 0.87 (s, 1H), 1.37-1.51 (m, 2H), 1.51-1.66 (m, 4H), 1.78 (t, J=13.87 Hz, 2H), 1.84-1.93 (m, 0.5H), 1.95-2.05 (m, 0.5H), 2.10-2.20 (m, 1.5H), 2.21-2.31 (m, 0.5H), 2.34-2.45 (m, 1H), 2.63-2.75 (m, 1H), 2.79-2.90 (m, 2H), 3.38-3.47 (m, 2.5H), 3.50 (m, 0.5H), 3.64 (s, 3H), 3.66-3.76 (m, 1.5H), 3.78-3.91 (m, 1.5H), 3.99 (dd, J=11.33, 3.52 Hz, 2H), 4.22-4.30 (m, 0.5H), 4.43-4.51 (m, 0.5H), 7.24-7.37 (m, 2 H), 7.63 (d, J=17.97 Hz, 1H); MS (ESI) (M+H)⁺=450.2; Accurate mass: (M+H)=450.276.

### Step B: tert-Butyl (3S)-1-(9-methyl-3-(tetrahydro-2H-pyran-4-yl)-2,3,4,9-tetrahydro-1H-carbazole-6-carbonyl)pyrrolidin-3-ylcarbamate

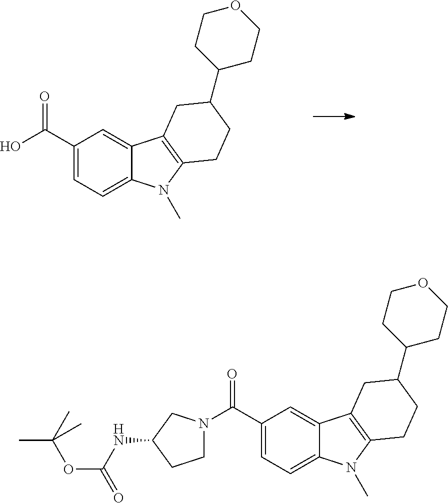

9-Methyl-3-(tetrahydro-2H-pyran-4-yl)-2,3,4,9-tetrahydro-1H-carbazole-6-carboxylic acid (100 mg, 0.32 mmol), (S)-(−)-3-(Boc-amino)pyrrolidine (71.3 mg, 0.38 mmol) and O-(7-azabenzotriazol-1-yl)-N,N,N′,N′-tetramethyluronium hexafluorophosphate (146 mg, 0.38 mmol) were stirred in DMF (5 mL) containing N,N-diisopropylethylamine (0.083 mL, 0.48 mmol) at 23° C. for 1 h. The solvent was evaporated. The residue was dissolved in EtOAc and washed with saturated aqueous NaHCO₃, brine and dried over anhydrous Na₂SO₄. The product was purified by flash chromatography using EtOAc as eluent. Yield: 150 mg (98%); ¹H NMR (400 MHz, CHLOROFORM-D) δ 1.36-1.51 (m, 11H), 1.53-1.66 (m, 5H), 1.75 (t, J=10.35 Hz, 2H), 1.88 (s, 1H), 2.12-2.28 (m, 2H), 2.37-2.48 (m, 1H), 2.63-2.75 (m, 1H), 2.79-2.90 (m, 3H), 3.51 (s, 1H), 3.63 (s, 3H), 3.70-3.96 (m, 2H), 4.05 (dd, J=11.52, 3.71 Hz, 2H), 4.16-4.38 (m, 1H), 4.52-4.79 (m, 1H), 7.23 (d, J=8.59 Hz, 1H), 7.34 (s, 1H), 7.69 (s, 1H); MS (ESI) (M+H)⁺=492.33.

### Example 64

### Step A: (3S)—N-(2-Fluoroethyl)-1-(9-methyl-3-(tetrahydro-2H-pyran-4-yl)-2,3,4,9-tetrahydro-1H-carbazole-6-carbonyl)pyrrolidine-3-carboxamide (Isomers 1 and 2)

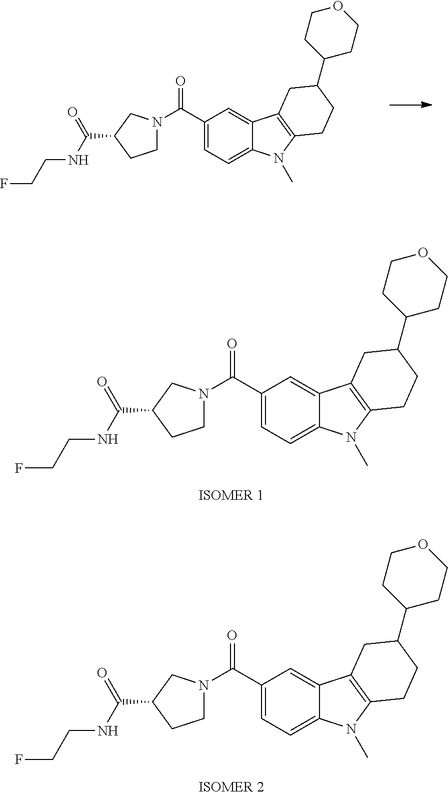

Chiral separation of (3S)—N-(2-fluoroethyl)-1-(9-methyl-3-(tetrahydro-2H-pyran-4-yl)-2,3,4,9-tetrahydro-1H-carbazole-6-carbonyl)pyrrolidine-3-carboxamide (65 mg, 0.14 mmol). Products had to be repurified by reversed-phase HPLC after chiral separation using 30-50% B. Chiral Purification: Gilson system equipped with a Chiracel AD column, 5 cm ID×50 cm L, 20 u using 60% EtOH/40% hexanes with 0.1% diethylamine v/v; 100 mL/min, 60 min run, rt. Chiral analytical HPLC: ChiraPak AD column, 60% EtOH/40% hexanes, 1 mL/min, 30 min run, 25° C. Reversed-phase purification: Gilson system equipped with Luna C-18 column, 250×21.2 mm, 15 u. Mobile phase: A: H₂O with 0.05% TFA v/v; B: CH₃CN; 30 mL/min, 25 min run, rt.

Yield: Isomer 1: 25 mg (39%)
Isomer 2: 26 mg (40%)

Isomer 1: ¹H NMR (400 MHz, METHANOL-D4) δ 1.26-1.36 (m, 1H), 1.40-1.51 (m, 2H), 1.54-1.64 (m, 3H), 1.79 (t, J=12.70 Hz, 2H), 2.05-2.15 (m, 1H), 2.15-2.28 (m, 2H), 2.35-2.45 (m, 1H), 2.65-2.76 (m, 1H), 2.82-2.92 (m, 2H), 2.96-3.05 (m, 0.5H), 3.09-3.20 (m, 0.5H), 3.38-3.50 (m, 3H), 3.55 (t, J=4.69 Hz, 0.5H), 3.58-3.63 (m, 0.5H), 3.65 (s, 3H), 3.67-3.74 (m, 1H), 3.74-3.88 (m, 2H), 4.00 (dd, J=11.13, 3.71 Hz, 2H), 4.34 (t, J=4.88 Hz, 0.5H), 4.42 (t, J=4.88 Hz, 0.5H), 4.46 (t, J=4.88 Hz, 0.5H), 4.53 (t, J=4.88 Hz, 0.5H), 7.26-7.31 (m, 1H), 7.31-7.36 (m, 1H), 7.62 (d, J=0.78 Hz, 1H); Chiral k′=3.35; MS (ESI) (M+H)⁺=456.2; accurate mass: (M+H)=456.265.

Isomer 2: ¹H NMR (400 MHz, METHANOL-D4) δ 1.27-1.35 (m, 1H), 1.38-1.52 (m, 2H), 1.54-1.65 (m, 3H), 1.80 (t, J=12.50 Hz, 2H), 2.12 (s, 1H), 2.15-2.27 (m, 2H), 2.36-2.47 (m, 1H), 2.66-2.76 (m, 1H), 2.82-2.92 (m, 2H), 2.97-3.06 (m, 0.5H), 3.10-3.18 (m, 0.5H), 3.39-3.50 (m, 3H), 3.55 (t, J=4.88 Hz, 0.5H), 3.59-3.64 (m, 0.5H), 3.65 (s, 3H), 3.68-3.76 (m, 1H), 3.75-3.90 (m, 2H), 4.00 (dd, J=11.13, 3.71 Hz, 2H) 4.34 (t, J=4.30 Hz, 0.5H), 4.42 (t, J=4.88 Hz, 0.5H), 4.46 (t, J=4.88 Hz, 0.5H), 4.53 (t, J=4.88 Hz, 0.5H), 7.25-7.31 (m, 1H), 7.31-7.37 (m, 1H), 7.62 (d, J=1.17 Hz, 1H); Chiral k′=5.54; MS (ESI) (M+H)⁺=456.2; accurate mass: (M+H)=456.265.

### Step B: (S)—N-(2-Fluoroethyl)pyrrolidine-3-carboxamide hydrochloride

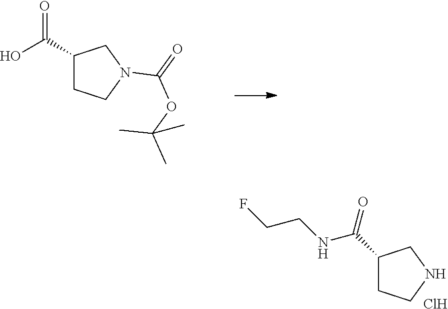

(S)-1-Boc-pyrrolidine-3-carboxylic acid (350 mg, 1.63 mmol), 2-fluoroethylamine hydrochloride (194 mg, 1.95 mmol) and HATU (742 mg, 1.95 mmol) were stirred in DMF (10 mL) containing N,N-diisopropylethylamine (0.708 mL, 4.07 mmol) at 23° C. for 1 h. The solvent was evaporated. The residue was dissolved in EtOAc and washed with 5% KHSO₄, saturated aqueous NaHCO₃, brine and dried over anhydrous Na₂SO₄. The product was quickly flashed through a silica plug using EtOAc as eluent. The solvent was evaporated. The product was then stirred in hydrogen chloride (16.26 mL, 16.26 mmol) (1M in AcOH) at 23° C. for 3-4 h. The solvent was evaporated and the product was dried under vacuum overnight. Yield: 215 mg (67%); ¹H NMR (400 MHz, METHANOL-D4) δ 2.07-2.18 (m, 1H), 2.26-2.37 (m, 1H), 3.17-3.25 (m, 1H), 3.32-3.37 (m, 1H), 3.37-3.44 (m, 2H), 3.44-3.50 (m, 2H), 3.51-3.54 (m, 1H), 4.38-4.42 (m, 1H), 4.52 (t, J=5.08 Hz, 1H).

### Step C: (3S)—N-(2-Fluoroethyl)-1-(9-methyl-3-(tetrahydro-2H-pyran-4-yl)-2,3,4,9-tetrahydro-1H-carbazole-6-carbonyl)pyrrolidine-3-carboxamide

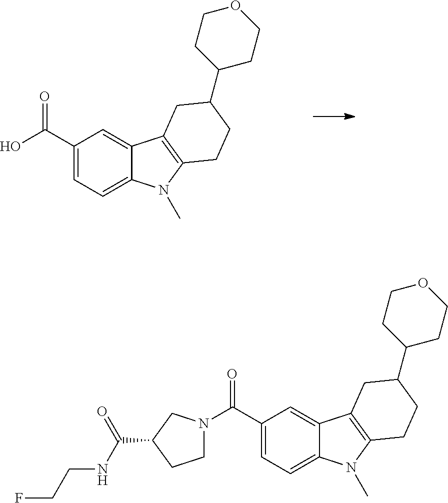

9-Methyl-3-(tetrahydro-2H-pyran-4-yl)-2,3,4,9-tetrahydro-1H-carbazole-6-carboxylic acid (120 mg, 0.38 mmol), (S)—N-(2-fluoroethyl)pyrrolidine-3-carboxamide hydrochloride (113 mg, 0.57 mmol) and HATU (218 mg, 0.57 mmol) were stirred in DMF (5 mL) containing N,N-diisopropylethylamine (0.167 mL, 0.96 mmol) at 23° C. for 1 h. The solvent was evaporated. The residue was dissolved in EtOAc and washed with saturated aqueous NaHCO₃, brine and dried over anhydrous Na₂SO₄. The product was purified by reversed-phase HPLC using 30-50% B and lyophilized. Purification: Gilson system equipped with Luna C-18 column, 250×21.2 mm, 15 u. Mobile phase: A: H₂O with 0.05% TFA v/v; B: CH₃CN; 30 mL/min, 25 min run, rt. Yield: 65 mg (37%); ¹H NMR (400 MHz, METHANOL-D4) δ 1.39-1.49 (m, 2H), 1.54-1.62 (m, 3H), 1.79 (t, J=12.50 Hz, 2H), 2.11 (m, 1H), 2.14-2.21 (m, 1.5H), 2.21-2.28 (m, 0.5H), 2.36-2.45 (m, 1H), 2.65-2.75 (m, 1H), 2.82-2.86 (m, 1H), 2.86-2.91 (m, 1H), 2.97-3.06 (m, 0.5H), 3.11-3.19 (m, 0.5H, 3.38-3.42 (m, 1H), 3.42-3.45 (m, 1H), 3.45-3.50 (m, 1H), 3.55 (t, J=4.30 Hz, 0.5H), 3.58-3.62 (m, 0.5H,) 3.64 (s, 3H), 3.66-3.72 (m, 1H), 3.73-3.80 (m, 1.5H), 3.81-3.88 (m, 0.5H), 4.00 (dd, J=10.94, 3.52 Hz, 2H), 4.34 (t, J=4.88 Hz, 0.5H), 4.41 (t, J=4.69 Hz, 0.5H), 4.44-4.49 (m, 0.5H), 4.53 (t, J=4.69 Hz, 0.5H), 7.26-7.30 (m, 1H), 7.31-7.35 (m, 1H), 7.62 (d, J=0.78 Hz, 1H); MS (ESI) (M+H)⁺=456.45.

### Example 65

### Step A: (3S)—N-(Cyclopropylmethyl)-1-(9-methyl-3-(tetrahydro-2H-pyran-4-yl)-2,3,4,9-tetrahydro-1H-carbazole-6-carbonyl)pyrrolidine-3-carboxamide

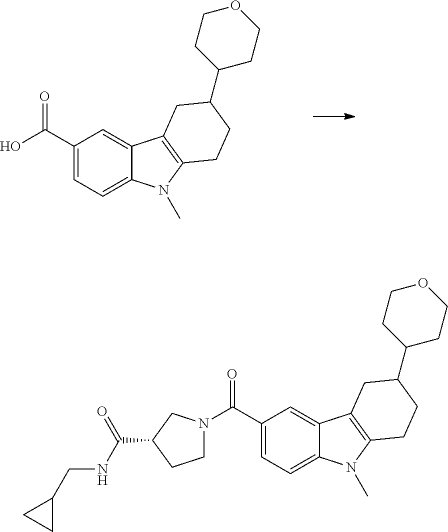

9-Methyl-3-(tetrahydro-2H-pyran-4-yl)-2,3,4,9-tetrahydro-1H-carbazole-6-carboxylic acid (100 mg, 0.32 mmol), (S)—N-(cyclopropylmethyl)pyrrolidine-3-carboxamide hydrochloride (78 mg, 0.38 mmol) and HATU (146 mg, 0.38 mmol) were stirred in DMF (5 mL) containing N,N-diisopropylethylamine (0.139 mL, 0.80 mmol) at 23° C. for 1 h. The solvent was evaporated. The residue was dissolved in EtOAc and washed with saturated aqueous NaHCO₃, brine and dried over anhydrous Na₂SO₄. The product was purified by reversed-phase HPLC using 30-50% B and lyophilized. Reversed-phase purification: Gilson system equipped with Luna C-18 column, 250×21.2 mm, 15 u. Mobile phase: A: H₂O with 0.05% TFA v/v; B: CH₃CN; 30 mL/min, 25 min run, rt. Yield: 95 mg (64%); ¹H NMR (400 MHz, METHANOL-D4) δ 0.18 (m, 2H) 0.47 (m, 2 H), 0.84-1.03 (m, 1H), 1.34-1.48 (m, 2H), 1.55 (s, 3H), 1.76 (t, J=12.89 Hz, 2H), 2.03-2.11 (m, 1H), 2.11-2.24 (m, 2H), 2.31-2.42 (m, 1H), 2.61-2.72 (m, 1H), 2.79-2.87 (m, 2H), 2.92-3.02 (m, 1.5H), 3.04-3.14 (m, 1.5H), 3.37-3.47 (m, 2H), 3.58-3.66 (m, 4H), 3.68-3.79 (m, 2H), 3.79-3.88 (m, 1H), 3.98 (dd, J=11.13, 3.71 Hz, 2H), 7.25-7.30 (m, 1H), 7.30-7.34 (m, 1H), 7.62 (s, 1H); MS (ESI) (M+H)⁺=464.2; Accurate mass (M+H)=464.290.

### Step B: (S)—N-(Cyclopropylmethyl)pyrrolidine-3-carboxamide hydrochloride

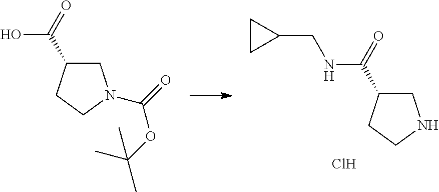

(S)-1-(tert-Butoxycarbonyl)pyrrolidine-3-carboxylic acid (200 mg, 0.93 mmol), O-(7-azabenzotriazol-1-yl)-N,N,N′,N′-tetramethyluronium hexafluorophosphate (424 mg, 1.12 mmol) and (aminomethyl)cyclopropane (0.097 mL, 1.12 mmol) were stirred in DMF (10 mL) containing N,N-diisopropylethylamine (0.243 mL, 1.39 mmol) at 23° C. for 1 h. The solvent was evaporated. The residue was dissolved in EtOAc and washed with 5% KHSO₄, saturated aqueous NaHCO₃, brine and dried over anhydrous Na₂SO₄. The product was purified by a short flash column using 50% EtOAc/hexanes to 100% EtOAc. The fractions were concentrated. The product was then dissolved in hydrogen chloride (4.65 mL, 4.65 mmol) (1M in AcOH) and stirred at 23° C. for 2-3 h. The solvent was evaporated. The residue was washed with ether. The product was dried under vacuum overnight. Yield: 180 mg (95%); ¹H NMR (400 MHz, METHANOL-D4) δ 0.17-0.23 (m, 2H), 0.46-0.53 (m, 2H), 0.90-1.01 (m, 1H), 2.06-2.17 (m, 1H), 2.25-2.36 (m, 1H), 3.02-3.08 (m, 2H), 3.14-3.24 (m, 1H), 3.27-3.35 (m, 1H), 3.36-3.43 (m, 2H), 3.43-3.50 (m, 1H).

### Example 66

### Step A: N-(1-(9-Methyl-3-(tetrahydro-2H-pyran-4-yl)-2,3,4,9-tetrahydro-1H-carbazole-6-carbonyl)piperidin-4-yl)cyclopropanecarboxamide

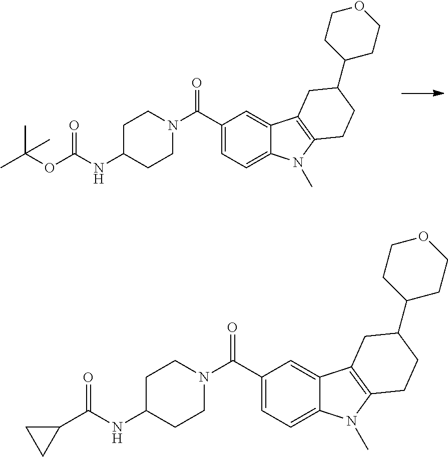

tert-Butyl 1-(9-methyl-3-(tetrahydro-2H-pyran-4-yl)-2,3,4,9-tetrahydro-1H-carbazole-6-carbonyl)piperidin-4-ylcarbamate (135 mg, 0.27 mmol) was stirred in hydrogen chloride (6.81 mL, 6.81 mmol) (1M/AcOH) at 23° C. for 2 h. The solvent was evaporated. The residue was dissolved in dichloromethane (5 mL) containing triethylamine (0.190 mL, 1.36 mmol). Cyclopropanecarbonyl chloride (0.030 mL, 0.33 mmol) was added dropwise and the solution was stirred at 23° C. for 1 h. The solution was washed with 5% KHSO₄, saturated aqueous NaHCO₃, brine and dried over anhydrous Na₂SO₄. The product was purified by reversed-phase HPLC using 30-50% B and lyophilized. Reversed-phase purification: Gilson system equipped with Luna C-18 column, 250×21.2 mm, 15 u. Mobile phase: A: H₂O with 0.05% TFA v/v; B: CH₃CN; 30 mL/min, 25 min run, rt. Yield: 70 mg (55%); ¹H NMR (400 MHz, METHANOL-D4) δ 0.69-0.77 (m, 2H), 0.79-0.87 (m, 2H), 1.37-1.51 (m, 4H), 1.50-1.62 (m, 5H), 1.77 (t, J=13.87 Hz, 2H), 1.90 (s, 2H), 2.14-2.21 (m, 1H), 2.39 (dd, J=15.23, 7.03 Hz, 1H), 2.62-2.75 (m, 1H), 2.80-2.91 (m, 2H), 3.13 (s, 2H), 3.37-3.48 (m, 2H), 3.64 (s, 3H), 3.87-3.96 (m, 1H), 3.99 (dd, J=11.52, 3.71 Hz, 2H), 4.52 (s, 1H), 7.15 (dd, J=8.59, 1.56 Hz, 1H), 7.33 (d, J=8.20 Hz, 1H), 7.49 (d, J=0.78 Hz, 1H); MS (ESI) (M+H)⁺=464.2; accurate mass: (M+H)=464.291.

### Step B: tert-Butyl 1-(9-methyl-3-(tetrahydro-2H-pyran-4-yl)-2,3,4,9-tetrahydro-1H-carbazole-6-carbonyl)piperidin-4-ylcarbamate

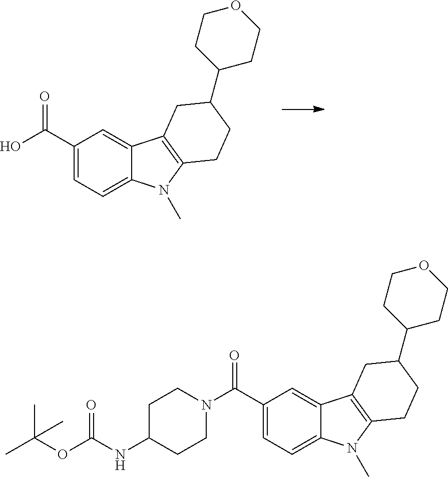

9-Methyl-3-(tetrahydro-2H-pyran-4-yl)-2,3,4,9-tetrahydro-1H-carbazole-6-carboxylic acid (100 mg, 0.32 mmol), 4-(N-Boc amino)-piperidine (77 mg, 0.38 mmol) and O-(7-azabenzotriazol-1-yl)-N,N,N′N-tetramethyluronium hexafluorophosphate (146 mg, 0.38 mmol) were stirred in DMF (5 mL) containing N,N-diisopropylethylamine (0.083 mL, 0.48 mmol) at 23° C. for 1 h. The solvent was evaporated. The residue was dissolved in EtOAc and washed with 5% KHSO₄, saturated aqueous NaHCO₃, brine and dried over anhydrous Na₂SO₄. The product was purified by flash chromatography using a gradient 50% EtOAc/heptane to 100% EtOAc. Yield: 140 mg (89%); ¹H NMR (400 MHz, DMSO-D6) δ 1.24-1.33 (m, 4H), 1.37 (s, 9H), 1.45-1.54 (m, 3H), 1.66-1.75 (m, 4 H), 2.05-2.11 (m, 1H), 2.28-2.36 (m, 1H), 2.62-2.69 (m, 1H), 2.74-2.86 (m, 2H), 2.97 (s, 2H), 3.25-3.30 (m, 4H), 3.50 (s, 1H), 3.61 (s, 3H), 3.89 (dd, J=10.74, 2.54 Hz, 2H), 6.86 (d, J=8.20 Hz, 1H), 7.05 (dd, J=8.40, 1.37 Hz, 1H), 7.37 (d, J=8.59 Hz, 1 H), 7.39 (s, 1H); MS (ESI) (M+H)⁺=496.41.

### Example 67

### Step A: N-(1-(9-Methyl-3-(tetrahydro-2H-pyran-4-yl)-2,3,4,9-tetrahydro-1H-carbazole-6-carbonyl)piperidin-3-yl)cyclopropanecarboxamide

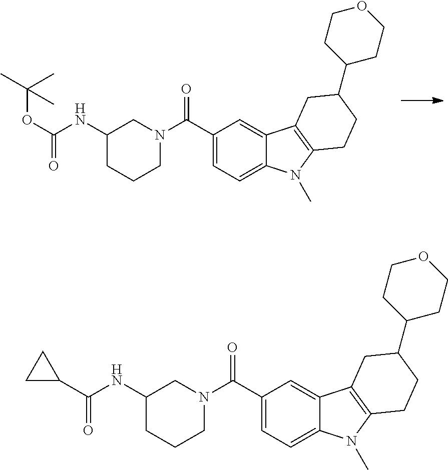

tert-Butyl 1-(9-methyl-3-(tetrahydro-2H-pyran-4-yl)-2,3,4,9-tetrahydro-1H-carbazole-6-carbonyl)piperidin-3-ylcarbamate (140 mg, 0.28 mmol) was stirred in hydrogen chloride (2.82 mL, 2.82 mmol) (1M/AcOH) at 23° C. for 2 h. The solvent was evaporated. The residue was dissolved in EtOAc and washed with saturated aqueous NaHCO₃, brine and dried over anhydrous Na₂SO₄. The solvent was evaporated. The residue was dissolved in dichloromethane (5 mL) containing triethylamine (0.098 mL, 0.71 mmol) and cyclopropanecarbonyl chloride (0.031 mL, 0.34 mmol) was added dropwise. The solution was stirred at 23° C. for 1 h. The solution was washed with 5% KHSO₄, saturated aqueous NaHCO₃, brine and dried over anhydrous Na₂SO₄. The product was purified by reversed-phase HPLC using 30-50% B and lyophilized. Reversed-phase purification: Gilson system equipped with Luna C-18 column, 250×21.2 mm, 15 u. Mobile phase: A: H₂O with 0.05% TFA v/v; B: CH₃CN; 30 mL/min, 25 min run, rt. Yield: 60 mg (46%); ¹H NMR (400 MHz, METHANOL-D4) δ 0.62-0.80 (m, 4H), 1.37-1.48 (m, 2H), 1.51-1.63 (m, 6H), 1.77 (t, J=11.33 Hz, 2H), 1.93-2.02 (m, 1H), 2.11-2.20 (m, 1H), 2.30-2.44 (m, 1H), 2.63-2.73 (m, 1H), 2.78-2.89 (m, 2H), 3.15 (s, 2H), 3.36-3.47 (m, 3H), 3.62 (s, 3H), 3.83 (s, 2H), 3.97 (dd, J=11.13, 3.32 Hz, 2H), 7.14 (ddd, J=8.40, 1.37, 1.17 Hz, 1H), 7.29 (d, J=8.20 Hz, 1H), 7.48 (s, 1H); MS (ESI) (M+H)⁺=464.2; accurate mass: (M+H)=464.290.

### Step B: tert-Butyl 1-(9-methyl-3-(tetrahydro-2H-pyran-4-yl)-2,3,4,9-tetrahydro-1H-carbazole-6-carbonyl)piperidin-3-ylcarbamate

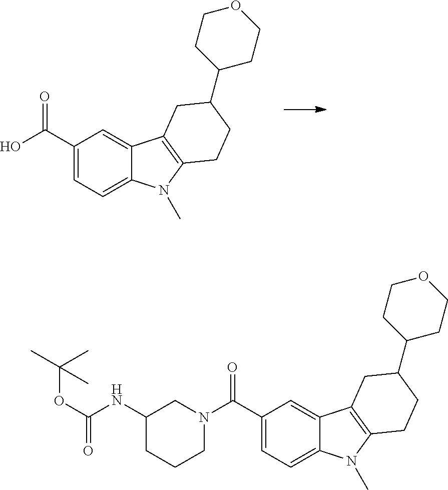

9-Methyl-3-(tetrahydro-2H-pyran-4-yl)-2,3,4,9-tetrahydro-1H-carbazole-6-carboxylic acid (100 mg, 0.32 mmol), 3-N-Boc-amino piperidine (77 mg, 0.38 mmol) and HATU (146 mg, 0.38 mmol) were stirred in DMF (5 mL) containing N,N-diisopropylethylamine (0.083 mL, 0.48 mmol) at 23° C. for 1 h. The solvent was evaporated. The residue was dissolved in EtOAc and washed with 5% KHSO₄, saturated aqueous NaHCO₃, brine and dried over anhydrous Na₂SO₄. The product was purified by flash chromatography using a gradient of 50% EtOAc/heptane to 100% EtOAc. Yield: 150 mg (95%); ¹H NMR (400 MHz, CHLOROFORM-D) δ 1.38-1.44 (m, 9H), 1.45-1.56 (m, 4H), 1.56-1.65 (m, 5H), 1.71-1.80 (m, 3H), 1.95 (s, 1H), 2.12-2.19 (m, 1H), 2.38-2.49 (m, 1H), 2.63-2.75 (m, 1H), 2.78-2.87 (m, 2H), 2.88-2.97 (m, 1H), 3.37-3.47 (m, 3H), 3.63 (s, 3H), 3.75 (s, 1H), 3.81-3.91 (m, 1H), 4.04 (dd, J=11.33, 2.73 Hz, 2H), 7.24 (s, 2 H), 7.61 (s, 1H); MS (ESI) (M+H)⁺=496.59.

### Example 68

### Step A: (R)-(3S)—N-(2,2-Difluoroethyl)-1-(9-methyl-3-(tetrahydro-2H-pyran-4-yl)-2,3,4,9-tetrahydro-1H-carbazole-6-carbonyl)pyrrolidine-3-carboxamide

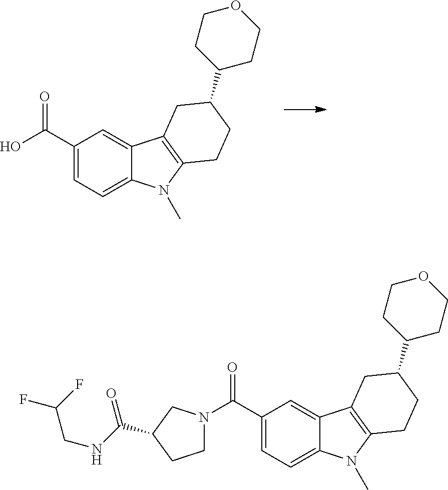

(R)-9-Methyl-3-(tetrahydro-2H-pyran-4-yl)-2,3,4,9-tetrahydro-1H-carbazole-6-carboxylic acid (100 mg, 0.32 mmol), (S)—N-(2,2-difluoroethyl)pyrrolidine-3-carboxamide hydrochloride (82 mg, 0.38 mmol) and HATU (146 mg, 0.38 mmol) were stirred in DMF (8 mL) containing N,N-diisopropylethylamine (0.139 mL, 0.80 mmol) at 23° C. for 1 h. The solvent was evaporated. The residue was dissolved in EtOAc and washed with saturated aqueous NaHCO₃, brine and dried over anhydrous Na₂SO₄. The product was purified by reversed-phase HPLC and lyophilized. Reversed-phase purification: Gilson system equipped with Luna C-18 column, 250×21.2 mm, 15 u. Mobile phase: 40-60% B; A: H₂O with 0.05% TFA v/v; B: CH₃CN; 30 mL/min, 25 min run, rt. Yield: 48 mg (32%); ¹H NMR (400 MHz, METHANOL-D4) δ 1.35-1.47 (m, 2 H), 1.49-1.60 (m, 3H), 1.76 (t, J=12.70 Hz, 2H), 2.03-2.11 (m, 1H), 2.12-2.27 (m, 2 H), 2.32-2.42 (m, 1H), 2.61-2.73 (m, 1H), 2.78-2.88 (m, 2H), 2.96-3.05 (m, 0.5H), 3.09-3.19 (m, 0.5H), 3.36-3.46 (m, 2H), 3.46-3.59 (m, 3H), 3.61 (s, 3H), 3.64-3.73 (m, 1H), 3.71-3.87 (m, 2H), 3.97 (dd, J=11.13, 3.71 Hz, 2H), 5.65-6.06 (m, 1H), 7.23-7.28 (m, 1H), 7.28-7.33 (m, 1H), 7.60 (s, 1H), 8.44 (d, J=53.91 Hz, 1H); MS (ESI)⁺=474.2; accurate mass: (M+H)=474.56.

### Step B: (S)—N-(2,2-Difluoroethyl)pyrrolidine-3-carboxamide hydrochloride

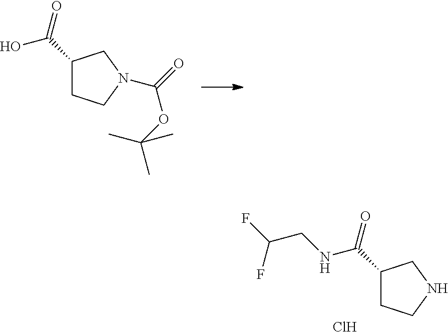

(S)-1-(tort-Butoxycarbonyl)pyrrolidine-3-carboxylic acid (500 mg, 2.32 mmol), 2,2-difluoroethanamine (226 mg, 2.79 mmol) and HATU (1060 mg, 2.79 mmol) were stirred in DMF (10 mL) containing N,N-diisopropylethylamine (0.607 mL, 3.48 mmol) at 23° C. for 1 h. The solvent was concentrated. The residue was dissolved in EtOAc and washed with 5% KHSO₄, saturated aqueous NaHCO₃, brine and dried over anhydrous Na₂SO₄. The product was purified by flash chromatography using EtOAc as eluent. The product was then stirred in hydrogen chloride (11.61 mL, 11.61 mmol) (1M/AcOH) at 23° C. for 2 h. The solvent was evaporated. The residue was precipitated in ether, filtered and dried. Yield: 375 mg (75%); ¹H NMR (400 MHz, METHANOL-D4) δ 2.04-2.17 (m, 1 H), 2.23-2.37 (m, 1H), 3.15-3.25 (m, 1H), 3.30-3.41 (m, 3H), 3.45-3.53 (m, 1H), 3.52-3.61 (m, 2H), 5.68-6.07 (m, 1H).

### Step C: (R)-Methyl 9-methyl-3-(tetrahydro-2H-pyran-4-yl)-2,3,4,9-tetrahydro-1H-carbazole-6-carboxylate and (S)-Methyl 9-methyl-3-(tetrahydro-2H-pyran-4-yl)-2,3,4,9-tetrahydro-1H-carbazole-6-carboxylate

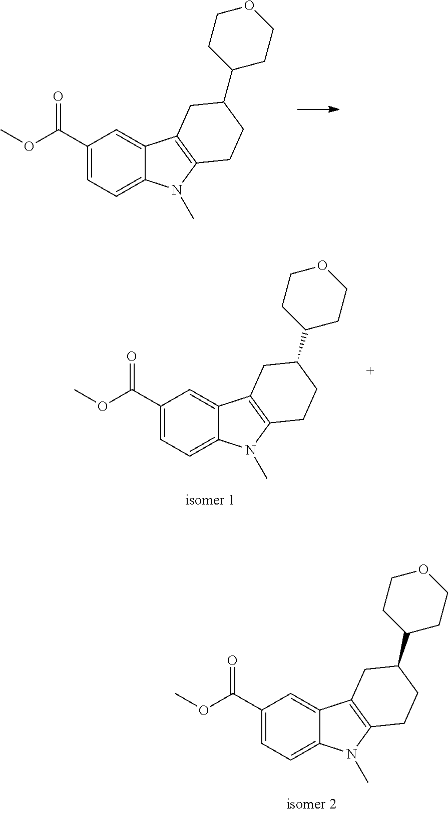

Chiral separation of methyl 9-methyl-3-(tetrahydro-2H-pyran-4-yl)-2,3,4,9-tetrahydro-1H-carbazole-6-carboxylate (16.28 g, 49.7 mmol) was done by using a Chiralcel OD column with an eluent of 60/40 hexanes/EtOH at rt. Analytical chiral HPLC: OD-H column, 4.6×250 mm, 60/40 hexanes/EtOH.
(R)-Methyl 9-methyl-3-(tetrahydro-2H-pyran-4-yl)-2,3,4,9-tetrahydro-1H-carbazole-6-carboxylate (isomer 1, 7.13 g, 88%):

¹H NMR (400 MHz, DMSO-D6) δ 1.21-1.36 (m, 2H), 1.40-1.54 (m, 3H), 1.63-1.73 (m, 2H), 2.00-2.08 (m, 1H), 2.25-2.36 (m, 1H), 2.56-2.67 (m, 1H), 2.73-2.84 (m, 2H), 3.21-3.31 (m, 2H), 3.60 (s, 3H), 3.79 (s, 3H), 3.83-3.91 (m, 2H), 7.40 (d, J=8.59 Hz, 1H), 7.66 (dd, J=8.59, 1.56 Hz, 1H), 8.04 (d, J=1.56 Hz, 1H); MS (ESI) (M+H)⁺=328.30; chiral HPLC k′=2.43.

(S)-Methyl 9-methyl-3-(tetrahydro-2H-pyran-4-yl)-2,3,4,9-tetrahydro-1H-carbazole-6-carboxylate (isomer 2, 7.01 g, 86%): NMR (400 MHz, DMSO-D6) δ 1.23-1.35 (m, 2H), 1.41-1.51 (m, 3H), 1.68 (m, 2H), 2.01-2.08 (m, 1H), 2.27-2.36 (m, 1H), 2.58-2.67 (m, 1H), 2.75-2.84 (m, 2H), 3.22-3.30 (m, 2H), 3.60 (s, 3H), 3.79 (s, 3H), 3.84-3.90 (m, 2H), 7.40 (d, J=8.59 Hz, 1H), 7.66 (dd, J=8.59, 1.56 Hz, 1H), 8.04 (d, J=1.56 Hz, 1H); MS (ESI) (M+H)⁺=328.31; chiral HPLC k′=3.70.

### Step D: (R)-9-Methyl-3-(tetrahydro-2H-pyran-4-yl)-2,3,4,9-tetrahydro-1H-carbazole-6-carboxylic acid

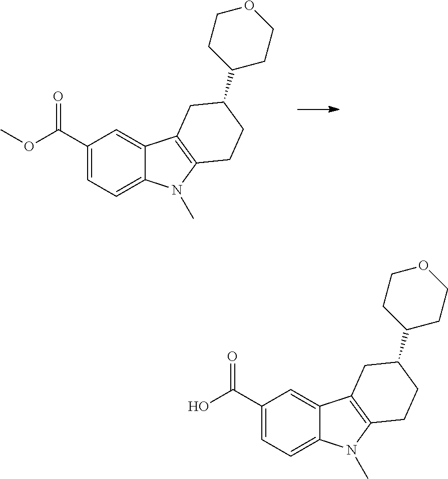

(R)-Methyl 9-methyl-3-(tetrahydro-2H-pyran-4-yl)-2,3,4,9-tetrahydro-1H-carbazole-6-carboxylate (1.00 g, 3.05 mmol) was stirred in dioxane (100 mL) containing lithium hydroxide (9.16 mL, 9.16 mmol) (1M) at 50° C. overnight. The solvent was concentrated. The aqueous layer was washed with ether. The aqueous layer was then acidified with 2M HCl. The product precipitated and was filtered and dried under vacuum. Yield: 875 mg (91%); ¹H NMR (400 MHz, DMSO-D6) δ 1.23-1.36 (m, 2H), 1.42-1.52 (m, 3H), 1.65-1.72 (m, 2H), 2.02-2.09 (m, 1H), 2.28-2.36 (m, 1H), 2.58-2.68 (m, 1H), 2.74-2.85 (m, 2H), 3.23-3.30 (m, 2H), 3.60 (s, 3H), 3.83-3.90 (m, 2H), 7.37 (d, J=8.59 Hz, 1H), 7.65 (dd, J=8.59, 1.56 Hz, 1H), 8.02 (d, J=1.56 Hz, 1H); MS (ESI) (M+H)⁺=314.21.

### Example 69

### Step A: (R)-(3S)—N-Ethyl-1-(9-methyl-3-(tetrahydro-2H-pyran-4-yl)-2,3,4,9-tetrahydro-1H-carbazole-6-carbonyl)pyrrolidine-3-carboxamide

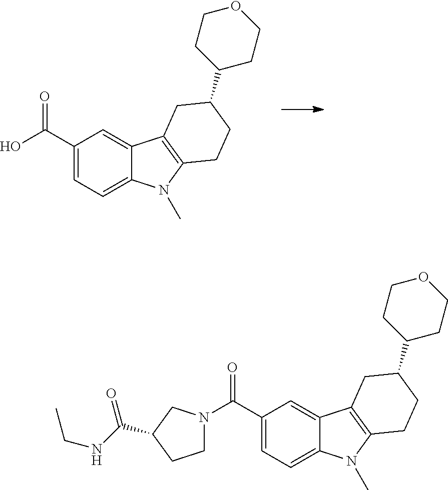

(R)-9-Methyl-3-(tetrahydro-2H-pyran-4-yl)-2,3,4,9-tetrahydro-1H-carbazole-6-carboxylic acid (100 mg, 0.32 mmol), (S)—N-ethylpyrrolidine-3-carboxamide hydrochloride (68.4 mg, 0.38 mmol) and HATU (146 mg, 0.38 mmol) were stirred in DMF (5 mL) containing N,N-diisopropylethylamine (0.139 mL, 0.80 mmol) at 23° C. for 1 h. The solvent was evaporated. The residue was dissolved in EtOAc and washed with saturated aqueous NaHCO₃, brine and dried over anhydrous Na₂SO₄. The product was purified by reversed-phase HPLC and lyophilized. Reversed-phase purification: Gilson system equipped with Luna C-18 column, 250×21.2 mm, 15 u. Mobile phase: 30-50% B; A: H₂O with 0.05% TFA v/v; B: CH₃CN; 30 mL/min, 25 min run, rt. Yield: 100 mg (71%); ¹H NMR (400 MHz, METHANOL-D4) δ 1.05 (t, J=7.23 Hz, 1H), 1.12 (t, J=7.23 Hz, 1H), 1.35-1.48 (m, 2H), 1.50-1.60 (m, 3H), 1.76 (t, J=12.70 Hz, 2H), 2.02-2.10 (m, 1H), 2.11-2.21 (m, 2H), 2.32-2.41 (m, 1H), 2.62-2.73 (m, 1H), 2.79-2.87 (m, 2H), 2.88-2.96 (m, 0.5H), 3.02-3.08 (m, 0.5H), 3.10-3.18 (m, 1H), 3.18-3.26 (m, 1H), 3.37-3.45 (m, 2H), 3.56-3.61 (m, 1H), 3.62 (s, 3H), 3.64-3.74 (m, 2 H), 3.72-3.84 (m, 1H), 3.97 (dd, J=11.33, 3.52 Hz, 2H), 7.24-7.28 (m, 1H), 7.28-7.33 (m, 1H), 7.60 (s, 1H); MS (ESI) (M+H)⁺=438.3; accurate mass (M+H)=438.275.

### Step B: (S)—N-Ethylpyrrolidine-3-carboxamide hydrochloride

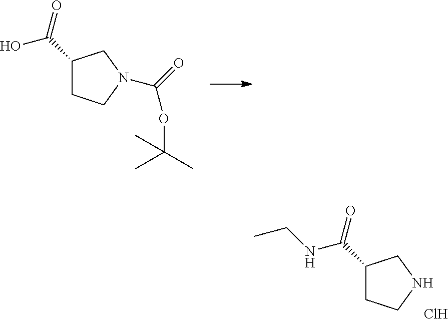

S)-1-(tert-Butoxycarbonyl)pyrrolidine-3-carboxylic acid (500 mg, 2.32 mmol), ethylamine (1.742 mL, 3.48 mmol) and HATU (1060 mg, 2.79 mmol) were stirred in DMF (10 mL) containing N,N-diisopropylethylaminc (0.607 mL, 3.48 mmol) at 23° C. for 1 h. The solvent was evaporated. The residue was dissolved in EtOAc and washed with 5% KHSO₄, aqueous saturated NaHCO₃, brine and dried over anhydrous Na₂SO₄. The product was purified by flash using EtOAc as eluent. The product was then stirred in hydrogen chloride (11.61 mL, 11.61 mmol) (1M/AcOH) at 23° C. for 2 h. The solvent was evaporated. The product was rinsed several times with ether, filtered and dried. Yield: 375 mg (90%); ¹H NMR (400 MHz, METHANOL-D4) δ 1.10 (t, J=7.42 Hz, 3H), 2.08 (ddd, J=13.67, 6.64 Hz, 1H), 2.28 (ddd, J=20.70, 7.81, 7.42 Hz, 1H), 3.07-3.15 (m, 1H), 3.19 (q, J=7.29 Hz, 2H), 3.32-3.40 (m, 2H), 3.40-3.48 (m, 2H).

### Example 70

### Step 1: N-(1-(9-Methyl-3-(tetrahydro-2H-pyran-4-yl)-2,3,4,9-tetrahydro-1H-carbazole-6-carbonyl)piperidin-3-yl)propionamide

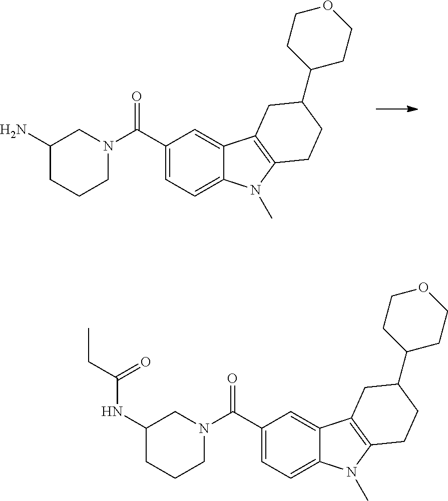

(3-Aminopiperidin-1-yl)(9-methyl-3-(tetrahydro-2H-pyran-4-yl)-2,3,4,9-tetrahydro-1H-carbazol-6-yl)methanone (75 mg, 0.19 mmol) and propionyl chloride (0.020 mL, 0.23 mmol) were stirred in dichloromethane (5 mL) containing triethylamine (0.040 mL, 0.28 mmol) at 23° C. for 1 h. The solvent was evaporated and the product was directly purified by reversed-phase HPLC. Reversed-phase purification: Gilson system equipped with Luna C-18 column, 250×21.2 mm, 15 u. Mobile phase: 30-50% B; A: H₂O with 0.05% TFA v/v; B: CH₃CN; 30 mL/min, 25 min run, rt. Yield: 70 mg (82%); 1H NMR (400 MHz, METHANOL-D4) δ 1.03 (s, 2H), 1.37-1.51 (m, 2H), 1.52-1.65 (m, 5H), 1.79 (t, J=10.94 Hz, 3H), 1.95-2.03 (m, 1H), 2.08-2.21 (m, 3H), 2.32-2.46 (m, 1H), 2.64-2.75 (m, 1H), 2.79-2.91 (m, 2H), 3.16 (s, 1H), 3.38-3.49 (m, 2H), 3.64 (s, 3H), 3.84 (s, 2H), 3.99 (dd, J=11.13, 3.71 Hz, 2H), 7.16 (dt, J=8.30, 1.51 Hz, 1H), 7.32 (d, J=8.59 Hz, 1H), 7.50 (s, 1H); MS (ESI) (M+H)⁺=452.2; Accurate mass: (M+H)=452.290.

### Step B: (3-Aminopiperidin-1-yl)(9-methyl-3-(tetrahydro-2H-pyran-4-yl)-2,3,4,9-tetrahydro-1H-carbazol-6-yl)methanone

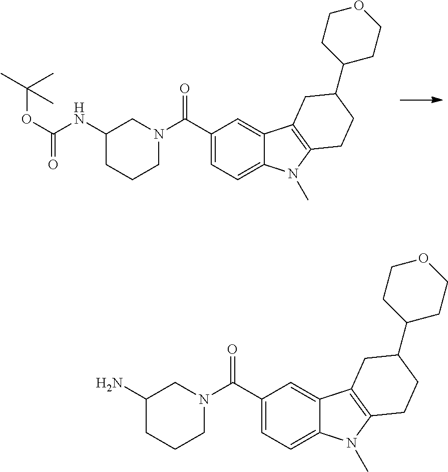

tert-Butyl 1-(9-methyl-3-(tetrahydro-2H-pyran-4-yl)-2,3,4,9-tetrahydro-1H-carbazole-6-carbonyl)piperidin-3-ylcarbamate (see Example 18, Step B) (750 mg, 1.51 mmol) was stirred in hydrogen chloride (7566 μL, 7.57 mmol) (1M/AcOH) at 23° C. for 2 h. The solvent was evaporated. The residue was dissolved in EtOAc and CH₂Cl₂ and washed with saturated aqueous NaHCO₃, brine and dried over anhydrous Na₂SO₄. The solvent was evaporated. Yield: 502 mg (84%); ¹H NMR (400 MHz, METHANOL-D4) δ 1.35-1.49 (m, 4H), 1.51-1.61 (m, 4H), 1.77 (t, J=13.09 Hz, 3H), 1.99-2.07 (m, 1H), 2.13-2.19 (m, 1H), 2.34-2.42 (m, 1H), 2.63-2.74 (m, 1H), 2.79-2.89 (m, 4H), 3.36-3.46 (m, 2H), 3.62 (s, 3H), 3.97 (dd, J=11.13, 4.10 Hz, 2H), 7.13 (dd, J=8.40, 1.76 Hz, 1H), 7.31 (d, J=8.59 Hz, 1H), 7.47 (d, J=1.17 Hz, 1H); MS (ESI) (M+H)⁺=396.31.

### Example 71

### N-(1-(9-Methyl-3-(tetrahydro-2H-pyran-4-yl)-2,3,4,9-tetrahydro-1H-carbazole-6-carbonyl)piperidin-3-yl)isobutyramide

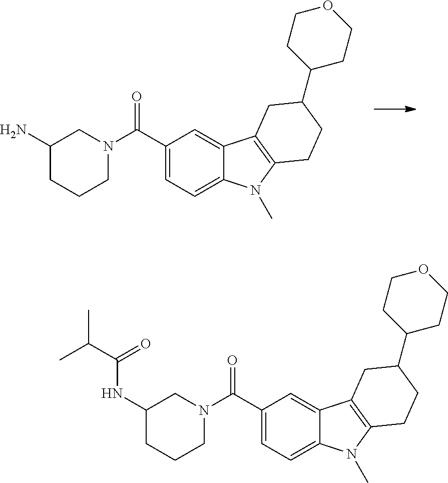

(3-Aminopiperidin-1-yl)(9-methyl-3-(tetrahydro-2H-pyran-4-yl)-2,3,4,9-tetrahydro-1H-carbazol-6-yl)methanone (75 mg, 0.19 mmol) and isobutyryl chloride (0.024 mL, 0.23 mmol) were stirred in CH₂Cl₂ (5 mL) containing triethylamine (0.040 mL, 0.28 mmol) at 23° C. for 1 h. The solvent was evaporated and the product was directly purified by reversed-phase HPLC and lyophilized. Reversed-phase purification: Gilson system equipped with Luna C-18 column, 250×21.2 mm, 15 u. Mobile phase: 30-50% B; A: H₂O with 0.05% TFA v/v; B: CH₃CN; 30 mL/min, 25 min run, rt. Yield: 75 mg (85%); ¹H NMR (400 MHz, METHANOL-D4) δ 1.03 (s, 6H), 1.37-1.50 (m, 2H), 1.52-1.64 (m, 6H), 1.73-1.84 (m, 3H), 1.92-2.02 (m, 1H), 2.11-2.20 (m, 1H), 2.30-2.45 (m, 2H), 2.61-2.74 (m, 1H), 2.77-2.91 (m, 2H), 3.03-3.15 (m, 1H), 3.22 (s, 1H), 3.35-3.48 (m, 2H), 3.62 (s, 3H), 3.82 (s, 1H), 3.98 (dd, J=11.52, 3.71 Hz, 2H), 7.14 (dt, J=8.30, 1.51 Hz, 1H), 7.30 (d, J=8.20 Hz, 1H), 7.49 (s, 1H); MS (ESI) (M+H)⁺=466.2; accurate mass (M+H)=466.307.

### Example 72

### 2-Cyclopropyl-N-(1-(9-methyl-3-(tetrahydro-2H-pyran-4-yl)-2,3,4,9-tetrahydro-1H-carbazole-6-carbonyl)piperidin-3-yl)acetamide

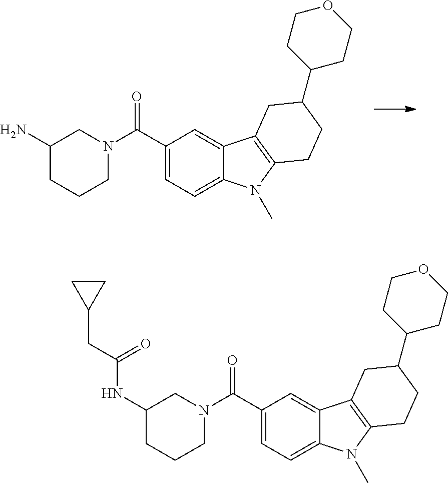

(3-Aminopiperidin-1-yl)(9-methyl-3-(tetrahydro-2H-pyran-4-yl)-2,3,4,9-tetrahydro-1H-carbazol-6-yl)methanone (75 mg, 0.19 mmol), HATU (87 mg, 0.23 mmol) and cyclopropylacetic acid (22.78 mg, 0.23 mmol) were stirred in DMF (5 mL) containing N,N-diisopropylethylamine (0.050 mL, 0.28 mmol) at 23° C. for 1 h. The solvent was evaporated. The product was directly purified by reversed-phase HPLC and lyophilized. Reversed-phase purification: Gilson system equipped with Luna C-18 column, 250×21.2 mm, 15 u. Mobile phase: 30-50% B; A: H₂O with 0.05% TFA v/v; B: CH₃CN; 30 mL/min, 25 min run, rt. Yield: 72 mg (79%); ¹H NMR (400 MHz, METHANOL-D4) δ 0.11 (s, 2H), 0.45 (s, 2H), 0.92 (s, 1H), 1.36-1.49 (m, 2H), 1.52-1.64 (m, 5H), 1.77 (t, J=10.94 Hz, 3H), 1.92-2.05 (m, 3H), 2.16 (t, J=7.62 Hz, 1H), 2.32-2.46 (m, 1H), 2.62-2.74 (m, 1H), 2.77-2.89 (m, 2H), 3.13-3.23 (m, 1H), 3.37-3.47 (m, 2H), 3.62 (s, 3H), 3.85 (s, 2H), 3.98 (dd, J=10.94, 3.52 Hz, 2H), 7.14 (ddd, J=8.40, 1.56, 1.37 Hz, 1H), 7.30 (d, J=8.59 Hz, 1H), 7.48 (s, 1H); MS (ESI) (M+H)⁺=478.3; accurate mass: (M+H)=478.307.

### Example 73

### Step A: (R)—N-(4-(2-Hydroxyethylamino)-4-oxobutyl)-N,9-dimethyl-3-(tetrahydro-2H-pyran-4-yl)-2,3,4,9-tetrahydro-1H-carbazole-6-carboxamide

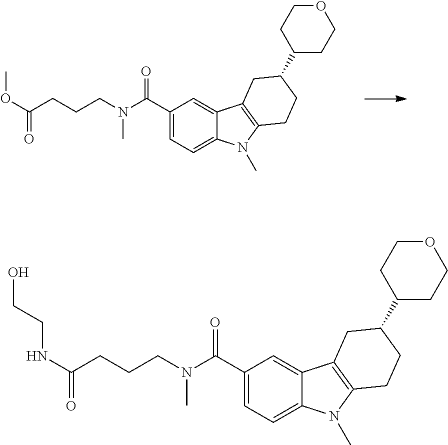

(R)-Methyl 4-(N,9-dimethyl-3-(tetrahydro-2H-pyran-4-yl)-2,3,4,9-tetrahydro-1H-carbazole-6-carboxamido)butanoate (100 mg, 0.23 mmol) was stirred in dioxane (5 mL) containing lithium hydroxide (0.469 mL, 0.47 mmol) (1M) at 23° C. for 2 h. The solvent was evaporated. The residue was dissolved in EtOAc and washed with 5% KHSO₄, brine and dried over anhydrous Na₂SO₄. The solvent was evaporated. The product was dissolved in DMF (5.00 mL) containing N,N-diisopropylethylamine (0.102 mL, 0.59 mmol) and ethanolamine (0.017 mL, 0.28 mmol) along with HATU (107 mg, 0.28 mmol) were added. The solution was stirred at 23° C. for 1 h. The solvent was evaporated. The product was directly purified by reversed-phase HPLC and lyophilized. Reversed-phase purification: Gilson system equipped with Luna C-18 column, 250×21.2 mm, 15 u. Mobile phase: 20-40% B; A: H₂O with 0.05% TFA v/v; B: CH₃CN; 30 mL/min, 25 min run, rt. Yield: 55 mg (52%); ¹H NMR (400 MHz, METHANOL-D4) δ 1.37-1.49 (m, 2 H), 1.51-1.62 (m, 3H), 1.76 (t, J=12.50 Hz, 2H), 1.86 (s, 1H), 1.99 (d, J=11.72 Hz, 2 H), 2.11-2.20 (m, 1H), 2.30 (s, 1H), 2.34-2.43 (m, 1H), 2.62-2.73 (m, 1H), 2.79-2.90 (m, 2H), 3.04 (s, 4H), 3.36-3.46 (m, 4H), 3.57 (s, 2H), 3.62 (s, 3H), 3.97 (dd, J=11.13, 3.71 Hz, 2H), 7.13 (s, 1H), 7.31 (d, J=8.59 Hz, 1H), 7.46 (s, 1H); MS (ESI) (M+H)⁺=456.2; accurate mass: (M+H)=456.286.

### Step B: Methyl 4-(tert-butoxycarbonyl(methyl)amino)butanoate

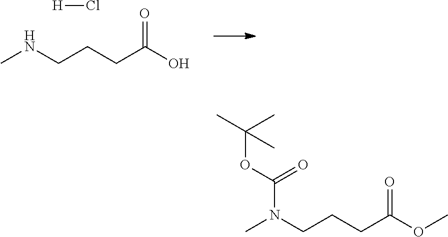

4-(Methylamino)butyric acid hydrochloride (1.0 g, 6.51 mmol) and di-tert-butyl dicarbonate (1.797 mL, 7.81 mmol) were stirred in dioxane (50 mL) and water (10 mL) containing triethylamine (1.361 mL, 9.77 mmol) at 23° C. for 3 h. The solvent was evaporated. The residue was dissolved in EtOAc and washed with 5% KHSO₄, brine and dried over anhydrous Na₂SO₄. The solvent was evaporated. The residue was then dissolved in methanol (50.0 mL) at 0° C. and (trimethylsilyl)diazomethane (9.77 mL, 19.53 mmol) was added dropwise to the stirring solution until a light yellow color persisted. The solution was then stirred at 23° C. for 15 min. The solvent was evaporated. The residue was dissolved in EtOAc and washed with 5% KHSO₄, aqueous saturated NaHCO₃, brine and dried over anhydrous Na₂SO₄. The product was purified by flash chromatography using a gradient: 20% to 50% EtOAc/heptane. Yield: 1.17 g (78%); ¹H NMR (400 MHz, CHLOROFORM-D) δ 1.45 (s, 9H), 1.84 (ddd, J=14.26, 7.42, 7.23 Hz, 2H), 2.32 (t, J=7.42 Hz, 2H), 2.84 (s, 3H), 3.25 (t, J=6.84 Hz, 2H), 3.68 (s, 3H); MS (ESI) (M+H)⁺=232.23.

### Step C: Methyl 4-(methylamino)butanoate hydrochloride

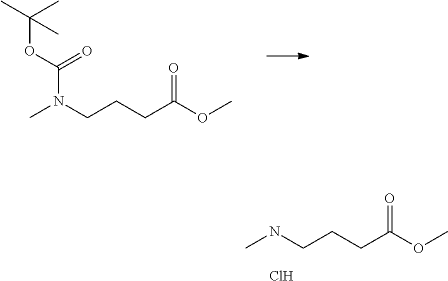

Methyl 4-(tert-butoxycarbonyl(methyl)amino)butanoate (1.15 g, 4.97 mmol) was stirred in hydrogen chloride (14.92 ml, 14.92 mmol) (1M/AcOH) at 23° C. for 2 h. The solvent was evaporated. The product was precipitated in ether, filtered and dried. Yield: 740 mg (89%); ¹H NMR (400 MHz, METHANOL-D4) δ 1.96 (ddd, J=15.04, 7.42, 7.23 Hz, 2H), 2.49 (t, J=7.03 Hz, 2H), 2.70 (s, 3H), 3.01-3.08 (m, 2H), 3.68 (s, 3H).

### Step D: (R)-Methyl 4-(N,9-dimethyl-3-(tetrahydro-2H-pyran-4-yl)-2,3,4,9-tetrahydro-1H-carbazole-6-carboxamido)butanoate (Isomer 1)

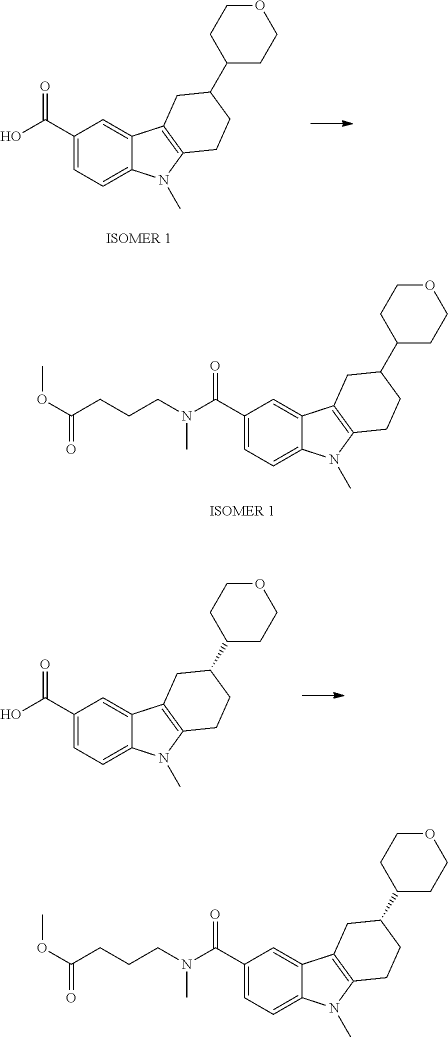

(R)-9-Methyl-3-(tetrahydro-2H-pyran-4-yl)-2,3,4,9-tetrahydro-1H-carbazole-6-carboxylic acid (185 mg, 0.59 mmol), methyl 4-(methylamino)butanoate hydrochloride (119 mg, 0.71 mmol) and HATU (269 mg, 0.71 mmol) were stirred in DMF (10 mL) containing N,N-diisopropylethylamine (0.257 mL, 1.48 mmol) at 23° C. for 1 h. The solvent was evaporated. The residue was dissolved in EtOAc and washed with 5% KHSO₄, saturated aqueous NaHCO₃, brine and dried over anhydrous Na₂SO₄. The product was purified by flash chromatography using EtOAc as eluent. Yield: 240 mg (95%); ¹H NMR (400 MHz, CHLOROFORM-D) δ 1.41-1.52 (m, 2H), 1.52-1.63 (m, 5H), 1.71-1.81 (m, 2H), 1.91-2.02 (m, 2H), 2.11-2.19 (m, 1H), 2.36-2.47 (m, 2H), 2.63-2.74 (m, 1H), 2.78-2.90 (m, 2H), 3.05 (s, 3H), 3.37-3.47 (m, 3H), 3.58-3.70 (m, 6H), 4.04 (dd, J=11.52, 2.93 Hz, 2H), 7.17-7.21 (m, 1H), 7.21-7.25 (m, 1H), 7.54 (s, 1H); MS (ESI) (M+H)⁺=427.41.

### Example 74

### Step A: (R)—N-((3S)-1-(9-Methyl-3-(tetrahydro-2H-pyran-4-yl)-2,3,4,9-tetrahydro-1H-carbazole-6-carbonyl)piperidin-3-yl)cyclopropanecarboxamide

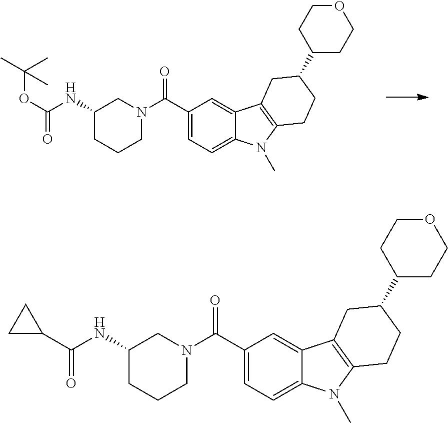

(R)-tert-Butyl (3S)-1-(9-methyl-3-(tetrahydro-2H-pyran-4-yl)-2,3,4,9-tetrahydro-1H-carbazole-6-carbonyl)piperidin-3-ylcarbamate (75 mg, 0.15 mmol) was stirred in hydrogen chloride (0.757 mL, 0.76 mmol) (1M/AcOH) at 23° C. for 1 h. The solvent was evaporated. The residue was dissolved in EtOAc and washed with saturated aqueous NaHCO₃, brine and dried over anhydrous Na₂SO₄. The solvent was evaporated. The product was dissolved in DCM (5 mL) containing triethylamine (0.032 mL, 0.23 mmol) and cyclopropanecarbonyl chloride (0.016 mL, 0.18 mmol) was added dropwise. The solution was stirred at 23° C. for 1 h. The solvent was evaporated and the product was purified by reversed-phase HPLC and lyophilized. Reversed-phase purification: Gilson system equipped with Luna C-18 column, 250×21.2 mm, 15 u. Mobile phase: 30-50% B; A: H₂O with 0.05% TFA v/v; B: CH₃CN; 30 mL/min, 25 min run, rt. Yield: 48 mg (68%); ¹H NMR (400 MHz, METHANOL-D4) δ 0.68 (s, 4H), 1.36-1.49 (m, 2H), 1.50-1.63 (m, 6H), 1.72-1.82 (m, 2H), 1.93-2.02 (m, 1H), 2.11-2.19 (m, 1H), 2.35-2.44 (m, 1H), 2.63-2.73 (m, 1H), 2.78-2.89 (m, 2H), 3.18 (s, 2H), 3.37-3.46 (m, 3H), 3.62 (s, 3H), 3.82 (s, 2H), 3.98 (dd, J=10.55, 3.52 Hz, 2H), 7.14 (dd, J=8.59, 1.56 Hz, 1H), 7.29 (d, J=8.20 Hz, 1H), 7.48 (d, J=1.17 Hz, 1H); MS (ESI) (M+H)⁺=464.2; accurate mass: (M+H)=464.291.

### Step B: (R)-tert-Butyl (3S)-1-(9-methyl-3-(tetrahydro-2H-pyran-4-yl)-2,3,4,9-tetrahydro-1H-carbazole-6-carbonyl)piperidin-3-ylcarbamate

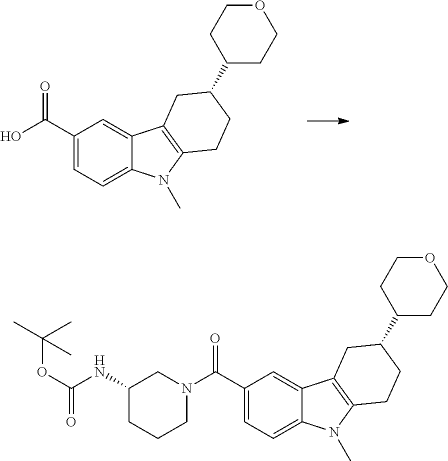

(R)-9-Methyl-3-(tetrahydro-2H-pyran-4-yl)-2,3,4,9-tetrahydro-1H-carbazole-6-carboxylic acid (100 mg, 0.32 mmol), (S)-3-N-Boc-amino piperidine (77 mg, 0.38 mmol) and HATU (146 mg, 0.38 mmol) were stirred in DMF (5 mL) at 23° C. for 1 h. The solvent was evaporated. The residue was dissolved in EtOAc and washed with 5% KHSO₄, saturated aqueous NaHCO₃, brine and dried over anhydrous Na₂SO₄. The product was purified by flash chromatography using a gradient: 30% EtOAc/heptane to 100% EtOAc. Yield: 148 mg (94%); ¹H NMR (400 MHz, CHLOROFORM-D) δ 1.41 (s, 9H), 1.44-1.55 (m, 4H), 1.55-1.66 (m, 5H), 1.71-1.80 (m, 3H), 1.95 (s, 1H), 2.10-2.19 (m, 1H), 2.39-2.49 (m, 1H), 2.63-2.75 (m, 1H), 2.78-2.90 (m, 2H), 3.42 (t, J=11.72 Hz, 3H), 3.63 (s, 3H), 3.70-3.77 (m, 1H), 3.80-3.90 (m, 1H), 4.04 (dd, J=10.94, 3.12 Hz, 2H), 7.24 (s, 2H), 7.61 (s, 1H); MS (ESI) (M+H)⁺=496.51.

### Example 75

### Step A: (R)—N,9-Dimethyl-N-(4-oxo-4-((S)-tetrahydrofuran-3-ylamino)butyl)-3-(tetrahydro-2H-pyran-4-yl)-2,3,4,9-tetrahydro-1H-carbazole-6-carboxamide

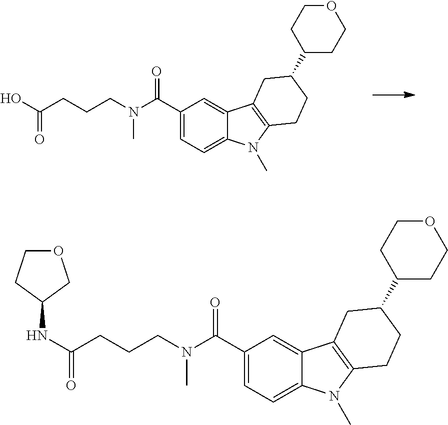

(R)-4-(N,9-Dimethyl-3-(tetrahydro-2H-pyran-4-yl)-2,3,4,9-tetrahydro-1H-carbazole-6-carboxamido)butanoic acid (75 mg, 0.18 mmol), (R)-tetrahydrofuran-3-amine hydrochloride (22.47 mg, 0.18 mmol) and HATU (83 mg, 0.22 mmol) were stirred in DMF (5 mL) containing N,N-diisopropylethylamine (0.079 mL, 0.45 mmol) at 23° C. for 1 h. The solvent was evaporated. The product was purified by reversed-phase HPLC and lyophilized. Reversed-phase purification: Gilson system equipped with Luna C-18 column, 250×21.2 mm, 15 u. Mobile phase: 30-50% B; A: H₂O with 0.05% TFA v/v; B: CH₃CN; 30 mL/min, 25 min run, rt. Yield: 72 mg (82%); ¹H NMR (400 MHz, METHANOL-D4) δ 1.35-1.49 (m, 2H), 1.50-1.61 (m, 3H), 1.77 (t, J=12.11 Hz, 2H), 1.85 (s, 1H), 1.98 (d, 2H), 2.11-2.19 (m, 2H), 2.27 (s, 1H), 2.32-2.44 (m, 1H), 2.60-2.75 (m, 1H), 2.79-2.89 (m, 2H), 3.04 (s, 3H), 3.34-3.46 (m, 3H), 3.56 (s, 2H), 3.62 (s, 3H), 3.70-3.89 (m, 2H), 3.97 (dd, J=11.33, 3.52 Hz, 2H), 4.07 (s, 0.5H), 4.36 (s, 0.5H), 7.12 (s, 1H), 7.31 (d, J=8.20 Hz, 1H), 7.46 (s, 1H); MS (ESI) (M+H)⁺=482.2; accurate mass: (M+H)=482.301.

### Step B: (R)-4-(N,9-Dimethyl-3-(tetrahydro-2H-pyran-4-yl)-2,3,4,9-tetrahydro-1H-carbazole-6-carboxamido)butanoic acid (Isomer 1)

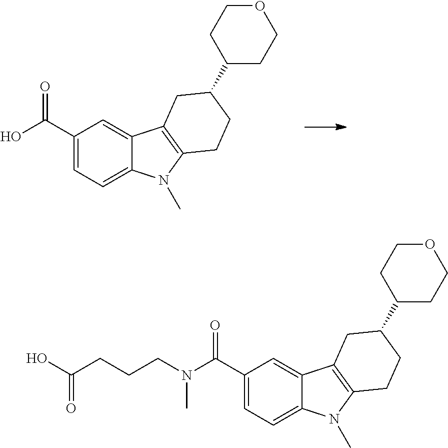

(R)-9-Methyl-3-(tetrahydro-2H-pyran-4-yl)-2,3,4,9-tetrahydro-1H-carbazole-6-carboxylic acid (250 mg, 0.80 mmol), methyl 4-(methylamino)butanoate hydrochloride (160 mg, 0.96 mmol) and HATU (364 mg, 0.96 mmol) were stirred in DMF (10 mL) containing N,N-diisopropylethylamine (0.347 mL, 1.99 mmol) at 23° C. for 1 h. The solvent was evaporated. The residue was dissolved in EtOAc and washed with 5% KHSO₄, saturated aqueous NaHCO₃, brine and dried over anhydrous Na₂SO₄. The product was purified by flash chromatography using EtOAc as eluent. The product was then dissolved in dioxane (5 mL) containing lithium hydroxide (1.595 mL, 1.60 mmol) (1M) and the solution was stirred at 23° C. for 2 h. The solvent was evaporated. The residue was dissolved in EtOAc and washed with 5% KHSO₄, brine and dried over anhydrous Na₂SO₄. Yield: 300 mg (91%); ¹H NMR (400 MHz, DMSO-D6) δ 1.22-1.37 (m, 2H), 1.41-1.53 (m, 3H), 1.68 (t, J=12.30 Hz, 2H), 1.72-1.80 (m, 2H), 2.01-2.08 (m, 1H), 2.10-2.23 (m, 1H), 2.29 (dd, J=14.84, 8.20 Hz, 1H), 2.57-2.68 (m, 1H), 2.71-2.85 (m, 2H), 2.90 (s, 3H), 3.21-3.31 (m, 4H), 3.59 (s, 3H), 3.87 (dd, J=9.37, 1.95 Hz, 2H), 7.05 (d, J=8.20 Hz, 1H), 7.33 (d, J=8.59 Hz, 1H), 7.38 (s, 1H); MS (ESI) (M+H)⁺=413.42.

### Example 76

### (R)—N,9-Dimethyl-N-(4-(oxetan-3-ylamino)-4-oxobutyl)-3-(tetrahydro-2H-pyran-4-yl)-2,3,4,9-tetrahydro-1H-carbazole-6-carboxamide

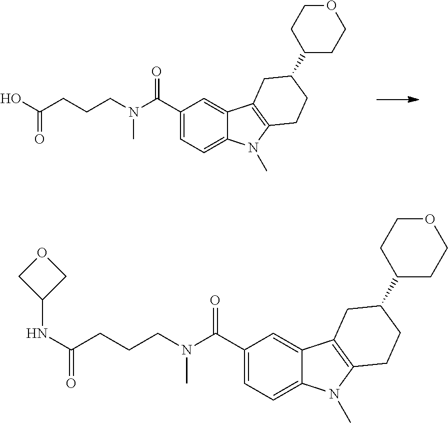

(R)-4-(N,9-Dimethyl-3-(tetrahydro-2H-pyran-4-yl)-2,3,4,9-tetrahydro-1H-carbazole-6-carboxamido)butanoic acid (75 mg, 0.18 mmol), oxetan-3-amine hydrochloride (23.90 mg, 0.22 mmol) and HATU (83 mg, 0.22 mmol) were stirred in DMF (5 mL) containing N,N-diisopropylethylamine (0.079 mL, 0.45 mmol) at 23° C. for 1 h. The solvent was concentrated. The residue was purified by reversed-phase HPLC and lyophilized.

Reversed-phase purification: Gilson system equipped with Synergi Polar-RP, 30×50 mm, 4 mm particle size. Mobile phase: 30-50% B; A: H₂O with 15 mM NH₄CO₃ and 0.375% NH₄OH v/v, B: CH₃CN; 45 mL/min, 15 min run, rt. Yield: 45 mg (53%); ¹H NMR (400 MHz, METHANOL-D4) δ 1.37-1.51 (m, 2H), 1.53-1.62 (m, 3H), 1.78 (t, J=12.30 Hz, 2H), 1.86 (s, 1H), 1.99 (s, 2H), 2.13-2.20 (m, 1H), 2.31 (s, 1H), 2.35-2.46 (m, 1H), 2.64-2.75 (m, 1H), 2.81-2.90 (m, 2H), 3.04 (s, 3H), 3.36-3.47 (m, 3 H), 3.57 (s, 1H), 3.63 (s, 3H), 3.98 (dd, J=10.94, 3.52 Hz, 2H), 4.26 (s, 1H), 4.51 (s, 1 H), 4.62 (s, 1H), 4.79 (s, 1H), 7.12 (s, 1H), 7.31 (d, J=8.20 Hz, 1H), 7.46 (s, 1H); MS (ESI) (M+H)⁺=468.2; accurate mass: (M+H)=468.286.

### Example 77

### (R)—N-(4-(3-Hydroxypropylamino)-4-oxobutyl)-N,9-dimethyl-3-(tetrahydro-2H-pyran-4-yl)-2,3,4,9-tetrahydro-1H-carbazole-6-carboxamide

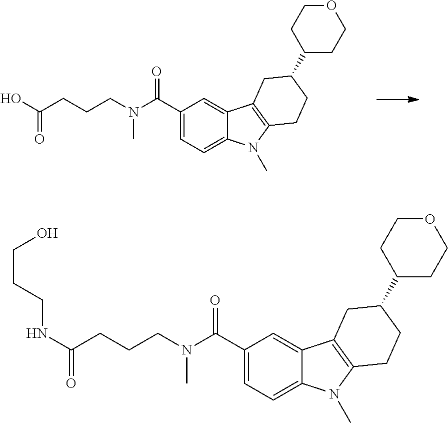

(R)-4-(N,9-Dimethyl-3-(tetrahydro-2H-pyran-4-yl)-2,3,4,9-tetrahydro-1H-carbazole-6-carboxamido)butanoic acid (73 mg, 0.18 mmol), HATU (81 mg, 0.21 mmol) and 3-amino-1-propanol (0.016 mL, 0.21 mmol) were stirred in DMF (5 mL) containing N,N-diisopropylethylamine (0.046 mL, 0.27 mmol) at 23° C. for 1 h. The solvent was evaporated. The product was purified directly by reversed-phase HPLC and lyophilized. Reversed-phase purification: Gilson system equipped with a Synergi Polar-RP, 30×50 mm, 4 mm particle size. Mobile phase: 30-50% B; A: H₂O with 15 mM NH₄CO₃ and 0.375% NH₄OH v/v, B: CH₃CN; 45 mL/min, 15 min run, rt. Yield: 35 mg (42%); ¹H NMR (400 MHz, METHANOL-D4) δ 1.37-1.50 (m, 3H), 1.52-1.62 (m, 3H), 1.69 (s, 1H), 1.77 (t, J=12.30 Hz, 2H), 1.86 (s, 1H), 1.96 (s, 2H), 2.12-2.20 (m, 1H), 2.27 (s, 1H), 2.34-2.44 (m, 1H), 2.63-2.74 (m, 1H), 2.79-2.90 (m, 2H), 3.04 (s, 4H), 3.24 (s, 1H), 3.35-3.47 (m, 4H), 3.56 (s, 2H), 3.62 (s, 3H), 3.98 (dd, J=11.13, 3.71 Hz, 2 H), 7.12 (s, 1H), 7.31 (d, J=8.59 Hz, 1H), 7.46 (s, 1H); MS (ESI) (M+H)⁺=470.2; accurate mass: (M+H)=470.301

### Example 78

### Step A: (R)—N-Ethyl-9-(ethylsulfonyl)-N-(2-(2-hydroxyethylamino)-2-oxoethyl)-3-(tetrahydro-2H-pyran-4-yl)-2,3,4,9-tetrahydro-1H-carbazole-6-carboxamide

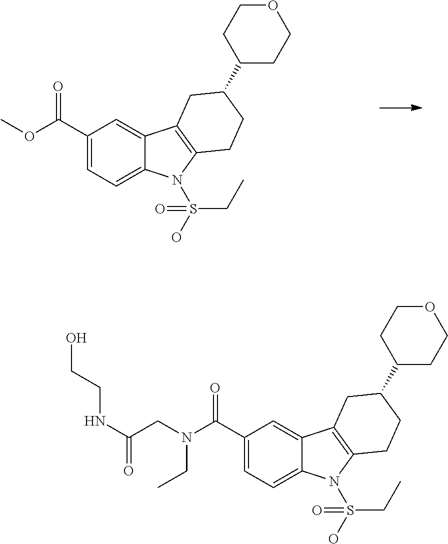

(R)-Methyl 9-(ethylsulfonyl)-3-(tetrahydro-2H-pyran-4-yl)-2,3,4,9-tetrahydro-1H-carbazole-6-carboxylate (45 mg, 0.11 mmol) was stirred in dioxane (5 mL) containing lithium hydroxide (0.222 mL, 0.22 mmol) (1M) at 50° C. for 2-3 h. The solvent was evaporated. The residue was dissolved in DMF (5.00 mL) containing N,N-diisopropylethylamine (0.048 mL, 0.28 mmol) and N-ethyl-2-(2-hydroxyethylamino)-2-oxoethanaminium 2,2,2-trifluoroacetate (34.7 mg, 0.13 mmol) along with HATU (63.3 mg, 0.17 mmol) were added. The solution was stirred at 23° C. for 1 h. More N-ethyl-2-(2-hydroxyethylamino)-2-oxoethanaminium 2,2,2-trifluoroacetate (34.7 mg, 0.13 mmol) was added and the solution was stirred at 23° C. for another 1 h. The solvent was concentrated. The residue was dissolved in EtOAc and washed with saturated aqueous NaHCO₃, brine and dried over anhydrous Na₂SO₄. The product was purified by reversed-phase HPLC using 30-50% B and lyophilized. Reversed-phase purification: Gilson system equipped with Luna C-18 column, 250×21.2 mm, 15 u. Mobile phase: A: H₂O with 0.05% TFA v/v; B: CH₃CN; 30 mL/min, 25 min run, rt. Yield: 15 mg (26%); ¹H NMR (400 MHz, METHANOL-D4) δ 1.14 (t, J=7.42 Hz, 4H), 1.20-1.27 (m, 2H), 1.27-1.37 (m, 2H), 1.40-1.50 (m, 2H), 1.53-1.66 (m, 3H), 1.74-1.85 (m, 2H), 2.13-2.22 (m, 1H), 2.32-2.44 (m, 1H), 2.77-2.92 (m, 2H), 3.11-3.21 (m, 1H), 3.31-3.40 (m, 3H), 3.39-3.49 (m, 3H), 3.53-3.67 (m, 2H), 4.00 (dd, J=11.13, 3.32 Hz, 3H), 4.19 (s, 1H), 7.31-7.40 (m, 1H), 7.55-7.66 (m, 1H), 7.93-8.04 (m, 1H); MS (ESI) (M+H)⁺=520.2; Accurate mass: (M+H)=520.247.

### Step B: Methyl 3-(tetrahydro-2H-pyran-4-yl)-2,3,4,9-tetrahydro-1H-carbazole-6-carboxylate

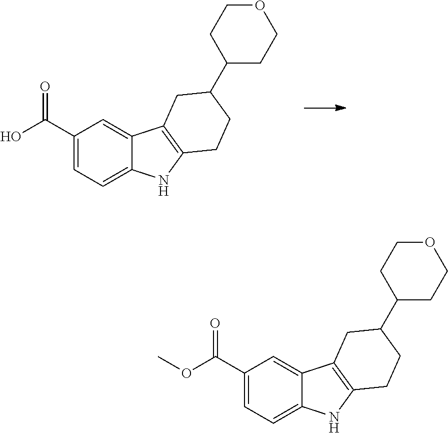

3-(Tetrahydro-2H-pyran-4-yl)-2,3,4,9-tetrahydro-1H-carbazole-6-carboxylic acid (250 mg, 0.84 mmol) was dissolved in MeOH (20 mL). (Trimethylsilyl)diazomethane (2.088 mL, 4.18 mmol) was added dropwise at 0° C. until a light yellow color persisted. The solution was then stirred at 23° C. for 30 min. The solvent was evaporated. The product was purified by flash chromatography using a gradient of 20%-80% EtOAc/heptane. Yield: 170 mg (65%); ¹H NMR (400 MHz, CHLOROFORM-D) δ 1.42-1.52 (m, 2H), 1.52-1.60 (m, 1H), 1.60-1.69 (m, 2H), 1.76 (d, J=12.11 Hz, 2H), 2.07-2.10-2.15 (m, 1H), 2.39-2.45 (m, 1H), 2.77 (s, 2H), 2.89 (dd, J=15.23, 4.30 Hz, 1H), 3.43 (t, J=11.72 Hz, 2H), 3.93 (s, 3H), 4.05 (dd, J=11.13, 2.54 Hz, 2H), 7.24-7.30 (m, 1H), 7.84 (d, J=8.59 Hz, 1H), 7.94 (s, 1H), 8.22 (s, 1H); MS (ESI) (M+H)⁺=314.28.

### Step C: (R)-Methyl 3-(tetrahydro-2H-pyran-4-yl)-2,3,4,9-tetrahydro-1H-carbazole-6-carboxylate and (S)-Methyl 3-(tetrahydro-2H-pyran-4-yl)-2,3,4,9-tetrahydro-1H-carbazole-6-carboxylate

Chiral separation of methyl 3-(tetrahydro-2H-pyran-4-yl)-2,3,4,9-tetrahydro-1H-carbazole-6-carboxylate (150 mg, 0.48 mmol) was done as the following: Gilson system equipped with a Chiracel OD column, 5 cm ID×50 cm L, 20 u using 15% EtOH/hexanes with 0.1% diethylamine v/v; 100 mL/min, 60 min run, rt. Chiral analytical HPLC: ChiraCel OD column, 20% EtOH/hexanes, 1 mL/min, 30 min run, 25° C.

(R)-Methyl 3-(tetrahydro-2H-pyran-4-yl)-2,3,4,9-tetrahydro-1H-carbazole-6-carboxylate (isomer 1, 62 mg, 41%).

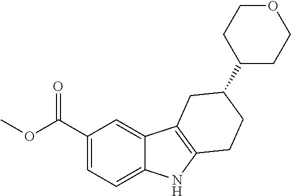

¹H NMR (400 MHz, CHLOROFORM-D) δ 1.43-1.52 (m, 2H), 1.52-1.60 (m, 2H), 1.59-1.70 (m, 1H), 1.76 (d, J=12.50 Hz, 2H), 2.07-2.15 (m, 1H), 2.37-2.47 (m, 1H), 2.75-2.81 (m, 2H), 2.89 (dd, J=15.23, 3.91 Hz, 1H), 3.38-3.47 (m, 2H), 3.93 (s, 3H), 4.05 (dd, J=11.52, 3.32 Hz, 2H), 7.29 (s, 1H), 7.84 (dd, J=8.59, 1.56 Hz, 1H), 7.92 (s, 1H), 8.22 (d, J=0.78 Hz, 1H); MS (ESI) (M+H)⁺=314.21; chiral HPLC k′=2.36.

(S)-Methyl 3-(tetrahydro-2H-pyran-4-yl)-2,3,4,9-tetrahydro-1H-carbazole-6-carboxylate (isomer 2, 68 mg. 45%).

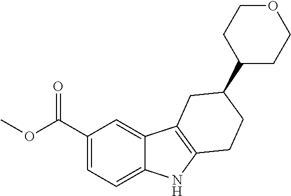

¹H NMR (400 MHz, CHLOROFORM-D) δ 1.42-1.52 (m, 2H), 1.52-1.60 (m, 2H), 1.60-1.69 (m, 1H), 1.76 (d, J=12.11 Hz, 2H), 2.07-2.16 (m, 1H), 2.37-2.47 (m, 1H), 2.75-2.81 (m, 2H), 2.90 (dd, J=15.23, 4.30 Hz, 1H), 3.38-3.47 (m, 2H), 3.93 (s, 3 H), 4.05 (dd, J=11.52, 3.32 Hz, 2H), 7.26-7.30 (m, 1H), 7.84 (dd, J=8.59, 1.56 Hz, 1H), 7.91 (s, 1H), 8.22 (s, 1H); MS (ESI) (M+H)⁺=314.20; chiral HPLC k′=2.72.

### Step D: (R)-Methyl 9-(ethylsulfonyl)-3-(tetrahydro-2H-pyran-4-yl)-2,3,4,9-tetrahydro-1H-carbazole-6-carboxylate

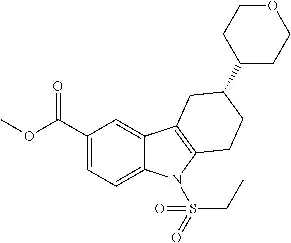

(R)-Methyl 3-(tetrahydro-2H-pyran-4-yl)-2,3,4,9-tetrahydro-1H-carbazole-6-carboxylate (62 mg, 0.20 mmol) was dissolved in DMF (5 mL) at 0° C. under N₂. Sodium hydride (39.6 mg, 0.99 mmol) was added slowly and the solution was stirred at 0° C. for 30 min. Ethanesulfonyl chloride (0.037 mL, 0.40 mmol) was added and the solution was stirred at rt for 1 h. Another 1 eq. of ethanesulfonyl chloride (0.037 mL, 0.40 mmol) was added and the solution was stirred at rt for another 2 h. LC/MS showed that the reaction was still not completed but reaction was quenched anyway at 0° C. with saturated aqueous NaHCO₃ and the solvent was evaporated. The residue was dissolved in EtOAc and washed with water, brine and dried over anhydrous Na₂SO₄. The product was purified by flash chromatography using a gradient of 20%-80% EtOAc/heptane. Yield: 45 mg (56%); ¹H NMR (400 MHz, CHLOROFORM-D) δ 1.22 (t, J=7.42 Hz, 3H), 1.42-1.51 (m, 2H), 1.50-1.58 (m, 2H), 1.57-1.67 (m, 1H), 1.71-1.79 (m, 2H), 2.10-2.18 (m, 1H), 2.32-2.43 (m, 1H), 2.80-2.90 (m, 2H), 3.12-3.21 (m, 1H), 3.25 (q, J=7.42 Hz, 2H), 3.42 (t, J=11.52 Hz, 2H), 3.95 (s, 3H), 4.05 (dd, J=11.13, 3.32 Hz, 2H), 7.94-7.98 (m, 1H), 7.98-8.02 (m, 1H), 8.15 (d, J=0.78 Hz, 1H); MS (ESI) (M+H)⁺=406.24.

### Example 79

### (9-(Ethylsulfonyl)-3-(tetrahydro-2H-pyran-4-yl)-2,3,4,9-tetrahydro-1H-carbazol-6-yl)((R)-3-hydroxypyrrolidin-1-yl)methanone

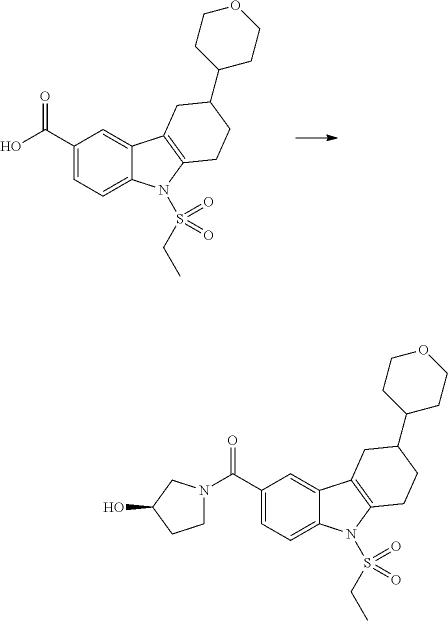

0.9-(Ethylsulfonyl)-3-(tetrahydro-2H-pyran-4-yl)-2,3,4,9-tetrahydro-1H-carbazole-6-carboxylic acid (150 mg, 0.38 mmol), (R)-pyrrolidin-3-ol (36.7 mg, 0.42 mmol) and N-ethyl-N-isopropylpropan-2-amine (0.147 mL, 0.84 mmol) are mixed in DMF (10.0 mL), and HATU (146 mg, 0.38 mmol) is added. The mixture is stirred at room temperature for 1 hour, and the solvent is evaporated. The mixture is diluted in 1N NaOH (50 mL) and extracted 3 times with EtOAc (3×50 mL). The combined organic phases are dried over sodium sulfate, the mixture is filtered, and the solvent is evaporated. The product is purified by HPLC: Gilson prep pumps, flow rate: 30 ml/min, Gemini (5 μm particle size) 21.2×50 mm, mobile phase A=10 mM ammonium bicarbonate, B=MeCN, (110 mg, 62%). ¹H NMR (400 MHz, CHLOROFORM-D) δ ppm 1.36-1.62 (m, 8H) 1.72 (t, J=13.87 Hz, 2H) 1.89-2.02 (m, 2H) 2.08-2.20 (m, 2H) 2.25-2.39 (m, 1H) 2.72-2.92 (m, J=16.41 Hz, 2H) 3.06-3.18 (m, 1H) 3.21 (q, J=7.42 Hz, 2H) 3.40 (t, J=11.72 Hz, 2H) 3.47-3.59 (m, 1H) 3.59-3.72 (m, 1H) 3.73-3.91 (m, 2H) 4.03 (dd, J=11.52, 3.32 Hz, 2H) 4.53 (d, J=61.33 Hz, 1H) 7.42 (dd, J=19.92, 8.98 Hz, 1H) 7.63 (d, J=8.98 Hz, 1H) 7.95 (d, J=8.59 Hz, 1H); MS (ESI) (M+H)⁺ 461.2.

### Example 80

### (9-(Ethylsulfonyl)-3-(tetrahydro-2H-pyran-4-yl)-2,3,4,9-tetrahydro-1H-carbazol-6-yl)((S)-3-hydroxypyrrolidin-1-yl)methanone

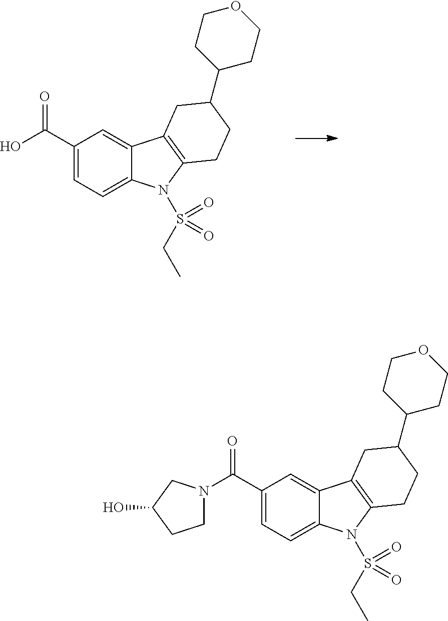

9-(Ethylsulfonyl)-3-(tetrahydro-2H-pyran-4-yl)-2,3,4,9-tetrahydro-1H-carbazole-6-carboxylic acid (160 mg, 0.41 mmol), (S)-pyrrolidin-3-ol (39.2 mg, 0.45 mmol), N-ethyl-N-isopropylpropan-2-amine (0.078 mL, 0.45 mmol) are mixed in DMF (10.0 mL) and HATU (171 mg, 0.45 mmol) is added. The mixture is stirred at room temperature for 2 hours, and the solvent is evaporated. EtOAc (75 mL) is added, and the mixture is washed with 1N NaOH (75 mL). The aqueous phase is extracted 3 times with EtOAc (3×75 mL). The organic phases are combined and dried over sodium sulfate. The mixture is filtered, and the solvent is evaporated. The product is purified by HPLC: Gilson prep pumps, flow rate: 30 ml/min, Gemini (5 μm particle size) 21.2×50 mm, mobile phase A=10 mM ammonium bicarbonate, B=MeCN, (118 mg, 63%). ¹H NMR (400 MHz, CHLOROFORM-D) δ ppm 1.18 (t, J=7.23 Hz, 3H) 1.35-1.64 (m, 6H) 1.72 (t, J=13.67 Hz, 2H) 1.92-2.07 (m, 2H) 2.07-2.18 (m, 1H) 2.25-2.42 (m, 1H) 2.68-2.95 (m, J=15.62 Hz, 2H) 3.02-3.19 (m, 1H) 3.21 (q, J=7.29 Hz, 2H) 3.40 (t, J=11.72 Hz, 2H) 3.45-3.60 (m, 1H) 3.59-3.73 (m, 1H) 3.72-3.92 (m, J=12.50, 8.98 Hz, 2H) 4.03 (dd, J=11.91, 2.93 Hz, 2H) 4.53 (d, J=62.11 Hz, 1H) 7.42 (dd, J=20.12, 8.79 Hz, 1H) 7.63 (d, J=8.98 Hz, 1H) 7.95 (d, J=8.59 Hz, 1H); MS (ESI) (M+H)⁺ 461.2.

### Example 81

### (9-(Ethylsulfonyl)-3-(tetrahydro-2H-pyran-4-yl)-2,3,4,9-tetrahydro-1H-carbazol-6-yl)((R)-3-hydroxypiperidin-1-yl)methanone

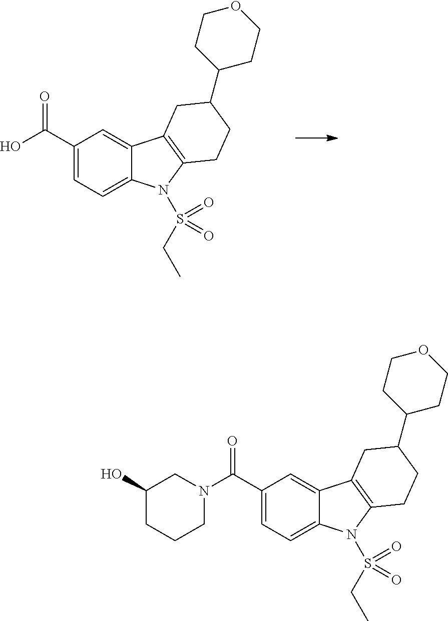

9-(Ethylsulfonyl)-3-(tetrahydro-2H-pyran-4-yl)-2,3,4,9-tetrahydro-1H-carbazole-6-carboxylic acid (160 mg, 0.41 mmol), (R)-piperidin-3-ol hydrochloride (56.2 mg, 0.41 mmol) and N-ethyl-N-isopropylpropan-2-amine (0.149 mL, 0.86 mmol) are mixed in DMF (10.0 mL), and HATU (155 mg, 0.41 mmol) is added. After 2 hours, the solvent is evaporated, and the residue is diluted in EtOAc (75 mL). The mixture is washed with 1N NaOH (75 mL), and the aqueous phase is extracted 3 times with EtOAc (3×75 mL). The organic phases are combined and dried over sodium sulfate. The mixture is filtered, and the solvent is evaporated. The product is purified by HPLC: Gilson prep pumps, flow rate: 30 mL/min, Gemini (5 μm particle size) 21.2×50 mm, mobile phase A=10 mM ammonium bicarbonate, B=MeCN, (132 mg, 68%). ¹H NMR (400 MHz, CHLOROFORM-D) δ ppm 1.19 (t, J=7.42 Hz, 3H) 1.36-1.66 (m, 10H) 1.72 (t, J=12.89 Hz, 2H) 2.08-2.19 (m, 1H) 2.26-2.39 (m, 1H) 2.78 (dd, J=16.02, 4.69 Hz, 1H) 2.82-2.93 (m, 1H) 3.06-3.18 (m, 1H) 3.21 (q, J=7.42 Hz, 2H) 3.40 (t, J=11.72 Hz, 2H) 3.92-4.00 (m, 1H) 4.03 (dd, J=11.52, 3.32 Hz, 2H) 7.27 (dd, J=8.20, 1.56 Hz, 1H) 7.50 (s, 1H) 7.95 (d, J=8.59 Hz, 1H); MS (ESI) (M+H)⁺ 475.2.

### Example 82

### (9-(Ethylsulfonyl)-3-(tetrahydro-2H-pyran-4-yl)-2,3,4,9-tetrahydro-1H-carbazol-6-yl)(4-hydroxypiperidin-1-yl)methanone

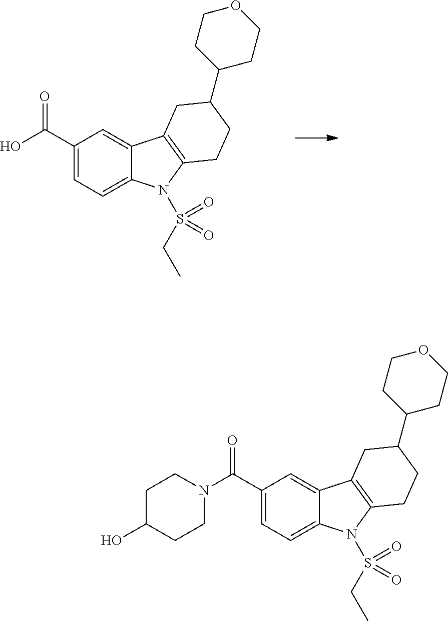

9-(Ethylsulfonyl)-3-(tetrahydro-2H-pyran-4-yl)-2,3,4,9-tetrahydro-1H-carbazole-6-carboxylic acid (160 mg, 0.41 mmol), piperidin-4-ol (55.0 mg, 0.54 mmol) and N-ethyl-N-isopropylpropan-2-amine (0.078 mL, 0.45 mmol) are mixed in DMF (10.0 mL), and HATU (155 mg, 0.41 mmol) is added. The mixture is stirred for 2 hours, and the solvent is evaporated. The residue is diluted in EtOAc (75 mL) and washed with 1N NaOH (75 mL). The aqueous phase is extracted 3 times with EtOAc (3×75 mL), and organic phases are combined and dried over sodium sulfate. The mixture is filtered, and the solvent is evaporated. The product is purified by HPLC: Gilson prep pumps, flow rate: 30 mL/min, Gemini (5 μm particle size) 21.2×50 mm, mobile phase A=10 mM ammonium bicarbonate, B=MeCN, (109 mg, 56%). ¹H NMR (400 MHz, CHLOROFORM-D) ppm 1.19 (t, J=7.42 Hz, 3H) 1.35-1.66 (m, 10H) 1.72 (t, J=13.28 Hz, 2H) 1.94 (s, 1H) 2.06-2.18 (m, 1H) 2.27-2.39 (m, J=9.77 Hz, 1H) 2.78 (dd, J=16.02, 3.91 Hz, 1H) 2.82-2.93 (m, 1H) 3.07-3.18 (m, 1H) 3.21 (q, J=7.42 Hz, 2H) 3.40 (t, J=11.72 Hz, 3H) 4.03 (dd, J=11.91, 2.93 Hz, 2H) 7.30 (dd, J=8.40, 1.37 Hz, 1H) 7.53 (s, 1H) 7.95 (d, J=8.59 Hz, 1H); MS (ESI) (M+H)⁺ 475.2.

### Example 83

### N6-Ethyl-N6-(2-(ethylamino)-2-oxoethyl)-N9,N9-dimethyl-3-(tetrahydro-2H-pyran-4-yl)-3,4-dihydro-1H-carbazole-6,9(2H)-dicarboxamide

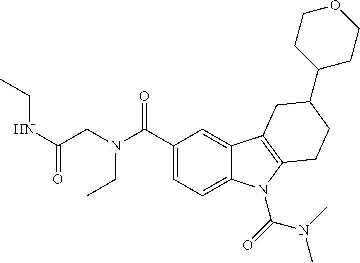

### Step A: 9-[(Dimethylamino)carbonyl]-3-(tetrahydro-2H-pyran-4-yl)-2,3,4,9-tetrahydro-1H-carbazole-6-carboxylic acid

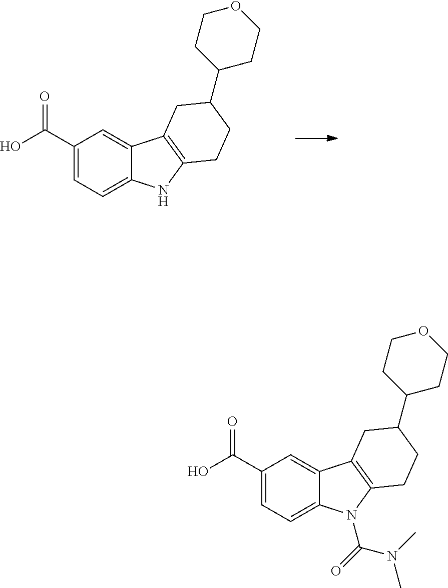

Solid KHMDS (800 mg, 4.00 mmol) is mixed in THF (10.0 mL) at −78° C., and 3-(tetrahydro-2H-pyran-4-yl)-2,3,4,9-tetrahydro-1H-carbazole-6-carboxylic acid (200 mg, 0.67 mmol) is added in one portion. The mixture is warmed to 0° C. and stirred for 10 minutes. The mixture is cooled to −78° C. and then stirred for 1 hour. Dimethylcarbamic chloride (0.55 mL, 6.00 mmol) is added, and the mixture is stirred for 3 hours at 0° C. Saturated ammonium chloride (100 mL) is added, and the aqueous phase is extracted 3 times with EtOAc (3×75 mL). The organic phases are combined and dried over sodium sulfate. The mixture is filtered, and the solvent is evaporated to yield a solid (205 mg), which is used without purification.

### Step B: N6-Ethyl-N6-(2-(ethylamino)-2-oxoethyl)-N9,N9-dimethyl-3-(tetrahydro-2H-pyran-4-yl)-3,4-dihydro-1H-carbazole-6,9(2H)-dicarboxamide

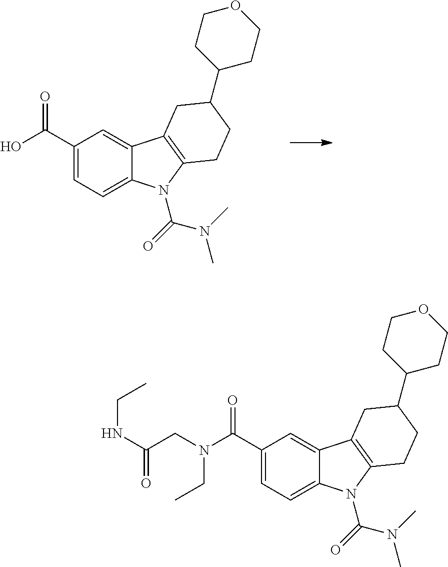

9-(Dimethylcarbamoyl)-3-(tetrahydro-2H-pyran-4-yl)-2,3,4,9-tetrahydro-1H-carbazole-6-carboxylic acid (205 mg, 0.55 mmol), N-ethyl-2-(ethylamino)acetamide (72.0 mg, 0.55 mmol) and N-ethyl-N-isopropylpropan-2-amine (0.096 mL, 0.55 mmol) are mixed in DMF (10.0 mL) at 0° C. HATU (210 mg, 0.55 mmol) is added, and the mixture is stirred for 1 hour. The solvent is evaporated, and the residue is diluted in EtOAc (75 mL). The mixture is washed with saturated ammonium chloride (75 mL), and the aqueous phase is extracted 3 times with EtOAc (3×75 mL). The organic phases are combined and dried over sodium sulfate. The solvent is evaporated, and the product is purified by HPLC: Gilson prep pumps, flow rate: 30 ml/min, Synergi Polar (4 μm particle size) 21.2×50 mm, mobile phase A=water (0.05% TFA), B=MeCN, (49 mg, 2 steps 18%). ¹H NMR (400 MHz, CHLOROFORM-D) δ ppm 1.09-1.26 (m, 6H) 1.37-1.95 (m, 10H) 2.02-2.16 (m, 1H) 2.27-2.44 (m, 1H) 2.72-2.94 (m, 3H) 3.04 (d, J=2.73 Hz, 6H) 3.26-3.37 (m, 2H) 3.41 (t, J=11.72 Hz, 2H) 3.48 (s, 1H) 4.03 (dd, J=11.13, 3.32 Hz, 2H) 4.10 (s, 1H) 7.22-7.26 (m, 2H) 7.53 (s, 1H); MS (ESI) (M+H)⁺ 483.3.

### Example 84

### N-ethyl-N-(2-(ethylamino)-2-oxoethyl)-3-(tetrahydro-2H-pyran-4-yl)-2,3,4,9-tetrahydro-1H-carbazole-6-carboxamide

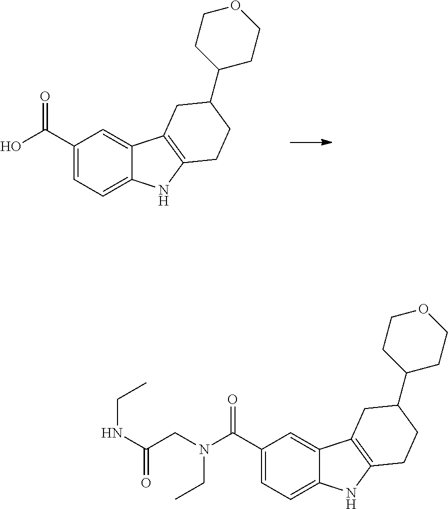

3-(Tetrahydro-2H-pyran-4-yl)-2,3,4,9-tetrahydro-1H-carbazole-6-carboxylic acid (1.51 g, 5.08 mmol) and N-ethyl-2-(ethylamino)acetamide (0.651 g, 5.08 mmol) are mixed in DMF (20.0 mL), and the resulting mixture is cooled to 0° C. N-Ethyl-N-isopropylpropan-2-amine (0.885 mL, 5.08 mmol) is added followed by HATU (1.931 g, 5.08 mmol). After 1 hour, the mixture is diluted with 1N HCl (100 mL). The aqueous phase is extracted 3 times with EtOAc (3×75 mL). The organic phases are combined and dried over sodium sulfate. The mixture is filtered, and the solvent is evaporated. The product is purified by HPLC: Gilson prep pumps, flow rate: 45 mL/min, X-Bridge Prep C18 OBD, 30×150 mm, 5 μm particle size, mobile phase A=10 mM ammonium bicarbonate, B=MeCN, (996 mg, 48%). ¹H NMR (400 MHz, CHLOROFORM-D) δ ppm 1.17 (t, J=7.42 Hz, 3H) 1.19-1.22 (m, 3H) 1.39-1.68 (m, 7H) 1.73 (t, J=12.11 Hz, 2H) 2.09b (d, J=11.72 Hz, 1H) 2.38 (dd, J=14.45, 9.77 Hz, 1H) 2.73-2.87 (m, 3H) 3.32 (dt, J=13.67, 7.03, 6.64 Hz, 2H) 3.49 (d, J=6.64 Hz, 2H) 4.03 (dd, J=11.33, 3.12 Hz, 2H) 4.07-4.16 (m, 2H) 7.17 (dd, J=8.20, 1.56 Hz, 1H) 7.27 (d, J=8.20 Hz, 1H) 7.55 (s, 1H) 7.90 (s, 1H); MS (ESI) (M+H)⁺ 412.3.

### Example 85

### Ethyl 2-(6-(ethyl(2-(ethylamino)-2-oxoethyl)carbamoyl)-3-(tetrahydro-2H-pyran-4-yl)-3,4-dihydro-1H-carbazol-9(2H)-yl)acetate

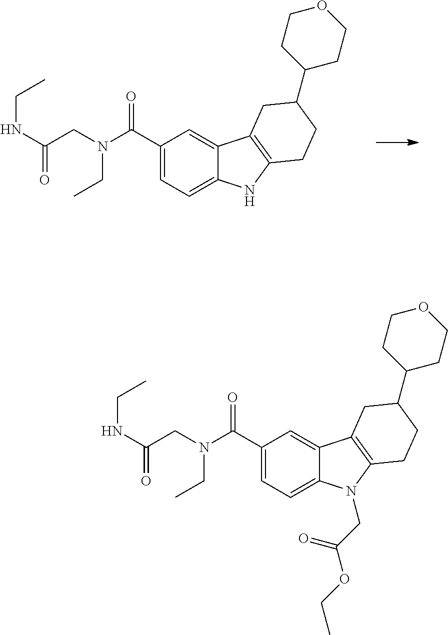

N-Ethyl-N-(2-(ethylamino)-2-oxoethyl)-3-(tetrahydro-2H-pyran-4-yl)-2,3,4,9-tetrahydro-1H-carbazole-6-carboxamide (0.996 g, 2.42 mmol) is mixed in THF (40.0 mL) and cooled to 0° C. Solid KHMDS (2.414 g, 12.10 mmol) is added, and the mixture is stirred at 0° C. for 30 minutes. Ethyl 2-iodoacetate (1.431 mL, 12.10 mmol) is added, and the mixture is stirred for 30 minutes. The mixture is diluted with a saturated solution of ammonium chloride (75 mL), and then extracted 3 times with EtOAc (3×75 mL). The organic phases are combined and dried over sodium sulfate. The mixture is filtered, and the solvent is evaporated. The product is purified by HPLC: Gilson prep pumps, flow rate: 45 ml/min, X-Bridge Prep C18 OBD, 30×150 mm, 5 μm particle size, mobile phase A=10 mM ammonium bicarbonate, B=MeCN, (618 mg, 51%). ¹H NMR (400 MHz, CHLOROFORM-D) δ ppm 1.11-1.20 (m, 6H) 1.24 (t, J=7.03 Hz, 2H) 1.30 (t, J=7.23 Hz, 1H) 1.37-1.65 (m, 5H) 1.71 (t, J=10.35 Hz, 2H) 2.07-2.21 (m, J=10.16 Hz, 1H) 2.39 (dd, J=14.06, 8.59 Hz, 1H) 2.57-2.77 (m, 2H) 2.82 (dd, J=15.23, 2.73 Hz, 1H) 3.30 (q, J=20.31, 14.06, 7.03 Hz, 2H) 3.40 (t, J=11.72 Hz, 2H) 3.48 (d, J=7.03 Hz, 2H) 4.02 (dd, J=11.13, 3.32 Hz, 2H) 4.08 (s, 1H) 4.17 (d, J=7.03 Hz, 1H) 4.21 (d, J=7.03 Hz, 1H) 4.22 (d, J=7.42 Hz, 1H) 4.25 (d, J=7.03 Hz, 1H) 4.72 (s, 2H) 7.15 (d, J=8.20 Hz, 1H) 7.20 (dd, J=8.59, 1.56 Hz, 1H) 7.56 (s, 1H); MS (ESI) (M+H)⁺ 498.4.

### Example 86

### 2-(6-(Ethyl(2-(ethylamino)-2-oxoethyl)carbamoyl)-3-(tetrahydro-2H-pyran-4-yl)-3,4-dihydro-1H-carbazol-9(2H)-yl)acetic acid

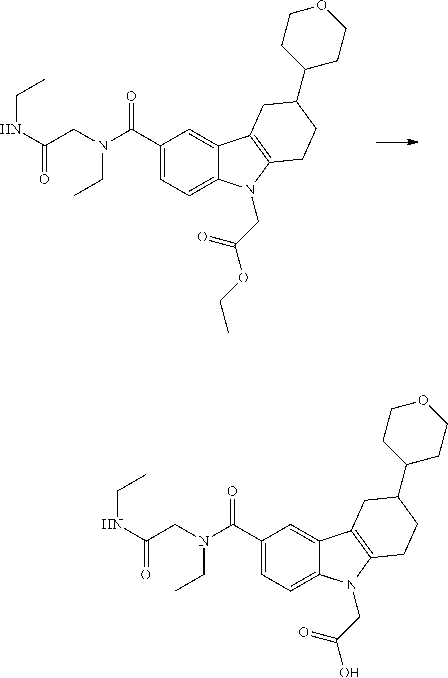

Ethyl 2-(6-(ethyl(2-(ethylamino)-2-oxoethyl)carbamoyl)-3-(tetrahydro-2H-pyran-4-yl)-3,4-dihydro-1H-carbazol-9(2H)-yl)acetate (50.0 mg, 0.10 mmol) is mixed in THF (5.00 mL), and 6N sodium hydroxide (5.00 mL, 30.00 mmol) is added. The mixture is stirred at room temperature for 12 hours. The solvents are evaporated, and the residue is dissolved in 1N NaOH (50 mL). The mixture is washed with EtOAc (50 mL), and the organic phase is extracted 3 times with 1N NaOH (3×50 mL). The aqueous phases are combined, and 6N HCl is added until the pH is acidic, as indicated by pH paper. The aqueous phase is extracted 3 times with EtOAc (3×50 mL). The organic phases are combined and dried over sodium sulfate. The mixture is filtered, and the solvent is evaporated. ¹H NMR (400 MHz, CHLOROFORM-D) δ ppm 1.13 (t, J=6.25 Hz, 6H) 1.24 (s, 1H) 1.38-1.61 (m, 5 H) 1.71 (t, J=10.55 Hz, 2H) 2.06 (s, 2H) 2.10 (d, J=8.59 Hz, 1H) 2.36 (s, 1H) 2.55-2.72 (m, 2H) 2.78 (t, J=7.03 Hz, 1H) 3.28 (t, J=6.25 Hz, 2H) 3.40 (t, J=11.33 Hz, 3H) 4.03 (d, J=10.16 Hz, 2H) 4.11 (s, 1H) 4.68 (s, 2H) 7.00-7.18 (m, 3H) 7.51 (s, 1H); MS (ESI) (M+H)⁺ 470.4.

### Example 87

### 9-(2-(diethylamino)-2-oxoethyl)-N-ethyl-N-(2-(ethylamino)-2-oxoethyl)-3-(tetrahydro-2H-pyran-4-yl)-2,3,4,9-tetrahydro-1H-carbazole-6-carboxamide

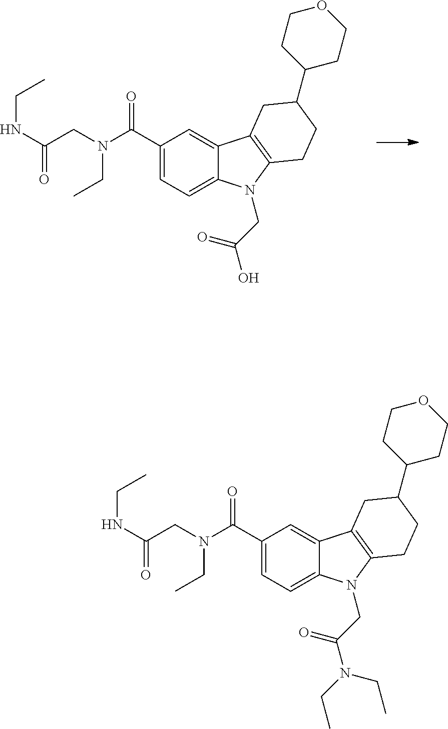

2-(6-(Ethyl(2-(ethylamino)-2-oxoethyl)carbamoyl)-3-(tetrahydro-2H-pyran-4-yl)-3,4-dihydro-1H-carbazol-9(2H)-yl)acetic acid (116 mg, 0.25 mmol) and diethylamine (0.038 mL, 0.37 mmol) are mixed in DMF (10.0 mL), and N-ethyl-N-isopropylpropan-2-amine (0.065 mL, 0.37 mmol) is added followed by HATU (141 mg, 0.37 mmol). The mixture is stirred at room temperature for 1 hour and then diluted with 1N HCl (75 mL). The aqueous phase is extracted 3 times with EtOAc (3×75 mL). The organic phases are combined and dried over sodium sulfate. The mixture is filtered, and the solvent is evaporated. The product is purified by HPLC: Gilson prep pumps, flow rate: 30 mL/min, Synergi Polar (4 μm particle size) 21.2×50 mm, mobile phase A=water (0.05% TFA), B=MeCN, (23 mg, 17%). ¹H NMR (400 MHz, CHLOROFORM-D) δ ppm 1.09-1.20 (m, 9H) 1.24 (t, J=7.03 Hz, 3H) 1.38-1.67 (m, 5H) 1.68-1.78 (m, 2H) 2.10 (d, J=11.72 Hz, 1H) 2.39 (dd, J=14.65, 9.57 Hz, 1H) 2.54-2.76 (m, 2H) 2.82 (dd, J=16.02, 4.30 Hz, 1H) 3.07 (s, 1H) 3.31 (dt, J=12.89, 7.42 Hz, 2H) 3.35-3.45 (m, 6H) 3.49 (d, J=7.03 Hz, 2H) 4.02 (dd, J=11.13, 3.32 Hz, 2H) 4.09 (s, 2H) 4.77 (d, J=2.34 Hz, 2H) 7.11 (d, J=8.59 Hz, 1H) 7.18 (dd, J=8.59, 1.56 Hz, 1H) 7.56 (d, J=0.78 Hz, 1H); MS (ESI) (M+H)⁺ 525.3.

### Example 88

### N-Ethyl-N-(2-(ethylamino)-2-oxoethyl)-9-(2-(methylamino)-2-oxoethyl)-3-(tetrahydro-2H-pyran-4-yl)-2,3,4,9-tetrahydro-1H-carbazole-6-carboxamide

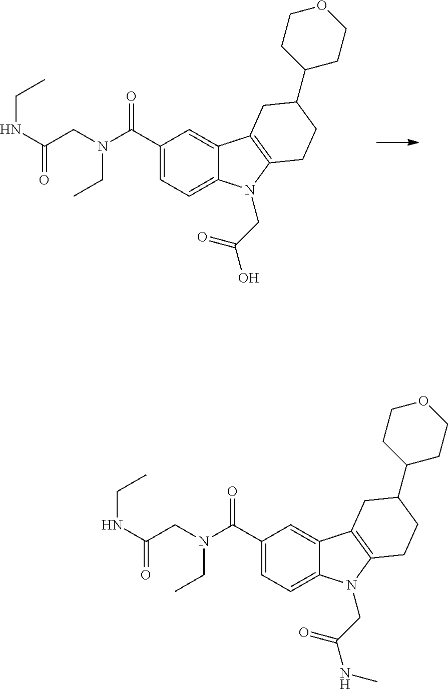

2-(6-(Ethyl(2-(ethylamino)-2-oxoethyl)carbamoyl)-3-(tetrahydro-2H-pyran-4-yl)-3,4-dihydro-1H-carbazol-9(2H)-yl)acetic acid (75.0 mg, 0.16 mmol) and methylamine hydrochloride (16.18 mg, 0.24 mmol) are mixed in DMF (10.0 mL), and N-ethyl-N-isopropylpropan-2-amine (0.042 mL, 0.24 mmol) is added followed by HATU (72.9 mg, 0.19 mmol). The mixture is stirred at room temperature for 30 minutes and then diluted with 1N HCl (75 mL). The aqueous phase is extracted 3 times with EtOAc (3×75 mL). The organic phases are combined and dried over sodium sulfate. The mixture is filtered, and the solvent is evaporated. The product is purified by HPLC: Gilson prep pumps, flow rate: 30 ml/min, Synergi Polar (4 μm particle size) 21.2×50 mm, mobile phase A=water (0.05% TFA), B=MeCN, (17.0 mg, 22%). ¹H NMR (400 MHz, CHLOROFORM-D) δ ppm 1.17 (t, J=7.23 Hz, 3H) 1.19-1.26 (m, 2H) 1.38-1.66 (m, 5H) 1.73 (t, J=8.98 Hz, 2H) 2.15 (d, J=10.94 Hz, 1H) 2.38 (dd, J=14.84, 9.37 Hz, 1H) 2.52-2.69 (m, 1H) 2.68-2.70 (m, 2H) 2.72 (d, J=4.69 Hz, 3H) 2.84 (dd, J=16.41, 3.13 Hz, 1H) 3.33 (q, J=14.06, 7.42, 7.42 Hz, 4H) 3.41 (t, J=11.33 Hz, 2H) 3.50 (d, J=5.47 Hz, 2H) 4.04 (dd, J=11.33, 3.12 Hz, 2H) 4.10 (s, 1H) 4.67 (s, 2H) 5.26-5.36 (m, 1H) 7.19 (d, J=8.20 Hz, 1H) 7.23 (dd, J=8.20, 0.78 Hz, 1H) 7.59 (s, 1H); MS (ESI) (M+H)⁺ 483.3.

### Example 89

### N-Ethyl-N-(2-(ethylamino)-2-oxoethyl)-9-(2-hydroxy-2-methylpropyl)-3-(tetrahydro-2H-pyran-4-yl)-2,3,4,9-tetrahydro-1H-carbazole-6-carboxamide

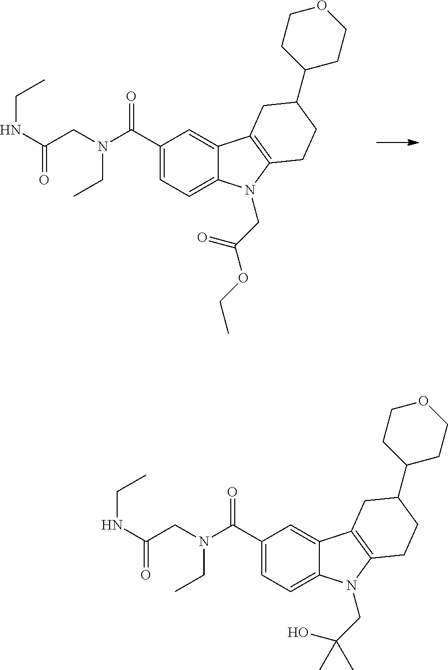

Ethyl 2-(6-(ethyl(2-(ethylamino)-2-oxoethyl)carbamoyl)-3-(tetrahydro-2H-pyran-4-yl)-3,4-dihydro-1H-carbazol-9(2H)-yl)acetate (105 mg, 0.21 mmol) is mixed in THF (10.0 mL) and cooled to 0° C. Methylmagnesium bromide (0.482 mL, 0.68 mmol) is added, and the mixture is stirred for 45 minutes. The mixture is diluted with a saturated solution of ammonium chloride (75 mL) and then extracted 3 times with EtOAc (3×75 mL). The organic phases are combined and dried over sodium sulfate. The mixture is filtered, and the solvent is evaporated. The product is purified by HPLC: Gilson prep pumps, flow rate: 45 ml/min, X-Bridge Prep C18 OBD, 30×150 mm, 5 μm particle size, mobile phase A=10 mM ammonium bicarbonate, B=MeCN, (16.95 mg, 17%). ¹H NMR (400 MHz, CHLOROFORM-D) δ ppm 1.13-1.23 (m, 5H) 1.28 (s, 6H) 1.38-1.80 (m, 9H) 2.13 (d, J=12.89 Hz, 1H) 2.39 (dd, J=14.84, 10.16 Hz, 1H) 2.65-2.77 (m, 1H) 2.85 (d, J=15.62 Hz, 2H) 3.32 (q, J=7.03, 6.25 Hz, 2H) 3.41 (dt, J=11.72, 1.95 Hz, 2H) 3.50 (q, J=6.51 Hz, 2H) 3.93-4.07 (m, 4H) 4.10 (s, 2H) 6.94 (s, 1H) 7.18 (dd, J=8.59, 1.56 Hz, 1H) 7.39 (d, J=8.59 Hz, 1H) 7.55 (d, J=1.17 Hz, 1H); MS (ESI) (M+H)⁺ 484.2.

### Example 90

### N-Ethyl-N-(2-(ethylamino)-2-oxoethyl)-9-(2-hydroxyethyl)-3-(tetrahydro-2H-pyran-4-yl)-2,3,4,9-tetrahydro-1H-carbazole-6-carboxamide

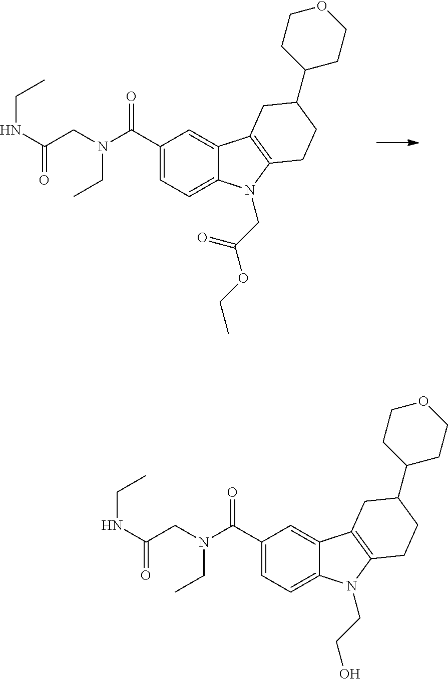

Ethyl 2-(6-(ethyl(2-(ethylamino)-2-oxoethyl)carbamoyl)-3-(tetrahydro-2H-pyran-4-yl)-3,4-dihydro-1H-carbazol-9(2H)-yl)acetate (105 mg, 0.21 mmol) is mixed in THF (10.0 mL) and cooled to 0° C. LAH (0.264 mL, 0.53 mmol) is added, and the mixture is stirred at 0° C. for 20 minutes. The mixture is diluted with a saturated solution of ammonium chloride (75 mL) and extracted 3 times with EtOAc (3×75 mL). The organic phases are combined and dried over sodium sulfate. The mixture is filtered, and the solvent is evaporated. The product is purified by HPLC: Gilson prep pumps, flow rate: 45 mL/min, X-Bridge Prep C18 OBD, 30×150 mm, 5 μm particle size, mobile phase A=10 mM ammonium bicarbonate, B=MeCN, (40.0 mg, 42%). ¹H NMR (400 MHz, CHLOROFORM-D) δ ppm 1.16 (t, J=7.23 Hz, 3H) 1.40-1.62 (m, 4H) 1.65 (s, 2H) 1.72 (t, J=11.33 Hz, 3H) 2.13 (d, J=11.72 Hz, 1H) 2.38 (dd, J=14.84, 9.37 Hz, 1H) 2.65-2.78 (m, 1H) 2.80-2.91 (m, J=5.86 Hz, 2H) 3.31 (ddd, J=13.28, 7.42, 5.86 Hz, 2H) 3.39 (t, J=11.72 Hz, 3H) 3.48 (q, J=14.06, 7.42, 6.64 Hz, 3H) 3.89 (t, J=5.08 Hz, 2H) 4.02 (dd, J=10.94, 3.13 Hz, 2H) 4.08 (s, 2H) 4.19 (q, J=9.77, 5.47, 4.30 Hz, 2H) 6.92 (s, 1H) 7.18 (dd, J=8.59, 1.56 Hz, 1H) 7.28 (d, J=8.59 Hz, 1H) 7.56 (s, 1H); MS (ESI) (M+H)⁺ 456.2.

### Example 91

### 2-(N-Ethyl-9-(ethylsulfonyl)-3-(tetrahydro-2H-pyran-4-yl)-2,3,4,9-tetrahydro-1H-carbazole-6-carboxamido)acetic acid

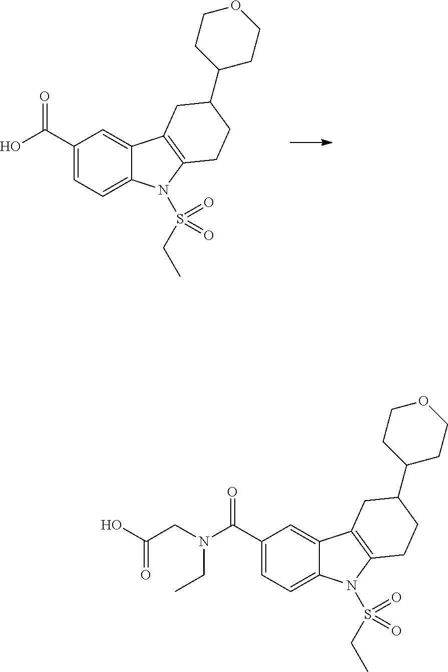

9-(Ethylsulfonyl)-3-(tetrahydro-2H-pyran-4-yl)-2,3,4,9-tetrahydro-1H-carbazole-6-carboxylic acid (668 mg, 1.71 mmol) is mixed in DMF (20.0 mL) and N-ethyl-N-isopropylpropan-2-amine (0.297 mL, 1.71 mmol) followed by HATU (649 mg, 1.71 mmol) are added. The mixture is stirred for 30 minutes and then diluted with 1N HCl (75 mL). The aqueous phase is extracted 3 times with DCM (3×75 mL). The organic phases are combined and dried over sodium sulfate. The mixture is filtered, and the solvent is evaporated. The product is purified by HPLC: Gilson prep pumps, flow rate: 45 ml/min, X-Bridge Prep C18 OBD, 30×150 mm, 5 μm particle size, mobile phase A=10 mM ammonium bicarbonate, B=MeCN, (413 mg, 51%). ¹H NMR (400 MHz, CHLOROFORM-D) δ ppm 1.11-1.28 (m, 6H) 1.37-1.65 (m, 5H) 1.67-1.79 (m, 2H) 1.98-2.01 (m, 1H) 2.07-2.17 (m, 2H) 2.23-2.47 (m, J=10.55 Hz, 1H) 2.69-2.91 (m, 2H) 3.06-3.29 (m, 3H) 3.41 (t, J=10.55 Hz, 3H) 3.98-4.11 (m, 2H) 4.26 (s, 1H) 7.31 (d, J=8.20 Hz, 1H) 7.54 (s, 1H) 7.74 (s, 1H) 7.95 (d, J=8.20 Hz, 1H); MS (ESI) (M+H)⁺ 477.4.

### Example 92

### N-Ethyl-9-(ethylsulfonyl)-N-(2-(2-hydroxypropylamino)-2-oxoethyl)-3-(tetrahydro-2H-pyran-4-yl)-2,3,4,9-tetrahydro-1H-carbazole-6-carboxamide

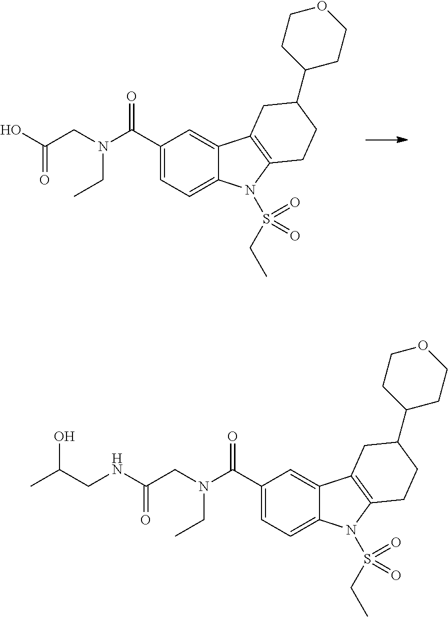

2-(N-Ethyl-9-(ethylsulfonyl)-3-(tetrahydro-2H-pyran-4-yl)-2,3,4,9-tetrahydro-1H-carbazole-6-carboxamido)acetic acid (189 mg, 0.40 mmol) and 1-aminopropan-2-ol (29.8 mg, 0.40 mmol) are mixed in DMF (10.0 mL), and N-ethyl-N-isopropylpropan-2-amine (0.069 mL, 0.40 mmol) is added followed by HATU (151 mg, 0.40 mmol). The mixture is stirred for 30 minutes and then diluted with 1N HCl (75 mL). The aqueous phase is extracted 3 times with EtOAc (3×75 mL). The organic phases are combined and dried over sodium sulfate. The mixture is filtered, and the solvent is evaporated. The product is purified the product by HPLC: Gilson prep pumps, flow rate: 45 ml/min, X-Bridge Prep C18 OBD, 30×150 mm, 5 μm particle size, mobile phase A=10 mM ammonium bicarbonate, B=MeCN, (116.1 mg, 55%). ¹H NMR (400 MHz, CHLOROFORM-D) δ ppm 1.19 (d, J=6.25 Hz, 6H) 1.20-1.25 (m, 2H) 1.37-1.65 (m, 5H) 1.72 (t, J=12.11 Hz, 2H) 2.13 (d, J=10.94 Hz, 1H) 2.32 (s, 1H) 2.73-2.90 (m, 2H) 3.08-3.28 (m, 5H) 3.40 (t, J=11.72 Hz, 2H) 3.44-3.57 (m, 2H) 3.92-3.99 (m, 1H) 4.03 (dd, J=11.52, 2.93 Hz, 2H) 4.08-4.17 (m, J=7.03 Hz, 4H) 7.05 (s, 1H) 7.30 (d, J=8.59 Hz, 1H) 7.47-7.56 (m, J=8.20 Hz, 1H) 7.97 (d, J=8.59 Hz, 1H); MS (ESI) (M+H)⁺ 534.4.

### Example 93

### N-Ethyl-9-(ethylsulfonyl)-N-(2-(2-methoxyethylamino)-2-oxoethyl)-3-(tetrahydro-2H-pyran-4-yl)-2,3,4,9-tetrahydro-1H-carbazole-6-carboxamide

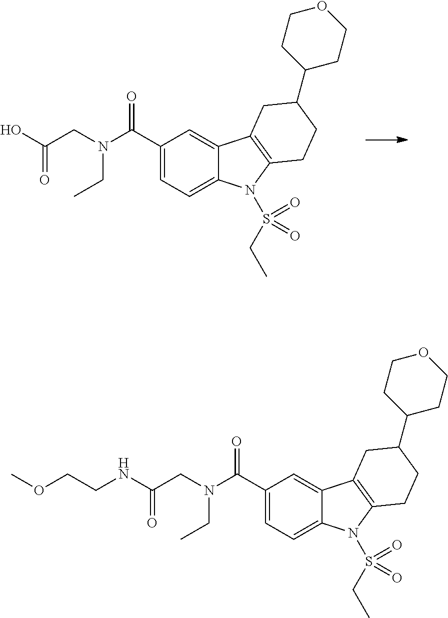

2-(N-Ethyl-9-(ethylsulfonyl)-3-(tetrahydro-2H-pyran-4-yl)-2,3,4,9-tetrahydro-1H-carbazole-6-carboxamido)acetic acid (80 mg, 0.17 mmol) and 2-methoxyethanamine (0.015 mL, 0.17 mmol) are mixed in DMF (10.0 mL), and N-ethyl-N-isopropylpropan-2-amine (0.029 mL, 0.17 mmol) is added followed by HATU (63.8 mg, 0.17 mmol). The mixture is stirred for 30 minutes and then diluted with 1N HCl (75 mL). The aqueous phase is extracted 3 times with EtOAc (3×75 mL). The organic phases are combined and dried over sodium sulfate. The mixture is filtered, and the solvent is evaporated. The product is purified by HPLC: Gilson prep pumps, flow rate: 45 ml/min, X-Bridge Prep C18 OBD, 30×150 mm, 5 μm particle size, mobile phase A=10 mM ammonium bicarbonate, B=MeCN, (49.3 mg, 55%). ¹H NMR (400 MHz, CHLOROFORM-D) δ ppm 1.14-1.17 (m, 1H) 1.20 (t, J=7.42 Hz, 6H) 1.35-1.64 (m, 5H) 1.66 (s, 2H) 1.71 (t, J=14.06 Hz, 2H) 2.12 (d, J=11.72 Hz, 1H) 2.32 (dd, J=15.43, 9.96 Hz, 1H) 2.71-2.90 (m, 2H) 3.11-3.18 (m, 1H) 3.22 (q, J=7.42 Hz, 2H) 3.37 (s, 3H) 3.41 (d, J=12.11 Hz, 2H) 3.48 (s, 4H) 4.03 (dd, J=11.33, 3.13 Hz, 2H) 4.14 (s, 1H) 6.84 (s, 1H) 7.30 (dd, J=8.59, 1.56 Hz, 1H) 7.52 (d, J=1.17 Hz, 1H) 7.96 (d, J=8.59 Hz, 1H); MS (ESI) (M+H)⁺ 534.4.

### Example 94

### N-Ethyl-9-(ethylsulfonyl)-N-(2-(oxetan-3-ylamino)-2-oxoethyl)-3-(tetrahydro-2H-pyran-4-yl)-2,3,4,9-tetrahydro-1H-carbazole-6-carboxamide

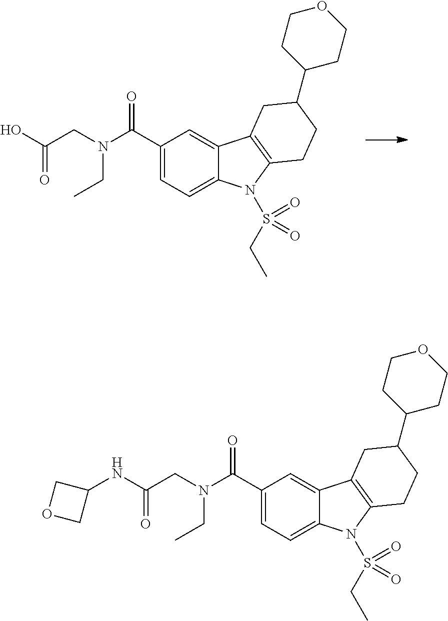

2-(N-Ethyl-9-(ethylsulfonyl)-3-(tetrahydro-2H-pyran-4-yl)-2,3,4,9-tetrahydro-1H-carbazole-6-carboxamido)acetic acid (100 mg, 0.21 mmol) and oxetan-3-amine hydrochloride (22.99 mg, 0.21 mmol) are mixed in DMF (10.0 mL) and N-ethyl-N-isopropylpropan-2-amine (0.037 mL, 0.21 mmol) is added followed by HATU (80 mg, 0.21 mmol). The mixture is stirred for 30 minutes and then diluted with 1N HCl (75 mL). The aqueous phase is extracted 3 times with EtOAc (3×75 mL). The organic phases are combined and dried over sodium sulfate. The mixture is filtered, and the solvent is evaporated. The product is purified by HPLC: Gilson prep pumps, flow rate: 45 ml/min, X-Bridge Prep C18 OBD, 30×150 mm, 5 μm particle size, mobile phase A=10 mM ammonium bicarbonate, B=MeCN, (69 mg, 62%). ¹H NMR (400 MHz, CHLOROFORM-D) δ ppm 1.16-1.25 (m, 5H) 1.37-1.58 (m, 5H) 1.62 (s, 4H) 1.72 (t, J=12.50 Hz, 2H) 2.14 (d, J=12.50 Hz, 1H) 2.27-2.40 (m, 1H) 2.73-2.91 (m, 2H) 3.09-3.20 (m, 1H) 3.23 (q, J=7.42 Hz, 2H) 3.40 (t, J=11.72 Hz, 2H) 3.44-3.53 (m, 1 H) 4.03 (dd, J=11.13, 2.93 Hz, 2H) 4.11 (s, 1H) 4.55 (t, J=6.64 Hz, 2H) 4.93 (t, J=7.03 Hz, 2H) 5.05 (quint, J=7.03 Hz, 1H) 7.30 (d, J=9.77 Hz, 1H) 7.53 (s, 1H) 7.98 (d, J=7.81 Hz, 1H); MS (ESI) (M+H)⁺ 532.3.

### Example 95

### N-[2-(cyclopropylamino)-2-oxoethyl]-9-(cyclopropylmethyl)-N-ethyl-3-(tetrahydro-2H-pyran-4-yl)-2,3,4,9-tetrahydro-1H-carbazole-6-carboxamide

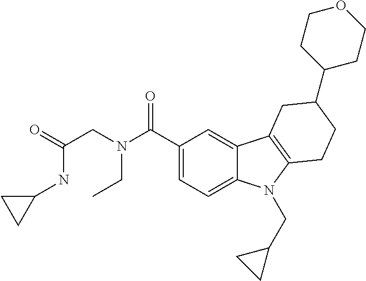

### Step A: N-[2-(cyclopropylamino)-2-oxoethyl]-9-(cyclopropylmethyl)-N-ethyl-3-(tetrahydro-2H-pyran-4-yl)-2,3,4,9-tetrahydro-1H-carbazole-6-carboxamide

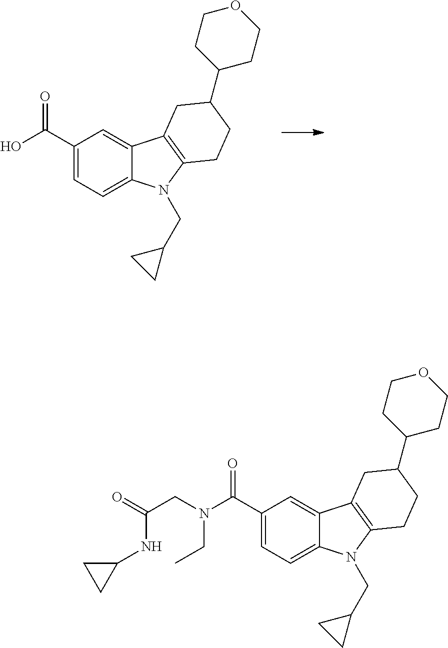

N,N-Diisopropylethylamine (89 μL, 0.51 mmol) was added to a solution of 9-(cyclopropylmethyl)-3-(tetrahydro-2H-pyran-4-yl)-2,3,4,9-tetrahydro-1H-carbazole-6-carboxylic acid (0.17 mmol) and N-cyclopropyl-2-(ethylamino)acetamide hydrochloride (61 mg, 0.34 mmol) in DMF (5 mL). Stirring for 20 min, HATU (97 mg, 0.26 mmol) was added at 0° C. The mixture was stirred for 3 h at room temperature, diluted with water (50 mL), and extracted with EtOAc (3×25 mL). The combined organic phases were washed with water (2×20 mL), saturated NaCl (20 mL) and dried with Na₂SO₄. Upon evaporation of the solvent, the crude product was purified by reverse-phase HPLC using high pH column 30-50% MeCN/H₂O to give 67.8 mg (83%) of a white solid as the title compound. ¹H NMR (400 MHz, METHANOL-D4) δ 0.29-0.38 (m, 2H), 0.40-0.60 (m, 4H), 0.72 (m, 2H), 1.08-1.30 (m, 4H), 1.38-1.66 (m, 5H), 1.74-1.88 (m, 2H), 2.19 (m, 1H), 2.32-2.49 (m, 1H), 2.62-2.97 (m, 3H), 3.38-3.66 (m, 4H), 3.90-4.16 (m, 7H), 7.13-7.25 (m, 1H), 7.31-7.43 (m, 1H), 7.45-7.60 (m, 1H); HRMS m/z calcd for [M+H]⁺ 478.30642. found 478.30499.

### Step B: methyl 3-chloro-4-(cyclopropylmethylamino)benzoate

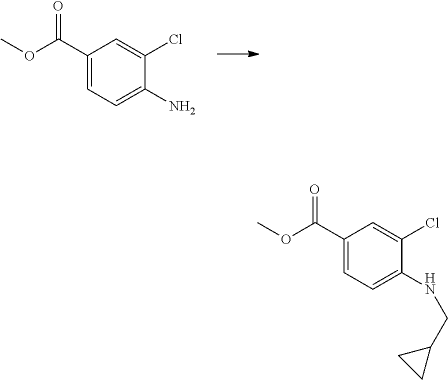

Sodium triacetoxyborohydride (3.47 g, 16.39 mmol) was added to a solution of methyl 4-amino-3-chlorobenzoate (1.014 g, 5.46 mmol), cyclopropanecarboxaldehyde (0.816 mL, 10.93 mmol), and acetic acid (1.876 mL, 32.78 mmol) in CH₂Cl₂ (40 mL). The reaction mixture was stirred at room temperature under nitrogen for 24 h. After concentration, the product was taken up with EtOAc (100 mL), washed with saturated NaHCO₃ (3×20 mL), NaCl (20 mL) and dried over Na₂SO₄. The crude product was purified by MPLC on silica gel using Hex/EtOAc (4:1) to give 1.154 g (88%) of a colorless oil. ¹H NMR (400 MHz, CHLOROFORM-D) δ 0.27-0.32 (m, 2H), 0.59-0.65 (m, 2H), 1.09-1.21 (m, 1H), 3.07 (dd, J=7.03, 5.08 Hz, 2H), 3.86 (s, 3H), 4.82-4.95 (m, 1H), 6.59 (d, J=8.59 Hz, 1H), 7.82 (dd, J=8.59, 1.95 Hz, 1H), 7.95 (d, J=1.95 Hz, 1H); MS (ESI) (M+H)⁺: 240.13.

### Step C: methyl 9-(cyclopropylmethyl)-3-(tetrahydro-2H-pyran-4-yl)-2,3,4,9-tetrahydro-1H-carbazole-6-carboxylate

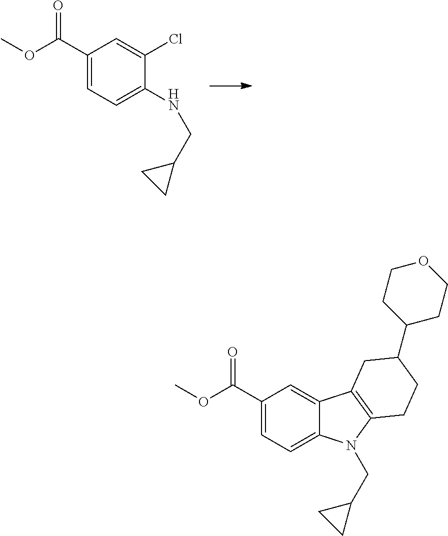

Methyl 3-chloro-4-(cyclopropylmethylamino)benzoate (0.360 g, 1.5 mmol), 4-(tetrahydro-2H-pyran-4-yl)cyclohexanone (0.547 g, 3.00 mmol), acetic acid (0.129 mL, 0.135 g, 2.25 mmol), and magnesium sulfate (0.090 g, 0.75 mmol) were suspended in DMA (5 mL). Nitrogen was bubbled through the solution for 10 min. Potassium phosphate (0.414 g, 1.95 mmol) and bis(tri-t-butylphosphine)palladium(0) (0.077 g, 0.15 mmol) were added, and nitrogen was bubbled through the mixture for an additional 5 min. The reaction mixture was heated for 3 h at 110° C. After cooling to room temperature, the reaction mixture was filtered through Celite. The filtrate was diluted with EtOAc (100 mL), washed with water (3×15 mL) and NaCl (2×15 mL), and dried with Na₂SO₄. The crude product was purified by MPLC on silica gel using Hex/EtOAc (4:1) to give 0.125 g (23%) of a white solid. ¹H NMR (400 MHz, CHLOROFORM-D) δ 0.33 (m, 2H), 0.51-0.58 (m, 2H), 1.11-1.22 (m, 1H), 1.40-1.69 (m, 5H), 1.71-1.82 (m, 2H), 2.10-2.21 (m, 1H), 2.39-2.49 (m, 1H), 2.65-2.87 (m, 2H), 2.88-2.97 (m, 1H), 3.37-3.49 (m, 2H), 3.84-3.91 (m, 1H), 3.93 (s, 3H), 3.96-4.01 (m, 1H), 4.02-4.08 (m, 2H), 7.28 (d, J=8.59 Hz, 1H), 7.82-7.89 (m, 1H), 8.23 (s, 1H); MS (ESI) (M+H)⁺: 368.24.

### Step D: 9-(cyclopropylmethyl)-3-(tetrahydro-2H-pyran-4-yl)-2,3,4,9-tetrahydro-1H-carbazole-6-carboxylic acid

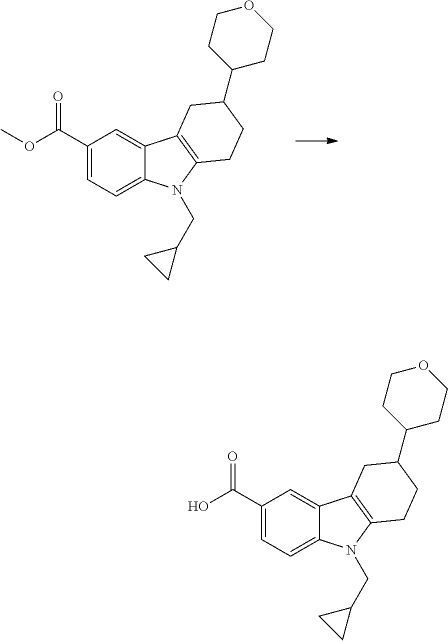

Lithium hydroxide (16 mg, 0.68 mmol) was added to a solution of methyl 9-(cyclopropylmethyl)-3-(tetrahydro-2H-pyran-4-yl)-2,3,4,9-tetrahydro-1H-carbazole-6-carboxylate (125 mg, 0.34 mmol) in ethanol (5 mL) and water (0.5 mL). The reaction mixture was heated for 4 h at 80° C. After cooling to room temperature, 2 N HCl (2 mL) was added. Upon evaporation and dried in vacuo, the white solid was dissolved in DMF (10 mL) and used directly for next step. MS (ESI) (M+H)⁺: 354.23.

### Example 96

### 9-(Cyclopropylmethyl)-N-ethyl-N-(2-(ethylamino)-2-oxoethyl)-3-(tetrahydro-2H-pyran-4-yl)-2,3,4,9-tetrahydro-1H-carbazole-6-carboxamide

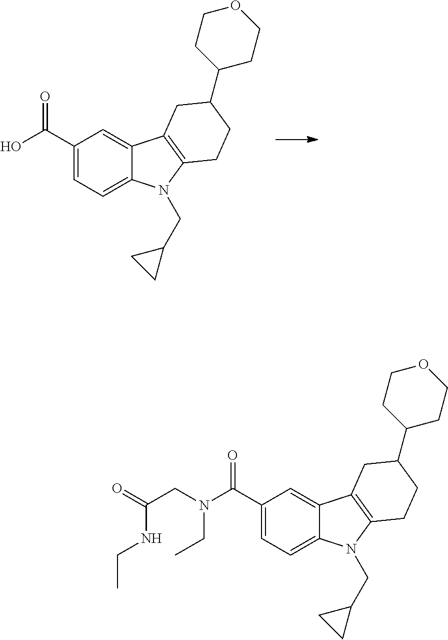

N,N-Diisopropylethylamine (89 μL, 0.51 mmol) was added to a solution of 9-(cyclopropylmethyl)-3-(tetrahydro-2H-pyran-4-yl)-2,3,4,9-tetrahydro-1H-carbazole-6-carboxylic acid (0.17 mmol) and N-ethyl-2-(ethylamino)acetamide (44 mg, 0.34 mmol) in DMF (5 mL). Stirring for 20 min, HATU (97 mg, 0.26 mmol) was added at 0° C. The mixture was stirred for 3 h at room temperature, diluted with water (50 mL), and extracted with EtOAc (3×25 mL). The combined organic phases were washed with water (2×20 mL), saturated NaCl (20 mL) and dried with Na₂SO₄. Upon evaporation of the solvent, the crude product was purified by reverse-phase HPLC using high pH column 30-50% MeCN/H₂O to give 62.8 mg (79%) of a white solid as the title compound. ¹H NMR (400 MHz, METHANOL-D4): δ 0.28-0.38 (m, 2H), 0.46-0.56 (m, 2H), 1.05-1.31 (m, 6H), 1.38-1.52 (m, 2H), 1.53-1.67 (m, 3H), 1.79 (t, J=13.28 Hz, 2H), 2.13-2.25 (m, 1H), 2.33-2.48 (m, 1H), 2.67-2.81 (m, 1H), 2.79-2.95 (m, 2H), 3.19-3.27 (m, 3H), 3.38-3.64 (m, 4H), 3.89-4.19 (m, 6H), 7.14-7.24 (m, 1H), 7.33-7.42 (m, 1H), 7.49-7.62 (m, 1H); HRMS m/z calcd for [M+H]⁺ 466.30642. found 466.30549.

### Example 97

### 9-Cyclobutyl-N-ethyl-N-(2-(ethylamino)-2-oxoethyl)-3-(tetrahydro-2H-pyran-4-yl)-2,3,4,9-tetrahydro-1H-carbazole-6-carboxamide

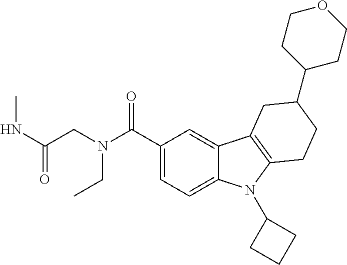

### Step A: 9-Cyclobutyl-N-ethyl-N-(2-(ethylamino)-2-oxoethyl)-3-(tetrahydro-2H-pyran-4-yl)-2,3,4,9-tetrahydro-1H-carbazole-6-carboxamide

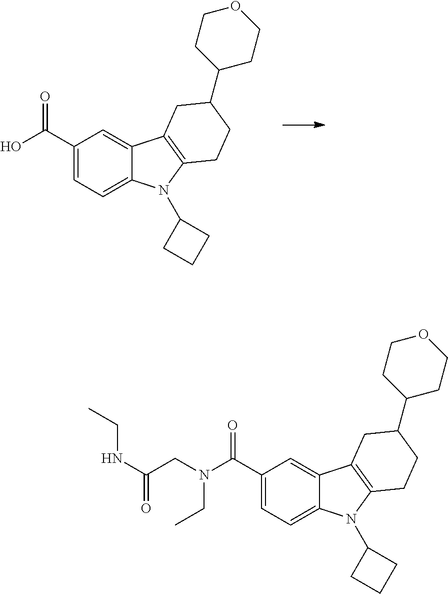

N,N-Diisopropylethylamine (89 μL, 0.51 mmol) was added to a solution of 9-cyclobutyl-3-(tetrahydro-2H-pyran-4-yl)-2,3,4,9-tetrahydro-1H-carbazole-6-carboxylic acid (0.17 mmol) and N-ethyl-2-(ethylamino)acetamide (44 mg, 0.34 mmol) in DMF (5 mL). Stirring for 20 min, HATU (97 mg, 0.26 mmol) was added at 0° C. The mixture was stirred for 3 h at room temperature, diluted with water (50 mL), and extracted with EtOAc (3×25 mL). The combined organic phases were washed with water (2×20 mL), saturated NaCl (20 mL) and dried with Na₂SO₄. Upon evaporation of the solvent, the crude product was purified by reverse-phase HPLC using high pH column 40-60% MeCN/H₂O to give 59.3 mg (75%) of a white solid as the title compound. ¹H NMR (400 MHz, METHANOL-D4): δ 1.06-1.29 (m, 6H), 1.36-1.50 (m, 2H), 1.50-1.63 (m, 3 H), 1.78 (t, J=13.48 Hz, 2H), 1.86-2.05 (m, 2H), 2.11-2.21 (m, 1H), 2.30-2.42 (m, 1H), 2.42-2.60 (m, 2H), 2.69-2.98 (m, 5H), 3.19-3.29 (m, 2H), 3.37-3.64 (m, 4H), 3.99 (dd, J=11.13, 3.71 Hz, 2H), 4.05-4.21 (m, 2H), 4.84-4.96 (m, 1H), 7.18 (d, J=8.59 Hz, 1H), 7.48-7.58 (m, 1H), 7.61 (d, J=8.20 Hz, 1H); MS (APPI) (M+H)⁺: 466.2; FIRMS m/z calcd for [M+H]⁺ 466.30642. found 466.30464.

### Step B: methyl 3-chloro-4-(cyclobutylamino)benzoate

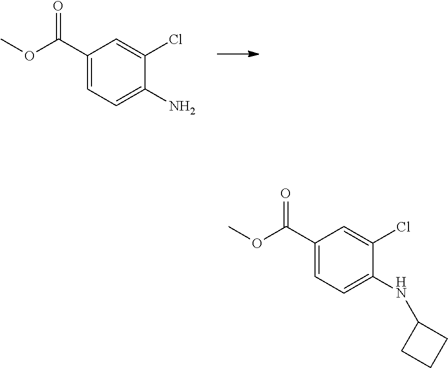

Sodium triacetoxyborohydride (1.75 g, 8.24 mmol) was added to a solution of methyl 4-amino-3-chlorobenzoate (0.51 g, 2.75 mmol), cyclobutanone (0.41 mL, 0.39 g, 5.50 mmol), and acetic acid (0.16 mL, 0.17 g, 2.75 mmol) were mixed in CH₂Cl₂ (20 mL). The reaction mixture was stirred at room temperature under nitrogen for a weekend. After concentration, the product was taken up with EtOAc (100 mL), washed with saturated NaHCO₃ (3×20 mL), NaCl (20 mL) and dried over Na₂SO₄. The crude product was purified by reverse-phase HPLC using high pH column 50-70% MeCN/H₂O to give 0.332 g (50%) of a white solid as the title compound. ¹H NMR (400 MHz, CHLOROFORM-D): δ 1.77-2.04 (m, 4H), 2.34-2.56 (m, 2H), 3.85 (s, 3H), 3.92-4.07 (m, 1H), 4.89 (d, J=5.47 Hz, 1H), 6.52 (d, J=8.59 Hz, 1H), 7.80 (dd, J=8.59, 1.56 Hz, 1H), 7.93 (d, J=1.95 Hz, 1H); MS (ESI) (M+H)⁺: 240.16.

### Step C: methyl 9-cyclobutyl-3-(tetrahydro-2H-pyran-4-yl)-2,3,4,9-tetrahydro-1H-carbazole-6-carboxylate

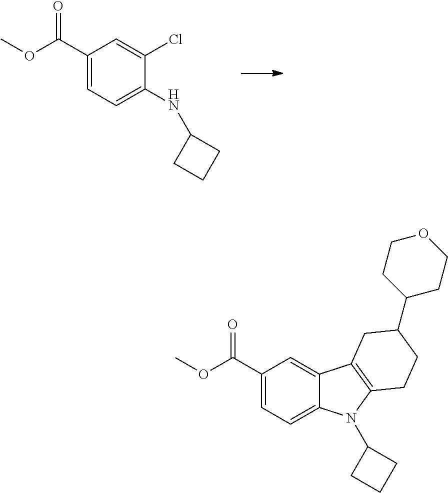

Methyl 3-chloro-4-(cyclobutylamino)benzoate (165 mg, 0.69 mmol), 4-(tetrahydro-2H-pyran-4-yl)cyclohexanone (376 mg, 2.07 mmol), acetic acid (59 μL, 62 mg 1.03 mmol), and magnesium sulfate (41 mg, 0.34 mmol) were suspended in DMA (3 mL). Nitrogen was bubbled through the solution for 10 min. Potassium phosphate (190 mg, 0.89 mmol) and Bis(tri-t-butylphosphine)palladium(0) (35 mg, 0.07 mmol) were added, and nitrogen was bubbled through the mixture for an additional 5 min. The reaction mixture was heated for 14 h at 140° C. After cooling to room temperature, the reaction mixture was diluted with water (15 mL), and extracted with EtOAc (4×20 mL). The combined organic phases were washed with water (2×15 mL) and NaCl (2×15 mL), and dried with Na₂SO₄. The crude product was purified by MPLC on silica gel using Hex/EtOAc (4:1) to give 195 mg (77%) of a white solid as the title compound. ¹H NMR (400 MHz, CHLOROFORM-D): δ 1.39-1.66 (m, 5H), 1.76 (d, J=12.11 Hz, 2H), 1.83-2.07 (m, 2H), 2.09-2.19 (m, 1H), 2.32-2.62 (m, 3H), 2.67-2.79 (m, 1H), 2.81-2.97 (m, 4H), 3.42 (t, J=11.52 Hz, 2H), 3.93 (s, 3H), 3.99-4.09 (m, 2H), 4.71-4.90 (m, 1H), 7.52 (d, J=8.59 Hz, 1H), 7.82 (dd, J=8.59, 1.56 Hz, 1H), 8.20 (s, 1H); MS (ESI) (M+H)⁺: 368.21.

### Step D: 9-(cyclopropylmethyl)-3-(tetrahydro-2H-pyran-4-yl)-2,3,4,9-tetrahydro-1H-carbazole-6-carboxylic acid

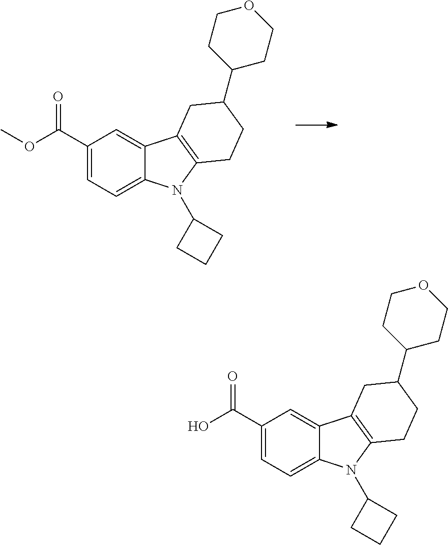

Lithium hydroxide (50 mg, 2.09 mmol) was added to a solution of methyl 9-cyclobutyl-3-(tetrahydro-2H-pyran-4-yl)-2,3,4,9-tetrahydro-1H-carbazole-6-carboxylate (195 mg, 0.53 mmol) in ethanol (5 mL) and water (0.5 mL). The reaction mixture was heated for 4 h at 80° C. After cooling to room temperature, 2 N HCl (2 mL) was added. After evaporation and dried in vacuo, a white solid was obtained, which was dissolved in DMF (15 mL) and used directly for next step without further purification. MS (ESI) (M+H)⁺: 354.22.

### Example 98

### 9-Cyclobutyl-N-ethyl-N-(2-(2-fluoroethylamino)-2-oxoethyl)-3-(tetrahydro-2H-pyran-4-yl)-2,3,4,9-tetrahydro-1H-carbazole-6-carboxamide

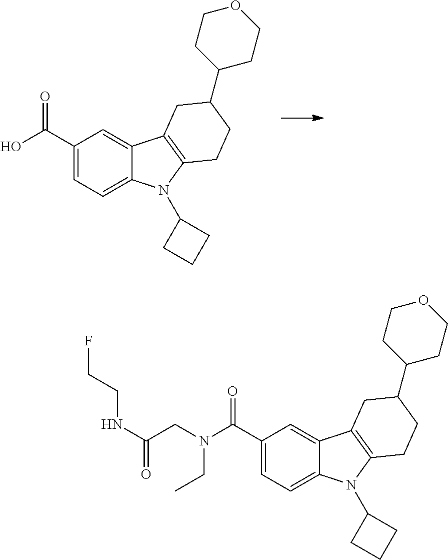

N,N-Diisopropylethylamine (89 μL, 0.51 mmol) was added to a solution of 9-cyclobutyl-3-(tetrahydro-2H-pyran-4-yl)-2,3,4,9-tetrahydro-1H-carbazole-6-carboxylic acid (0.17 mmol) and 2-(ethylamino)-N-(2-fluoroethyl)acetamide hydrochloride (63 mg, 0.34 mmol) in DMF (5 mL). Stirring for 20 min, HATU (97 mg, 0.26 mmol) was added at 0° C. The mixture was stirred for 3 h at room temperature, diluted with water (50 mL), and extracted with EtOAc (3×25 mL). The combined organic phases were washed with water (2×20 mL), saturated NaCl (20 mL) and dried with Na₂SO₄. Upon evaporation of the solvent, the crude product was purified by reverse-phase HPLC using high pH column 40-60% MeCN/H₂O to give 59.8 mg (73%) of a white solid as the title compound. 1H NMR (400 MHz, METHANOL-D4) δ 1.10-1.31 (m, 3H), 1.36-1.51 (m, 2H), 1.51-1.65 (m, 3H), 1.79 (t, J=12.89 Hz, 2H), 1.87-2.08 (m, 2H), 2.12-2.24 (m, 1H), 2.30-2.43 (m, 1H), 2.43-2.59 (m, 2H), 2.67-3.00 (m, 5H), 3.37-3.64 (m, 6H), 4.00 (dd, J=11.33, 3.12 Hz, 2H), 4.04-4.27 (m, 2H), 4.34-4.62 (m, 2H), 4.88-4.98 (m, 1H), 7.17 (d, J=7.42 Hz, 1H), 7.47-7.58 (m, 1H), 7.61 (d, J=7.81 Hz, 1H); MS (APPI) (M+H)⁺: 484.2; HRMS m/z calcd for [M+H]⁺ 484.29700. found 484.29615.

### Example 99

### 9-Cyclobutyl-N-ethyl-N-(2-(isopropylamino)-2-oxoethyl)-3-(tetrahydro-2H-pyran-4-yl)-2,3,4,9-tetrahydro-1H-carbazole-6-carboxamide

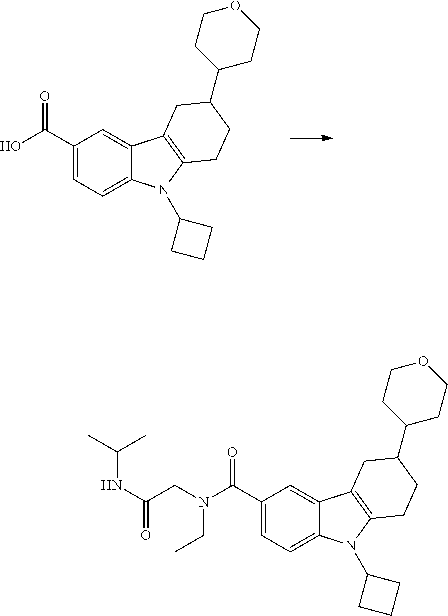

N,N-Diisopropylethylamine (89 μL, 0.51 mmol) was added to a solution of 9-cyclobutyl-3-(tetrahydro-2H-pyran-4-yl)-2,3,4,9-tetrahydro-1H-carbazole-6-carboxylic acid (0.17 mmol) and 2-(ethylamino)-N-isopropylacetamide (49 mg, 0.34 mmol) in DMF (5 mL). Stirring for 20 min, HATU (97 mg, 0.26 mmol) was added at 0° C. The mixture was stirred for 3 h at room temperature, diluted with water (50 mL), and extracted with EtOAc (3×25 mL). The combined organic phases were washed with water (2×20 mL), saturated NaCl (20 mL) and dried with Na₂SO₄. Upon evaporation of the solvent, the crude product was purified by reverse-phase HPLC using high pH column 40-60% MeCN/H₂O to give 61.0 mg (75%) of a white solid as the title compound. ¹H NMR (400 MHz, METHANOL-D4) δ 1.04-1.32 (m, 9H), 1.37-1.50 (m, 2H), 1.50-1.64 (m, 3H), 1.72-1.85 (m, 2H), 1.87-2.05 (m, 2H), 2.12-2.22 (m, 1H), 2.36 (dd, J=9.77, 3.91 Hz, 1H), 2.43-2.59 (m, 2H), 2.67-2.98 (m, 5H), 3.37-3.63 (m, 4H), 3.92-4.05 (m, 4H), 4.07-4.18 (m, 1H), 4.88-4.98 (m, 1H), 7.17 (d, J=8.59 Hz, 1H), 7.47-7.56 (m, 1H), 7.60 (d, J=8.59 Hz, 1H); MS (APPI) (M+H)⁺: 480.2; HRMS m/z calcd for [M+H]⁺ 480.32207. found 480.32120.

### Example 100

### 9-Ethyl-N-methyl-N-(4-(methylamino)-4-oxobutyl)-3-(tetrahydro-2H-pyran-4-yl)-2,3,4,9-tetrahydro-1H-carbazole-6-carboxamide

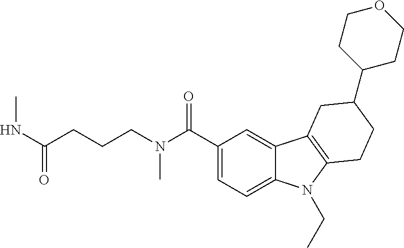

### Step A: 9-ethyl-N-methyl-N-(4-(methylamino)-4-oxobutyl)-3-(tetrahydro-2H-pyran-4-yl)-2,3,4,9-tetrahydro-1H-carbazole-6-carboxamide

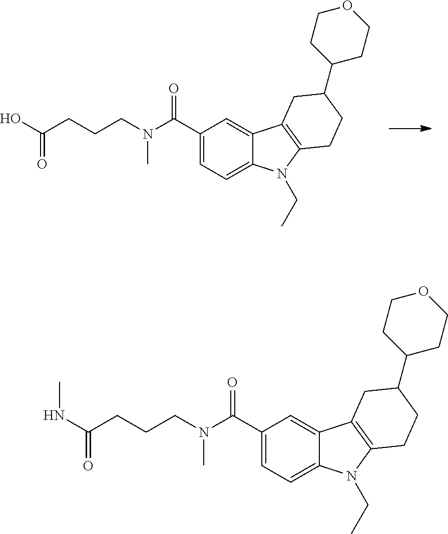

N,N-Diisopropylethylamine (105 μL, 0.60 mmol) was added to a solution of 4-(9-ethyl-N-methyl-3-(tetrahydro-2H-pyran-4-yl)-2,3,4,9-tetrahydro-1H-carbazole-6-carboxamido)butanoic acid (85 mg, 0.2 mmol) and methylamine (220 μL, 2.0 M in THF, 0.44 mmol) in DMF (5 mL). Stirring for 20 min, HATU (114 mg, 0.30 mmol) was added at 0° C. The mixture was stirred for 3 h at room temperature, and quenched with water (0.5 mL). After concentration, the crude product was purified by reverse-phase HPLC using high pH column 30-50% MeCN/H₂O to give 56.3 mg (64%) of a white solid as the title compound. ¹H NMR (400 MHz, CHLOROFORM-D) δ 1.33 (t, J=7.03 Hz, 3H), 1.39-1.68 (m, 6H), 1.70-1.81 (m, 2H), 1.92-2.07 (m, 2H), 2.11-2.20 (m, 1H), 2.25-2.36 (m, 1H), 2.37-2.46 (m, 1H), 2.64-2.76 (m, 1H), 2.77-2.93 (m, 5H), 3.06 (s, 3H), 3.36-3.47 (m, 2H), 3.55-3.71 (m, 2H), 3.99-4.17 (m, 4H), 7.08-7.16 (m, 1H), 7.17-7.22 (m, 1H), 7.24-7.29 (m, 1H), 7.55 (d, J=1.17 Hz, 1H); MS (APPI) (M+H)⁺: 440.2; HRMS m/z calcd for [M+H]⁺ 440.29077. found 440.29074.

### Step B: 9-ethyl-3-(tetrahydro-2H-pyran-4-yl)-2,3,4,9-tetrahydro-1H-carbazole-6-carboxylic acid

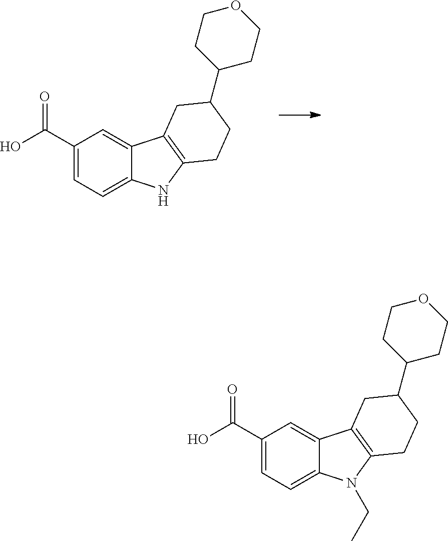

Sodium hydride (3.30 g, 83 mmol) was added to a solution of 3-(tetrahydro-2H-pyran-4-yl)-2,3,4,9-tetrahydro-1H-carbazole-6-carboxylic acid (4.49 g, 15 mmol) in DMF (100 mL) at 0° C. Stirring for 45 min at 0° C. and 1 h at room temperature, iodoethane (4.85 mL, 60 mmol) was added. The mixture was stirred for 40 h at room temperature and quenched with water (20 mL). After concentration, the residue was dissolved in water (200 mL) and extracted with EtOAc (3×100 mL). The aqueous was acidified with 2N HCl to pH˜5. The light yellow solid was collected by filtration and dried in vacuo to give 3.78 g (77%) of a white solid as the title compound ¹H NMR (400 MHz, CHLOROFORM-D) □ 1.35 (t, J=7.23 Hz, 3H), 1.43-1.69 (m, 5H), 1.77 (d, J=11.72 Hz, 2H), 2.12-2.24 (m, 1H), 2.45 (dd, J=15.23, 8.59 Hz, 1H), 2.63-3.02 (m, 3H), 3.44 (t, J=11.33 Hz, 2H), 3.95-4.20 (m, 4H), 7.29 (d, J=8.59 Hz, 1H), 7.93 (d, J=8.59 Hz, 1H), 8.32 (s, 1H); MS (ESI) (M+H)⁺: 328.21.

### Step C: methyl 4-(9-ethyl-N-methyl-3-(tetrahydro-2H-pyran-4-yl)-2,3,4,9-tetrahydro-1H-carbazole-6-carboxamido)butanoate

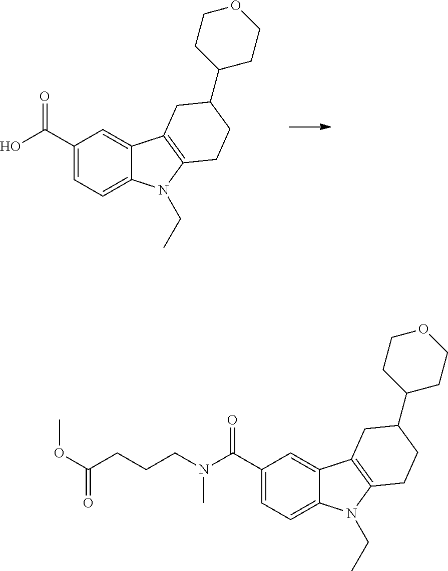

N,N-Diisopropylethylamine (2.09 mL, 12.0 mmol) was added to a solution of 9-ethyl-3-(tetrahydro-2H-pyran-4-yl)-2,3,4,9-tetrahydro-1H-carbazole-6-carboxylic acid (1.31 g, 4.0 mmol) and methyl 4-(methylamino)butanoate hydrochloride (1.34 g, 8.0 mmol) in DMF (30 mL). Stirring for 20 min, HATU (2.28 g, 6.0 mmol) was added at 0° C. The mixture was stirred for 3 h at room temperature, and quenched with water (10 mL). After concentration, added 50 mL of water and extracted with EtOAc (3×25 mL). The combined organic phases were washed with water (2×20 mL), saturated NaCl (20 mL) and dried with Na₂SO₄. Upon evaporation of the solvent, the crude product was purified by MPLC on silica gel using EtOAC to give 1.32 g (75%) of a white solid as the title compound. 1H NMR (400 MHz, METHANOL-D4) δ 1.32 (t, J=7.23 Hz, 3H), 1.39-1.67 (m, 5H), 1.70-1.82 (m, 2H), 1.86-2.06 (m, 2H), 2.10-2.20 (m, 1H), 2.27-2.57 (m, 2H), 2.64-2.76 (m, 1H), 2.76-2.91 (m, 2H), 3.06 (s, 3H), 3.33-3.79 (m, 7H), 3.99-4.11 (m, 5H), 7.14-7.21 (m, 1H), 7.20-7.26 (m, 1H), 7.52 (s, 1H); MS (ESI) (M+H)⁺: 441.33.

### Step D: 4-(9-ethyl-N-methyl-3-(tetrahydro-2H-pyran-4-yl)-2,3,4,9-tetrahydro-1H-carbazole-6-carboxamido)butanoic acid

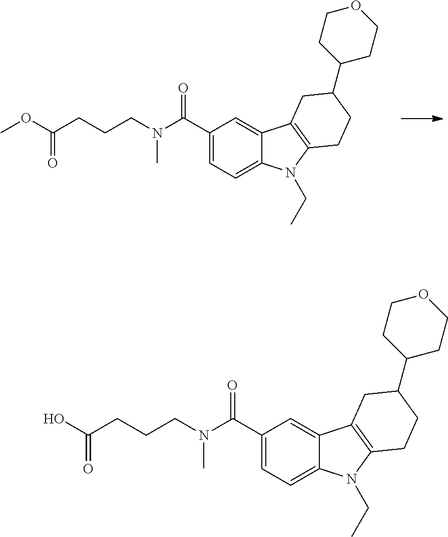

Lithium hydroxide (0.14 g, 5.98 mmol) was added to a solution of methyl 4-(9-ethyl-N-methyl-3-(tetrahydro-2H-pyran-4-yl)-2,3,4,9-tetrahydro-1H-carbazole-6-carboxamido)butanoate (1.32 g, 2.99 mmol) in methanol (20 mL) and water (10 mL). The reaction mixture was stirred for 4 h at room temperature, acidified with 2 N HCl (4 mL) to pH˜5-6. After concentration, the residue was dissolved in EtOAc (200 mL) washed with water (2×25 mL), saturated NaCl (2×25 mL) and dried with Na₂SO₄. After evaporation the solvent and dried in vacuo, 1.21 g (94%) of a light yellow solid was obtained. MS (ESI) (M+H)⁺: 427.35; ¹H NMR (400 MHz, CHLOROFORM-D) δ 1.33 (t, J=7.23 Hz, 3H), 1.40-1.68 (m, 6H), 1.70-1.83 (m, 2H), 1.91-2.06 (m, 2H), 2.12-2.21 (m, 1H), 2.30-2.57 (m, 2H), 2.64-2.76 (m, 1H), 2.77-2.93 (m, 2H), 3.08 (s, 3H), 3.43 (t, J=11.72 Hz, 2H), 3.53-3.77 (m, 2H), 3.97-4.13 (m, 4H), 7.16-7.26 (m, 2 H), 7.54 (s, 1H).

### Example 101

### 9-Ethyl-N-(4-(2-fluoroethylamino)-4-oxobutyl)-N-methyl-3-(tetrahydro-2H-Pyran-4-yl)-2,3,4,9-tetrahydro-1H-carbazole-6-carboxamide

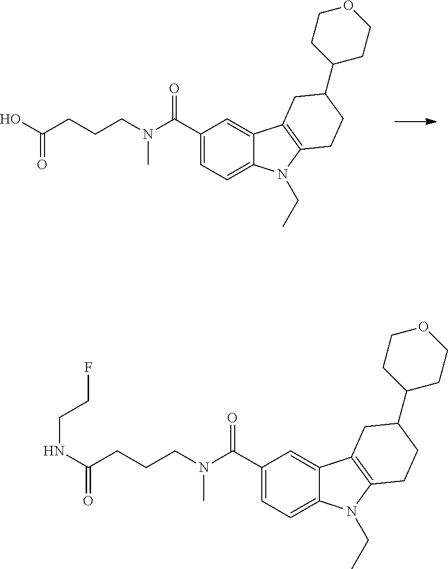

N,N-Diisopropylethylamine (105 μL, 0.60 mmol) was added to a solution of 4-(9-ethyl-N-methyl-3-(tetrahydro-2H-pyran-4-yl)-2,3,4,9-tetrahydro-1H-carbazole-6-carboxamido)butanoic acid (85 mg, 0.20 mmol) and 2-fluoroethylamine hydrochloride (40 mg, 0.40 mmol) in DMF (5 mL). Stirring for 20 min, HATU (114 mg, 0.30 mmol) was added at 0° C. The mixture was stirred for 3 h at room temperature, and quenched with water (0.5 mL). After concentration, the crude product was purified by reverse-phase HPLC using high pH column 30-50% MeCN/H₂O to give 65.8 mg (70%) of a white solid as the title compound. 1H NMR (400 MHz, CHLOROFORM-D) δ 1.33 (t, J=7.23 Hz, 3H), 1.40-1.67 (m, 6H), 1.71-1.81 (m, 2H), 1.92-2.08 (m, 2H), 2.12-2.21 (m, 1H), 2.27-2.46 (m, 2H), 2.65-2.77 (m, 1H), 2.77-2.92 (m, 2H), 3.06 (s, 3H), 3.37-3.48 (m, 2H), 3.49-3.74 (m, 4H), 4.00-4.14 (m, 4H), 4.39-4.64 (m, 2H,) 7.17-7.22 (m, 1H), 7.24-7.29 (m, 1H,) 7.30-7.44 (m, 1H), 7.55 (d, J=1.17 Hz, 1H); MS (APPI) (M+H)⁺: 472.2; HRMS m/z calcd for [M+H]⁺ 472.29700. found 472.29699.

### Example 102

### N-(4-(2,2-difluoroethylamino)-4-oxobutyl)-9-ethyl-N-methyl-3-(tetrahydro-2H-pyran-4-yl)-2,3,4,9-tetrahydro-1H-carbazole-6-carboxamide

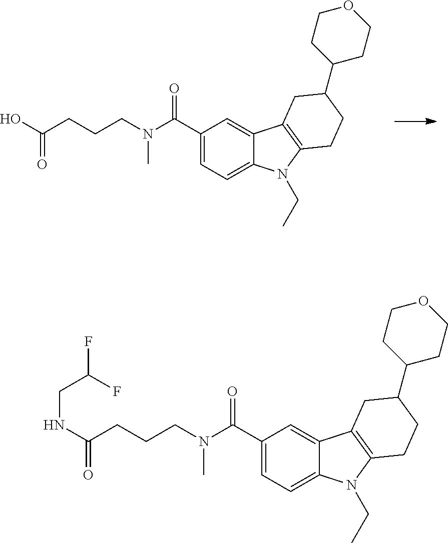

N,N-Diisopropylethylamine (105 μL, 0.60 mmol) was added to a solution of 4-(9-ethyl-N-methyl-3-(tetrahydro-2H-pyran-4-yl)-2,3,4,9-tetrahydro-1H-carbazole-6-carboxamido)butanoic acid (85 mg, 0.20 mmol) and 2,2-difluoroethylamine (32 mg, 0.40 mmol) in DMF (5 mL). Stirring for 20 min, HATU (114 mg, 0.30 mmol) was added at 0° C. The mixture was stirred for 3 h at room temperature, and quenched with water (0.5 mL). After concentration, the crude product was purified by reverse-phase HPLC using high pH column 30-50% MeCN/H₂O to give 63.9 mg (65%) of a white solid as the title compound. ¹H NMR (400 MHz, CHLOROFORM-D) δ 1.29-1.37 (m, 3H), 1.40-1.67 (m, 6H), 1.71-1.82 (m, 2H), 1.92-2.07 (m, 2H), 2.11-2.21 (m, 1H), 2.30-2.51 (m, 2H), 2.64-2.77 (m, 1H), 2.78-2.93 (m, 2H), 3.06 (s, 3H), 3.36-3.48 (m, 2H), 3.63 (dd, J=2.93, 1.76 Hz, 4H), 3.99-4.14 (m, 4H), 5.68-6.11 (m, 1H), 7.16-7.22 (m, 1H), 7.23-7.31 (m, 1H), 7.55 (d, J=1.17 Hz, 1H), 7.66-7.83 (m, J=5.27, 2.54 Hz, 1H); MS (APPI) (M+H)⁺: 490.2; HRMS m/z calcd for [M+H]⁺ 490.28757. found 490.28740.

### Example 103

### 9-Ethyl-N-methyl-N-(4-oxo-4-(2,2,2-trifluoroethylamino)butyl)-3-(tetrahydro-2H-pyran-4-yl)-2,3,4,9-tetrahydro-1H-carbazole-6-carboxamide

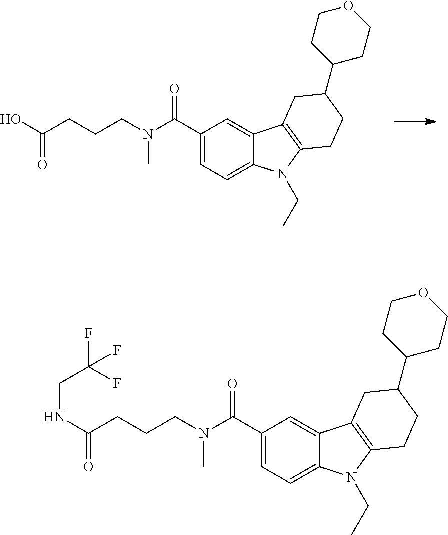

N,N-Diisopropylethylamine (105 μL, 0.60 mmol) was added to a solution of 4-(9-ethyl-N-methyl-3-(tetrahydro-2H-pyran-4-yl)-2,3,4,9-tetrahydro-1H-carbazole-6-carboxamido)butanoic acid (85 mg, 0.20 mmol) and 2,2,2-trifluoroethylamine (40 mg, 0.40 mmol) in DMF (5 mL). Stirring for 20 min, HATU (114 mg, 0.30 mmol) was added at 0° C. The mixture was stirred for 3 h at room temperature, and quenched with water (0.5 mL). After concentration, the crude product was purified by reverse-phase HPLC using high pH column 30-50% MeCN/H₂O to give 72.4 mg (71%) of a white solid as the title compound. ¹H NMR (400 MHz, CHLOROFORM-D) δ 1.33 (t, J=7.23 Hz, 3H), 1.40-1.68 (m, 6H), 1.71-1.82 (m, 2H), 1.95-2.06 (m, 2H), 2.12-2.21 (m, 1H), 2.31-2.47 (m, 2H), 2.65-2.77 (m, 1H), 2.78-2.92 (m, 2H), 3.06 (s, 3H), 3.38-3.47 (m, 2H), 3.58-3.71 (m, 2H), 3.88-4.01 (m, 2H), 4.02-4.13 (m, 4H), 7.18-7.22 (m, 1H), 7.25-7.29 (m, 1H), 7.55 (d, J=1.17 Hz, 1H), 8.06-8.19 (m, 1H); MS (APPI) (M+H)⁺: 508.3; HRMS m/z calcd for [M+H]⁺ 508.27815. found 508.27792.

### Example 104

### 9-Ethyl-N-(4-(2-hydroxyethylamino)-4-oxobutyl)-N-methyl-3-(tetrahydro-2H-pyran-4-yl)-2,3,4,9-tetrahydro-1H-carbazole-6-carboxamide

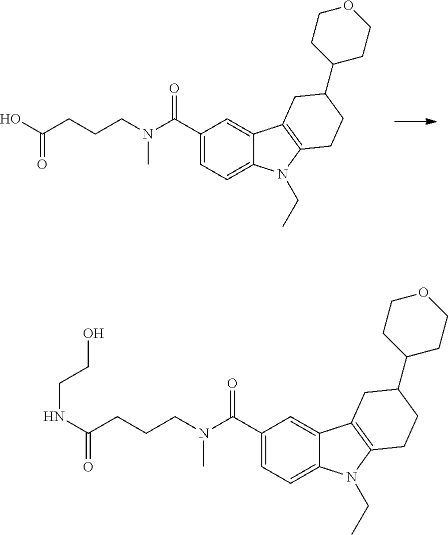

N,N-Diisopropylethylamine (105 μL, 0.60 mmol) was added to a solution of 4-(9-ethyl-N-methyl-3-(tetrahydro-2H-pyran-4-yl)-2,3,4,9-tetrahydro-1H-carbazole-6-carboxamido)butanoic acid (85 mg, 0.20 mmol) and ethanolamine (24 mg, 0.40 mmol) in DMF (5 mL). Stirring for 20 min, HATU (114 mg, 0.30 mmol) was added at 0° C. The mixture was stirred for 3 h at room temperature, and quenched with water (0.5 mL). After concentration, the crude product was purified by reverse-phase HPLC using high pH column 30-50% MeCN/H₂O to give 65.4 mg (70%) of a white solid as the title compound. ¹H NMR (400 MHz, CHLOROFORM-D) δ 1.33 (t, J=7.23 Hz, 3H), 1.41-1.68 (m, 6H), 1.72-1.80 (m, 2H), 1.97-2.08 (m, 2H), 2.12-2.20 (m, 1H), 2.28-2.37 (m, 2H), 2.37-2.46 (m, 1H), 2.65-2.77 (m, 1H), 2.78-2.92 (m, 2H), 3.08 (s, 3H), 3.37-3.49 (m, 4H), 3.58-3.68 (m, 2H), 3.69-3.79 (m, 2H), 4.01-4.13 (m, 4H), 7.19-7.23 (m, 1H), 7.24-7.30 (m, 1H), 7.39-7.49 (m, 1H), 7.55 (d, J=1.17 Hz, 1H); MS (APPI) (M+H)⁺: 470.2; HRMS m/z calcd for [M+H]⁺ 470.30133. found 470.30102.

### Example 105

### N-Ethyl-N-(2-(ethylamino)-2-oxoethyl)-9-(2-fluoroethyl)-3-(tetrahydro-2H-pyran-4-yl)-2,3,4,9-tetrahydro-1H-carbazole-6-carboxamide

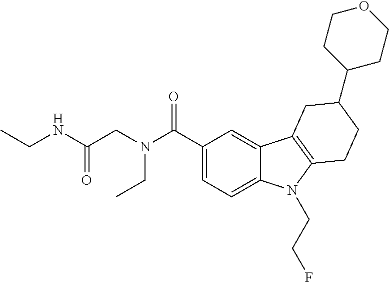

### Step A: N-ethyl-N-(2-(ethylamino)-2-oxoethyl)-9-(2-fluoroethyl)-3-(tetrahydro-2H-pyran-4-yl)-2,3,4,9-tetrahydro-1H-carbazole-6-carboxamide

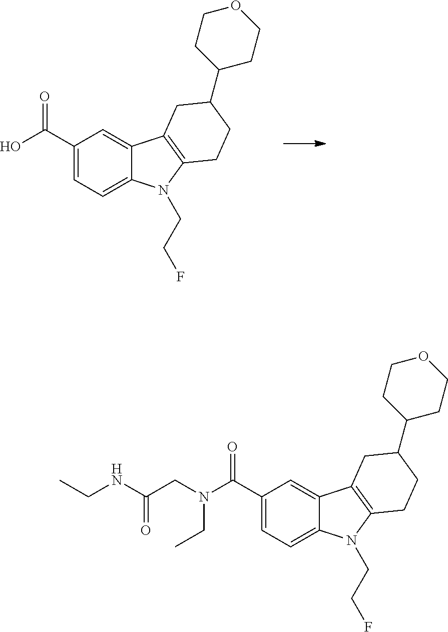

N,N-Diisopropylethylamine (115 μL, 0.66 mmol) was added to a solution of 9-(2-fluoroethyl)-3-(tetrahydro-2H-pyran-4-yl)-2,3,4,9-tetrahydro-1H-carbazole-6-carboxylic acid (0.22 mmol) and N-ethyl-2-(ethylamino)acetamide (57 mg, 0.44 mmol) in DMF (5 mL). Stirring for 20 min, HATU (125 mg, 0.33 mmol) was added at 0° C. The mixture was stirred for 3 h at room temperature, and quenched with water (0.5 mL). After concentration, the residue was dissolved in EtOAc (100 mL), washed with water (2×10 mL), NaCl (2×10 mL) and dried with Na₂SO₄. The crude product was purified by MPLC on silica gel using Hex/EtOAc (1:1) and then EtOAc/MeOH (20:1) to give three fractions.

Fraction-1: the ethyl ester from the Step D, yield: 54.3 mg (66%).

Fraction-2: the starting material from the Step D, 9.6 mg (13%).

Fraction-3: the desired product, which was purified again by reverse-phase HPLC using high pH column 30-50% MeCN/H₂O to give 15.5 mg (15%) of a white solid as the title compound. ¹H NMR (400 MHz, CHLOROFORM-D) δ 1.14-1.24 (m, 6H), 1.38-1.68 (m, 5H), 1.74 (t, J=10.94 Hz, 2H), 2.09-2.20 (m, 1H),2.36-2.46 (m, 1H), 2.64-2.78 (m, 1H), 2.78-2.91 (m, 2H), 3.28-3.38 (m, 2H), 3.42 (t, J=11.72 Hz, 2H), 3.47-3.57 (m, 2H), 4.04 (dd, J=11.33, 3.13 Hz, 2H), 4.11 (s, 2H), 4.27-4.41 (m, 2H), 4.54-4.77 (m, 2H), 6.85-7.07 (m, 1H), 7.17-7.26 (m, 2H), 7.59 (s, 1H); MS (APPI) (M+H)⁺: 458.3; FIRMS m/z calcd for [M+H]⁺ 458.28135. found 458.28116.

### Step B: 3-chloro-4-(2-fluoroethylamino)benzonitrile

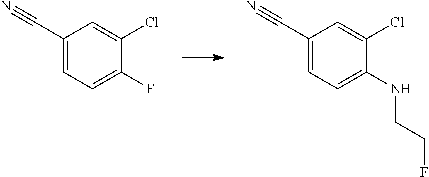

N,N-Diisopropylethylamine (4.37 mL, 25.1 mmol) was added to a solution of 2-fluoroethylamine hydrochloride (1.20 g, 12.0 mmol) and 3-chloro-4-fluorobenzonitrile (1.56 g, 10.0 mmol) in DMSO (15 mL) at room temperature. The reaction mixture was stirred over weekend at room temperature and 8 h at 45° C., diluted with water (150 mL), and extracted with EtOAc (3×50 mL). The combined organic phases were washed with water (2×20 mL), saturated NaCl (2×20 mL) and dried with Na₂SO₄. The crude product was purified by MPLC on silica gel using Hex/EtOAc (4:1) to give 0.82 g (41%) of a white solid as the title compound. ¹H NMR (400 MHz, CHLOROFORM-D) δ 3.50-3.64 (m, 2H), 4.57-4.77 (m, 2H), 5.13 (s broad, 1H), 6.67 (d, J=8.59 Hz, 1H), 7.44 (dd, J=8.59, 1.95 Hz, 1H), 7.55 (d, J=1.95 Hz, 1H); MS (ESI) (M+H)⁺: 199.15.

### Step C: 9-(2-fluoroethyl)-3-(tetrahydro-2H-pyran-4-yl)-2,3,4,9-tetrahydro-1H-carbazole-6-carbonitrile

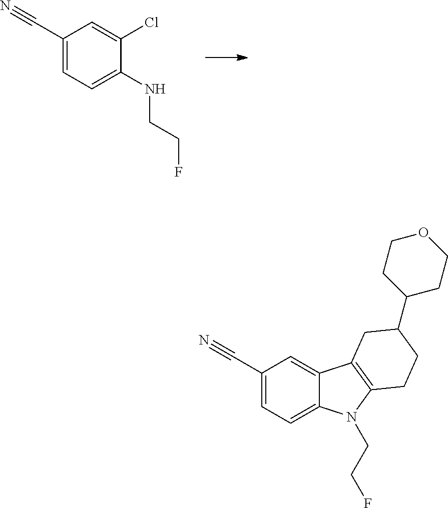

3-Chloro-4-(2-fluoroethylamino)benzonitrile (157 mg, 0.79 mmol), 4-(tetrahydro-2H-pyran-4-yl)cyclohexanone (433 mg, 2.38 mmol), acetic acid (68 μL, 71 mg, 1.19 mmol), and magnesium sulfate (48 mg, 0.40 mmol) were suspended in DMA (4 mL). Nitrogen was bubbled through the solution for 10 min. Potassium phosphate (218 mg, 1.03 mmol) and bis(tri-t-butylphosphine)palladium(0) (40 mg, 0.08 mmol) were added, and nitrogen was bubbled through the mixture for an additional 5 min. The reaction mixture was heated for 14 h at 140° C. After cooling to room temperature, the reaction mixture was diluted with water (15 mL), and extracted with EtOAc (4×20 mL). The combined organic phases were washed with water (2×15 mL) and NaCl (2×15 mL), and dried with Na₂SO₄. The crude product was purified by MPLC on silica gel using Hex/EtOAC (1:1) to give 80.2 mg (31%) of a white solid as the title compound. ¹H NMR (400 MHz, CHLOROFORM-D) δ 1.40-1.70 (m, 5H), 1.70-1.80 (m, 2H), 2.13-2.22 (m, 1H), 2.35-2.46 (m, 1H), 2.66-2.77 (m, 1H), 2.79-2.90 (m, 2H), 3.38-3.48 (m, 2H), 4.00-4.10 (m, 2H), 4.29-4.40 (m, 2H), 4.57-4.76 (m, 2H), 7.29 (s, 1H), 7.37-7.41 (m, 1 H), 7.80 (d, J=1.17 Hz, 1H); MS (ESI) (M+H)⁺: 327.21.

### Step D: 9-(2-fluoroethyl)-3-(tetrahydro-2H-pyran-4-yl)-2,3,4,9-tetrahydro-1H-carbazole-6-carboxylic acid and ethyl 9-(2-fluoroethyl)-3-(tetrahydro-2H-pyran-4-yl)-2,3,4,9-tetrahydro-1H-carbazole-6-carboxylate

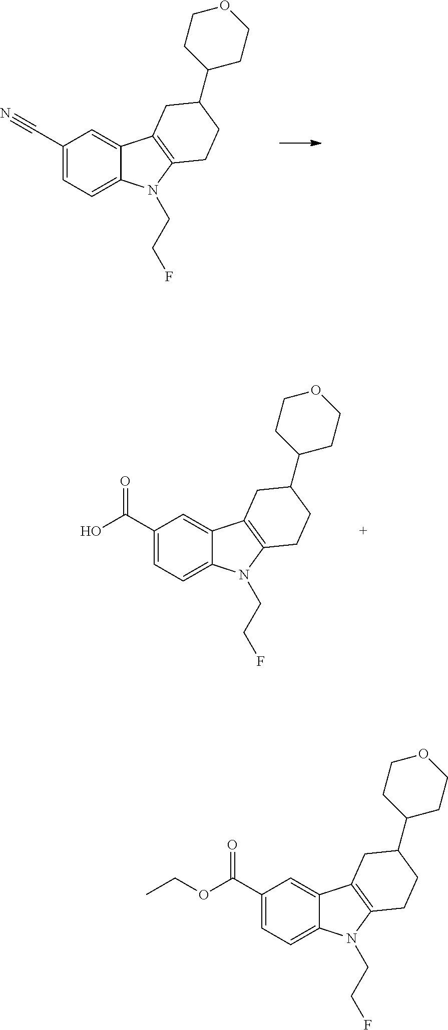

9-(2-Fluoroethyl)-3-(tetrahydro-2H-pyran-4-yl)-2,3,4,9-tetrahydro-1H-carbazole-6-carbonitrile (71.4 mg, 0.22 mmol) was heated in 6 N hydrochloric acid (2 ml) and ethanol (3 mL) in a sealed tube at 140° C. for 3 intervals 2 h using a Biotage (1-60) microwave instrument. Three major peaks were observed by LCMS: MS (ESI) (M+H)⁺ at 346.14 (30%), 374.27 (44%) and 327.21 (26%). The reaction mixture was diluted with water (20 mL) and extracted with EtOAC (3×20 mL). The combined organic phases was washed with saturated NaCl (2×10 mL) and dried with Na₂SO₄. After filtration and concentration, the crude product was used directly for next step without further purification.

### Example 106

### N-(4-(Ethylamino)-4-oxobutyl)-9-(2-fluoroethyl)-N-methyl-3-(tetrahydro-2H-pyran-4-yl)-2,3,4,9-tetrahydro-1H-carbazole-6-carboxamide

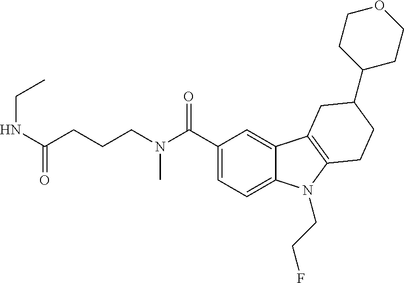

### Step A: N-(4-(ethylamino)-4-oxobutyl)-9-(2-fluoroethyl)-N-methyl-3-(tetrahydro-2H-pyran-4-yl)-2,3,4,9-tetrahydro-1H-carbazole-6-carboxamide

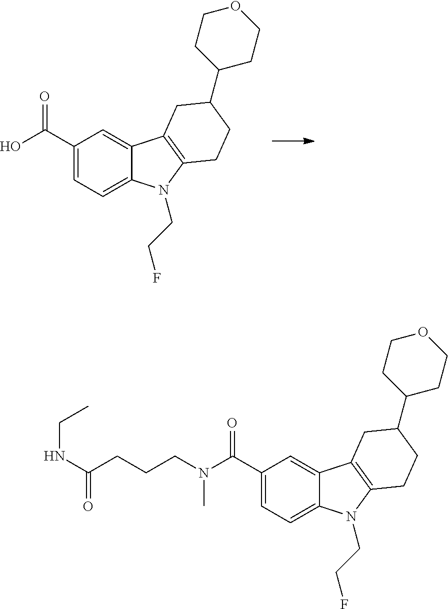

N,N-Diisopropylethylamine (105 μL, 0.60 mmol) was added to a solution of 9-(2-fluoroethyl)-3-(tetrahydro-2H-pyran-4-yl)-2,3,4,9-tetrahydro-1H-carbazole-6-carboxylic acid (0.15 mmol) and N-ethyl-4-(methylamino)butanamide hydrochloride (54 mg, 0.30 mmol) in DMF (5 mL). Stirring for 20 min, HATU (86 mg, 0.23 mmol) was added at 0° C. The mixture was stirred for 3 h at room temperature, and quenched with water (0.5 mL). After concentration, the residue was dissolved in EtOAc (100 mL), washed with water (2×10 mL), NaCl (2×10 mL) and dried with Na₂SO₄. The crude product was purified by MPLC on silica gel using EtOAc/MeOH (20:1), and then by reverse-phase HPLC using high pH column 30-50% MeCN/H₂O to give 11.7 mg (17%) of a white solid as the title compound. 1H NMR (400 MHz, CHLOROFORM-D) δ 1.05-1.24 (m, 2H), 1.37-1.68 (m, 7H), 1.69-1.83 (m, 2H), 1.87-2.08 (m, 2H), 2.11-2.21 (m, 1H), 2.22-2.35 (m, 1H), 2.36-2.46 (m, 1H), 2.63-2.77 (m, 1H), 2.78-2.94 (m, 2H), 3.04 (s, 3H) 3.22-3.37 (m, 2H) 3.42 (t, J=11.52 Hz, 2H), 3.53-3.73 (m, 2H), 4.04 (dd, J=10.74, 2.93 Hz, 2H), 4.24-4.43 (m, 2H), 4.55-4.77 (m, 2H), 6.85-7.02 (m, 1H), 7.15-7.25 (m, 2H), 7.55 (s, 1H); MS (APPI) (M+H)⁺: 472.2; HRMS m/z calcd for [M+H]⁺472.29700. found 472.29667.

### Step B: 9-(2-fluoroethyl)-3-(tetrahydro-2H-pyran-4-yl)-2,3,4,9-tetrahydro-1H-carbazole-6-carboxylic acid

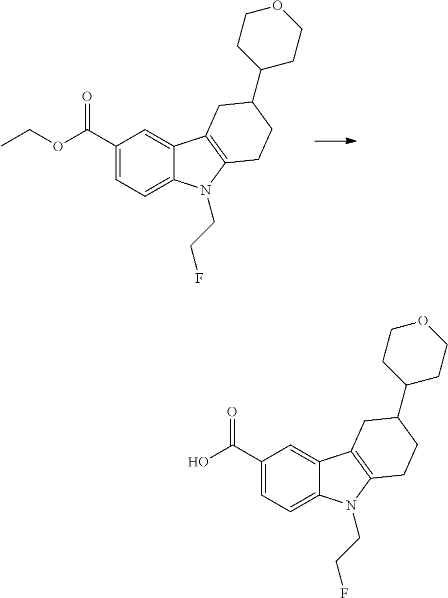

Lithium hydroxide (13.9 mg, 0.58 mmol) was added to a solution of ethyl 9-(2-fluoroethyl)-3-(tetrahydro-2H-pyran-4-yl)-2,3,4,9-tetrahydro-1H-carbazole-6-carboxy late (from Example 11) (54.3 mg, 0.15 mmol) in THF (5 mL) and water (0.5 ml). The reaction mixture was stirred for 5 h at 60° C. After cooling to room temperature, 2 N HCl (1 mL) was added. Upon concentration, the crude product was used directly for next step without purification. MS (ESI) (M+H)⁺: 346.20.

### Example 107

### N-ethyl-9-(ethylsulfonyl)-N-(2-(2-hydroxyethylamino)-2-oxoethyl)-3-(tetrahydro-2H-pyran-4-yl)-2,3,4,9-tetrahydro-1H-carbazole-6-carboxamide

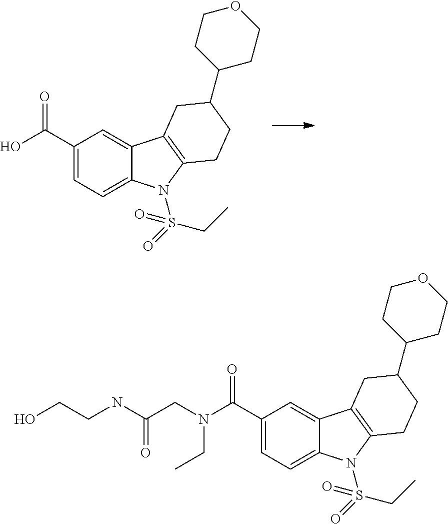

HATU (227 mg, 0.60 mmol) and 2-(ethylamino)-N-(2-hydroxyethyl)acetamide (202 mg, 1.38 mmol) were added slowly at 0° C. to a solution of 9-(ethylsulfonyl)-3-(tetrahydro-2H-pyran-4-yl)-2,3,4,9-tetrahydro-1H-carbazole-6-carboxylic acid (180 mg, 0.46 mmol) and N,N-diisopropylethylamine (0.240 mL, 1.38 mmol) in DMF (3.83 mL). Reaction mixture was stirred at room temperature for an O/N. The solvent was then removed in vacuo to provide the crude compound as oil. The residue was dissolved in AcOEt and washed with NH₄OH aq. in order to remove HATU. N-ethyl-9-(ethylsulfonyl)-N-(2-(2-hydroxyethylamino)-2-oxoethyl)-3-(tetrahydro-2H-pyran-4-yl)-2,3,4,9-tetrahydro-1H-carbazole-6-carboxamide (75 mg, 25.9%) was purified by Prep-HPLC reverse-phase with low pH 50-70% ACN/water system. ¹H NMR (400 MHz, CD₃OD) δ ppm 1.14 (t, J=7.23 Hz, 3H), 1.19-1.29 (m, 1H), 1.35-1.52 (m, 2H), 1.49-1.67 (m, 2H), 1.79 (t, J=10.35 Hz, 3H), 2.10-2.24 (m, 1H), 2.28-2.53 (m, 1H), 2.74-2.95 (m, 2H), 3.08-3.23 (m, 1H), 3.33-3.49 (m, 9H), 3.50-3.74 (m, 4H), 4.00 (dd, J=11.52, 2.93 Hz, 4H), 4.19 (s, 1H), 7.28-7.45 (m, 1H), 7.55-7.68 (m, 1H), 7.89-8.12 (m, 1H); MS (ESI) (M+H)+ 520.2.

### Example 108

### N-ethyl-9-(ethylsulfonyl)-N-(2-(3-hydroxypropylamino)-2-oxoethyl)-3-(tetrahydro-2H-pyran-4-yl)-2,3,4,9-tetrahydro-1H-carbazole-6-carboxamide

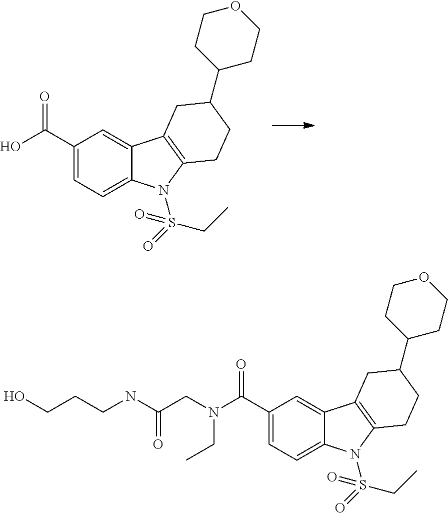

HATU (227 mg, 0.60 mmol) and 2-(ethylamino)-N-(3-hydroxypropyl)acetamide (147 mg, 0.92 mmol) were added slowly at 0° C. to a solution of 9-(ethylsulfonyl)-3-(tetrahydro-2H-pyran-4-yl)-2,3,4,9-tetrahydro-1H-carbazole-6-carboxylic acid (180 mg, 0.46 mmol) and N,N-diisopropylethylamine (0.240 mL, 1.38 mmol) in DMF (3.83 mL). Reaction mixture was stirred at room temperature for an O/N. The solvent was then removed in vacuo to provide the crude compound as oil. The residue was dissolved in AcOEt and washed with NH₄OH aq. in order to remove HATU. N-ethyl-9-(ethylsulfonyl)-N-(2-(3-hydroxypropylamino)-2-oxoethyl)-3-(tetrahydro-2H-pyran-4-yl)-2,3,4,9-tetrahydro-1H-carbazole-6-carboxamide (7.40 mg, 3.02%) was purified by Prep-HPLC reverse-phase using a low pH 50-70% ACN/water system. ¹H NMR (400 MHz, CD₃OD) δ ppm 1.11-1.18 (m, 5H), 1.20-1.28 (m, 2H), 1.33-1.39 (m, 1H), 1.39-1.51 (m, 2H), 1.52-1.67 (m, 4H), 1.79 (t, J=10.74 Hz, 3H), 2.13-2.22 (m, 1H), 2.31-2.42 (m, 1H), 2.79-2.90 (m, 1H), 3.11-3.20 (m, 1H), 3.32-3.39 (m, 4H), 3.40-3.48 (m, 4H), 3.53-3.75 (m, 3H), 3.93-3.97 (m, 1H), 4.00 (dd, J=11.13, 3.32 Hz, 2H), 4.16 (s, 1H), 7.29-7.41 (m, 1H), 7.53-7.66 (m, 1H), 7.93-8.04 (m, 1H); MS (ESI) (M+H)+ 534.3.

### Example 109

### N-ethyl-9-(ethylsulfonyl)-N-(2-(3-fluoropropylamino)-2-oxoethyl)-3-(tetrahydro-2H-pyran-4-yl)-2,3,4,9-tetrahydro-1H-carbazole-6-carboxamide

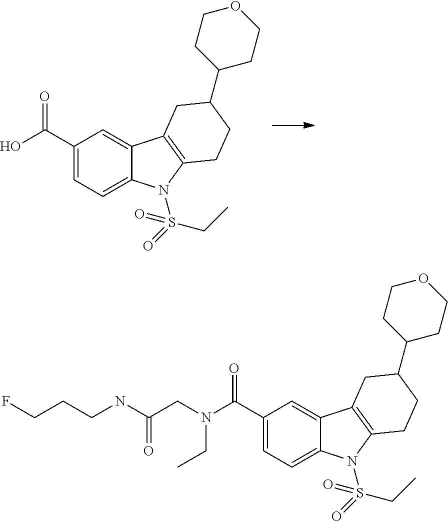

HATU (227 mg, 0.60 mmol) and 2-(ethylamino)-N-(3-fluoropropyl)acetamide (224 mg, 1.38 mmol) were added slowly at 0° C. to a solution of 9-(ethylsulfonyl)-3-(tetrahydro-2H-pyran-4-yl)-2,3,4,9-tetrahydro-1H-carbazole-6-carboxylic acid (180 mg, 0.46 mmol) and N,N-diisopropylethylamine (0.240 mL, 1.38 mmol) in DMF (3.83 mL). Reaction mixture was stirred at room temperature for an 5 hours. The solvent was then removed in vacuo to provide the crude compound as an oil. The residue was dissolved in AcOEt and washed with NH₄OH aq. in order to remove HATU. N-ethyl-9-(ethylsulfonyl)-N-(2-(3-fluoropropylamino)-2-oxoethyl)-3-(tetrahydro-2H-pyran-4-yl)-2,3,4,9-tetrahydro-1H-carbazole-6-carboxamide (117 mg, 39.1%) was purified by Prep-LCMS reverse-phase using a low pH 40-60% ACN/water system. ¹H NMR (400 MHz, CD₃OD) δ ppm 1.13 (t, J=7.23 Hz, 5H), 1.19-1.31 (m, 1H), 1.34-1.50 (m, 3H), 1.50-1.65 (m, 3H), 1.77 (t, J=11.33 Hz, 3H), 1.81-1.99 (m, 2H), 2.10-2.21 (m, 1H), 2.26-2.44 (m, 1H), 2.71-2.93 (m, 2H), 3.00-3.19 (m, 1H), 3.32-3.48 (m, 6H), 3.52-3.64 (m, 1H), 3.98 (dd, J=11.52, 3.32 Hz, 3H), 4.15 (s, 1H), 4.26-4.62 (m, 2H), 7.28-7.42 (m, 1H), 7.52-7.67 (m, 1H), 7.91-8.04 (m, 1H); MS (ESI) (M+H)+ 536.4.

### Example 110

### N-ethyl-9-(ethylsulfonyl)-N-(2-(2-fluoroethylamino)-2-oxoethyl)-3-(tetrahydro-2H-pyran-4-yl)-2,3,4,9-tetrahydro-1H-carbazole-6-carboxamide

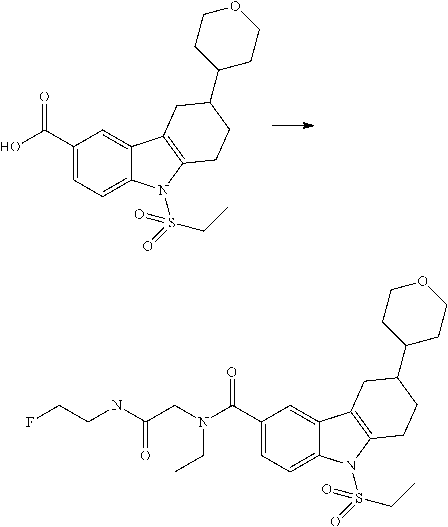

HATU (164 mg, 0.43 mmol) and 2-(ethylamino)-N-(2-fluoroethyl)acetamide (59.0 mg, 0.40 mmol) were added slowly at 0° C. to a solution of 9-(ethylsulfonyl)-3-(tetrahydro-2H-pyran-4-yl)-2,3,4,9-tetrahydro-1H-carbazole-6-carboxylic acid (130 mg, 0.33 mmol) and N,N-diisopropylethylamine (0.217 mL, 1.25 mmol) in DMF (2.64 mL). Reaction mixture was stirred at room temperature for an O/N. The solvent was then removed in vacuo to provide the crude compound as yellow oil. The N-ethyl-9-(ethylsulfonyl)-N-(2-(2-fluoroethylamino)-2-oxoethyl)-3-(tetrahydro-2H-pyran-4-yl)-2,3,4,9-tetrahydro-1H-carbazole-6-carboxamide (40.6%) was purified by Prep-HPLC reverse-phase using a low pH 50-70% ACN/water system. ¹H NMR (400 MHz, CDCl₃) δ ppm 1.17-1.26 (m, 7H), 1.36-1.65 (m, 6H), 1.72 (t, J=12.70 Hz, 2H), 2.07-2.18 (m, 1H), 2.31 (dd, J=17.38, 9.57 Hz, 1H), 2.69-2.97 (m, 2H), 3.09-3.19 (m, 1H), 3.22 (q, J=7.42 Hz, 2H), 3.34-3.46 (m, 3H), 3.44-3.50 (m, 1H), 3.57 (q, J=5.08 Hz, 1H), 3.64 (q, J=5.34 Hz, 1H), 4.03 (dd, J=11.33, 3.13 Hz, 2H), 4.16 (s, 1H), 4.45 (t, J=4.88 Hz, 1H), 4.57 (t, J=4.88 Hz, 1H), 7.27-7.33 (m, 1H), 7.49-7.56 (m, 1H), 7.92-8.03 (m, 1H); [M+H]+ calc.=522.2432, [M+H]+ obs.=522.2434

### Example 111

### 2-(N-ethyl-9-(ethylsulfonyl)-3-(tetrahydro-2H-pyran-4-yl)-2,3,4,9-tetrahydro-1H-carbazole-6-carboxamido)acetic acid

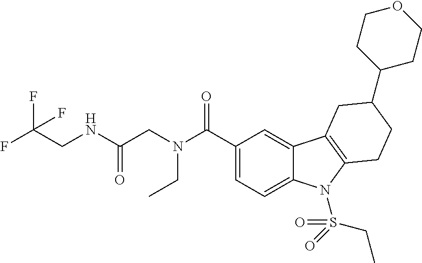

### Step A. N-ethyl-9-(ethylsulfonyl)-N-(2-oxo-2-(2,2,2-trifluoroethylamino)ethyl)-3-(tetrahydro-2H-pyran-4-yl)-2,3,4,9-tetrahydro-1H-carbazole-6-carboxamide

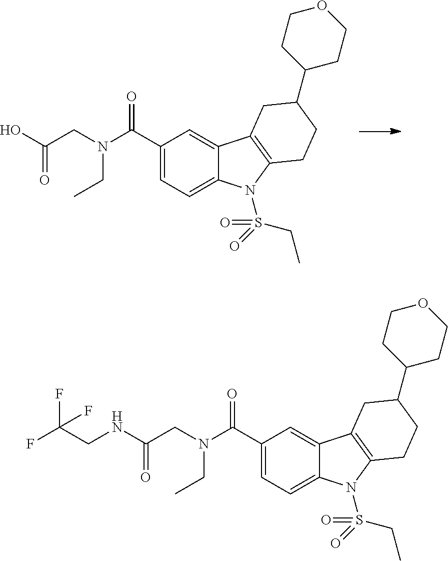

HATU (89 mg, 0.24 mmol) and 2,2,2-Trifluoroethylamine (0.018 mL, 0.24 mmol) were added slowly at 0° C. to a solution of 2-(N-ethyl-9-(ethylsulfonyl)-3-(tetrahydro-2H-pyran-4-yl)-2,3,4,9-tetrahydro-1H-carbazole-6-carboxamido)acetic acid (56 mg, 0.12 mmol) and N,N-diisopropylethylamine (0.125 mL, 0.72 mmol) in DMF (1.5 mL). Reaction mixture was stirred at room temperature for 2 h30. The solvent was then removed in vacuo to provide the crude compound as yellow oil. The N-ethyl-9-(ethylsulfonyl)-N-(2-oxo-2-(2,2,2-trifluoroethylamino)ethyl)-3-(tetrahydro-2H-pyran-4-yl)-2,3,4,9-tetrahydro-1H-carbazole-6-carboxamide (58.9 mg, 74.6%) was purified by Prep-HPLC reverse-phase using a low pH 60-80% ACN/water system. ¹H NMR (400 MHz, CD₃OD) δ ppm 1.06-1.20 (m, 6H), 1.19-1.32 (m, 1H), 1.36-1.50 (m, 2H), 1.50-1.68 (m, 3H), 1.79 (t, J=12.11 Hz, 2H), 2.11-2.23 (m, 1H), 2.28-2.48 (m, 1H), 2.72-2.96 (m, 2H), 3.09-3.23 (m, 1H), 3.32-3.39 (m, 2H), 3.38-3.50 (m, 4H), 3.53-3.67 (m, 1H), 3.84-4.11 (m, 4H), 4.23 (s, 1H), 7.25-7.45 (m, 1H), 7.49-7.69 (m, 1 H), 7.92-8.08 (m, 1H); [M+H]+ calc.=558.2244, [M+H]+ obs.=558.2233.

### Step B. N-(2-(cyclopropylamino)-2-oxoethyl)-9-(ethylsulfonyl)-N-methyl-3-(tetrahydro-2H-pyran-4-yl)-2,3,4,9-tetrahydro-1H-carbazole-6-carboxamide

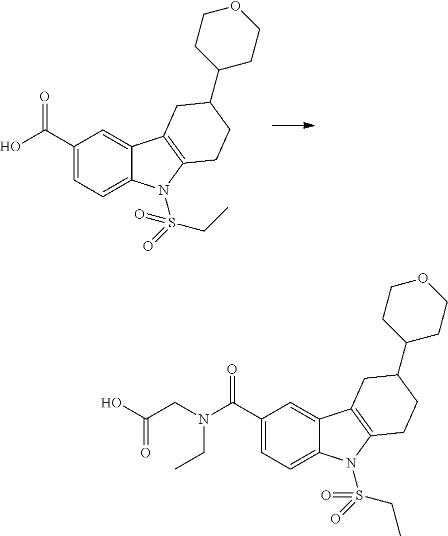

HATU (291 mg, 0.77 mmol) and N-Ethylglycine (119 mg, 1.15 mmol) were added slowly at 0° C. to a solution of 9-(ethylsulfonyl)-3-(tetrahydro-2H-pyran-4-yl)-2,3,4,9-tetrahydro-1H-carbazole-6-carboxylic acid (150 mg, 0.38 mmol) and N,N-diisopropylethylamine (0.200 mL, 1.15 mmol) in DMF (3.04 mL). Reaction mixture was stirred at room temperature for 3 h. The solvent was then removed in vacuo to provide the crude compound as yellow oil. The 2-(N-ethyl-9-(ethylsulfonyl)-3-(tetrahydro-2H-pyran-4-yl)-2,3,4,9-tetrahydro-1H-carbazole-6-carboxamido)acetic acid (61.1 mg, 27.0%) was purified by Prep-HPLC reverse-phase using a low pH 60-80% ACN/water system. ¹H NMR (400 MHz, CD₃OD) δ ppm 1.06-1.22 (m, 6H), 1.25 (t, J=7.03 Hz, 1 H), 1.32-1.50 (m, 2H), 1.50-1.64 (m, 3H), 1.78 (t, J=11.13 Hz, 2H), 2.09-2.24 (m, 1H), 2.27-2.53 (m, 1H), 2.74-2.95 (m, 2H), 3.04-3.24 (m, 1H), 3.31-3.52 (m, 4H), 3.60 (q, J=7.03 Hz, 1H), 3.99 (dd, J=11.13, 3.32 Hz, 2H), 4.04 (s, 1H), 4.23 (s, 2H), 7.18-7.44 (m, 1H), 7.44-7.67 (m, 1H), 7.86-8.09 (m, 1H); MS (ESI) (M+H)+ 477.4.

### Example 112

### N-(2-(cyclopropylamino)-2-oxoethyl)-9-(ethylsulfonyl)-N-methyl-3-(tetrahydro-2H-pyran-4-yl)-2,3,4,9-tetrahydro-1H-carbazole-6-carboxamide

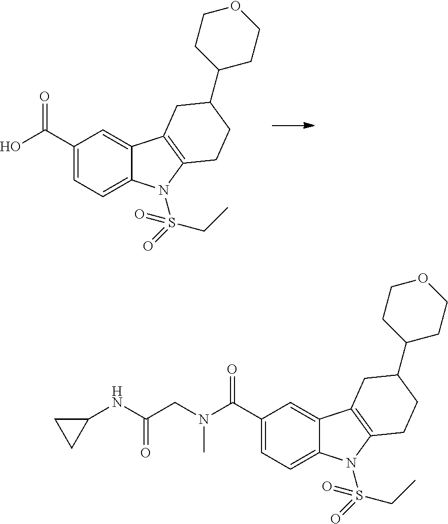

HATU (189 mg, 0.50 mmol) and N-cyclopropyl-2-(methylamino)acetamide (73.7 mg, 0.57 mmol) were added slowly at 0° C. to a solution of 9-(ethylsulfonyl)-3-(tetrahydro-2H-pyran-4-yl)-2,3,4,9-tetrahydro-1H-carbazole-6-carboxylic acid (150 mg, 0.38 mmol) and N,N-diisopropylethylamine (0.199 mL, 1.14 mmol) in DMF (3.19 mL). Reaction mixture was stirred at room temperature for an O/N. The solvent was then removed in vacuo to provide the crude compound as oil. The residue was dissolved in AcOEt and washed with NH₄OH aq. in order to remove HATU. N-(2-(cyclopropylamino)-2-oxoethyl)-9-(ethylsulfonyl)-N-methyl-3-(tetrahydro-2H-pyran-4-yl)-2,3,4,9-tetrahydro-1H-carbazole-6-carboxamide (109 mg, 38.9%) was purified by Prep-LCMS reverse-phase using a low pH 40-60% ACN/water system. ¹H NMR (400 MHz, CD₃OD) δ ppm 0.38-0.57 (m, 2H), 0.72 (dd, J=11.72, 6.64 Hz, 2H), 1.13 (t, J=7.42 Hz, 3H), 1.33-1.48 (m, 2H), 1.48-1.62 (m, 3H), 1.76 (t, J=10.16 Hz, 3H), 2.08-2.18 (m, 1H), 2.25-2.40 (m, 1H), 2.59-2.88 (m, 3H), 3.04-3.19 (m, 4H), 3.31-3.37 (m, 2H), 3.41 (t, J=11.91 Hz, 2H), 3.92 (s, 1H), 3.98 (dd, J=11.13, 2.93 Hz, 2H), 4.15 (s, 1H), 7.28-7.43 (m, 1H), 7.50-7.67 (m, 1H), 7.92-8.02 (m, 1H); MS (ESI) (M+H)+ 502.3.

### Example 113

### (2R)-1-(9-(ethylsulfonyl)-3-(tetrahydro-2H-pyran-4-yl)-2,3,4,9-tetrahydro-1H-carbazole-6-carbonyl)-N-(2-fluoroethyl)pyrrolidine-2-carboxamide

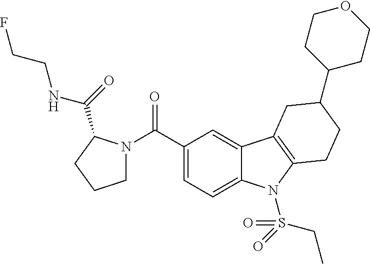

### Step A. (2R)-1-(9-(ethylsulfonyl)-3-(tetrahydro-2H-pyran-4-yl)-2,3,4,9-tetrahydro-1H-carbazole-6-carbonyl)-N-(2-fluoroethyl)pyrrolidine-2-carboxamide

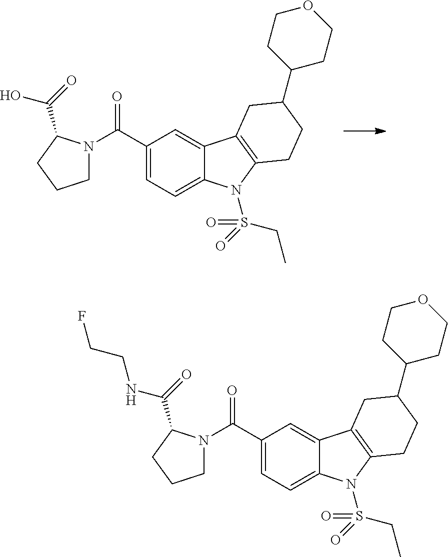

HATU (78 mg, 0.20 mmol) and 2-Fluoroethylamine hydrochloride (20.37 mg, 0.20 mmol) were added slowly at 0° C. to a solution of (2R)-1-(9-(ethylsulfonyl)-3-(tetrahydro-2H-pyran-4-yl)-2,3,4,9-tetrahydro-1H-carbazole-6-carbonyl)pyrrolidine-2-carboxylic acid (50 mg, 0.10 mmol) and N,N-diisopropylethylamine (0.0523 mL, 0.30 mmol) in DMF (0.812 mL). Reaction mixture was stirred at room temperature for 3 h. The solvent was then removed in vacuo to provide the crude compound as yellow oil. The (2R)-1-(9-(ethylsulfonyl)-3-(tetrahydro-2H-pyran-4-yl)-2,3,4,9-tetrahydro-1H-carbazole-6-carbonyl)-N-(2-fluoroethyl)pyrrolidine-2-carboxamide (69.8%) was purified by Prep-HPLC reverse-phase using a low pH 60-80% ACN/water system. ¹H NMR (400 MHz, CD₃OD) δ ppm 1.14 (t, J=7.42 Hz, 3H), 1.35-1.67 (m, 6H), 1.78 (t, J=10.74 Hz, 2H), 1.83-2.08 (m, 3H), 2.11-2.20 (m, 1H), 2.28-2.42 (m, 2H), 2.76-2.91 (m, 2H), 3.09-3.28 (m, 2H), 3.34 (q, J=7.68 Hz, 2H), 3.39-3.47 (m, 2H), 3.47-3.65 (m, 2 H), 3.67-3.82 (m, 2H), 3.99 (dd, J=11.13, 3.32 Hz, 2H), 4.42 (t, J=5.08 Hz, 1H), 4.52-4.61 (m, 1H), 7.47-7.54 (m, 1H), 7.74-7.81 (m, 1H), 7.91-8.02 (m, 1H); [M+H]+ calc.=534.2433, [M+H]+ obs.=534.2433.

### Step B. N-(2-(cyclopropylamino)-2-oxoethyl)-9-(ethylsulfonyl)-N-methyl-3-(tetrahydro-2H-pyran-4-yl)-2,3,4,9-tetrahydro-1H-carbazole-6-carboxamide

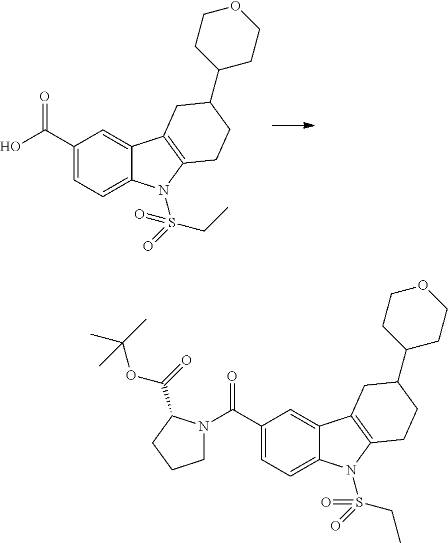

HATU (583 mg, 1.53 mmol) and (R)-tert-butyl pyrrolidine-2-carboxylate (262 mg, 1.53 mmol) were added slowly at 0° C. to a solution of 9-(ethylsulfonyl)-3-(tetrahydro-2H-pyran-4-yl)-2,3,4,9-tetrahydro-1H-carbazole-6-carboxylic acid (300 mg, 0.77 mmol) and N,N-diisopropylethylamine (0.400 mL, 2.30 mmol) in DMF (6.08 mL). Reaction mixture was stirred at room temperature for an O/N. The solvent was then removed in vacuo to provide the crude compound as yellow oil. The (2R)-tert-butyl 1-(9-(ethylsulfonyl)-3-(tetrahydro-2H-pyran-4-yl)-2,3,4,9-tetrahydro-1H-carbazole-6-carbonyl)pyrrolidine-2-carboxylate (53.0%) was purified by Prep-LCMS reverse-phase using a low pH 60-80% ACN/water system. MS (ESI) (M+H)+ 545.5.

### Step C. (2R)-1-(9-(ethylsulfonyl)-3-(tetrahydro-2H-pyran-4-yl)-2,3,4,9-tetrahydro-1H-carbazole-6-carbonyl)pyrrolidine-2-carboxylic acid

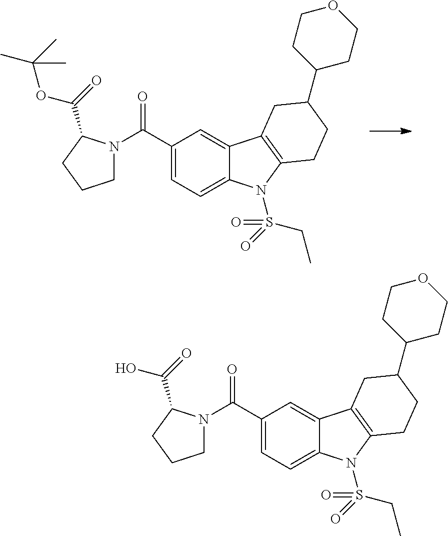

The (2R)-tert-butyl 1-(9-(ethylsulfonyl)-3-(tetrahydro-2H-pyran-4-yl)-2,3,4,9-tetrahydro-1H-carbazole-6-carbonyl)pyrrolidine-2-carboxylate (253.6 mg, 0.47 mmol) was diluted in MeOH (3.152 mL) and lithium hydroxide (111 mg, 4.66 mmol) in water (0.315 mL) was added. The reaction mixture was stirred at 50° C. until total completion of the reaction. Acetic acid was slowly added to obtain a pH of 5-6 and the mixture was concentrated in vacuo. The (2R)-1-(9-(ethylsulfonyl)-3-(tetrahydro-2H-pyran-4-yl)-2,3,4,9-tetrahydro-1H-carbazole-6-carbonyl)pyrrolidine-2-carboxylic acid (24.81%) and the (2R)-1-(3-(tetrahydro-2H-pyran-4-yl)-2,3,4,9-tetrahydro-1H-carbazole-6-carbonyl)pyrrolidine-2-carboxylic acid (69.6 mg, 34.6%) were purified by Prep-HPLC reverse-phase using a low pH 60-80% ACN/water system. 1H NMR (400 MHz, CD₃OD-D4) δ ppm 1.14 (t, J=7.42 Hz, 3H), 1.32-1.66 (m, 6H), 1.78 (t, J=10.16 Hz, 2H), 1.85-1.97 (m, 1H), 1.98-2.11 (m, 2H), 2.12-2.22 (m, 1H), 2.28-2.47 (m, 2H), 2.79-2.92 (m, 2H), 3.08-3.23 (m, 1H), 3.35 (q, J=7.68 Hz, 2H), 3.43 (t, J=11.91 Hz, 2H), 3.54-3.80 (m, 2H), 3.99 (dd, J=11.33, 3.13 Hz, 2H), 4.60 (dd, J=8.20, 5.47 Hz, 1H), 7.43-7.56 (m, 1H), 7.67-7.74 (m, 1H), 7.92-8.06 (m, 1H); MS (ESI) (M+H)+ 489.4.

### Example 114

### N-(2-(2,2-difluoroethylamino)-2-oxoethyl)-N-ethyl-9-methyl-3-(tetrahydro-2H-pyran-4-yl)-2,3,4,9-tetrahydro-1H-carbazole-6-carboxamide

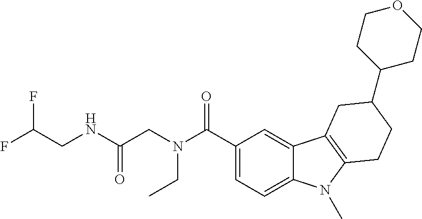

### Step A. N-(2-(2,2-difluoroethylamino)-2-oxoethyl)-N-ethyl-9-methyl-3-(tetrahydro-2H-pyran-4-yl)-2,3,4,9-tetrahydro-1H-carbazole-6-carboxamide

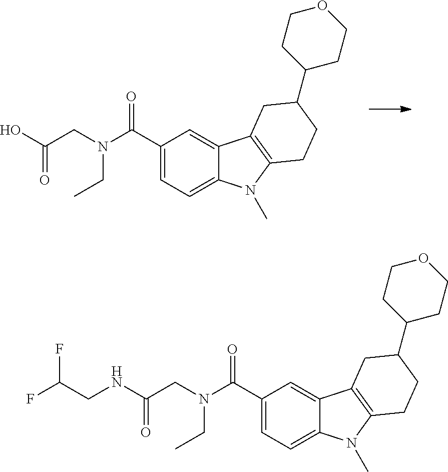

HATU (134 mg, 0.35 mmol) and 2,2-difluoroethanamine (0.024 mL, 0.35 mmol) were added slowly at 0° C. to a solution of 2-(N-ethyl-9-methyl-3-(tetrahydro-2H-pyran-4-yl)-2,3,4,9-tetrahydro-1H-carbazole-6-carboxamido)acetic acid (70 mg, 0.18 mmol) and N,N-diisopropylethylamine (0.118 mL, 0.68 mmol) in DMF (2 mL). Reaction mixture was stirred at room temperature for 3 h. The solvent was then removed in vacuo to provide the crude compound as yellow oil. The N-(2-(2,2-difluoroethylamino)-2-oxoethyl)-N-ethyl-9-methyl-3-(tetrahydro-2H-pyran-4-yl)-2,3,4,9-tetrahydro-1H-carbazole-6-carboxamide (78 mg, 77%) was purified by Prep-HPLC reverse-phase using a low pH 60-80% ACN/water system. 1H NMR (400 MHz, CD₃OD) δ ppm 1.08-1.28 (m, 4H), 1.31-1.48 (m, 2H), 1.47-1.61 (m, 3H), 1.74 (t, J=13.28 Hz, 2H), 2.08-2.20 (m, 1H), 2.29-2.46 (m, 1H), 2.57-2.72 (m, 1H), 2.74-2.90 (m, 2H), 3.35-3.46 (m, 2H), 3.45-3.58 (m, 4H), 3.60 (s, 3H), 3.97 (dd, J=11.13, 3.71 Hz, 2H), 4.02-4.29 (m, 2H), 5.60-6.13 (m, 1H), 7.13-7.23 (m, 1H), 7.25-7.36 (m, 1H), 7.45-7.61 (m, 1 H); [M+H]+ calc.=462.2563, [M+H]+ obs.=462.2559.

### Step B. 2-(N-ethyl-9-methyl-3-(tetrahydro-2H-pyran-4-yl)-2,3,4,9-tetrahydro-1H-carbazole-6-carboxamido)acetic acid

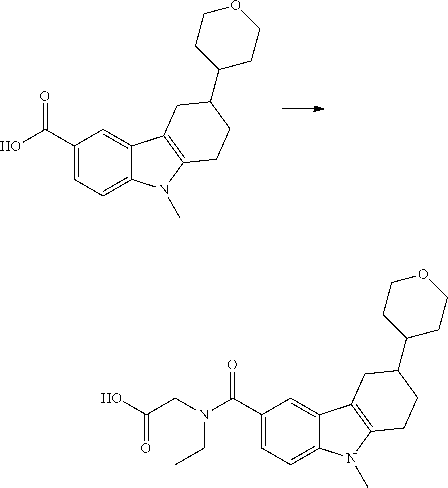

HATU (243 mg, 0.64 mmol) and N-Ethylglycine (99 mg, 0.96 mmol) were added slowly at 0° C. to a solution of 9-methyl-3-(tetrahydro-2H-pyran-4-yl)-2,3,4,9-tetrahydro-1H-carbazole-6-carboxylic acid (100 mg, 0.32 mmol) and N,N-diisopropylethylamine (0.167 mL, 0.96 mmol) in DMF (2.53 mL). Reaction mixture was stirred at room temperature for an O/N. The solvent was then removed in vacuo to provide the crude compound as yellow oil. The 2-(N-ethyl-9-methyl-3-(tetrahydro-2H-pyran-4-yl)-2,3,4,9-tetrahydro-1H-carbazole-6-carboxamido)acetic acid (115 mg, 70.3%) was purified by Prep-HPLC reverse-phase using a low pH 60-80% ACN/water system. MS (ESI) (M+H)+ 399.4.

### Example 115

### N-ethyl-N-(2-((R)-2-hydroxypropylamino)-2-oxoethyl)-9-methyl-3-(tetrahydro-2H-pyran-4-yl)-2,3,4,9-tetrahydro-1H-carbazole-6-carboxamide

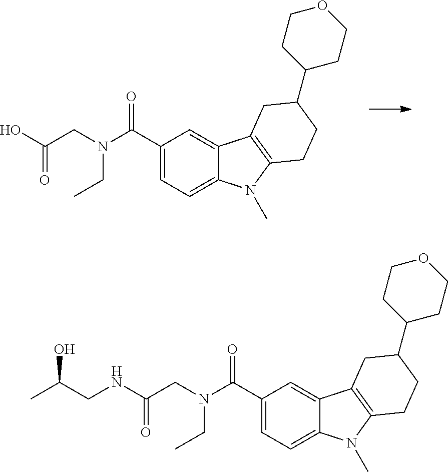

HATU (72.5 mg, 0.19 mmol) and (R)-(−)-1-amino-2-propanol (0.015 mL, 0.19 mmol) were added slowly at 0° C. to a solution of 2-(N-ethyl-9-methyl-3-(tetrahydro-2H-pyran-4-yl)-2,3,4,9-tetrahydro-1H-carbazole-6-carboxamido)acetic acid (38 mg, 0.10 mmol) and N,N-diisopropylethylaminc (0.0523 mL, 0.30 mmol) in DMF (0.757 mL). Reaction mixture was stirred at room temperature for 5 h. The solvent was then removed in vacuo to provide the crude compound as yellow oil. The N-ethyl-N-(2-((R)-2-hydroxypropylamino)-2-oxoethyl)-9-methyl-3-(tetrahydro-2H-pyran-4-yl)-2,3,4,9-tetrahydro-1H-carbazole-6-carboxamide (10.97 mg, 20.20%) was purified by Prep-HPLC reverse-phase using a low pH 60-80% ACN/water system. 1H NMR (400 MHz, CD₃OD-D4) δ ppm 1.05-1.30 (m, 7H), 1.37-1.53 (m, 3H), 1.53-1.67 (m, 4H), 1.78 (t, J=13.09 Hz, 2H), 2.11-2.25 (m, 1H), 2.33-2.45 (m, 1H), 2.63-2.77 (m, 1H), 2.79-2.93 (m, 2H), 3.08-3.23 (m, 1H), 3.37-3.60 (m, 5H), 3.64 (s, 3H), 3.76-3.92 (m, 1H), 3.99 (dd, J=11.33, 3.52 Hz, 2H), 4.09-4.22 (m, 1H), 7.16-7.25 (m, 1H), 7.27-7.38 (m, 1H), 7.49-7.60 (m, 1H); [M+H]+ calc.=456.2857, [M+H]+ obs.=456.2853.

### Example 116

### N-ethyl-N-(2-((S)-2-hydroxypropylamino)-2-oxoethyl)-9-methyl-3-(tetrahydro-2H-pyran-4-yl)-2,3,4,9-tetrahydro-1H-carbazole-6-carboxamide

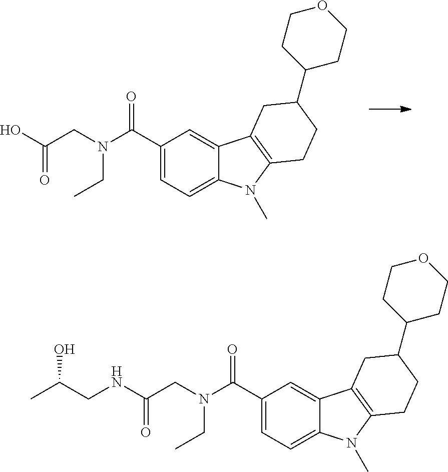

HATU (303 mg, 0.80 mmol) and (S)-(+)-1-amino-2-propanol (0.063 mL, 0.80 mmol) were added slowly at 0° C. to a solution of 2-(N-ethyl-9-methyl-3-(tetrahydro-2H-pyran-4-yl)-2,3,4,9-tetrahydro-1H-carbazole-6-carboxamido)acetic acid (159 mg, 0.40 mmol) and N,N-diisopropylethylamine (0.208 mL, 1.19 mmol) in DMF (3.17 mL). Reaction mixture was stirred at room temperature for 3 h. The solvent was then removed in vacuo to provide the crude compound as yellow oil. The N-ethyl-N-(2-((S)-2-hydroxypropylamino)-2-oxoethyl)-9-methyl-3-(tetrahydro-2H-pyran-4-yl)-2,3,4,9-tetrahydro-1H-carbazole-6-carboxamide (99 mg, 43.5%) was purified by Prep-HPLC reverse-phase using a low pH 50-70% ACN/water system. ¹H NMR (400 MHz, CD₃OD-D4) δ ppm 1.07-1.28 (m, 7H), 1.34-1.50 (m, 3H), 1.50-1.62 (m, 4H), 1.76 (t, J=13.09 Hz, 2H), 2.04-2.19 (m, 1H), 2.36 (dd, J=12.11, 5.47 Hz, 1H), 2.60-2.74 (m, 1H), 2.76-2.90 (m, 2H), 3.10-3.20 (m, 1H), 3.21-3.28 (m, 1H), 3.36-3.57 (m, 5 H), 3.62 (s, 3H), 3.75-3.90 (m, 1H), 3.98 (dd, J=11.13, 3.32 Hz, 2H), 4.05-4.20 (m, 1 H), 7.17-7.24 (m, 1H), 7.52-7.58 (m, 1H); [M+H]+ calc.=456.2857, [M+H]+ obs.=456.2853.

### Example 117

### N-ethyl-N-(2-(2-methoxyethylamino)-2-oxoethyl)-9-methyl-3-(tetrahydro-2H-pyran-4-yl)-2,3,4,9-tetrahydro-1H-carbazole-6-carboxamide

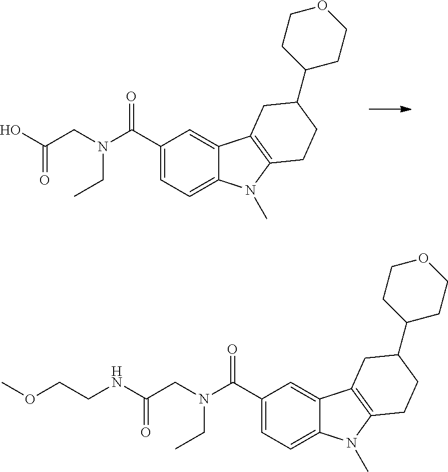

HATU (169 mg, 0.44 mmol) and 2-Methoxyethylamine (0.039 mL, 0.44 mmol) were added slowly at 0° C. to a solution of 2-(N-ethyl-9-methyl-3-(tetrahydro-2H-pyran-4-yl)-2,3,4,9-tetrahydro-1H-carbazole-6-carboxamido)acetic acid (88.4 mg, 0.22 mmol) and N,N-diisopropylethylamine (0.115 mL, 0.66 mmol) in DMF (1.761 mL). Reaction mixture was stirred at room temperature for 2 h30. The solvent was then removed in vacuo to provide the crude compound as yellow oil. The N-ethyl-N-(2-(2-methoxyethylamino)-2-oxoethyl)-9-methyl-3-(tetrahydro-2H-pyran-4-yl)-2,3,4,9-tetrahydro-1H-carbazole-6-carboxamide (59.8 mg, 47.3%) was purified by Prep-HPLC reverse-phase using a low pH 60-80% ACN/water system. ¹H NMR (400 MHz, DMSO-D6) δ ppm 0.99-1.15 (m, 3H), 1.23-1.38 (m, 2H), 1.42-1.55 (m, 3H), 1.69 (t, J=13.48 Hz, 2H), 2.00-2.11 (m, 1H), 2.25-2.37 (m, 1H), 2.57-2.89 (m, 3H), 3.19-3.40 (m, 11H), 3.60 (s, 3H), 3.89 (d, J=9.37 Hz, 2H), 3.92-4.02 (m, 1H), 4.10-4.28 (m, 2H), 7.06-7.16 (m, 1H), 7.29-7.40 (m, 1H), 7.93-8.04 (m, 1H); [M+H]+ calc.=456.2857, [M+H]+ obs.=456.2857.

### Example 118

### N—((R)-1-(cyclopropylamino)-1-oxopropan-2-yl)-N,9-dimethyl-3-(tetrahydro-2H-pyran-4-yl)-2,3,4,9-tetrahydro-1H-carbazole-6-carboxamide

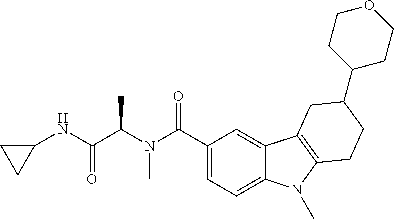

### Step A. (2R)-2-(N,9-dimethyl-3-(tetrahydro-2H-pyran-4-yl)-2,3,4,9-tetrahydro-1H-carbazole-6-carboxamido)propanoic acid

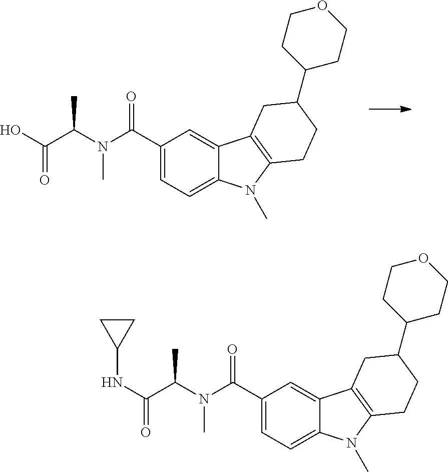

HATU (84 mg, 0.22 mmol) and cyclopropanamine (9.99 □L, 0.14 mmol) were added slowly at 0° C. to a solution of (2R)-2-(N,9-dimethyl-3-(tetrahydro-2H-pyran-4-yl)-2,3,4,9-tetrahydro-1H-carbazole-6-carboxamido)propanoic acid (67.9 mg, 0.17 mmol) and N,N-diisopropylethylamine (0.089 mL, 0.51 mmol) in DMF (1.352 mL). Reaction mixture was stirred at room temperature for an O/N. The solvent was then removed in vacuo to provide the crude compound yellow oil. The N—((R)-1-(cyclopropylamino)-1-oxopropan-2-yl)-N,9-dimethyl-3-(tetrahydro-2H-pyran-4-yl)-2,3,4,9-tetrahydro-1H-carbazole-6-carboxamide (74.6 mg, 47%) was purified by Prep-LCMS reverse-phase using a low pH 40-60% ACN/water system. ¹H NMR (400 MHz, CD₃OD) □ ppm 0.52 (s, 2H), 0.73 (dd, J=7.03, 1.17 Hz, 2H), 1.35-1.49 (m, 6H), 1.50-1.63 (m, 4H), 1.76 (t, J=12.30 Hz, 2H), 2.11-2.19 (m, 1H), 2.30-2.43 (m, 1H), 2.62-2.73 (m, 2H), 2.78-2.89 (m, 2H), 3.03 (s, 3H), 3.36-3.47 (m, 2H), 3.62 (s, 3H), 3.98 (dd, J=11.13, 3.71 Hz, 2H), 7.13-7.21 (m, 1H), 7.24-7.37 (m, 1H), 7.41-7.58 (m, 1H); MS (ESI) (M+H)+ 438.3.

### Step B. (2R)-2-(N,9-dimethyl-3-(tetrahydro-2H-pyran-4-yl)-2,3,4,9-tetrahydro-1H-carbazole-6-carboxamido)propanoic acid

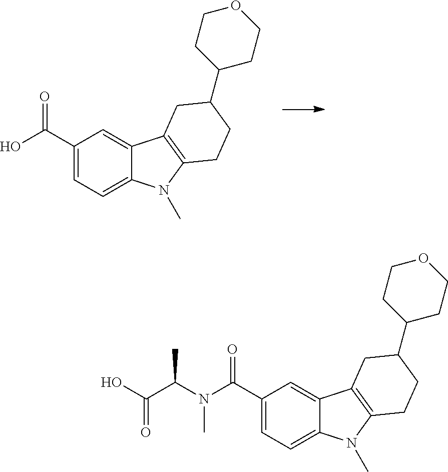

HATU (631 mg, 1.66 mmol) and N-methyl-D-alanine (395 mg, 3.83 mmol) were added slowly at 0° C. to a solution of 9-methyl-3-(tetrahydro-2H-pyran-4-yl)-2,3,4,9-tetrahydro-1H-carbazole-6-carboxylic acid (400 mg, 1.28 mmol) and N,N-diisopropylethylamine (0.667 mL, 3.83 mmol) in DMF (10.0 mL). Reaction mixture was stirred at room temperature for an O/N. The solvent was then removed in vacuo to provide the crude compound as yellow oil. The (2R)-2-(N,9-dimethyl-3-(tetrahydro-2H-pyran-4-yl)-2,3,4,9-tetrahydro-1H-carbazole-6-carboxamido)propanoic acid (210 mg, 41%) was purified by Prep-LCMS reverse-phase using a low pH 40-60% ACN/water system. 1H NMR (400 MHz, CD₃OD) □ ppm 1.28-1.47 (m, 5H), 1.47-1.64 (m, 5H), 1.65-1.81 (m, 2H), 2.02-2.23 (m, 1H), 2.26-2.49 (m, 1H), 2.67 (s, 1H), 2.74-2.92 (m, 2H), 2.97-3.08 (m, 3H), 3.33-3.51 (m, 2H), 3.62 (s, 3H), 3.97 (d, J=10.55 Hz, 2H), 7.05-7.26 (m, 1H), 7.29-7.40 (m, 1H), 7.44-7.65 (m, 1H); MS (ESI) (M+H)+ 399.4.

### Example 119

### Step A

### (R)—N—((S)-1-Hydroxy-5-(oxetan-3-ylamino)-5-oxopentan-2-yl)-N,9-dimethyl-3-(tetrahydro-2H-pyran-4-yl)-2,3,4,9-tetrahydro-1H-carbazole-6-carboxamide

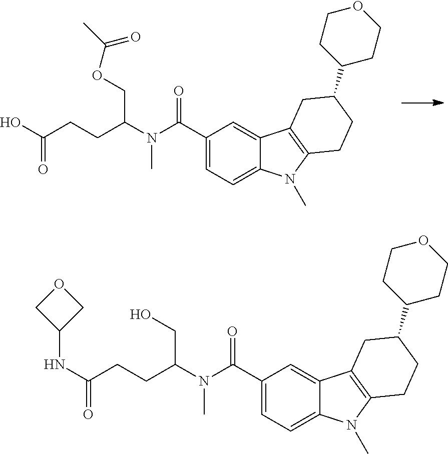

(S)-5-Acetoxy-4((R)—N,9-dimethyl-3-(tetrahydro-2H-pyran-4-yl)-2,3,4,9-tetrahydro-1H-carbazole-6-carboxamido)pentanoic acid (53 mg, 0.11 mmol), oxetan-3-amine hydrochloride (13.18 mg, 0.12 mmol) and O-(7-azabenzotriazol-1-yl)-N,N,N′,N′-tetramethyluronium hexafluorophosphate (45.7 mg, 0.12 mmol) were stirred in DMF (5 mL) at 23° C. for 1 h. The solvent was evaporated. The residue was dissolved in EtOAc and washed with 5% KHSO₄, saturated aqueous NaHCO₃, brine and dried over anhydrous MgSO₄. The solvent was evaporated. The residue was dissolved in MeOH (2 mL) and sodium methoxide (0.025 mL, 0.11 mmol) (25% w/v in MeOH) was added. The solution was stirred at 23° C. for 5 min. The product was purified by reversed-phase HPLC and lyophilized (37 mg, 68%). Reversed-phase purification: Gilson system equipped with X-Bridge Prep C18 OBD, 30×50 mm, 5 mm particle size. Mobile phase: 20-40% B; A: H₂O with 15 mM NH₄CO₃ and 0.375% NH₄OH v/v, B: CH₃CN; 45 mL/min, 15 min run, rt. ¹H NMR (400 MHz, METHANOL-D4) δ 1.29-1.43 (m, 2H), 1.45-1.55 (m, 3H), 1.57-1.66 (m, 1H), 1.70 (t, J=11.91 Hz, 2H), 1.76-1.85 (m, 1 H), 1.88-1.98 (m, 0.5H), 2.01-2.07 (m, 0.5H), 2.07-2.13 (m, 1H), 2.22-2.29 (m, 1 H), 2.29-2.37 (m, 1H), 2.57-2.68 (m, 1H), 2.78 (d, J=16.02 Hz, 2H), 2.85 (d, J=27.73 Hz, 3H), 3.30-3.39 (m, 2H), 3.47 (dd, J=1.91, 4.88 Hz, 0.5H), 3.55 (s, 3H), 3.58-3.67 (m, 1.5H), 3.91 (dd, J=11.13, 4.10 Hz, 2H), 3.96-4.04 (m, 0.5H), 4.19-4.28 (m, 1H), 4.41-4.50 (m, 1H), 4.52-4.60 (m, 1.5H), 4.60-4.67 (m, 0.5H), 4.69-4.78 (m, 1 H), 4.81-4.89 (m, 0.5H), 7.09-7.18 (m, 1H), 7.19-7.27 (m, 1H), 7.47 (d, J=15.62 Hz, 1H); (M+H)=498.2; Accurate mass: calculated (M+H)+ for C28H39N3O5: 498.29625. Found: 498.29580.

### Step B

### (S)-Benzyl 5-acetoxy-4-(tert-butoxycarbonyl(methyl)amino)pentanoate

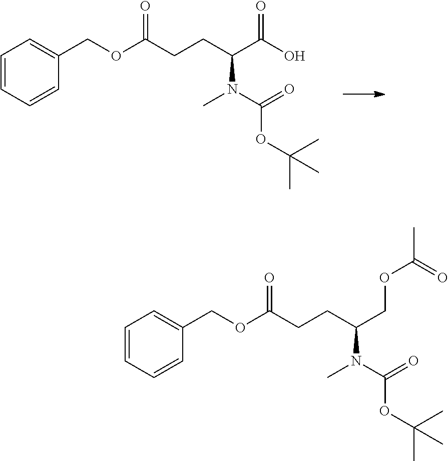

Boc-N-Me-Glu(OBzl)-OH (2.0 g, 5.69 mmol) was dissolved in DME (25 mL) at 0° C. 4-Methylmorpholine (0.626 mL, 5.69 mmol) was added dropwise followed by isobutyl chloroformate (0.738 mL, 5.69 mmol). The solution was stirred at 0° C. for 5 min. The white precipitate was filtered and rinsed with DME. The filtrate was placed back at 0° C. and a solution of sodium borohydride (0.301 mL, 8.54 mmol) in water (10 mL) was added slowly. The solution was then stirred at 0° C. for 30 min. The solvent was concentrated. The residue was dissolved in EtOAc and washed with 5% KHSO₄, saturated aqueous NaHCO₃, brine and dried over anhydrous MgSO₄. The solvent was concentrated. The residue was dissolved in DCM (10 mL) containing triethylamine (1.190 mL, 8.54 mmol) at 0° C. and acetyl chloride (0.445 mL, 6.26 mmol) was added dropwise. The solution was stirred at 0° C. for 30 min. The solution was washed with 5% KHSO₄, saturated aqueous NaHCO₃, brine and dried over anhydrous MgSO₄. The product was purified by flash chromatography (1.73 g, 80%). Flash chromatography is done using a 80 g RediSep column on an Isco Companion system with a gradient of 20-50% EtOAc in heptane. ¹H NMR (400 MHz, CHLOROFORM-D) δ 1.44 (d, J=6.64 Hz, 9H), 1.76-1.87 (m, 2H), 2.01-2.06 (m, 3H), 2.33-2.41 (m, 2H), 2.70 (d, J=15.23 Hz, 3H), 4.01-4.14 (m, 2H), 4.21-4.31 (m, 0.5H), 4.36-4.46 (m, 0.5H), 5.12 (s, 2H), 7.31-7.40 (m, 5H); (M+H)=380.22.

### Step C

### (S)-Benzyl 5-acetoxy-4-(methylamino)pentanoate hydrochloride

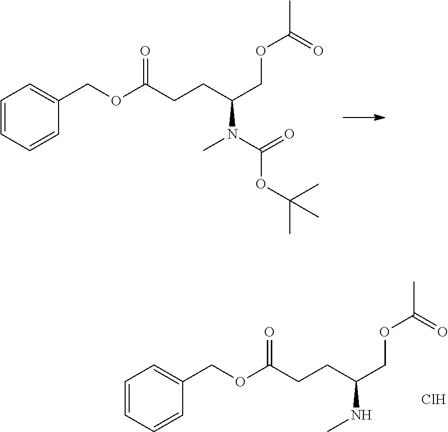

(S)-Benzyl 5-acetoxy-4-(tert-butoxycarbonyl(methyl)amino)pentanoate (1.70 g, 4.48 mmol) was stirred in hydrogen chloride (13.44 mL, 13.44 mmol) (1M in AcOH) at 23° C. for 1 h. The solvent was concentrated. The residue was washed a few times with ether and dried under vacuum. The product was used directly for the next step (1.45 g, 102%) (high yield is probably due to the presence of AcOH in the product). (M+H)=280.22

### Step D

### (S)-Benzyl 5-acetoxy-4-((R)—N,9-dimethyl-3-(tetrahydro-2H-pyran-4-yl)-2,3,4,9-tetrahydro-1H-carbazole-6-carboxamido)pentanoate

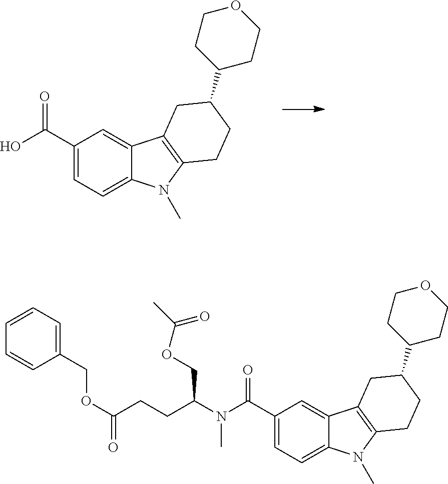

(R)-9-Methyl-3-(tetrahydro-2H-pyran-4-yl)-2,3,4,9-tetrahydro-1H-carbazole-6-carboxylic acid (300 mg, 0.96 mmol), (S)-benzyl 5-acetoxy-4-(methylamino)pentanoate hydrochloride (333 mg, 1.05 mmol) and O-(7-azabenzotriazol-1-yl)-N,N,N′,N′-tetramethyluronium hexafluorophosphate (400 mg, 1.05 mmol) were stirred in DMF (10 mL) containing N,N-diisopropylethylamine (0.417 mL, 2.39 mmol) at 23° C. for 1 h. The solvent was concentrated. The residue was dissolved in EtOAc and washed with 5% KHSO₄, aqueous saturated NaHCO₃, brine and dried over MgSO₄. The product was purified by flash chromatography (395 mg, 72%). Flash chromatography is done using a 40 g RediSep column using an Isco Companion system using a gradient of 50-90% EtOAc in heptane. ¹H NMR (400 MHz, CHLOROFORM-D) δ 1.45 (m, 2H), 1.51-1.63 (m, 3H), 1.67-1.79 (m, 3H), 1.82-1.94 (m, 1H), 1.99 (s, 1H), 2.10 (s, 3H), 2.14 (s, 1H), 2.33-2.44 (m, 2H), 2.53 (s, 1H), 2.63-2.74 (m, 1H), 2.76-2.84 (m, 2H), 2.91 (m, 3H), 3.36-3.47 (m, 2H), 3.62 (s, 3H), 4.03 (d, J=11.33 Hz, 2H), 4.27 (d, 2H), 5.01 (s, 1H), 5.10-5.18 (m, 1H), 7.12-7.18 (m, 1H), 7.19-7.23 (m, 1H), 7.28-7.40 (m, 5H), 7.52 (s, 1H); (M+H)=575.39.

### Step E

### (S)-5-Acetoxy-4((R)—N,9-dimethyl-3-(tetrahydro-2H-pyran-4-yl)-2,3,4,9-tetrahydro-1H-carbazole-6-carboxamido)pentanoic acid

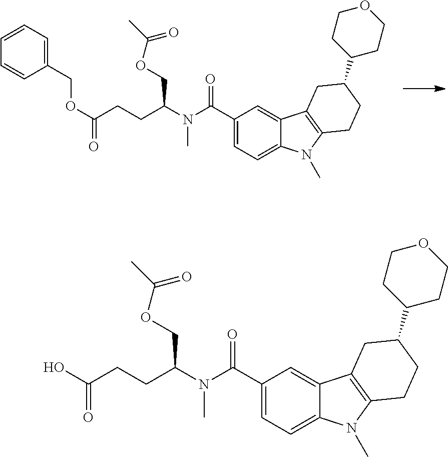

(S)-Benzyl 5-acetoxy-4-((R)—N,9-dimethyl-3-(tetrahydro-2H-pyran-4-yl)-2,3,4,9-tetrahydro-1H-carbazole-6-carboxamido)pentanoate (315 mg, 0.55 mmol) was shaken in ethyl acetate (25 mL) containing palladium (29.2 mg, 0.03 mmol) (10% Pd/C) under hydrogen atmosphere 45 psi at 23° C. for 4 h. The solution was filtered through celite and the solvent was concentrated. The product was purified by flash chromatography (260 mg, 98%). Flash chromatography is done using a 12 g RediSep column using an Isco Companion system with a gradient of 5% MeOH in DCM. ¹H NMR (400 MHz, CHLOROFORM-D) δ 1.41-1.51 (m, 2H), 1.50-1.56 (m, 1H), 1.56-1.64 (m, 2H), 1.69-1.79 (m, 2H), 1.88-2.00 (m, 2H), 2.12 (s, 3H), 2.14-2.19 (m, 1H), 2.35-2.44 (m, 1H), 2.49 (s, 1H), 2.64-2.75 (m, 1H), 2.77-2.87 (m, 2H), 2.88-2.98 (m, 3H), 3.36-3.48 (m, 2H), 3.64 (s, 3H), 4.04 (dd, J=11.52, 2.93 Hz, 2H), 4.21 (s, 1H), 4.34 (s, 0.5H), 4.99 (s, 0.5H), 7.16-7.21 (m, 1H), 7.22-7.26 (m, 1H), 7.52-7.57 (m, 1H); (M+H)=485.35.

### Example 120

### (R)—N—((S)-5-(2,2-Difluoroethylamino)-1-hydroxy-5-oxopentan-2-yl)-N,9-dimethyl-3-(tetrahydro-2H-pyran-4-yl)-2,3,4,9-tetrahydro-1H-carbazole-6-carboxamide

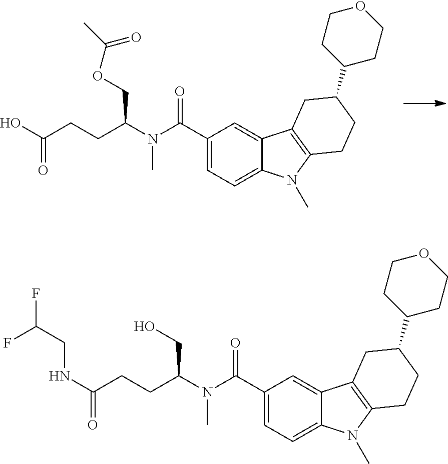

(S)-5-Acetoxy-4-((R)—N,9-dimethyl-3-(tetrahydro-2H-pyran-4-yl)-2,3,4,9-tetrahydro-1H-carbazole-6-carboxamido)pentanoic acid (100 mg, 0.21 mmol), 2,2-difluoroethylamine (18.40 mg, 0.23 mmol) and O-(7-azabenzotriazol-1-yl)-N,N,N′,N′-tetramethyluronium hexafluorophosphate (86 mg, 0.23 mmol) were stirred in DMF (8 mL) containing N,N-diisopropylethylamine (0.072 mL, 0.41 mmol) at 23° C. for 1 h. The solvent was concentrated. The residue was dissolved in EtOAc and washed with 5% KHSO₄, saturated aqueous NaHCO₃, brine and dried over anhydrous MgSO₄. The solvent was evaporated. The residue was dissolved in methanol (5 mL) and sodium methoxide (0.047 mL, 0.21 mmol) (25% solution in MeOH) was added. The solution was stirred at 23° C. for 5 min. The solvent was concentrated. The product was purified by reversed-phase HPLC and lyophilized. Reversed-phase purification: Gilson system equipped with X-Bridge Prep C18 OBD, 30×50 mm, 5 mm particle size. Mobile phase: 30-50% B; A: H₂O with 15 mM NH₄CO₃ and 0.375% NH₄OH v/v, B: CH₃CN; 45 mL/min, 15 min run, rt. (45 mg, 43%). ¹H NMR (400 MHz, METHANOL-D4) δ 1.35-1.50 (m, 2H), 1.57 (s, 3H), 1.65-1.74 (m, 1H), 1.77 (t, J=11.72 Hz, 2H), 1.84-1.93 (m, 1H), 1.96-2.08 (m, 0.5H), 2.10-2.20 (m, 1.5H), 2.30-2.44 (m, 2H), 2.61-2.73 (m, 1H), 2.84 (d, J=15.62 Hz, 2H), 2.92 (d, J=26.95 Hz, 3H), 3.13-3.24 (m, 1H), 3.36-3.47 (m, 2H), 3.48-3.58 (m, 1.5H), 3.62 (s, 3H), 3.64-3.74 (m, 1.5H), 3.97 (dd, J=10.35, 2.93 Hz, 2H), 4.05 (s, 1H), 4.69 (s, 1H), 5.44-5.50 (m, 0.2H), 5.61 (s, 0.2H) 5.70-5.79 (m, 0.2H), 5.86 (s, 0.2H), 6.00 (s, 0.2H), 7.14-7.24 (m, 1H), 7.25-7.34 (m, 1H), 7.52 (d, J=6.25 Hz, 1H); (M+H)=506.2; Accurate mass: calculated (M+H)+ for C27H37F2N3O4: 506.28249. Found: 506.28214.

### Example 121

### (R)—N—((S)-5-(2-Fluoroethylamino)-1-hydroxy-5-oxopentan-2-yl)-N,9-dimethyl-3-(tetrahydro-2H-pyran-4-yl)-2,3,4,9-tetrahydro-1H-carbazole-6-carboxamide

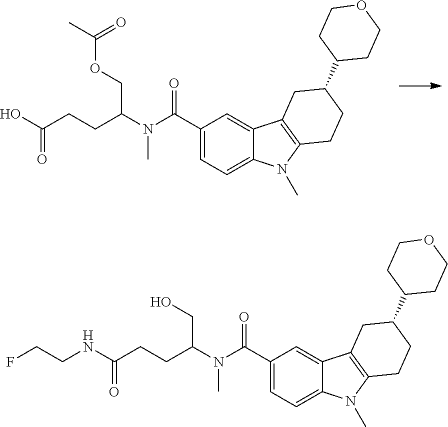

(R)-((4S)-5-Acetoxy-4-(N,9-dimethyl-3-(tetrahydro-2H-pyran-4-yl)-2,3,4,9-tetrahydro-1H-carbazole-6-carboxamido)pentanoic acid (200 mg, 0.41 mmol), 2-fluoroethylamine hydrochloride (49.3 mg, 0.50 mmol) and O-(7-azabenzotriazol-1-yl)-N,N,N′,N-tetramethyluronium hexafluorophosphate (173 mg, 0.45 mmol) were stirred in DMF (5 mL) containing N,N-diisopropylethylamine (0.180 mL, 1.03 mmol) at 23° C. for 1 h. The solvent was concentrated. The residue was dissolved in EtOAc and washed with 5% KHSO₄, saturated aqueous NaHCO₃, brine and dried over anhydrous MgSO₄. The solvent was evaporated. The residue was dissolved in methanol (2 mL) and sodium methoxide (0.094 mL, 0.41 mmol) (25% NaOMe/MeOH) was added. The solution was stirred at 23° C. for 5 min. The product was purified by reversed-phase HPLC and lyophilized. Reversed-phase purification: Gilson system equipped with X-Bridge Prep C18 OBD, 30×50 mm, 5 mm particle size. Mobile phase: 30-50% B; A: H₂O with 15 mM NH₄CO₃ and 0.375% NH₄OH v/v, B: CH₃CN; 45 mL/min, 15 min run, rt. (145 mg, 72%). ¹H NMR (400 MHz, METHANOL-D4) δ 1.35-1.48 (m, 2H), 1.55 (s, 3H), 1.63-1.71 (m, 1H), 1.76 (t, J=11.91 Hz, 2H), 1.95-2.04 (m, 0.5H), 2.08-2.18 (m, 1.5H), 2.29-2.33 (m, 1H), 2.34-2.41 (m, 1H), 2.62-2.72 (m, 1H), 2.83 (d, J=15.23 Hz, 2H), 2.92 (d, J=27.34 Hz, 3H), 3.20-3.26 (m, 1H), 3.29-3.35 (m, 0.5H), 3.36-3.45 (m, 2.5H), 3.47-3.51 (m, 0.5H), 3.53 (d, J=4.69 Hz, 0.5H), 3.61 (s, 3H), 3.64-3.73 (m, 1.5H), 3.97 (dd, J=10.94, 3.91 Hz, 2H), 4.01-4.10 (m, 0.5H), 4.21 (t, J=4.88 Hz, 0.5H), 4.32 (t, J=4.88 Hz, 0.5H), 4.37 (t, J=4.88 Hz, 0.5H), 4.49 (t, J=4.88 Hz, 0.5H), 4.65-4.74 (m, 0.5H), 7.16-7.23 (m, 1H), 7.25-7.33 (m, 1H), 7.53 (d, J=7.42 Hz, 1H); (M+H)=488.3; Accurate mass: calculated (M+H)+ for C27H38FN3O4: 488.28191. Found: 488.29164.

### Example 122

### (R)—N-(4-(Cyanomethylamino)-4-oxobutyl)-N,9-dimethyl-3-(tetrahydro-2H-pyran-4-yl)-2,3,4,9-tetrahydro-1H-carbazole-6-carboxamide

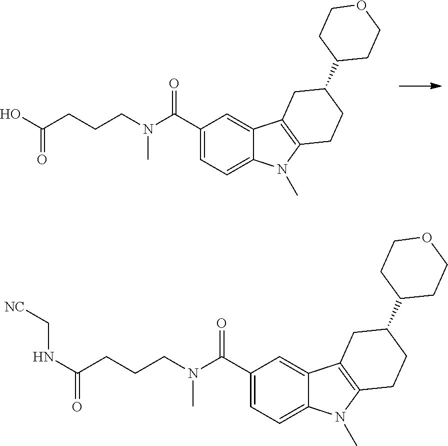

(R)-4-(N,9-Dimethyl-3-(tetrahydro-2H-pyran-4-yl)-2,3,4,9-tetrahydro-1H-carbazole-6-carboxamido)butanoic acid (100 mg, 0.24 mmol), aminoacetonitrile hydrochloride (33.6 mg, 0.36 mmol) and O-(7-azabenzotriazol-1-yl)-N,N,N′,N′-tetramethyluronium hexafluorophosphate (138 mg, 0.36 mmol) were stirred in DMF (5 mL) containing N,N-diisopropylethylamine (0.127 mL, 0.73 mmol) at 23° C. for 1 h. The solvent was concentrated. The product was purified by reversed-phase HPLC and lyophilized. Reversed-phase purification: Gilson system equipped with X-Bridge Prep C18 OD, 30×50 mm, 5 mm particle size. Mobile phase: 20-40% B; A: H₂O with 15 mM NH₄CO₃ and 0.375% NH₄OH v/v, B: CH₃CN; 45 mL/min, 15 min run, rt. (55 mg, 50%). ¹H NMR (400 MHz, METHANOL-D4) δ 1.36-1.49 (m, 2H), 1.51-1.60 (m, 3H), 1.77 (t, J=12.50 Hz, 2H), 1.86 (s, 1H), 1.99 (s, 2H), 2.12-2.19 (m, 1H), 2.30-2.42 (m, 2H), 2.62-2.73 (m, 1H), 2.78-2.89 (m, 2H), 3.04 (s, 3H), 3.36-3.46 (m, 3H), 3.56 (s, 1H), 3.62 (s, 3H), 3.87 (s, 1H), 3.97 (dd, J=10.94, 3.52 Hz, 2H), 4.12 (s, 1H), 7.12 (s, 1 H), 7.31 (d, J=8.59 Hz, 1H), 7.46 (s, 1H); (M+H)=451.2; Accurate mass: calculated (M+H)+ for C26H34N₄O3: 451.27037. Found: 451.26987.

### Example 123

### (R)—N—((S)-1-Hydroxy-5-(methylamino)-5-oxopentan-2-yl)-N,9-dimethyl-3-(tetrahydro-2H-pyran-4-yl)-2,3,4,9-tetrahydro-1H-carbazole-6-carboxamide

### Step A

### (R)—N—((S)-1-Hydroxy-5-(methylamino)-5-oxopentan-2-yl)-N,9-dimethyl-3-(tetrahydro-2H-pyran-4-yl)-2,3,4,9-tetrahydro-1H-carbazole-6-carboxamide

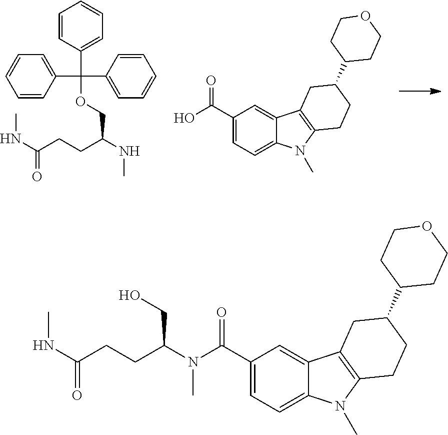

(S)—N-Methyl-4-(methylamino)-5-(trityloxy)pentanamide (184 mg, 0.46 mmol) (R)-9-methyl-3-(tetrahydro-2H-pyran-4-yl)-2,3,4,9-tetrahydro-1H-carbazole-6-carboxylic acid (130 mg, 0.41 mmol) and O-(7-azabenzotriazol-1-yl)-N,N,N′,N¹-tetramethyluronium hexafluorophosphate (173 mg, 0.46 mmol) were stirred in DMF (8 mL) containing N,N-diisopropylethylamine (0.145 mL, 0.83 mmol) at 23° C. for 24 h. The solvent was concentrated. The residue was dissolved in EtOAc and washed with saturated aqueous NaHCO₃, 5% KHSO₄, brine and dried over anhydrous MgSO₄. The product was purified by flash chromatography. The product was then stirred in dioxane (8.00 mL) containing hydrogen chloride (0.519 mL, 2.07 mmol) (4M in dioxane) at 23° C. for 1 h. The solvent was concentrated. The residue was purified by reversed-phase HPLC and lyophilized. Flash chromatography was done using a 40 g RediSep column using an Isco Companion system using a gradient of 5% MeOH/EtOAc. Reversed-phase purification: Gilson system equipped with X-Bridge Prep C18 OBD, 30×50 mm, 5 mm particle size. Mobile phase: 20-40% B; A: H₂O with 15 mM NH₄CO₃ and 0.375% NH₄OH v/v, B: CH₃CN; 45 mL/min, 15 min run, rt. (107 mg, 57%). ¹H NMR (400 MHz, METHANOL-D4) δ 1.35-1.49 (m, 2H), 1.51-1.61 (m, 3H), 1.64-1.72 (m, 1H), 1.76 (t, J=11.91 Hz, 2H), 1.81-1.89 (m, 1H), 1.90-1.99 (m, 0.5H), 2.04-2.11 (m, 0.5H), 2.12-2.18 (m, 1H), 2.24-2.31 (m, 1H), 2.38 (dd, J=15.23, 7.03 Hz, 1H), 2.48 (s, 2H), 2.63-2.74 (m, 2H), 2.80-2.86 (m, 2H), 2.87-2.96 (m, 3H), 3.35-3.46 (m, 2H), 3.51 (dd, 0.5H), 3.61 (s, 3H), 3.64-3.72 (m, 1.5H), 3.97 (dd, J=11.13, 4.10 Hz, 2H), 4.01-4.08 (m, 0.5H), 4.69 (s, 0.5H), 7.15-7.22 (m, 1H), 7.25-7.33 (m, 1H) 7.52 (d, J=9.37 Hz, 1H); (M+H)=456.2; Accurate mass: calculated (M+H)+ for C26H37N3O4: 456.28568. Found: 456.28608.

### Step B

### (S)-5-(trityloxymethyl)pyrrolidin-2-one

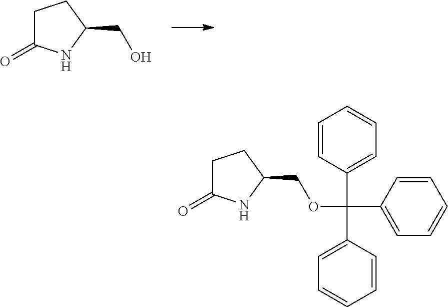

A mixture of (S)-5-(hydroxymethyl)pyrrolidin-2-one (2.07 g, 17.98 mmol), triethylamine (2.506 mL, 17.98 mmol) and N,N-dimethylpyridin-4-amine (0.220 g, 1.80 mmol) in DCM (55.0 mL) was stirred at room temperature for 5 minutes. Chloromethanetriyltribenzene (5.01 g, 17.98 mmol) was added in portions, and the mixture was stirred at room temperature for 60 hours. Water (100 mL) was added, and the phases were separated. The organic extract was washed with water (2×100 mL) and brine (3×100 mL), dried over sodium sulfate, filtered and concentrated under reduced pressure. The product, S)-5-(trityloxymethyl)pyrrolidin-2-one, was sufficiently pure to be used in the next step. ¹H NMR (400 MHz, CHLOROFORM-D) □ ppm 1.54-1.75 (m, 1H) 2.03-2.21 (m, 1H) 2.28 (t, J=8.20 Hz, 2H) 2.93-3.04 (m, 1H), 3.18 (dd, J=8.98, 3.91 Hz, 1H) 3.76-3.91 (m, J=5.66, 4.10 Hz, 1H) 5.87 (s, 1H) 7.20-7.25 (m, 3H) 7.26-7.31 (m, 6H) 7.35-7.41 (m, 6H).

### Step C

### (S)-1-methyl-5-(trityloxymethyl)pyrrolidin-2-one

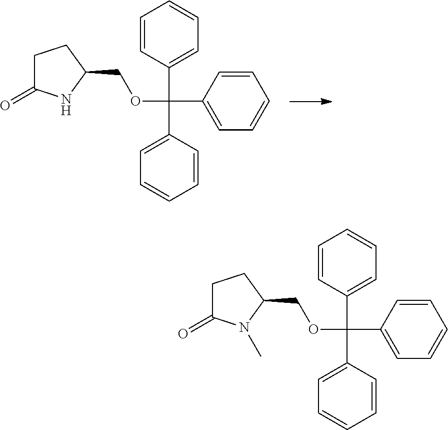

A mixture of (S)-5-(trityloxymethyl)pyrrolidin-2-one (6.43 g, 17.98 mmol) and iodomethane (2.244 mL, 35.96 mmol) in DMF (75.0 mL) under a nitrogen atmosphere was stirred at −15° C. for 5 minutes. NaHMDS (21.58 mL, 21.58 mmol) was added, and the resulting mixture was stirred at −15° C. for 20 minutes and at room temperature for 3 hours. A saturated solution of ammonium chloride (75 mL) and water (100 mL) were added to the reaction mixture, and the phases were separated. The aqueous phase was extracted with DCM (3×100 mL). The combined organic extracts were washed with water (3×100 mL) and brine (2×100 mL), dried over sodium sulfate, filtered and concentrated under reduced pressure. The residue was diluted in Et₂O, and the resulting slurry was filtered. The filtrate was concentrated under reduced pressure, and the residue was purified by flash chromatography on silica gel, eluting with mixtures of EtOAc in heptane (0 to 100%) to afford (S)-1-methyl-5-(trityloxymethyl)pyrrolidin-2-one (3.76 g, 56% for 2 steps). ¹H NMR (400 MHz, CHLOROFORM-D) □ ppm 1.73-1.90 (m, 1H) 2.03-2.17 (m, 1H) 2.22-2.38 (m, 1H) 2.42-2.61 (m, 1H) 2.74 (s, 3H) 3.12 (dd, J=9.96, 4.49 Hz, 1H) 3.27 (dd, J=9.96, 3.71 Hz, 1H) 3.53-3.61 (m, 1H) 7.19-7.25 (m, 3H) 7.26-7.32 (m, 6H) 7.36-7.41 (m, 6H).

### Step D

### (S)—N-Methyl-4-(methylamino)-5-(trityloxy)pentanamide

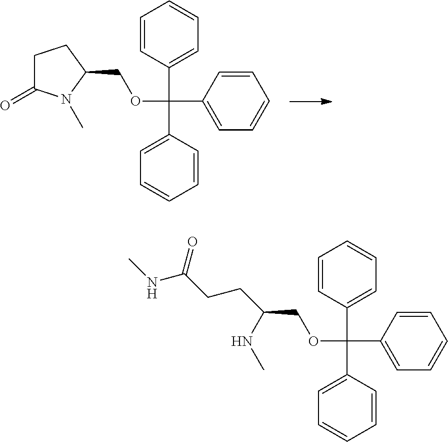

A mixture of methanamine hydrochloride (0.852 g, 12.63 mmol) in THF (20.0 mL) under a nitrogen atmosphere was stirred at room temperature for 5 minutes. Butyllithium (12.63 mL, 25.25 mmol) was added, and the resulting mixture was stirred for 30 minutes. A solution of (S)-1-methyl-5-(trityloxymethyl)pyrrolidin-2-one (0.938 g, 2.53 mmol) in THF (20.00 mL) was added, and the resulting mixture was stirred at room temperature for 2 hours. A saturated solution of ammonium chloride (100 mL) was added to the reaction mixture, and the phases were separated. The aqueous phase was extracted with EtOAc (4×75 mL), and the combined organic extracts were washed with brine, dried over sodium sulfate, filtered and concentrated under reduced pressure. The residue was purified by flash chromatography on silica gel, eluting with mixtures of EtOAc, Et₃N and McOH (8:1:1) to afford (S)—N-Methyl-4-(methylamino)-5-(trityloxy)pentanamide (421 mg, 41%). ¹H NMR (400 MHz, CHLOROFORM-D) □ ppm 1.74 (qd, J=6.64, 2.73 Hz, 2H) 2.06-2.19 (m, 2H) 2.20-2.23 (m, 3H) 2.52-2.61 (m, 1H) 2.70 (d, J=4.69 Hz, 3H) 3.03 (dd, J=9.37, 5.86 Hz, 1H) 3.15 (dd, J=9.37, 4.30 Hz, 1H) 6.22 (s, 1H) 7.17-7.24 (m, 3H) 7.25-7.31 (m, J=7.23, 7.23 Hz, 6H) 7.36-7.42 (m, J=6.84, 6.84 Hz, 6H); (M+H)=403.3.

### Example 124

### (R)—N—((S)-1-Hydroxy-5-(isopropylamino)-5-oxopentan-2-yl)-N,9-dimethyl-3-(tetrahydro-2H-pyran-4-yl)-2,3,4,9-tetrahydro-1H-carbazole-6-carboxamide

### Step A

### (R)—N—((S)-1-Hydroxy-5-(isopropylamino)-5-oxopentan-2-yl)-N,9-dimethyl-3-(tetrahydro-2H-pyran-4-yl)-2,3,4,9-tetrahydro-1H-carbazole-6-carboxamide

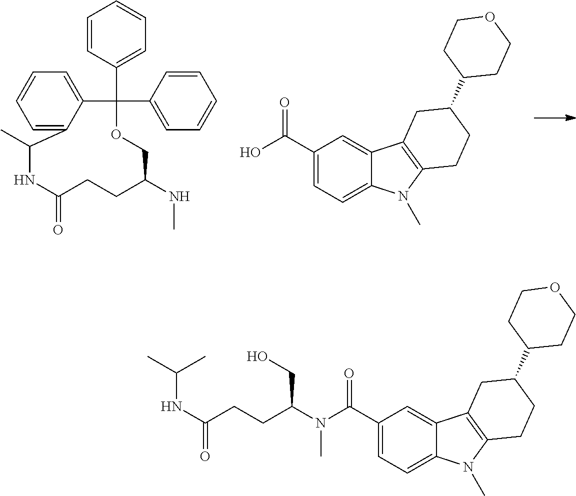

(S)—N-isopropyl-4-(methylamino)-5-(trityloxy)pentanamide (227 mg, 0.53 mmol), (R)-9-methyl-3-(tetrahydro-2H-pyran-4-yl)-2,3,4,9-tetrahydro-1H-carbazole-6-carboxylic acid (150 mg, 0.48 mmol) and O-(7-azabenzotriazol-1-yl)-N,N,N′,N′-tetramethyluronium hexafluorophosphate (200 mg, 0.53 mmol) were stirred in DMF (8 mL) containing N,N-diisopropylethylamine (0.167 mL, 0.96 mmol) at 23° C. for 24 h. The solvent was concentrated. The residue was dissolved in EtOAc and washed with saturated aqueous NaHCO₃, 5% KHSO₄, brine and dried over anhydrous MgSO₄. The product was purified by flash chromatography. The product was then dissolved in dioxane (10 mL) and hydrogen chloride (0.598 mL, 2.39 mmol) (4M/dioxane) was added. The solution was stirred at 23° C. for 4-5 h. The solvent was concentrated. The product was purified by reversed-phase HPLC and lyophilized. Flash chromatography is done using a 40 g RediSep column using an Isco Companion system using a gradient of EtOAc. Reversed-phase purification: Gilson system equipped with X-Bridge Prep C18 OBD, 30×50 mm, 5 mm particle size. Mobile phase: 30-50% B; A: H₂O with 15 mM NH₄CO₃ and 0.375% NH₄OH v/v, B: CH₃CN; 45 mL/min, 15 min run, rt. (110 mg, 48%). ¹H NMR (400 MHz, METHANOL-D4) δ 0.96 (d, J=5.86 Hz, 3H), 1.11 (t, J=7.23 Hz, 3H), 1.35-1.47 (m, 2H), 1.55 (s, 3H), 1.60-1.69 (m, 0.5H), 1.75 (t, J=12.30 Hz, 3H), 1.82-1.95 (m, 1H), 2.00-2.10 (m, 0.5H), 2.11-2.18 (m, 1H), 2.21-2.29 (m, 1H), 2.32-2.41 (m, 1H), 2.61-2.71 (m, 1H), 2.79-2.87 (m, 2H), 2.87-2.97 (m, 3H), 3.35-3.45 (m, 2H), 3.51 (m, 0.5H), 3.60 (s, 3H), 3.63-3.71 (m, 1H), 3.72-3.80 (m, 0.5H), 3.96 (d, J=10.94 Hz, 2H), 4.00-4.07 (m, 0.5H), 4.63-4.72 (m, 0.5H), 7.16-7.22 (m, 1H), 7.25-7.33 (m, 1H), 7.50-7.55 (m, 1H); (M+H)=484.2; Accurate mass: calculated (M+H)+ for C28H41N3O4: 484.31698. Found: 484.31682.

### Step B

### (S)—N-isopropyl-4-(methylamino)-5-(trityloxy)pentanamide

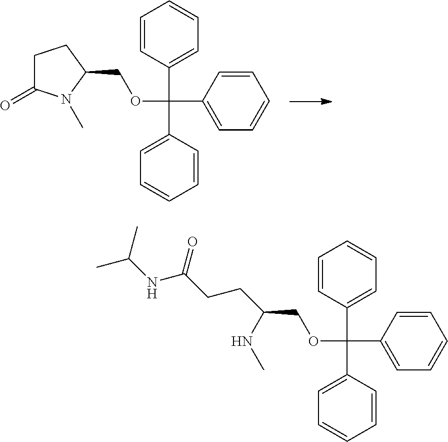

A mixture of propan-2-amine hydrochloride (643 mg, 6.73 mmol) in THF (20.0 mL) under a nitrogen atmosphere was stirred at room temperature for 5 minutes. Butylithium (6.73 mL, 13.46 mmol) was added, and the resulting mixture was stirred for 30 minutes. A solution of (S)-1-methyl-5-(trityloxymethyl)pyrrolidin-2-one (500 mg, 1.35 mmol, see example 5 for synthesis) in THF (20.00 mL) was added, and the mixture was stirred at room temperature for 12 hours. A saturated solution of ammonium chloride (100 mL) and a 5% solution of KHSO₄ (10 mL) were added to the reaction mixture, and the phases were separated. The aqueous phase was extracted with EtOAc (4×75 mL). The combined organic extracts were washed with brine, dried over sodium sulfate, filtered and concentrated under reduced pressure. The residue was purified by flash chromatography on silica gel, eluting with mixtures of EtOAc, Et₃N and MeOH (8:1:1) to afford (S)—N-isopropyl-4-(methylamino)-5-(trityloxy)pentanamide (284 mg, 49%). ¹H NMR (400 MHz, METHANOL-D4) □ ppm 1.00 (d, J=5.86 Hz, 5H) 1.55-1.75 (m, 2H) 1.98 (t, J=7.42 Hz, 2H) 2.15 (s, 3H) 2.42-2.53 (m, 1H) 3.00 (dd, J=9.57, 6.05 Hz, 1H) 3.11 (dd, J=9.77, 4.69 Hz, 1H) 3.17-3.24 (m, 1H) 3.75-3.87 (m, 1H) 7.12 (tt, 3H) 7.16-7.22 (m, J=7.42, 7.42 Hz, 6H) 7.30-7.37 (m, 6H); (M+H)=431.4.

### Example 125

### (R)—N—((S)-5-(Ethylamino)-1-hydroxy-5-oxopentan-2-yl)-N,9-dimethyl-3-(tetrahydro-2H-pyran-4-yl)-2,3,4,9-tetrahydro-1H-carbazole-6-carboxamide

### Step A

### (R)—N—((S)-5-(Ethylamino)-1-hydroxy-5-oxopentan-2-yl)-N,9-dimethyl-3-(tetrahydro-2H-pyran-4-yl)-2,3,4,9-tetrahydro-1H-carbazole-6-carboxamide

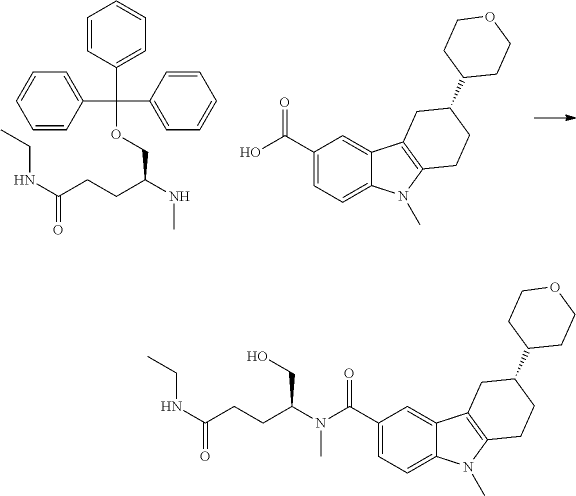

(S)—N-Ethyl-4-(methylamino)-5-(trityloxy)pentanamide (439 mg, 1.05 mmol), (R)-9-methyl-3-(tetrahydro-2H-pyran-4-yl)-2,3,4,9-tetrahydro-1H-carbazole-6-carboxylic acid (300 mg, 0.96 mmol) and O-(7-azabenzotriazol-1-yl)-N,N,N′,N′-tetramethyluronium hexafluorophosphate (400 mg, 1.05 mmol) were stirred in DMF (10 mL) containing N,N-diisopropylethylamine (0.333 mL, 1.91 mmol) at 23° C. for 24 h. The solution was then diluted with saturated aqueous NH₄Cl and extracted (3×) with DCM. The organic layer was dried over anhydrous MgSO₄ and purified by flash chromatography. The product was then stirred in 1M HCl/AcOH at it for 1 h. Some acetylated product was observed. The solvent was concentrated. The residue was dissolved in MeOH (10 mL) and NaOMe (28% w/v) was added. The solution was stirred at it for 10 min. The product was purified by reversed-phase HPLC and lyophilized. Flash chromatography is done using a 40 g RediSep column using an Isco Companion system with a gradient of EtOAc. Reversed-phase purification: Gilson system equipped with X-Bridge Prep C18 OBD, 30×50 mm, 5 mm particle size. Mobile phase: 20-40% B; A: H₂O with 15 mM NH₄CO₃ and 0.375% NH₄OH v/v, B: CH₃CN; 45 mL/min, 15 min run, rt. (215 mg, 48%). ¹H NMR (400 MHz, METHANOL-D4) δ 0.96 (t, J=7.23 Hz, 1H), 1.10 (t, J=7.23 Hz, 1H), 1.35-1.48 (m, 2H), 1.55 (s, 3H), 1.62-1.71 (m, 1H), 1.76 (t, J=12.30 Hz, 2H), 1.81-1.89 (m, 1H), 1.90-1.97 (m, 0.5H), 2.02-2.11 (m, 0.5H), 2.11-2.18 (m, 1H), 2.23-2.31 (m, 1H), 2.33-2.42 (m, 1H), 2.61-2.72 (m, 1H), 2.78-2.90 (m, 3.5H), 2.95 (s, 2H), 2.97-3.06 (m, 0.5H), 3.14-3.22 (m, 1H), 3.36-3.45 (m, 2H), 3.51 (dd, J=11.72, 4.69 Hz, 0.5H), 3.61 (s, 3H), 3.63-3.72 (m, 1.5H), 3.93-4.00 (m, 2H), 4.00-4.09 (m, 0.5H), 4.64-4.73 (m, 0.5H), 7.15-7.23 (m, 1H), 7.25-7.32 (m, 1H), 7.53 (d, J=9.37 Hz, 1H); (M+H)=470.2; Accurate mass: calculated (M+H)+ for C27H39N3O4: 470.30133. Found: 470.30112.

### Step B

### (S)—N-Ethyl-4-(methylamino)-5-(trityloxy)pentanamide

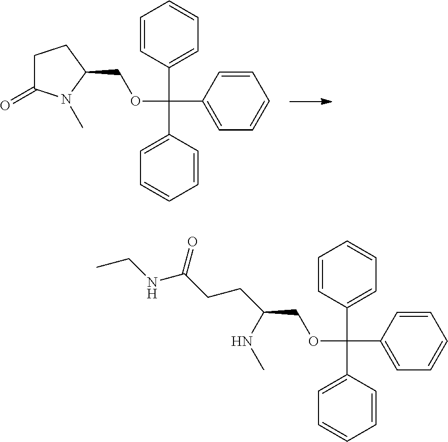

A mixture of ethanamine hydrochloride (659 mg, 8.08 mmol) in THF (20.0 mL) under a nitrogen atmosphere was stirred at room temperature for 5 minutes. Butyllithium (8.08 mL, 16.15 mmol) was added, and the resulting mixture was stirred for 20 minutes. A solution of (S)-1-methyl-5-(trityloxymethyl)pyrrolidin-2-one (600 mg, 1.62 mmol, see example 5 for synthesis) in THF (20.00 mL) was added, and the mixture was stirred at room temperature for 12 hours. A saturated solution of ammonium chloride (100 mL) and a 5% solution of KHSO₄ (10 mL) were added to the reaction mixture, and the phases were separated. The aqueous phase was extracted with EtOAc (4×75 mL), and the combined organic extracts were washed with brine, dried over sodium sulfate, filtered and concentrated under reduced pressure. The residue was purified by flash chromatography on silica gel, eluting with mixtures of EtOAc, MeOH and Et₃N (8:1:1) to afford (S)—N-Ethyl-4-(methylamino)-5-(trityloxy)pentanamide (531 mg, 79%). ¹H NMR (400 MHz, METHANOL-D4) □ ppm 0.98 (t, J=7.42 Hz, 2H) 1.54-1.76 (m, 2H) 1.99 (t, J=7.81 Hz, 2H) 2.04 (s, 3H) 2.15 (s, 3H) 2.85-3.16 (m, 4H) 7.13 (tt, J=7.03, 1.56 Hz, 2H) 7.17-7.23 (m, 6H) 7.32-7.37 (m, 6H); (M+H)=417.4.

### Example 126

### (R)—N-(4-(Methoxyamino)-4-oxobutyl)-N,9-dimethyl-3-(tetrahydro-2H-pyran-4-yl)-2,3,4,9-tetrahydro-1H-carbazole-6-carboxamide

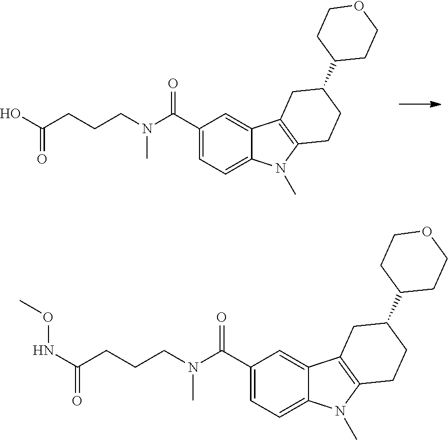

(R)-4-(N,9-Dimethyl-3-(tetrahydro-2H-pyran-4-yl)-2,3,4,9-tetrahydro-1H-carbazole-6-carboxamido)butanoic acid (80 mg, 0.19 mmol), methoxylamine hydrochloride (17.82 mg, 0.21 mmol) and O-(7-azabenzotriazol-1-yl)-N,N,N′,N′-tetramethyluronium hexafluorophosphate (81 mg, 0.21 mmol) were stirred in DMF (5 mL) containing N,N-diisopropylethylamine (0.084 mL, 0.48 mmol) at 23° C. for 1 h. The solvent was concentrated. The product was directly purified by reversed phase HPLC and lyophilized. Reversed-phase purification: Gilson system equipped with X-Bridge Prep C18 OBD, 30×50 mm, 5 mm particle size. Mobile phase: 30-50% B; A: H₂O with 15 mM NH₄CO₃ and 0.375% NH₄OH v/v, B: CH₃CN; 45 mL/min, 15 min run, rt. (55 mg, 64%). ¹H NMR (400 MHz, METHANOL-D4) δ 1.37-1.49 (m, 2H), 1.51-1.61 (m, 3H), 1.77 (t, J=12.50 Hz, 2H), 1.86 (s, 2H), 1.97 (s, 1H), 2.12-2.20 (m, 2H), 2.34-2.43 (m, 1H), 2.63-2.73 (m, 1H), 2.80-2.89 (m, 2H), 3.03 (s, 3H), 3.36-3.46 (m, 4H), 3.57 (s, 2H), 3.62 (s, 3H), 3.67 (s, 1H), 3.97 (dd, J=11.52, 4.10 Hz, 2H), 7.13 (s, 1H), 7.31 (d, J=8.98 Hz, 1H), 7.46 (s, 1H); (M+H)=442.3; Accurate mass: calculated (M+H)+ for C25H35N3O4: 442.27003. Found: 442.27043.

### Example 127

### (R)—N-(4-(2,2-Dimethylhydrazinyl)-4-oxobutyl)-N,9-dimethyl-3-(tetrahydro-2H-pyran-4-yl)-2,3,4,9-tetrahydro-1H-carbazole-6-carboxamide

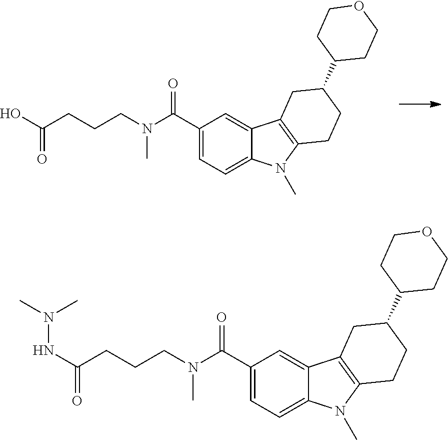

(R)-4-(N,9-Dimethyl-3-(tetrahydro-2H-pyran-4-yl)-2,3,4,9-tetrahydro-1H-carbazole-6-carboxamido)butanoic acid (80 mg, 0.19 mmol), 1,1-dimethylhydrazine (0.018 mL, 0.23 mmol) and O-(7-azabenzotriazol-1-yl)-N,N,N′,N′-tetramethyluronium hexafluorophosphate (88 mg, 0.23 mmol) were stirred in DMF (5 mL) containing N,N-diisopropylethylamine (0.084 mL, 0.48 mmol) at 23° C. for 1 h. The solvent was concentrated. The product was purified by reversed-phase HPLC and lyophilized. Reversed-phase purification: Gilson system equipped with X-Bridge Prep C18 OBD, 30×50 mm, 5 mm particle size. Mobile phase: 30-50% B; A: H₂O with 15 mM NH₄CO₃ and 0.375% NH₄OH v/v, B: CH₃CN; 45 mL/min, 15 min run, rt. (57 mg, 65%). ¹H NMR (400 MHz, METHANOL-D4) δ 1.37-1.49 (m, 2H), 1.51-1.61 (m, 3H), 1.77 (t, J=12.70 Hz, 2H), 1.87 (s, 2H), 1.95 (s, 1H), 2.12-2.21 (m, 2H), 2.32 (s, 2H), 2.34-2.43 (m, 2H), 2.46-2.55 (m, 3H), 2.64-2.73 (m, 1H), 2.79-2.89 (m, 2H), 3.04 (s, 3H), 3.36-3.46 (m, 3H), 3.56 (s, 1H), 3.62 (s, 3H), 3.97 (dd, J=11.33, 3.91 Hz, 2H), 7.13 (s, 1H), 7.31 (d, J=8.59 Hz, 1H), 7.45 (s, 1H); (M+H)=455.3; Accurate mass: calculated (M+H)+ for C26H38N4O3: 455.30167. Found: 455.30119.

### Example 128

### (R)—N-(4-(2-Methoxyethylamino)-4-oxobutyl)-N,9-dimethyl-3-(tetrahydro-2H-pyran-4-yl)-2,3,4,9-tetrahydro-1H-carbazole-6-carboxamide

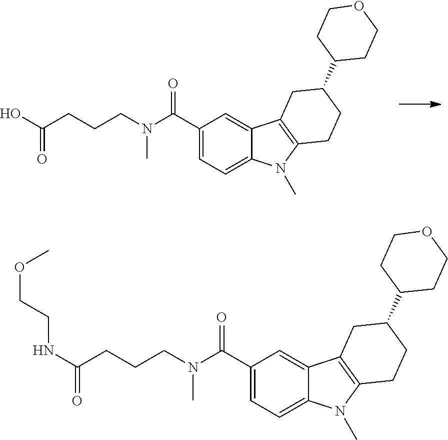

(R)-4-(N,9-Dimethyl-3-(tetrahydro-2H-1-pyran-4-yl)-2,3,4,9-tetrahydro-1H-carbazole-6-carboxamido)butanoic acid (80 mg, 0.19 mmol), 2-methoxyethylamine (0.020 mL, 0.23 mmol) and O-(7-azabenzotriazol-1-yl)-N,N,N′,N′-tetramethyluronium hexafluorophosphate (88 mg, 0.23 mmol) were stirred in DMF (5 mL) containing N,N-diisopropylethylamine (0.084 mL, 0.48 mmol) at 23° C. for 1 h. The solvent was concentrated. The product was directly purified by reversed-phase HPLC and lyophilized. Reversed-phase purification: Gilson system equipped with X-Bridge Prep C18 OBD, 30×50 mm, 5 mm particle size. Mobile phase: 30-50% B; A: H₂O with 15 mM NH₄CO₃ and 0.375% NH₄OH v/v, B: CH₃CN; 45 mL/min, 15 min run, rt. (60 mg, 66%). ¹H NMR (400 MHz, METHANOL-D4) δ 1.36-1.50 (m, 2H), 1.51-1.61 (m, 3H), 1.77 (t, J=12.11 Hz, 2H), 1.87 (s, 1H), 1.97 (s, 2H), 2.11-2.19 (m, 1H), 2.28 (s, 1H), 2.34-2.43 (m, 1H), 2.62-2.73 (m, 1H), 2.80-2.89 (m, 2H), 3.04 (s, 3H), 3.12 (s, 1H), 3.23 (s, 2H), 3.30-3.36 (m, 3H), 3.36-3.46 (m, 4H), 3.56 (s, 1H), 3.62 (s, 3H), 3.97 (dd, J=11.33, 3.91 Hz, 2H), 7.12 (s, 1H), 7.30 (d, J=8.98 Hz, 1H), 7.46 (s, 1H); (M+H)=470.2; Accurate mass: calculated (M+H)+ for C27H39N3O4: 470.30133. Found: 470.30124.

### Example 129

### (R)—N-(4-(1H-Pyrrol-1-ylamino)-4-oxobutyl)-N,9-dimethyl-3-(tetrahydro-2H-pyran-4-yl)-2,3,4,9-tetrahydro-1H-carbazole-6-carboxamide

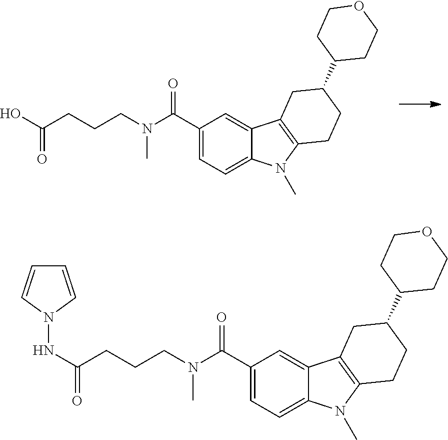

(R)-4-(N,9-Dimethyl-3-(tetrahydro-2H-pyran-4-yl)-2,3,4,9-tetrahydro-1H-carbazole-6-carboxamido)butanoic acid (80 mg, 0.19 mmol), 1-aminopyrrole (0.018 mL, 0.23 mmol) and O-(7-azabenzotriazol-1-yl)-N,N,N′,N′-tetramethyluronium hexafluorophosphate (88 mg, 0.23 mmol) were stirred in DMF (5 mL) containing N,N-diisopropylethylamine (0.084 mL, 0.48 mmol) at 23° C. for 1 h. The solvent was concentrated. The product was directly purified by reversed-phase HPLC and lyophilized. Reversed-phase purification: Gilson system equipped with X-Bridge Prep C18 OBD, 30×50 mm, 5 mm particle size. Mobile phase: 30-50% B; A: H₂O with 15 mM NH₄CO₃ and 0.375% NH₄OH v/v, B: CH₃CN; 45 mL/min, 15 min run, rt. (47 mg, 50%). ¹H NMR (400 MHz, METHANOL-D4) δ 1.36-1.49 (m, 2H), 1.50-1.62 (m, 3H), 1.76 (t, J=13.09 Hz, 2H), 1.96 (s, 1H), 2.07 (s, 1H), 2.11-2.21 (m, 2H), 2.34-2.49 (m, 2H), 2.63-2.76 (m, 1H), 2.84 (t, J=14.65 Hz, 2H), 3.07 (s, 3H), 3.36-3.51 (m, 3H), 3.64 (s, 4H), 3.93-4.03 (m, 2H), 5.94 (s, 1H), 6.04 (s, 1H), 6.20 (s, 1H), 6.64 (s, 1H), 7.16 (d, J=7.03 Hz, 1H), 7.33 (d, J=8.59 Hz, 1H), 7.51 (s, 1H); (M+H)=477.2; Accurate mass: calculated (M+H)+ for C28H36N4O3: 477.28602. Found: 477.28622.

### Example 130

### Step A

### (R)—N-Ethyl-N-(4-(2-hydroxyethylamino)-4-oxobutyl)-9-methyl-3-(tetrahydro-2H-pyran-4-yl)-2,3,4,9-tetrahydro-1H-carbazole-6-carboxamide

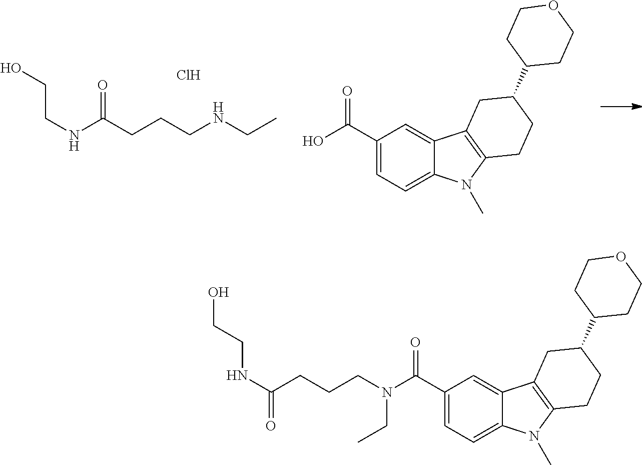

(R)-9-Methyl-3-(tetrahydro-2H-pyran-4-yl)-2,3,4,9-tetrahydro-1H-carbazole-6-carboxylic acid (100 mg, 0.32 mmol), 4-(ethylamino)-N-(2-hydroxyethyl)butanamide hydrochloride (81 mg, 0.38 mmol) and O-(7-azabenzotriazol-1-yl)-N,N,N′,N′-tetramethyluronium hexafluorophosphate (146 mg, 0.38 mmol) were stirred in DMF (8 mL) containing N,N-diisopropylethylamine (0.139 mL, 0.80 mmol) at 23° C. for 1 h. Another 1.2 eq of 4-(ethylamino)-N-(2-hydroxyethyl)butanamide hydrochloride (81 mg, 0.38 mmol) was added and the solution was stirred for another 1 h. The solvent was concentrated. The residue was dissolved in EtOAc and washed with 5% KHSO₄, saturated aqueous NaHCO₃, brine and dried over anhydrous MgSO₄. LC/MS showed presence of product at 512, probably acylated product from residual AcOH. The residue was then stirred in 5 mL of MeOH containing some NaOMe at rt for 15 min. LC/MS showed only the presence of the desired product. Purified by reversed-phase HPLC and lyophilized. Reversed-phase purification: Gilson system equipped with X-Bridge Prep C18 OBD, 30×50 mm, 5 mm particle size. Mobile phase: 30-50% B; A: H₂O with 15 mM NH₄CO₃ and 0.375% NH₄OH v/v, B: CH₃CN; 45 mL/min, 15 min run, rt. (75 mg, 50%). ¹H NMR (400 MHz, METHANOL-D4) δ 1.10 (s, 2H), 1.23 (s, 1H), 1.37-1.48 (m, 2H), 1.52-1.60 (m, 3H), 1.77 (t, J=12.50 Hz, 2H), 1.82-1.90 (m, 1H), 1.92-2.02 (m, 2H), 2.12-2.19 (m, 1H), 2.29 (s, 1H), 2.33-2.42 (m, 1H), 2.63-2.73 (m, 1H), 2.79-2.89 (m, 2H), 3.03-3.16 (m, 1H), 3.30-3.37 (m, 2H), 3.36-3.46 (m, 4H), 3.49-3.60 (m, 3H), 3.62 (s, 3H), 3.97 (dd, J=11.13, 3.71 Hz, 2H), 7.08 (d, J=8.20 Hz, 1H), 7.31 (d, J=8.20 Hz, 1H), 7.41 (s, 1H); (M+H)=470.2; Accurate mass: calculated (M+H)+ for C27H39N3O4: 470.30133. Found: 470.30192.

### Step B

### 4-(Ethylamino)butanoic acid

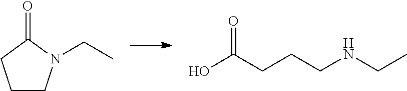

1-Ethyl-2-pyrrolidinone (2.016 mL, 17.67 mmol) and barium hydroxide hydrate (3.35 g, 17.67 mmol) were refluxed in water (20 mL) at 110° C. for 12 h. The solution was cooled to 0° C. and CO₂ gas was bubbled through the solution for 15 min to precipitate the barium hydroxide. The solution was filtered and the filtrate was concentrated to dryness. The solid obtained was triturated with MeCN, filtered and washed with ether. The product was dried under vacuum. (1.20 g, 52%). ¹H NMR (400 MHz, DEUTERIUM OXIDE) δ 1.21 (t, J=7.42 Hz, 3H), 1.77-1.90 (m, 2H), 2.23 (t, J=7.23 Hz, 2H), 2.91-3.07 (m, 4H).

### Step C

### 4-(tert-Butoxycarbonyl(ethyl)amino)butanoic acid

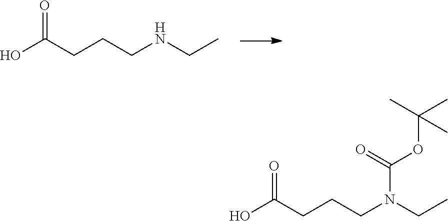

4-(Ethylamino)butanoic acid (1.15 g, 8.77 mmol) was dissolved in a mixture of dioxane (50 mL) and water (50.0 mL) containing potassium carbonate (0.997 mL, 17.53 mmol) at 0° C. Di-tert-butyl dicarbonate (2.218 mL, 9.64 mmol) was added and the solution was stirred at 23° C. overnight. The solvent was concentrated. The aqueous residue was washed with ether. The aqueous layer was then acidified with 5% KHSO₄ and extracted (2×) with EtOAc. The organic phase was dried over anhydrous MgSO₄ and evaporated. (1.55 g, 76%). ¹H NMR (400 MHz, CHLOROFORM-D) □ 1.11 (t, J=7.03 Hz, 3H), 1.46 (s, 9H), 1.85 (dt, J=14.06, 7.03 Hz, 2H), 2.37 (t, J=7.03 Hz, 2H), 3.16-3.32 (m, 4H); (M+H)=232.27.

### Step D

### 4-(Ethylamino)-N-(2-hydroxyethyl)butanamide hydrochloride

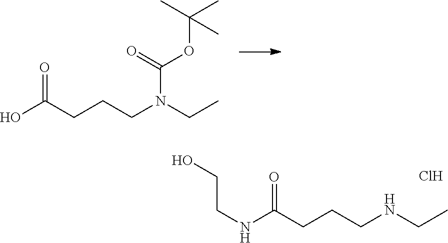

4-(tert-Butoxycarbonyl(ethyl)amino)butanoic acid (300 mg, 1.30 mmol), O-(7-azabenzotriazol-1-yl)-N,N,N′,N′-tetramethyluronium hexafluorophosphate (592 mg, 1.56 mmol) and ethanolamine (0.094 mL, 1.56 mmol) were stirred in DMF (8 mL) containing N,N-diisopropylethylamine (0.339 mL, 1.95 mmol) at 23° C. for 1 h. The solvent was evaporated. The residue was dissolved in EtOAc and washed with 5% KHSO₄, saturated aqueous NaHCO₃, brine and dried over anhydrous MgSO₄. The product was purified by flash chromatography. The product was then stirred in hydrogen chloride (12.97 mL, 12.97 mmol) (1M in AcOH) at 23° C. for 1 h. The solvent was concentrated. The product was washed a few times with ether and dried under vacuum. Still some AcOH left in the product. Used directly for the next step. Yield: 275 mg (122%); (M+H)=289.29 (Boc product; de-Boc product could not be observed by LC/MS).

### Example 131

### (R)—N-(4-(2-Hydroxyethylamino)-4-oxobutyl)-N,9-dimethyl-3-(tetrahydro-2H-pyran-4-yl)-2,3,4,9-tetrahydro-1H-carbazole-6-carboxamide

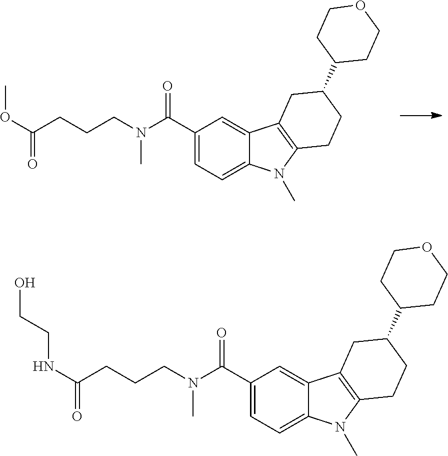

(R)-Methyl 4-(N,9-dimethyl-3-(tetrahydro-2H-pyran-4-yl)-2,3,4,9-tetrahydro-1H-carbazole-6-carboxamido)butanoate (100 mg, 0.23 mmol) was stirred in dioxane (5 mL) containing lithium hydroxide (0.469 mL, 0.47 mmol) (1M) at 23° C. for 2 h. The solvent was evaporated. The residue was dissolved in EtOAc and washed with 5% KHSO₄, brine and dried over anhydrous Na₂SO₄. The solvent was evaporated. The product was dissolved in DMF (5.00 mL) containing N,N-diisopropylethylamine (0.102 mL, 0.59 mmol) and ethanolamine (0.017 mL, 0.28 mmol) along with O-(7-azabenzotriazol-1-yl)-N,N,N′,N′-tetramethyluronium hexafluorophosphate (107 mg, 0.28 mmol) were added. The solution was stirred at 23° C. for 1 h. The solvent was evaporated. The product was directly purified by reversed-phase HPLC and lyophilized. Reversed-phase purification: Gilson system equipped with Luna C-18 column, 250×21.2 mm, 15 u. Mobile phase: 20-40% B; A: H₂O with 0.05% TFA v/v; B: CH₃CN; 30 mL/min, 25 min run, rt. (55 mg, 52%). ¹H NMR (400 MHz, METHANOL-D4) δ 1.37-1.49 (m, 2H), 1.51-1.62 (m, 3H), 1.76 (t, J=12.50 Hz, 2H), 1.86 (s, 1H), 1.99 (d, J=11.72 Hz, 2H), 2.11-2.20 (m, 1 H), 2.30 (s, 1H), 2.34-2.43 (m, 1H), 2.62-2.73 (m, 1H), 2.79-2.90 (m, 2H), 3.04 (s, 4H), 3.36-3.46 (m, 4H), 3.57 (s, 2H), 3.62 (s, 3H), 3.97 (dd, J=11.13, 3.71 Hz, 2H), 7.13 (s, 1H), 7.31 (d, J=8.59 Hz, 1H), 7.46 (s, 1H); (M+H)=456.2; Accurate mass: calculated (M+H)+ for C26H37N3O4: 456.28568. Found: 456.286.

1-25. (canceled)

26. A compound of formula I, a pharmaceutically acceptable salt thereof, a diastereomer thereof, an enantiomer thereof, or a mixture of any of the foregoing: 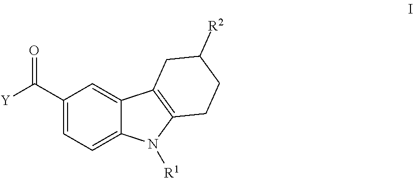
wherein
Y is selected from the group consisting of 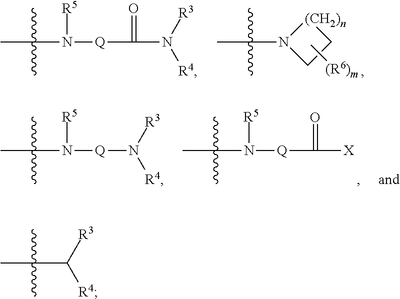
R¹ is selected from the group consisting of —H, C₁₋₆alkyl, C₂₋₆alkenyl, C₃₋₆cycloalkyl, C₃₋₆cycloalkyl-C₁₋₄alkyl, —C(═O)—NR¹⁴R¹⁵, —S(═O)₂—NR¹⁴R¹⁵, —S(═O)₂—C₁₋₆alkyl, —S(═O)₂—C₆₋₁₀aryl, —S(═O)₂—C₂₋₅heteroaryl, —C(═O)—C₁₋₆alkyl, —C(═O)—O—C₁₋₆alkyl, C₆₋₁₀aryl-C₁₋₄alkyl and C₂₋₅heteroaryl-C₁₋₄alkyl, wherein said C₁₋₆alkyl, C₂₋₆alkenyl, —S(═O)₂—C₁₋₆alkyl, —S(═O)₂—C₆₋₁₀aryl, —S(═O)₂—C₂₋₅ heteroaryl, —C(═O)—C₁₋₆alkyl, C₆₋₁₀ aryl-C₁₋₄alkyl and C₂₋₅ heteroaryl-C₁₋₄alkyl used in defining R¹ are optionally substituted with one or more groups selected from the group consisting of —OR, R, —CO₂H, —CO₂—R, —SO₂—R, halogen, —NO₂, —OH, —NH₂, —NHR, —CN, —C(═O)—NH₂, —C(═O)—NR₂ and —C(═O)—NHR;
R² is selected from the group consisting of C₃₋₆heterocycloalkyl, C₃₋₆heterocycloalkyl-C₁₋₄alkyl, C₃₋₆cycloalkyl, C₃₋₆cycloalkyl-C₁₋₄alkyl, C₁₋₆alkyl, C₂₋₆alkenyl, C₆₋₁₀aryl, C₆₋₁₀ aryl-C₁₋₄alkyl, C₂₋₆heteroaryl, C₂₋₆heteroaryl-C₁₋₄alkyl, —C(═O)—C₁₋₆alkyl, —C(═O)—C₃₋₆cycloalkyl and —C(═NH)—C₁₋₆alkyl, wherein said C₃₋₆heterocycloalkyl, C₃₋₆heterocycloalkyl-C₁₋₄alkyl, C₃₋₆cycloalkyl, C₃₋₆cycloalkyl-C₁₋₄alkyl, C₁₋₆alkyl, C₂₋₆alkenyl, C₆₋₁₀aryl, C₆₋₁₀aryl-C₁₋₄alkyl, C₂₋₆heteroaryl, C₂₋₆heteroaryl-C₁₋₄alkyl, —C(═O)—C₁₋₆alkyl, —C(═O)—C₃₋₆cycloalkyl and —C(═NH)—C₁₋₆alkyl used in defining R² are optionally substituted with one or more groups selected from the group consisting of —OR, R, NO₂, —CO₂H, —CO₂—R, —SO₂—R, halogen, —OH, —NH₂, —NHR, —CN, —C(═O)—NH₂ and —C(═O)—NHR;
R³ and R⁴ are independently selected from the group consisting of —H, C₃₋₆cycloalkyl, C₃₋₆heterocycloalkyl, C₂₋₅heteroaryl, C₆₋₁₀aryl, C₁₋₆alkyl, C₁₋₆alkoxy, amino, C₁₋₆alkylamino, diC₁₋₆alkylamino, —C(═O)—C₁₋₆alkyl, —C(═O)—O—C₁₋₆alkyl, —C(═O)—C₃₋₆cyclo alkyl, —C(═O)—NR¹⁴R¹⁵ and —S(═O)₂—NR¹⁴R¹⁵, wherein said C₃₋₆cycloalkyl, C₃₋₆heterocycloalkyl, C₂₋₅heteroaryl, C₆₋₁₀ aryl, C₁₋₆alkyl, C₁₋₆alkoxy, C₁₋₆alkylamino, diC₁₋₆alkylamino, —C(═O)—C₁₋₆alkyl, —C(═O)—O—C₁₋₆alkyl, and —C(═O)—C₃₋₆cycloalkyl used in defining R³ and R⁴ are optionally substituted with one or more groups selected from the group consisting of —OR, R, NO₂, —CO₂H, —CO₂—R, —SO₂—R, halogen, —OH, —NH₂, —NHR, —C(═O)—NH₂, —CN, —C(═O)—NR₂ and —C(═O)—NHR;
R⁵ is selected from the group consisting of —H, C₁₋₆alkyl, and C₃₋₆cycloalkyl;
R⁶ is independently selected from the group consisting of —H, —CN, —NO₂, C₁₋₆alkoxy, halogen, C₁₋₆alkyl, —OH, —NH₂, —NHC(═O)R¹² and —C(═O)NR¹²R¹³;
R¹² and R¹³ are independently selected from the group consisting of —H, C₁₋₆alkyl, C₁₋₆alkoxy, C₃₋₆heterocycloalkyl, C₃₋₆cycloalkyl-C₁₋₄alkyl, and C₃₋₆cycloalkyl wherein said C₁₋₆alkyl, C₁₋₆alkoxy, C₃₋₆heterocycloalkyl, C₃₋₆cycloalkyl-C₁₋₄alkyl and C₃₋₆cycloalkyl used in defining R¹² and R¹³ are optionally substituted with one or more halogens or —OH;
R¹⁴ and R¹⁵ are independently selected from the group consisting of —H, C₁₋₆alkyl, C₆₋₁₀aryl, C₆₋₁₀aryl-C₁₋₄alkyl, C₂₋₅heterocyclyl, C₂₋₅heterocyclyl-C₁₋₄alkyl, C₂₋₆alkenyl, C₃₋₆cycloalkyl, and C₃₋₆cycloalkyl-C₁₋₄alkyl, N,N-di(C₁₋₄alkyl)amido-C₁₋₆alkyl, hydroxy-C₁₋₆alkyl and C₁₋₆alkoxy-C₁₋₆alkyl that are optionally substituted with one or groups selected from the group consisting of halogen, —OH, —CN, —NH₂ and methoxy;
Q is independently selected from the group consisting of C₁₋₆alkylene, C₁₋₆alkylidene and 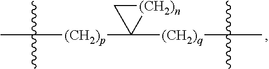
wherein said C₁₋₆alkylene or C₁₋₆alkylidene are optionally substituted with one or more groups selected from the group consisting of —OR, —R, hydroxy-C₁₋₆alkyl, NO₂, —CO₂H, —CO₂—R, —SO₂—R, halogen, —OH, —NH₂, —NHR, —C(═O)—NH₂, —CN, —C(═O)—NR₂ and —C(═O)—NHR;
X is selected from the group consisting of —OH, halogen and —OR;
n is at each occurrence 1, 2 or 3;
p, q and m are independently at each occurrence 0, 1, 2 or 3; and
R is independently at each occurrence C₁₋₆alkyl.

27. A compound, pharmaceutically acceptable salt, diastereomer, or enantiomer as claimed in claim 26, wherein
R¹ is selected from the group consisting of methyl, ethyl, 1-propyl, 2-propyl, 1-butyl, 2-butyl, t-butyl, allyl, —S(═O)₂—CH₃, —S(═O)₂—CH₂CH₃, 2-methoxyethyl, tetrahydropyran-4-yl-methyl, 1-propylsulfonyl, 2-propylsulfonyl, cyclopropylsulfonyl, phenyl, phenylsulfonyl, 2-(methoxycarbonyl)-phenylsulfonyl; 2-(hydroxycarbonyl)-phenylsulfonyl, 1-methyl-1H-imidazol-4-yl-sulfonyl, 1H-imidazol-1-yl-sulfonyl, (5-methylisoxazol-4-yl)sulfonyl, morpholin-4-ylcarbonyl, 4-amino-phenyl, —CH₂—C(═O)—N(CH₃)₂, —C(═O)—N(CH₃)₂, —S(═O)₂—N(CH₃)₂, —S(═O)₂—NHCH₂CH₃, —C(═O)—CH₂CH₂CH₃, —CH₂—C(═O)—OCH₃, —CH₂—C(═O)—OCH₂CH₃, —CH₂—CO₂H, benzyl, 4-aminobenzyl, 4-nitrobenzyl, 4-methylsulfonyl-benzyl, 4-methylthio-benzyl, 4-acetylamino-benzyl, 4-methoxy-benzyl, 4-ethoxy-benzyl, 2,6-difluorobenzyl, (6-chloro-1,3-benzodioxol-5-yl)methyl, (5-ethoxycarbonyl)-fur-2-yl-methyl, (2-methyl-1,3-thiazol-4-yl)-methyl, (5-methyl-isoxazol-4-yl)-methyl, pyridin-2-ylmethyl, cyclobutylmethyl, and cyclopropylmethyl; and
R² is selected from the group consisting of methyl, ethyl, isopropyl, propyl, 2-methyl-propyl, 1-butyl, tert-butyl, 1-pentyl, 1-acetyl-piperidin-4-yl, tetrahydrothien-3-yl, cyclopropylmethyl, cyclobutylmethyl, cyclopentylmethyl, cyclobutyl, cyclopentyl, cyclohexyl, 4-tetrahydro-2H-pyranyl, tetrahydro-thiopyran-4-yl, 2-pyrimidinyl, 1-iminoethyl, 2-pyridinyl, 3,4,5,6-tetrahydropyrdin-2-yl, 3,4-dihydro-2H-pyrrol-5-yl, 2-pyridinyl-methyl, 3-pyridinylmethyl, 4-pyridinylmethyl, 1-methyl-4-piperidinyl, 4-piperidinyl, (6-methyl-pyridin-2-yl)methyl, (2-ethyl-4-methyl-1H-imidazol-5-yl)methyl, tetrahydrofuran-2-yl, tetrahydrofuran-3-yl, tetrahydrofuran-3-ylmethyl, 1-ethyl-1H-pyrazol-4-yl, 1,3-dimethyl-1H-pyrazol-5-yl, (3-methylpyridin-4-yl)methyl, 1,3-oxazol-2-ylmethyl, 1,3-oxazol-5-ylmethyl, 2-(tetrahydro-2H-pyran-4-yl)ethyl, tetrahydro-2H-pyran-4-ylmethyl, 2-phenylethyl, 2-methoxybenzyl, 3,3,3-trifluoropropyl, 2,2-difluoroethyl, 2-hydroxycyclopentyl, (1-ethyl-3-methyl-1H-pyrazol-5-yl)methyl, 2,1,3-benzoxadiazol-5-ylmethyl, 3-thienylmethyl, 2-trifluoromethyl-benzyl, 3-methylbutyl, cyclohex-3-en-1-ylmethyl, 2-fluoro-6-methoxybenzyl, 2-phenyl-propyl, 2-ethyl-butyl, cyclobutylcarbonyl, 2,2-difluoropropanoyl, cyclopentylcarbonyl, tetrahydro-2H-pyran-4-ylcarbonyl, cyclopropylcarbonyl, propylcarbonyl, N-ethylaminocarbonyl, N-isopropylaminocarbonyl, cyclopropylsulfonyl, and ethylsulfonyl.

28. A compound, pharmaceutically acceptable salt, diastereomer, or enantiomer as claimed in claim 26, wherein R² is tetrahydropyranyl.

29. A compound, pharmaceutically acceptable salt, diastereomer, or enantiomer as claimed in claim 26, wherein R² is 4-tetrahydropyranyl.

30. A compound according to claim 26 that is selected from the group consisting of:
N-[2-(Cyclopropylamino)-2-oxoethyl]-N,9-dimethyl-3-(tetrahydro-2H-pyran-4-yl)-2,3,4,9-tetrahydro-1H-carbazole-6-carboxamide;
(+)-N-[2-(Cyclopropylamino)-2-oxoethyl]-N,9-dimethyl-3-(tetrahydro-2H-pyran-4-yl)-2,3,4,9-tetrahydro-1H-carbazole-6-carboxamide;
(−)-N-[2-(Cyclopropylamino)-2-oxoethyl]-N,9-dimethyl-3-(tetrahydro-2H-pyran-4-yl)-2,3,4,9-tetrahydro-1H-carbazole-6-carboxamide;
N-Ethyl-N-[2-(ethylamino)-2-oxoethyl]-9-methyl-3-(tetrahydro-2H-pyran-4-yl)-2,3,4,9-tetrahydro-1H-carbazole-6-carboxamide;
N-Ethyl-N-[2-(isopropylamino)-2-oxoethyl]-9-methyl-3-(tetrahydro-2H-pyran-4-yl)-2,3,4,9-tetrahydro-1H-carbazole-6-carboxamide;
N-Cyclopropyl-1-(9-methyl-3-(tetrahydro-2H-pyran-4-yl)-2,3,4,9-tetrahydro-1H-carbazole-6-carbonyl)piperidine-4-carboxamide;
N-Ethyl-N-{2-[(2-fluoroethyl)amino]-2-oxoethyl}-9-methyl-3-(tetrahydro-2H-pyran-4-yl)-2,3,4,9-tetrahydro-1H-carbazole-6-carboxamide;
N-[2-(Cyclopropylamino)-2-oxoethyl]-N-ethyl-9-methyl-3-(tetrahydro-2H-pyran-4-yl)-2,3,4,9-tetrahydro-1H-carbazole-6-carboxamide;
N-Cyclopropyl-2-(1-(9-methyl-3-(tetrahydro-2H-pyran-4-yl)-2,3,4,9-tetrahydro-1H-carbazole-6-carbonyl)azetidin-3-yl)acetamide;
N,9-Dimethyl-N-(2-(methylamino)ethyl)-3-(tetrahydro-2H-pyran-4-yl)-2,3,4,9-tetrahydro-1H-carbazole-6-carboxamide;
N-(2-(3-Cyclopropyl-1-methylureido)ethyl)-N,9-dimethyl-3-(tetrahydro-2H-pyran-4-yl)-2,3,4,9-tetrahydro-1H-carbazole-6-carboxamide;
N-(2-(3-Cyclopropyl-1-methylthioureido)ethyl)-N,9-dimethyl-3-(tetrahydro-2H-pyran-4-yl)-2,3,4,9-tetrahydro-1H-carbazole-6-carboxamide;
N-{2-[(2-Fluoroethyl)amino]-2-oxoethyl}-N,9-dimethyl-3-(tetrahydro-2H-pyran-4-yl)-2,3,4,9-tetrahydro-1H-carbazole-6-carboxamide;
N-Ethyl-9-methyl-N-[2-(methylamino)-2-oxoethyl]-3-(tetrahydro-2H-pyran-4-yl)-2,3,4,9-tetrahydro-1H-carbazole-6-carboxamide;
Methyl, methyl [2-(methyl {[9-methyl-3-(tetrahydro-2H-pyran-4-yl)-2,3,4,9-tetrahydro-1H-carbazol-6-yl]carbonyl}amino)ethyl]carbamate;
N-{2-[(Cyclopropylcarbonyl)(methyl)amino]ethyl}-N,9-dimethyl-3-(tetrahydro-2H-pyran-4-yl)-2,3,4,9-tetrahydro-1H-carbazole-6-carboxamide;
N-Cyclopropyl-1-{[9-methyl-3-(tetrahydro-2H-pyran-4-yl)-2,3,4,9-tetrahydro-1H-carbazol-6-yl]carbonyl}piperidine-3-carboxamide;
N-Cyclopropyl-1-{[9-methyl-3-(tetrahydro-2H-pyran-4-yl)-2,3,4,9-tetrahydro-1H-carbazol-6-yl]carbonyl}azetidine-3-carboxamide;
N-Ethyl-N-{2-[(1-isocyanocyclopropyl)amino]-2-oxoethyl}-9-methyl-3-(tetrahydro-2H-pyran-4-yl)-2,3,4,9-tetrahydro-1H-carbazole-6-carboxamide;
N-Ethyl-N-(2-hydroxyethyl)-9-methyl-3-(tetrahydro-2H-pyran-4-yl)-2,3,4,9-tetrahydro-1H-carbazole-6-carboxamide;
N-(3-(cyclopropylamino)-3-oxopropyl)-N,9-dimethyl-3-(tetrahydro-2H-pyran-4-yl)-2,3,4,9-tetrahydro-1H-carbazole-6-carboxamide;
N-(4-(Cyclopropylamino)-4-oxobutyl)-N-ethyl-9-methyl-3-(tetrahydro-2H-pyran-4-yl)-2,3,4,9-tetrahydro-1H-carbazole-6-carboxamide;
N-(4-(Cyclopropylamino)-4-oxobutyl)-N-methyl-9-methyl-3-(tetrahydro-2H-pyran-4-yl)-2,3,4,9-tetrahydro-1H-carbazole-6-carboxamide;
N-(4-(Methylamino)-4-oxobutyl)-N-methyl-9-methyl-3-(tetrahydro-2H-pyran-4-yl)-2,3,4,9-tetrahydro-1H-carbazole-6-carboxamide;
N-(4-(Ethylamino)-4-oxobutyl)-N-methyl-9-methyl-3-(tetrahydro-2H-pyran-4-yl)-2,3,4,9-tetrahydro-1H-carbazole-6-carboxamide;
N-(4-(2-Fluoro ethylamino)-4-oxobutyl)-N-methyl-9-methyl-3-(tetrahydro-2H-pyran-4-yl)-2,3,4,9-tetrahydro-1H-carbazole-6-carboxamide;
3-cyclohexyl-9-methyl-6-[(4-methylpiperidin-1-yl)carbonyl]-2,3,4,9-tetrahydro-1H-carbazole;
3-cyclohexyl-9-ethyl-6-[(4-methylpiperidin-1-yl)carbonyl]-2,3,4,9-tetrahydro-1H-carbazole;
3-cyclohexyl-6-[(4-methylpiperidin-1-yl)carbonyl]-9-(methylsulfonyl)-2,3,4,9-tetrahydro-1H-carbazole;
3-cyclohexyl-9-(ethylsulfonyl)-6-[(4-methylpiperidin-1-yl)carbonyl]-2,3,4,9-tetrahydro-1H-carbazole;
3-cyclohexyl-N-[2-(cyclopropylamino)-2-oxoethyl]-N-methyl-9-(methylsulfonyl)-2,3,4,9-tetrahydro-1H-carbazole-6-carboxamide;
3-cyclohexyl-N-[2-(cyclopropylamino)-2-oxoethyl]-9-(isopropylsulfonyl)-N-methyl-2,3,4,9-tetrahydro-1H-carbazole-6-carboxamide;
N-Ethyl-9-methyl-N-(2-oxo-2-(tetrahydro-2H-pyran-4-ylamino)ethyl)-3-(tetrahydro-2H-pyran-4-yl)-2,3,4,9-tetrahydro-1H-carbazole-6-carboxamide;
N-Ethyl-9-methyl-N-(2-oxo-2-((S)-tetrahydrofuran-3-ylamino)ethyl)-3-(tetrahydro-2H-pyran-4-yl)-2,3,4,9-tetrahydro-1H-carbazole-6-carboxamide;
N-Ethyl-9-methyl-N-(2-oxo-2-((R)-tetrahydrofuran-3-ylamino)ethyl)-3-(tetrahydro-2H-pyran-4-yl)-2,3,4,9-tetrahydro-1H-carbazole-6-carboxamide;
N-Ethyl-9-methyl-N-(2-(oxetan-3-ylamino)-2-oxoethyl)-3-(tetrahydro-2H-pyran-4-yl)-2,3,4,9-tetrahydro-1H-carbazole-6-carboxamide;
N-Ethyl-N-(4-hydroxybutyl)-9-methyl-3-(tetrahydro-2H-pyran-4-yl)-2,3,4,9-tetrahydro-1H-carbazole-6-carboxamide;
N-(2-(Cyanomethylamino)-2-oxoethyl)-N-ethyl-9-methyl-3-(tetrahydro-2H-pyran-4-yl)-2,3,4,9-tetrahydro-1H-carbazole-6-carboxamide;
N-(2-(Cyclopropylamino)-2-oxoethyl)-N-ethyl-3-(tetrahydro-2H-pyran-4-yl)-2,3,4,9-tetrahydro-1H-carbazole-6-carboxamide;
N—((S)-1-(2-Fluoroethylamino)-1-oxopropan-2-yl)-N,9-dimethyl-3-(tetrahydro-2H-pyran-4-yl)-2,3,4,9-tetrahydro-1H-carbazole-6-carboxamide;
N—((S)-1-(Cyclopropylamino)-1-oxopropan-2-yl)-N,9-dimethyl-3-(tetrahydro-2H-pyran-4-yl)-2,3,4,9-tetrahydro-1H-carbazole-6-carboxamide;
N-(4-(Cyclopropylamino)-4-oxobutyl)-9-methyl-3-(tetrahydro-2H-pyran-4-yl)-2,3,4,9-tetrahydro-1H-carbazole-6-carboxamide;
N-(1-(Cyclopropylcarbamoyl)cyclopropyl)-N,9-dimethyl-3-(tetrahydro-2H-pyran-4-yl)-2,3,4,9-tetrahydro-1H-carbazole-6-carboxamide;
N-(2-Fluoroethyl)-9-methyl-3-(tetrahydro-2H-pyran-4-yl)-2,3,4,9-tetrahydro-1H-carbazole-6-carboxamide;
N-Ethyl-N-(4-(2-fluoroethylamino)-4-oxobutyl)-9-methyl-3-(tetrahydro-2H-pyran-4-yl)-2,3,4,9-tetrahydro-1H-carbazole-6-carboxamide;
N—((R)-1-(2-fluoroethylamino)-1-oxopropan-2-yl)-N,9-dimethyl-3-(tetrahydro-2H-pyran-4-yl)-2,3,4,9-tetrahydro-1H-carbazole-6-carboxamide;
N—((R)-1-(ethylamino)-1-oxopropan-2-yl)-N,9-dimethyl-3-(tetrahydro-2H-pyran-4-yl)-2,3,4,9-tetrahydro-1H-carbazole-6-carboxamide;
N-ethyl-N-(2-hydroxypropyl)-9-methyl-3-(tetrahydro-2H-pyran-4-yl)-2,3,4,9-tetrahydro-1H-carbazole-6-carboxamide;
N-(2-(2-Cyano ethylamino)-2-oxo ethyl)-N-ethyl-9-(ethylsulfonyl)-3-(tetrahydro-2H-pyran-4-yl)-2,3,4,9-tetrahydro-1H-carbazole-6-carboxamide;
(3S)—N-Cyclopropyl-1-(9-methyl-3-(tetrahydro-2H-pyran-4-yl)-2,3,4,9-tetrahydro-1H-carbazole-6-carbonyl)piperidine-3-carboxamide;
(3S)—N-cyclopropyl-1-(9-methyl-3-(tetrahydro-2H-pyran-4-yl)-2,3,4,9-tetrahydro-1H-carbazole-6-carbonyl)piperidine-3-carboxamide;
N,9-Dimethyl-N-(4-(methylamino)-4-oxobutyl)-3-(tetrahydro-2H-pyran-4-yl)-2,3,4,9-tetrahydro-1H-carbazole-6-carboxamide;
N-Ethyl-N-(2-(2-fluoroethylamino)-2-oxoethyl)-9-methyl-3-(tetrahydro-2H-pyran-4-yl)-2,3,4,9-tetrahydro-1H-carbazole-6-carboxamide;
N-(2-(Cyclopropylamino)-2-oxoethyl)-N-ethyl-9-methyl-3-(tetrahydro-2H-pyran-4-yl)-2,3,4,9-tetrahydro-1H-carbazole-6-carboxamide;
N-(4-(2-Fluoroethylamino)-4-oxobutyl)-N,9-dimethyl-3-(tetrahydro-2H-pyran-4-yl)-2,3,4,9-tetrahydro-1H-carbazole-6-carboxamide;
N-ethyl-N-(2-hydroxyethyl)-9-methyl-3-(tetrahydro-2H-pyran-4-yl)-2,3,4,9-tetrahydro-1H-carbazole-6-carboxamide;
N-Cyclopropyl-1-(9-methyl-3-(tetrahydro-2H-pyran-4-yl)-2,3,4,9-tetrahydro-1H-carbazole-6-carbonyl)pyrrolidine-3-carboxamide;
(3S)—N-Cyclopropyl-1-(9-methyl-3-(tetrahydro-2H-pyran-4-yl)-2,3,4,9-tetrahydro-1H-carbazole-6-carbonyl)pyrrolidine-3-carboxamide;
(3R)—N-Cyclopropyl-1-(9-methyl-3-(tetrahydro-2H-pyran-4-yl)-2,3,4,9-tetrahydro-1H-carbazole-6-carbonyl)pyrrolidine-3-carboxamide;
N-(2-Fluoroethyl)-1-(9-methyl-3-(tetrahydro-2H-pyran-4-yl)-2,3,4,9-tetrahydro-1H-carbazole-6-carbonyl)pyrrolidine-3-carboxamide;
N-Ethyl-1-(9-methyl-3-(tetrahydro-2H-pyran-4-yl)-2,3,4,9-tetrahydro-1H-carbazole-6-carbonyl)pyrrolidine-3-carboxamide;
N-Cyclopropyl-2-((3R)-1-(9-methyl-3-(tetrahydro-2H-pyran-4-yl)-2,3,4,9-tetrahydro-1H-carbazole-6-carbonyl)pyrrolidin-3-yl)acetamide;
N-((3S)-1-(9-Methyl-3-(tetrahydro-2H-pyran-4-yl)-2,3,4,9-tetrahydro-1H-carbazole-6-carbonyl)pyrrolidin-3-yl)cyclopropanecarboxamide;
(3S)—N-(2-Fluoroethyl)-1-(9-methyl-3-(tetrahydro-2H-pyran-4-yl)-2,3,4,9-tetrahydro-1H-carbazole-6-carbonyl)pyrrolidine-3-carboxamide;
(3S)—N-(Cyclopropylmethyl)-1-(9-methyl-3-(tetrahydro-2H-pyran-4-yl)-2,3,4,9-tetrahydro-1H-carbazole-6-carbonyl)pyrrolidine-3-carboxamide;
N-(1-(9-Methyl-3-(tetrahydro-2H-pyran-4-yl)-2,3,4,9-tetrahydro-1H-carbazole-6-carbonyl)piperidin-4-yl)cyclopropanecarboxamide;
N-(1-(9-Methyl-3-(tetrahydro-2H-pyran-4-yl)-2,3,4,9-tetrahydro-1H-carbazole-6-carbonyl)piperidin-3-yl)cyclopropanecarboxamide;
(3S)—N-(2,2-Difluoroethyl)-1-(9-methyl-3-(tetrahydro-2H-pyran-4-yl)-2,3,4,9-tetrahydro-1H-carbazole-6-carbonyl)pyrrolidine-3-carboxamide;
(3S)—N-Ethyl-1-(9-methyl-3-(tetrahydro-2H-pyran-4-yl)-2,3,4,9-tetrahydro-1H-carbazole-6-carbonyl)pyrrolidine-3-carboxamide;
N-(1-(9-Methyl-3-(tetrahydro-2H-pyran-4-yl)-2,3,4,9-tetrahydro-1H-carbazole-6-carbonyl)piperidin-3-yl)propionamide;
N-(1-(9-Methyl-3-(tetrahydro-2H-pyran-4-yl)-2,3,4,9-tetrahydro-1H-carbazole-6-carbonyl)piperidin-3-yl)isobutyramide;
2-Cyclopropyl-N-(1-(9-methyl-3-(tetrahydro-2H-pyran-4-yl)-2,3,4,9-tetrahydro-1H-carbazole-6-carbonyl)piperidin-3-yl)acetamide;
N-(4-(2-Hydroxyethylamino)-4-oxobutyl)-N,9-dimethyl-3-(tetrahydro-2H-pyran-4-yl)-2,3,4,9-tetrahydro-1H-carbazole-6-carboxamide;
N-((3S)-1-(9-Methyl-3-(tetrahydro-2H-pyran-4-yl)-2,3,4,9-tetrahydro-1H-carbazole-6-carbonyl)piperidin-3-yl)cyclopropanecarboxamide;
N,9-Dimethyl-N-(4-oxo-4-((S)-tetrahydrofuran-3-ylamino)butyl)-3-(tetrahydro-2H-pyran-4-yl)-2,3,4,9-tetrahydro-1H-carbazole-6-carboxamide;
N,9-Dimethyl-N-(4-(oxetan-3-ylamino)-4-oxobutyl)-3-(tetrahydro-2H-pyran-4-yl)-2,3,4,9-tetrahydro-1H-carbazole-6-carboxamide;
N-(4-(3-Hydroxypropylamino)-4-oxobutyl)-N,9-dimethyl-3-(tetrahydro-2H-pyran-4-yl)-2,3,4,9-tetrahydro-1H-carbazole-6-carboxamide;
N-Ethyl-9-(ethylsulfonyl)-N-(2-(2-hydroxyethylamino)-2-oxoethyl)-3-(tetrahydro-2H-pyran-4-yl)-2,3,4,9-tetrahydro-1H-carbazole-6-carboxamide;
(9-(Ethylsulfonyl)-3-(tetrahydro-2H-pyran-4-yl)-2,3,4,9-tetrahydro-1H-carbazol-6-yl)((R)-3-hydroxypyrrolidin-1-yl)methanone;
(9-(Ethylsulfonyl)-3-(tetrahydro-2H-pyran-4-yl)-2,3,4,9-tetrahydro-1H-carbazol-6-yl)((S)-3-hydroxypyrrolidin-1-yl)methanone;
(9-(Ethylsulfonyl)-3-(tetrahydro-2H-pyran-4-yl)-2,3,4,9-tetrahydro-1H-carbazol-6-yl)((R)-3-hydroxypiperidin-1-yl)methanone;
(9-(Ethylsulfonyl)-3-(tetrahydro-2H-pyran-4-yl)-2,3,4,9-tetrahydro-1H-carbazol-6-yl)(4-hydroxypiperidin-1-yl)methanone;
N6-Ethyl-N6-(2-(ethylamino)-2-oxoethyl)-N9,N9-dimethyl-3-(tetrahydro-2H-pyran-4-yl)-3,4-dihydro-1H-carbazole-6,9(2H)-dicarboxamide;
N-ethyl-N-(2-(ethylamino)-2-oxoethyl)-3-(tetrahydro-2H-pyran-4-yl)-2,3,4,9-tetrahydro-1H-carbazole-6-carboxamide;
Ethyl 2-(6-(ethyl(2-(ethylamino)-2-oxoethyl)carbamoyl)-3-(tetrahydro-2H-pyran-4-yl)-3,4-dihydro-1H-carbazol-9(2H)-yl)acetate;
2-(6-(Ethyl(2-(ethylamino)-2-oxoethyl)carbamoyl)-3-(tetrahydro-2H-pyran-4-yl)-3,4-dihydro-1H-carbazol-9(2H)-yl)acetic acid;
9-(2-(diethylamino)-2-oxoethyl)-N-ethyl-N-(2-(ethylamino)-2-oxoethyl)-3-(tetrahydro-2H-pyran-4-yl)-2,3,4,9-tetrahydro-1H-carbazole-6-carboxamide;
N-Ethyl-N-(2-(ethylamino)-2-oxoethyl)-9-(2-(methylamino)-2-oxoethyl)-3-(tetrahydro-2H-pyran-4-yl)-2,3,4,9-tetrahydro-1H-carbazole-6-carboxamide;
N-Ethyl-N-(2-(ethylamino)-2-oxoethyl)-9-(2-hydroxy-2-methylpropyl)-3-(tetrahydro-2H-pyran-4-yl)-2,3,4,9-tetrahydro-1H-carbazole-6-carboxamide;
N-Ethyl-N-(2-(ethylamino)-2-oxoethyl)-9-(2-hydroxyethyl)-3-(tetrahydro-2H-pyran-4-yl)-2,3,4,9-tetrahydro-1H-carbazole-6-carboxamide;
2-(N-Ethyl-9-(ethylsulfonyl)-3-(tetrahydro-2H-pyran-4-yl)-2,3,4,9-tetrahydro-1H-carbazole-6-carboxamido)acetic acid;
N-Ethyl-9-(ethylsulfonyl)-N-(2-(2-hydroxypropylamino)-2-oxoethyl)-3-(tetrahydro-2H-pyran-4-yl)-2,3,4,9-tetrahydro-1H-carbazole-6-carboxamide;
N-Ethyl-9-(ethylsulfonyl)-N-(2-(2-methoxyethylamino)-2-oxoethyl)-3-(tetrahydro-2H-pyran-4-yl)-2,3,4,9-tetrahydro-1H-carbazole-6-carboxamide;
N-Ethyl-9-(ethylsulfonyl)-N-(2-(oxetan-3-ylamino)-2-oxoethyl)-3-(tetrahydro-2H-pyran-4-yl)-2,3,4,9-tetrahydro-1H-carbazole-6-carboxamide;
N-[2-(cyclopropylamino)-2-oxoethyl]-9-(cyclopropylmethyl)-N-ethyl-3-(tetrahydro-2H-pyran-4-yl)-2,3,4,9-tetrahydro-1H-carbazole-6-carboxamide;
9-(Cyclopropylmethyl)-N-ethyl-N-(2-(ethylamino)-2-oxoethyl)-3-(tetrahydro-2H-pyran-4-yl)-2,3,4,9-tetrahydro-1H-carbazole-6-carboxamide;
9-Cyclobutyl-N-ethyl-N-(2-(ethylamino)-2-oxoethyl)-3-(tetrahydro-2H-pyran-4-yl)-2,3,4,9-tetrahydro-1H-carbazole-6-carboxamide;
9-Cyclobutyl-N-ethyl-N-(2-(2-fluoroethylamino)-2-oxoethyl)-3-(tetrahydro-2H-pyran-4-yl)-2,3,4,9-tetrahydro-1H-carbazole-6-carboxamide;
9-Cyclobutyl-N-ethyl-N-(2-(isopropylamino)-2-oxoethyl)-3-(tetrahydro-2H-pyran-4-yl)-2,3,4,9-tetrahydro-1H-carbazole-6-carboxamide;
9-Ethyl-N-methyl-N-(4-(methylamino)-4-oxobutyl)-3-(tetrahydro-2H-pyran-4-yl)-2,3,4,9-tetrahydro-1H-carbazole-6-carboxamide;
9-Ethyl-N-(4-(2-fluoroethylamino)-4-oxobutyl)-N-methyl-3-(tetrahydro-2H-pyran-4-yl)-2,3,4,9-tetrahydro-1H-carbazole-6-carboxamide;
N-(4-(2,2-difluoroethylamino)-4-oxobutyl)-9-ethyl-N-methyl-3-(tetrahydro-2H-pyran-4-yl)-2,3,4,9-tetrahydro-1H-carbazole-6-carboxamide;
9-Ethyl-N-methyl-N-(4-oxo-4-(2,2,2-trifluoroethylamino)butyl)-3-(tetrahydro-2H-pyran-4-yl)-2,3,4,9-tetrahydro-1H-carbazole-6-carboxamide;
9-Ethyl-N-(4-(2-hydroxyethylamino)-4-oxobutyl)-N-methyl-3-(tetrahydro-2H-pyran-4-yl)-2,3,4,9-tetrahydro-1H-carbazole-6-carboxamide;
N-Ethyl-N-(2-(ethylamino)-2-oxoethyl)-9-(2-fluoroethyl)-3-(tetrahydro-2H-pyran-4-yl)-2,3,4,9-tetrahydro-1H-carbazole-6-carboxamide;
N-(4-(Ethylamino)-4-oxobutyl)-9-(2-fluoroethyl)-N-methyl-3-(tetrahydro-2H-pyran-4-yl)-2,3,4,9-tetrahydro-1H-carbazole-6-carboxamide;
N-ethyl-9-(ethylsulfonyl)-N-(2-(2-hydroxyethylamino)-2-oxoethyl)-3-(tetrahydro-2H-pyran-4-yl)-2,3,4,9-tetrahydro-1H-carbazole-6-carboxamide;
N-ethyl-9-(ethylsulfonyl)-N-(2-(3-hydroxypropylamino)-2-oxoethyl)-3-(tetrahydro-2H-pyran-4-yl)-2,3,4,9-tetrahydro-1H-carbazole-6-carboxamide;
N-ethyl-9-(ethylsulfonyl)-N-(2-(3-fluoropropylamino)-2-oxoethyl)-3-(tetrahydro-2H-pyran-4-yl)-2,3,4,9-tetrahydro-1H-carbazole-6-carboxamide;
N-ethyl-9-(ethylsulfonyl)-N-(2-(2-fluoroethylamino)-2-oxoethyl)-3-(tetrahydro-2H-pyran-4-yl)-2,3,4,9-tetrahydro-1H-carbazole-6-carboxamide;
2-(N-ethyl-9-(ethylsulfonyl)-3-(tetrahydro-2H-pyran-4-yl)-2,3,4,9-tetrahydro-1H-carbazole-6-carboxamido)acetic acid;
N-(2-(cyclopropylamino)-2-oxoethyl)-9-(ethylsulfonyl)-N-methyl-3-(tetrahydro-2H-pyran-4-yl)-2,3,4,9-tetrahydro-1H-carbazole-6-carboxamide;
(2R)-1-(9-(ethylsulfonyl)-3-(tetrahydro-2H-pyran-4-yl)-2,3,4,9-tetrahydro-1H-carbazole-6-carbonyl)-N-(2-fluoroethyl)pyrrolidine-2-carboxamide;
N-(2-(2,2-difluoroethylamino)-2-oxoethyl)-N-ethyl-9-methyl-3-(tetrahydro-2H-pyran-4-yl)-2,3,4,9-tetrahydro-1H-carbazole-6-carboxamide;
N-ethyl-N-(2-((R)-2-hydroxypropylamino)-2-oxoethyl)-9-methyl-3-(tetrahydro-2H-pyran-4-yl)-2,3,4,9-tetrahydro-1H-carbazole-6-carboxamide;
N-ethyl-N-(2-((S)-2-hydroxypropylamino)-2-oxoethyl)-9-methyl-3-(tetrahydro-2H-pyran-4-yl)-2,3,4,9-tetrahydro-1H-carbazole-6-carboxamide;
N-ethyl-N-(2-(2-methoxyethylamino)-2-oxoethyl)-9-methyl-3-(tetrahydro-2H-pyran-4-yl)-2,3,4,9-tetrahydro-1H-carbazole-6-carboxamide, and
N—((R)-1-(cyclopropylamino)-1-oxopropan-2-yl)-N,9-dimethyl-3-(tetrahydro-2H-pyran-4-yl)-2,3,4,9-tetrahydro-1H-carbazole-6-carboxamide;
or a pharmaceutically acceptable salt of any foregoing compound.

31. A compound according to claim 26 that is selected from thr group consisting of:
(R)—N—((S)-1-Hydroxy-5-(oxetan-3-ylamino)-5-oxopentan-2-yl)-N,9-dimethyl-3-(tetrahydro-2H-pyran-4-yl)-2,3,4,9-tetrahydro-1H-carbazole-6-carboxamide;
(R)—N—((S)-5-(2,2-Difluoroethylamino)-1-hydroxy-5-oxopentan-2-yl)-N,9-dimethyl-3-(tetrahydro-2H-pyran-4-yl)-2,3,4,9-tetrahydro-1H-carbazole-6-carboxamide;
(R)—N—((S)-5-(2-Fluoroethylamino)-1-hydroxy-5-oxopentan-2-yl)-N,9-dimethyl-3-(tetrahydro-2H-pyran-4-yl)-2,3,4,9-tetrahydro-1H-carbazole-6-carboxamide;
(R)—N-(4-(Cyanomethylamino)-4-oxobutyl)-N,9-dimethyl-3-(tetrahydro-2H-pyran-4-yl)-2,3,4,9-tetrahydro-1H-carbazole-6-carboxamide;
(R)—N—((S)-1-Hydroxy-5-(methylamino)-5-oxopentan-2-yl)-N,9-dimethyl-3-(tetrahydro-2H-pyran-4-yl)-2,3,4,9-tetrahydro-1H-carbazole-6-carboxamide;
(R)—N—((S)-1-Hydroxy-5-(isopropylamino)-5-oxopentan-2-yl)-N,9-dimethyl-3-(tetrahydro-2H-pyran-4-yl)-2,3,4,9-tetrahydro-1H-carbazole-6-carboxamide;
(R)—N—((S)-5-(Ethylamino)-1-hydroxy-5-oxopentan-2-yl)-N,9-dimethyl-3-(tetrahydro-2H-pyran-4-yl)-2,3,4,9-tetrahydro-1H-carbazole-6-carboxamide;
(R)—N-(4-(Methoxyamino)-4-oxobutyl)-N,9-dimethyl-3-(tetrahydro-2H-pyran-4-yl)-2,3,4,9-tetrahydro-1H-carbazole-6-carboxamide;
(R)—N-(4-(2,2-Dimethylhydrazinyl)-4-oxobutyl)-N,9-dimethyl-3-(tetrahydro-2H-pyran-4-yl)-2,3,4,9-tetrahydro-1H-carbazole-6-carboxamide;
(R)—N-(4-(2-Methoxyethylamino)-4-oxobutyl)-N,9-dimethyl-3-(tetrahydro-2H-pyran-4-yl)-2,3,4,9-tetrahydro-1H-carbazole-6-carboxamide;
(R)—N-(4-(1H-Pyrrol-1-ylamino)-4-oxobutyl)-N,9-dimethyl-3-(tetrahydro-2H-pyran-4-yl)-2,3,4,9-tetrahydro-1H-carbazole-6-carboxamide;
(R)—N-Ethyl-N-(4-(2-hydroxyethylamino)-4-oxobutyl)-9-methyl-3-(tetrahydro-2H-pyran-4-yl)-2,3,4,9-tetrahydro-1H-carbazole-6-carboxamide, and
(R)—N-(4-(2-Hydroxyethylamino)-4-oxobutyl)-N,9-dimethyl-3-(tetrahydro-2H-pyran-4-yl)-2,3,4,9-tetrahydro-1H-carbazole-6-carboxamide;
or a pharmaceutically acceptable salt of any foregoing compound.

32. A pharmaceutical composition comprising a compound, pharmaceutically acceptable salt, diastereomer, or enantiomer according to claim 26 and a pharmaceutically acceptable carrier.

33. A pharmaceutical composition comprising a compound, pharmaceutically acceptable salt, diastereomer, or enantiomer according to claim 30 and a pharmaceutically acceptable carrier.

34. A pharmaceutical composition comprising a compound, pharmaceutically acceptable salt, diastereomer, or enantiomer according to claim 31 and a pharmaceutically acceptable carrier.

35. A method for the therapy of pain in a warm-blooded animal, comprising the step of administering to said animal in need of such therapy a therapeutically effective amount of a compound, pharmaceutically acceptable salt, diastereomer, or enantiomer according to claim 26.

36. A process for preparing a compound of Formula I comprising:
reacting a compound of Formula II with Y—H, 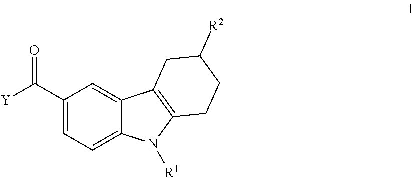
or comprising reacting a compound of Formula II with Y—H, 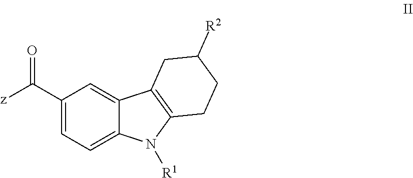
wherein
Y is selected from the group consisting of 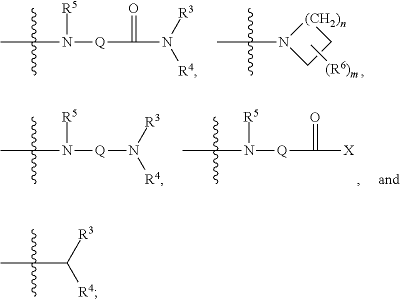
R¹ is selected from the group consisting of —H, C₁₋₆alkyl, C₂₋₆alkenyl, C₃₋₆cycloalkyl, C₃₋₆cycloalkyl-C₁₋₄alkyl, —C(═O)—NR¹⁴R¹⁵, —S(═O)₂—NR¹⁴R¹⁵, —S(═O)₂—C₁₋₆alkyl, —S(═O)₂—C₆₋₁₀aryl, —S(═O)₂—C₂₋₅heteroaryl, —C(═O)—C₁₋₆alkyl, —C(═O)—O—C₁₋₆alkyl, C₆₋₁₀aryl-C₁₋₄alkyl and C₂₋₅heteroaryl-C₁₋₄alkyl, wherein said C₁₋₆alkyl, C₂₋₆alkenyl, —S(═O)₂—C₁₋₆alkyl, —S(═O)₂—C₆₋₁₀aryl, —S(═O)₂—C₂₋₅heteroaryl, —C(═O)—C₁₋₆alkyl, C₆₋₁₀aryl-C₁₋₄alkyl and C₂₋₅heteroaryl-C₁₋₄alkyl used in defining R¹ are optionally substituted with one or more groups selected from the group consisting of —OR, R, —CO₂H, —CO₂—R, —SO₂—R, halogen, —NO₂, —OH, —NH₂, —NHR, —CN, —C(═O)—NH₂, —C(═O)—NR₂ and —C(═O)—NHR;
R² is selected from the group consisting of C₃₋₆heterocycloalkyl, C₃₋₆heterocycloalkyl-C₁₋₄alkyl, C₃₋₆cycloalkyl, C₃₋₆cycloalkyl-C₁₋₄alkyl, C₁₋₆alkyl, C₂₋₆alkenyl, C₆₋₁₀aryl, C₆₋₁₀aryl-C₁₋₄alkyl, C₂₋₆heteroaryl, C₂₋₆heteroaryl-C₁₋₄alkyl, —C(═O)—C₁₋₆alkyl, —C(═O)—C₃₋₆cycloalkyl and —C(═NH)—C₁₋₆alkyl, wherein said C₃₋₆heterocycloalkyl, C₃₋₆heterocycloalkyl-C₁₋₄alkyl, C₃₋₆cycloalkyl, C₃₋₆cycloalkyl-C₁₋₄alkyl, C₁₋₆alkyl, C₂₋₆alkenyl, C₆₋₁₀aryl, C₆₋₁₀aryl-C₁₋₄alkyl, C₂₋₆heteroaryl, C₂₋₆heteroaryl-C₁₋₄alkyl, —C(═O)—C₁₋₆alkyl, —C(═O)—C₃₋₆cycloalkyl and —C(═NH)—C₁₋₆alkyl used in defining R² are optionally substituted with one or more groups selected from the group consisting of —OR, R, NO₂, —CO₂H, —CO₂—R, —SO₂—R, halogen, —OH, —NH₂, —NHR, —CN, —C(═O)—NH₂ and —C(═O)—NHR;
R³ and R⁴ are independently selected from the group consisting of —H, C₃₋₆cycloalkyl, C₃₋₆heterocycloalkyl, C₂₋₅heteroaryl, C₆₋₁₀aryl, C₁₋₆alkyl, C₁₋₆alkoxy, amino, C₁₋₆alkylamino, diC₁₋₆alkylamino, —C(═O)—C₁₋₆alkyl, —C(═O)—O—C₁₋₆alkyl, —C(═O)—C₃₋₆cyclo alkyl, —C(═O)—NR¹⁴R¹⁵ and —S(═O)₂—NR¹⁴R¹⁵, wherein said C₃₋₆cycloalkyl, C₃₋₆heterocycloalkyl, C₂₋₅heteroaryl, C₆₋₁₀aryl, C₁₋₆alkyl, C₁₋₆alkoxy, C₁₋₆alkylamino, diC₁₋₆alkylamino, —C(═O)—C₁₋₆alkyl, —C(═O)—O—C₁₋₆alkyl, and —C(═O)—C₃₋₆cycloalkyl used in defining R³ and R⁴ are optionally substituted with one or more groups selected from the group consisting of —OR, R, NO₂, —CO₂H, —CO₂—R, —SO₂—R, halogen, —OH, —NH₂, —NHR, —C(═O)—NH₂, —CN, —C(═O)—NR₂ and —C(═O)—NHR;
R⁵ is selected from the group consisting of —H, C₁₋₆alkyl, and C₃₋₆cycloalkyl;
R⁶ is independently selected from the group consisting of —H, —CN, —NO₂, C₁₋₆alkoxy, halogen, C₁₋₆alkyl, —OH, —NH₂, —NHC(═O)R¹² and —C(═O)NR¹²R¹³;
R¹² and R¹³ are independently selected from the group consisting of —H, C₁₋₆alkyl, C₁₋₆alkoxy, C₃₋₆heterocycloalkyl, C₃₋₆cycloalkyl-C₁₋₄alkyl, and C₃₋₆cycloalkyl wherein said C₁₋₆alkyl, C₁₋₆alkoxy, C₃₋₆heterocycloalkyl, C₃₋₆cycloalkyl-C₁₋₄alkyl and C₃₋₆cycloalkyl used in defining R¹² and R¹³ are optionally substituted with one or more halogens or —OH;
R¹⁴ and R¹⁵ are independently selected from the group consisting of —H, C₁₋₆alkyl, C₆₋₁₀aryl, C₆₋₁₀aryl-C₁₋₄alkyl, C₂₋₅heterocyclyl, C₂₋₅heterocyclyl-C₁₋₄alkyl, C₂₋₆alkenyl, C₃₋₆cycloalkyl, and C₃₋₆cycloalkyl-C₁₋₄alkyl, N,N-di(C₁₋₄alkyl)amido-C₁₋₆alkyl, hydroxy-C₁₋₆alkyl and C₁₋₆alkoxy-C₁₋₆alkyl that are optionally substituted with one or groups selected from the group consisting of halogen, —OH, —CN, —NH₂ and methoxy;
Q is independently selected from the group consisting of C₁₋₆alkylene, C₁₋₆alkylidene and 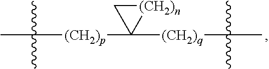
wherein said C₁₋₆alkylene and C₁₋₆alkylidene are optionally substituted with on or more groups selected from the group consisting of —OR, —R, hydroxy-C₁₋₆alkyl, NO₂, —CO₂H, —CO₂—R, —SO₂—R, halogen, —OH, —NH₂, —NHR, —C(═O)—NH₂, —CN, —C(═O)—NR₂ and —C(═O)—NHR;
X is selected from the group consisting of —OH, halogen and —OR;
n is 1, 2 or 3;
p, q and m are independently 0, 1, 2 or 3;
R is C₁₋₆alkyl; and
Z is a halogen or —OH.

37. A process for preparing a compound of Formula I 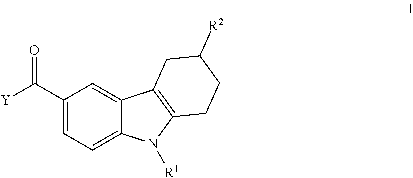
or comprising reacting a compound of formula III with R¹—X¹, 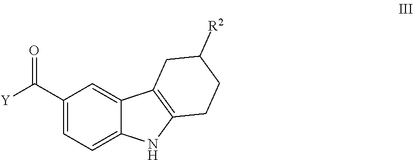
wherein,
X¹ is selected from halogen and OH;
Y is selected from the group consisting of 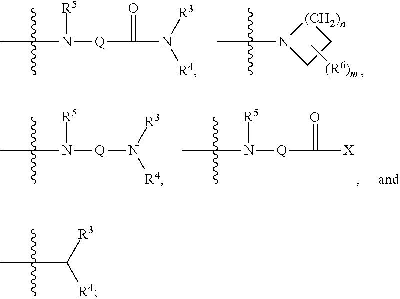
R¹ is selected from the group consisting of —H, C₁₋₆alkyl, C₂₋₆alkenyl, C₃₋₆cycloalkyl, C₃₋₆cycloalkyl-C₁₋₄alkyl, —C(═O)—NR¹⁴R¹⁵, —S(═O)₂—NR¹⁴R¹⁵, —S(═O)₂—C₁₋₆alkyl, —S(═O)₂—C₆₋₁₀aryl, —S(═O)₂—C₂₋₅heteroaryl, —C(═O)—C₁₋₆alkyl, —C(═O)—O—C₁₋₆alkyl, C₆₋₁₀aryl-C₁₋₄alkyl and C₂₋₅heteroaryl-C₁₋₄alkyl, wherein said C₁₋₆alkyl, C₂₋₆alkenyl, —S(═O)₂—C₁₋₆alkyl, —S(═O)₂—C₆₋₁₀aryl, —S(═O)₂—C₂₋₅heteroaryl, —C(═O)—C₁₋₆alkyl, C₆₋₁₀aryl-C₁₋₄alkyl and C₂₋₅heteroaryl-C₁₋₄alkyl used in defining R¹ are optionally substituted with one or more groups selected from the group consisting of —OR, R, —CO₂H, —CO₂—R, —SO₂—R, halogen, —NO₂, —OH, —NH₂, —NHR, —CN, —C(═O)—NH₂, —C(═O)—NR₂ and —C(═O)—NHR;
R² is selected from the group consisting of C₃₋₆heterocycloalkyl, C₃₋₆heterocycloalkyl-C₁₋₄alkyl, C₃₋₆cycloalkyl, C₃₋₆cycloalkyl-C₁₋₄alkyl, C₁₋₆alkyl, C₂₋₆alkenyl, C₆₋₁₀aryl, C₆₋₁₀aryl-C₁₋₄alkyl, C₂₋₆heteroaryl, C₂₋₆heteroaryl-C₁₋₄alkyl, —C(═O)—C₁₋₆alkyl, —C(═O)—C₃₋₆cycloalkyl and —C(═NH)—C₁₋₆alkyl, wherein said C₃₋₆heterocycloalkyl, C₃₋₆heterocycloalkyl-C₁₋₄alkyl, C₃₋₆cycloalkyl, C₃₋₆cycloalkyl-C₁₋₄alkyl, C₁₋₆alkyl, C₂₋₆alkenyl, C₆₋₁₀aryl, C₆₋₁₀aryl-C₁₋₄alkyl, C₂₋₆heteroaryl, C₂₋₆heteroaryl-C₁₋₄alkyl, —C(═O)—C₁₋₆alkyl, —C(═O)—C₃₋₆cycloalkyl and —C(═NH)—C₁₋₆alkyl used in defining R² are optionally substituted with one or more groups selected from the group consisting of —OR, R, NO₂, —CO₂H, —CO₂—R, —SO₂—R, halogen, —OH, —NH₂, —NHR, —CN, —C(═O)—NH₂ and —C(═O)—NHR;
R³ and R⁴ are independently selected from the group consisting of —H, C₃₋₆cycloalkyl, C₃₋₆heterocycloalkyl, C₂₋₅heteroaryl, C₆₋₁₀aryl, C₁₋₆alkyl, C₁₋₆alkoxy, amino, C₁₋₆alkylamino, diC₁₋₆alkylamino, —C(═O)—C₁₋₆alkyl, —C(═O)—O—C₁₋₆alkyl, —C(═O)—C₃₋₆cyclo alkyl, —C(═O)—NR¹⁴R¹⁵ and —S(═O)₂—NR¹⁴R¹⁵, wherein said C₃₋₆cycloalkyl, C₃₋₆heterocycloalkyl, C₂₋₅heteroaryl, C₆₋₁₀aryl, C₁₋₆alkyl, C₁₋₆alkoxy, C₁₋₆alkylamino, diC₁₋₆alkylamino, —C(═O)—C₁₋₆alkyl, —C(═O)—O—C₁₋₆alkyl, and —C(═O)—C₃₋₆cycloalkyl used in defining R³ and R⁴ are optionally substituted with one or more groups selected from the group consisting of —OR, R, NO₂, —CO₂H, —CO₂—R, —SO₂—R, halogen, —OH, —NH₂, —NHR, —C(═O)—NH₂, —CN, —C(═O)—NR₂ and —C(═O)—NHR;
R⁵ is selected from the group consisting of —H, C₁₋₆alkyl, and C₃₋₆cycloalkyl;
R⁶ is independently selected from the group consisting of —H, —CN, —NO₂, C₁₋₆alkoxy, halogen, C₁₋₆alkyl, —OH, —NH₂, —NHC(═O)R¹² and —C(═O)NR¹²R¹³;
R¹² and R¹³ are independently selected from the group consisting of —H, C₁₋₆alkyl, C₁₋₆alkoxy, C₃₋₆heterocycloalkyl, C₃₋₆cycloalkyl-C₁₋₄alkyl, and C₃₋₆cycloalkyl wherein said C₁₋₆alkyl, C₁₋₆alkoxy, C₃₋₆heterocycloalkyl, C₃₋₆cycloalkyl-C₁₋₄alkyl and C₃₋₆cycloalkyl used in defining R¹² and R¹³ are optionally substituted with one or more halogens or —OH;
R¹⁴ and R¹⁵ are independently selected from the group consisting of —H, C₁₋₆alkyl, C₆₋₁₀aryl, C₆₋₁₀aryl-C₁₋₄alkyl, C₂₋₅heterocyclyl, C₂₋₅heterocyclyl-C₁₋₄alkyl, C₂₋₆alkenyl, C₃₋₆cycloalkyl, and C₃₋₆cycloalkyl-C₁₋₄alkyl, N,N-di(C₁₋₄alkyl)amido-C₁₋₆alkyl, hydroxy-C₁₋₆alkyl and C₁₋₆alkoxy-C₁₋₆alkyl that are optionally substituted with one or groups selected from the group consisting of halogen, —OH, —CN, —NH₂ and methoxy;
Q is independently selected from the group consisting of C₁₋₆alkylene, C₁₋₆alkylidene and 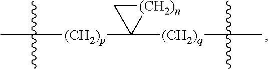
wherein said C₁₋₆alkylene and C₁₋₆alkylidene are optionally substituted with on or more groups selected from the group consisting of —OR, —R, hydroxy-C₁₋₆alkyl, NO₂, —CO₂H, —CO₂—R, —SO₂—R, halogen, —OH, —NH₂, —NHR, —C(═O)—NH₂, —CN, —C(═O)—NR₂ and —C(═O)—NHR;
X is selected from the group consisting of —OH, halogen or —OR;
n is 1, 2 or 3;
p, q and m are 0, 1, 2 or 3; and
R is C₁₋₆alkyl.
